(19) United States
(12) Patent Application Publication
(10) Pub. No.: US 2021/0115109 A1
(43) Pub. Date: Apr. 22, 2021

(54) CONJUGATES AND METHODS OF USE THEREOF FOR SELECTIVE DELIVERY OF IMMUNE-MODULATORY AGENTS

(71) Applicant: Silverback Therapeutics, Inc., Seattle, WA (US)

(72) Inventors: Peter Armstrong Thompson, Bellevue, WA (US); Robert Finley Dubose, Seattle, WA (US); Peter Robert Baum, Seattle, WA (US); Valerie Odegard, Seattle, WA (US)

(21) Appl. No.: 16/856,689

(22) Filed: Apr. 23, 2020

Related U.S. Application Data
(63) Continuation of application No. PCT/US2018/057175, filed on Oct. 23, 2018.
(60) Provisional application No. 62/576,545, filed on Oct. 24, 2017.

(51) Int. Cl. C07K 14/735; C07K 16/00

(52) U.S. Cl. CPC C07K 14/70535; C07K 2317/92; C07K 16/00

(57) ABSTRACT

Various compositions are disclosed. The compositions of conjugates comprising immune-modulatory agents are also provided. Additionally provided are the methods of preparation and use of the conjugates. This includes methods for treating disorders, such as cancer and fibrosis.

### CROSS-REFERENCE

This application is a continuation application of Serial No. PCT/US2018/057175, filed Oct. 23, 2018, which claims the benefit of U.S. Provisional Application No. 62/576,545, filed Oct. 24, 2017, each of which is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 860234_423C1_Sequence_Listing.txt. The text file is 634 KB, was created on Jan. 4, 2021 and is being submitted electronically via EFS-Web.

### BACKGROUND

One of the leading causes of death in the United States is cancer. Conventional methods of cancer treatment, like traditional chemotherapy, surgery, or radiation therapy, tend to be highly toxic, nonspecific to a cancer, or both, resulting in limited efficacy and harmful side effects. The immune system has the potential to be a powerful, specific tool in fighting cancers. In many cases tumors can specifically express genes whose products are required for inducing or maintaining a malignant state. These proteins may serve as antigen markers for the development and establishment of more specific anti-cancer immune response. The immune response may include the recruitment of immune cells that target tumors expressing these antigen markers. Additionally, the immune cells may express genes whose products are important to proper immune function and may serve as markers for specific types of immune cells. The boosting of this specific immune response has the potential to be a powerful anti-cancer treatment that can be more effective than conventional methods of cancer treatment and can have fewer side effects. Immune modulatory conjugates are one way to provide a specific immune response. Such conjugates can be targeted to certain cell populations by selectively binding to markers, such as proteins or other cellular markers, on tumor or other target cells. There remains, however, a need to further target immune modulatory conjugates to activate an immune response at the target cell population.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Provided herein are immune-modulatory conjugates and methods for providing benefit to patient populations for two areas of unmet needs, namely in the treatment of cancers such as solid tumors and in autoimmune/inflammatory disease, particularly for the latter in disease with distinct disease sites such as fibrotic disease. The immune-modulatory conjugates are designed to selectively interact with certain cell populations and/or to selectively release an immune modulatory payload at the desired target cells or in the extracellular microenviroment adjacent such target cells.

In exemplary embodiments, an immune-modulatory conjugate comprises (a) an antibody construct comprising an antigen binding domain and an Fc domain, wherein the antigen binding domain specifically binds to a first antigen expressed on target cells associated with a disease; (b) at least one immune-modulatory agent; and (c) at least one linker, wherein each linker is covalently attached to the antibody construct and to at least one immune-modulatory agent, wherein in the conjugate: (i) the linker is a cleavable linker having a protease cleavage site cleavable by a protease that is preferentially localized in the extracellular microenvironment of the target cells, whereby cleavage of the protease cleavage site releases an active form of the immune-modulatory agent in the extracellular microenvironment; or (ii) the Fc domain comprises an amino acid sequence having a Kd for a first Fc receptor that is at least 10 fold higher than a Kd of the amino acid sequence for a wild-type IgG1 Fc domain for the first Fc receptor and having a Kd for a second F receptor that is the same or lower than a Kd of the amino acid sequence for a wild-type IgG1 Fc domain for the second Fc receptor, thereby allowing the Fc domain to preferentially bind to cells expressing the second Fc receptor; or (iii) the linker is as set forth in (i) and the Fc domain is as set forth in (ii).

Also provided are immune-modulatory conjugates comprising: (a) an immune-modulatory agent; (b) an antibody construct comprising an antigen binding domain and an Fc domain, wherein the antigen binding domain binds to a first antigen expressed on cells associated with a target disease having an extracellular microenvironment associated with the disease; and (c) a linker, wherein the linker is covalently bound to the antibody construct and the linker is covalently bound to the immune-modulatory agent, the linker further comprising a protease cleavage site cleavable by a disease-associated protease that is preferentially present within the extracellular microenvironment of the disease cells; wherein the immune-modulatory agent is inactive when covalently bound to the antibody construct, and wherein an active form of the immune-modulatory agent is released upon cleavage of the protease cleavage site by the protease

In exemplary embodiments, the conjugate can be represented by the following formula:

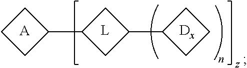

wherein: A is the antibody construct having the antigen binding domain and the Fc domain; L is the linker; Dₓ is the immune-modulatory agent; n is selected from 1 to 20; and z is selected from 1 to 20. In some exemplary embodiments, n is from 1 to 5 and z is from 1 to 8, n is 1 and z is from 1 to 8, or n is 1 and z is from 1 to 5. In some aspects, the immune-modulatory is as set forth in (iii).

In some exemplary embodiments, the linker, L, has the following formula: Rx-Aₙ-protease cleavage site-Y_{y}—, wherein Rx is a reactive moiety attached to the antibody construct; A is a stretcher group and n is 0 or 1; and Y is a self immolative group and y is 0, 1, or 2. In some aspects, Rx is a succinimide group or a hydrolyzed succinmide group and/or Y is a self-immolative group and is present. The self-immolative group can be, for example, a PABA or PABC group. The linker can be, for example, -maleimidocaproyl-protease cleave site-PABC-.

In some exemplary embodiments wherein the linker is a cleavable linker having a protease cleavage site cleavable by a protease, the protease cleavage site is selected from a site cleavable by a protease selected from the group consisting of legumain, plasmin, TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA. In other embodiments, the protease cleavage site is selected from a site cleavable by a protease selected from one of the groups consisting of: Legumain and plasmin; TMPRSS3, TMPRSS4, and TMPRSS6; MMP1, MMP2, MMP-3, MMP-9, MMP-8, MMP-14, and MT1-MMP; CATHEPSIN D, CATHEPSIN K, and CATHEPSIN S; ADAM10, ADAM12 and ADAMTS; Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and Caspase-14; and TACE, human neutrophil elastase, beta-secretase, uPA, fibroblast associated protein, matriptase, PSMA and PSA. In some aspects, the protease cleavage site is selected from a site cleavable by a protease selected from TMPRSS3, TMPRSS4, and TMPRSS6. In other aspects, the protease cleavage site is selected from a site cleavable by a protease selected from MMP1, MMP2, MMP-3, MMP-9, MMP-8, MMP-14, and MT1-MMP.

In some exemplary embodiments, the protease is preferentially localized in the extracellular microenvironment of the target cells as compared to the extracellular microenvironment of normal cells by a factor of at least 5:1, 10:1, 25:1, 50:1 or 100:1. In some exemplary embodiments, the active form of the immune-modulatory agent is preferentially released in the extracellular microenvironment as compared to the extracellular microenvironment of normal cells by a factor of at least 5:1, 10:1, 25:1, 50:1 or 100:1.

In some exemplary embodiments, the linker of the immune-modulatory conjugate is not a cleavable linker. The non-cleavable linker can be, for example, represented by the formula:

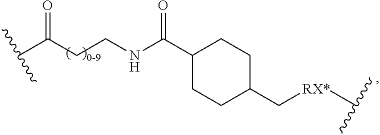

wherein RX* is a bond, a succinimide moiety, or a hydrolyzed succinimide moiety, wherein

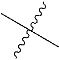

on RX* represents the point of attachment to the residue of the antibody construct.

In some exemplary embodiments, the immune-modulatory conjugates comprise a Fc domain comprising an amino acid sequence having a Kd for a first Fc receptor. The first Fc receptor can be, for example, an FcγRI, FcγRIIA, FcγRIIB, FcγRIIIA, or FcγRIIIB receptor and the second Fc receptor can be, for example, an FcRn receptor. In some embodiments, the first Fc receptor is an FcγRI, FcγRIIA and FcγRIIIA and the second Fc receptor is an FcRn receptor. In some aspects, the Kd of the Fc domain for the FcRn is at least 5 fold lower than the binding of a wild-type IgG1 to FcRn. In some aspects, the cells expressing the second Fc receptor are dendritic cells. In some aspects, the Fc domain is an Fcnun. In some aspects, the Fc domain amino acid sequence has a Kd for binding to FcRn that is at least 10 fold higher than a Kd of the amino acid sequence for a wild-type IgG1 Fc domain for FcRn receptors. In some aspects, the Fc domain comprises an amino acid sequence having at least one amino acid residue change selected from a group consisting of: a) F243L, R292P, Y300L, L235V, and P396L, wherein numbering of amino acid residues in the Fc domain is according to the EU index; b) S239D and 1332E, wherein numbering of amino acid residues in the Fc domain is according to the EU index; c) S298A, E333A, and K334A, wherein numbering of amino acid residues in the Fc domain is according to the EU index; and d) H435A, wherein numbering of amino acid residues in the Fc domain is according to the EU index.

Exemplary immune-modulatory conjugates comprise an antibody construct that comprises an antigen binding domain that binds to a first antigen. The first antigen can be selected, for example, from the group consisting of GD2, GD3, GM2, Le^{y}, sLe, polysialic acid, fucosyl GM1, Tn, STn, BM3, GloboH, CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), STN1, TNC, CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), and VISTA. In some aspects, the first antigen is selected from the group consisting of EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1, and STRA6. In some aspects, the first antigen is LRRC15, FAP or MUC16. In some aspects, the first antigen is selected from the group consisting of Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ31, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, MMP14, GPX8, and F2RL2. The first antigen is typically expressed on cells associated with a disease. In some aspects, the disease is a cancer. The cancer can be, for example, selected from the group consisting of metastatic pancreatic adenocarcinoma, metastatic colorectal adenocarcinoma, breast invasive carcinoma, squamous cell lung cancer and metastatic head and neck squamous cell carcinoma. In some aspects, the disease is a fibrotic or inflammatory disease of the liver, kidney or lung, systemic scleroderma, idiopathic pulmonary fibrosis (IPF), NASH, cardiomyopathy, renal fibrosis, liver fibrosis, lung fibrosis or systemic scleroderma. The first antigen can be, for example, a ligand bound to a receptor on cells in the microenvironment of the disease

The immune-modulatory conjugates comprise at least one immune-modulatory agent. The immune-modulatory agent can be, for example, a toll-like receptor agonist, STING agonist, RIG-I agonist, PAMP agonist, DAMP agonist or a kinase inhibitor. The toll-like receptor agonist can be, for example, selected from a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, or a TLR10 agonist. The TLR7 agonist can be, for example, selected from the group consisting of an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, and a benzonaphthyridine. The TLR8 agonist can be selected, for example, from the group consisting of a benzazepine, an imidazoquinoline, a thiazoloquinoline, an aminoquinoline, an aminoquinazoline, a pyrido [3,2-d]pyrimidine-2,4-diamine, a pyrimidine-2,4-diamine, a 2-aminoimidazole, a 1-alkyl-1H-benzimidazol-2-amine, and a tetrahydropyridopyrimidine. In some aspects, the immune-modulatory agent is an inhibitor of a GCPR, an ion channel, a membrane transporter, a phosphatase or an ER protein. In some aspects, the immune-modulatory agent is an antagonist of the GPCR A2aR, the sphingosine 1-phosphate receptor 1, prostaglandin receptor EP3, prostanglandin receptor E2, Frizzled, CXCR4 or an LPA receptor. The ion channel agonist can be, for example, an ion channel agonist for CRAC, Kv1.3 or KCa3.1. In some aspects, the immune-modulatory agent is an inhibitor of HSP90 or AAA-ATPase p97; an inhibitor of TGFβ, the TGFβ signaling pathway, or the β-Catenin pathway; or an inhibitor of TNIK, Tankyrase, PI3K-beta, STAT3, IL-10, IDO, or TDO.

The immune-modulatory conjugates typically comprise an antigen binding domain. In some aspects, the antigen binding domain comprises at least 80% sequence identity to a set of variable region CDR sequences set forth in TABLE 1, wherein the assignment of CDR residues are defined according to the IMGT (the international ImMunoGeneTics information system). In other aspects, the antigen binding domain comprises a variable region comprising V_{H} and V_{L} sequences at least 80% sequence identity to a pair of V_{H} and V_{L} sequences set forth in TABLE 2. In even other aspects, the antigen binding domain comprises a variable region having V_{H} and V_{L} sequences having at least 80% sequence identity to a V_{H} or V_{L} sequence set forth in TABLE 6.

Also provided herein are pharmaceutical compositions comprising the immune-modulatory conjugates described herein and a pharmaceutically acceptable carrier as well as methods for the treatment of a subject in need thereof, comprising administering a therapeutic dose of a conjugate described herein or a pharmaceutical composition described herein. The conjugate can be, for example, administered intravenously, cutaneously, subcutaneously, or injected at a site of affliction. Immune-modulatory conjugates and pharmaceutical compositions described herein can be used as a medicament and for use in the treatment of disease, including cancer, fibrotic or inflammatory disease of the liver, kidney or lung, systemic scleroderma, idiopathic pulmonary fibrosis (IPF), NASH, cardiomyopathy, renal fibrosis, liver fibrosis, lung fibrosis, or systemic scleroderma.

### BRIEF DESCRIPTION OF THE DRAWINGS

FIG. 1A-FIG. 1B: show stimulation of TNFα production by monocytes (FIG. 1A) and macrophages (FIG. 1B) in response to a wild-type (filled circles), FcγR null (filled squares), FcγR/FcRN Null (open squares) and FcRN null (open circles) Her2 TLR8 benzazepine agonist.

FIG. 2: shows stimulation of TNFα production in the presence of wild-type Her2 TLR8 benzazepine agonist control (filled circles) and in the presence of Her2 TLR8 benzazepine agonist with anti CD16 antibody (filled triangles), anti-CD32 antibody (bolded open squares), anti-CD64 antibody (half-filled squares), and a combination of anti-CD16, 32, and 64 antibodies (filled squares).

FIG. 3: shows stimulation of IL12 production in the presence of wild-type Her2 TLR8 benzazepine agonist control (open squares) and in the presence of Her2 TLR8 benzazepine agonist with anti CD16 antibody (filled triangles), anti-CD32 antibody (half-side filled squares), anti-CD64 antibody (half-top filled squares), and a combination of anti-CD16, 32, and 64 antibodies (filled squares).

### DETAILED DESCRIPTION

Additional aspects and advantages of the present disclosure will become apparent to those skilled in this art from the following detailed description, wherein illustrative aspects of the present disclosure are shown and described. As will be realized, the present disclosure is capable of other and different aspects, and its several details are capable of modifications in various respects, all without departing from the disclosure. Accordingly, the drawings and description are to be regarded as illustrative in nature, and not as restrictive.

As used herein, “identical” or “percent (%) identity” refer to the identity between a DNA, RNA, nucleotide, amino acid, peptide, polypeptide or protein sequence to another DNA, RNA, nucleotide, amino acid, peptide, polypeptide or protein sequence. Identity is expressed in terms of a percentage of sequence identity of a first sequence to a second sequence. Percent (%) sequence identity with respect to a reference DNA sequence is the percentage of DNA nucleotides in a candidate sequence that are identical with the DNA nucleotides in the reference DNA sequence after aligning the sequences. Percent (%) sequence identity with respect to a reference amino acid sequence is the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, or humanized or a heteroconjugate, bispecific, diabody, triabody, or tetrabody. The antigen binding fragment can include, for example, Fab′, F(ab′)2, Fab, Fv, rIgG, scFv, hcAbs (heavy chain antibodies), a single domain antibody, VHH, VNAR, sdAbs, or nanobody.

As used herein, “recognize”, “specifically binds” and “specifically bind” refer to the specific association or binding between an antigen binding domain and an antigen, as compared to a non-specific association between an antigen binding domain and a non-antigen. In some embodiments, an antigen binding domain that recognizes or specifically binds to an antigen has a dissociation constant (KD) of <<100 nM, <10 nM, <1 nM, <0.1 nM, <0.01 nM, or <0.001 nM (e.g. 10⁻⁸M or less, e.g. from 10⁻⁸M to 10⁻¹³M, e.g., from 10⁻⁹M to 10⁻¹³M).

As used herein, a “tumor antigen” refers to an antigenic substance associated with a tumor or cancer cell, and can trigger an immune response in a host.

As used herein, an “antibody construct” refers to a construct that contains at least one antigen binding domain and an Fc domain.

As used herein, an “antigen binding domain” refers to a binding domain from an antibody or from a non-antibody that can specifically bind to the antigen. Antigen binding domains can be numbered when there is more than one antigen binding domain in a given conjugate or antibody construct (e.g., first antigen binding domain, second antigen binding domain, third antigen binding domain, etc.). Different antigen binding domains in the same conjugate or construct can target the same antigen or different antigens (e.g., first antigen binding domain that can specifically bind to a tumor antigen, a second antigen binding domain that can specifically bind to a tumor antigen and a third antigen binding domain that can specifically bind to an APC antigen; or a first antigen binding domain that can specifically bind a tumor antigen, a second antigen binding domain that can specifically bind to an antigen on an antigen presenting cell (APC antigen), and a third antigen binding domain that can specifically bind to an APC antigen).

As used herein, a “Fc domain” refers to an Fc domain from an antibody or from a non-antibody that can bind to an Fc receptor.

As used herein, a “target binding domain” refers to a construct that contains an antigen binding domain from an antibody or from a non-antibody that can bind to the antigen.

As used herein, a “disease” refers to a disorder of structure or function in a subject (e.g., a human or animal), especially one that produces specific signs or symptoms or that affects a specific location and is not simply a direct result of physical injury.

As used herein, a “microenvironment” refers to the extracellular environment in which cells of a disease are located and that have an extracellular space(s) in which proteases and other soluble proteins and factors are located. A microenvironment can contain, for example, blood vessels, immune cells, fibroblasts, bone marrow-derived inflammatory cells, lymphocytes, signaling molecules and the extracellular matrix (ECM).

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

The terms “salt” or “pharmaceutically acceptable salt” refer to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “C_{x-y}alkenyl” and “C_{X-y}alkynyl” refer to substituted or unsubstituted unsaturated aliphatic groups analogous in length and possible substitution to the alkyls described above, but that contain at least one double or triple bond respectively.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings wherein at least one of the rings includes a heteroatom. In an exemplary embodiment, an aromatic ring, e.g., pyridyl, may be fused to a saturated or unsaturated ring, e.g., cyclohexane, cyclopentane, morpholine, piperidine or cyclohexene. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also include polycyclic ring systems having two or more cyclic rings in which two or more carbons are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO2), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

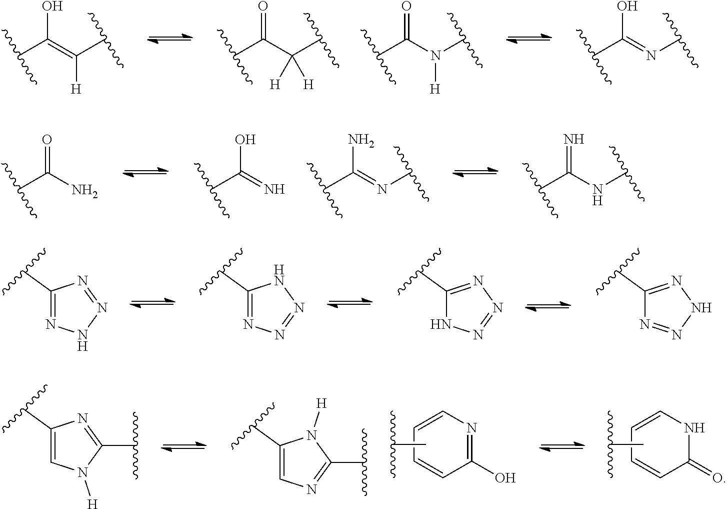

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### Antigens

An antigen can be a protein, polysaccharide, lipid, or glycolipid, which can be recognized by an antibody or other antigen binding domain or by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

In cancer, there are four general groups of tumor antigens: (i) viral tumor antigens which can be identical for any viral tumor of this type, (ii) carcinogenic tumor antigens which can be specific for patients and for the tumors, (iii) isoantigens of the transplantation type or tumor-specific transplantation antigens which can be different in all individual types of tumor but can be the same in different tumors; and (iv) embryonic antigens.

As a result of the discovery of tumor antigens, tumor antigens have become important in the development of cancer treatments that can specifically target the cancer. This has led to the development of antibodies and other antigen binding constructs directed against these tumor antigens.

In addition to the development of antibodies against tumor antigens for cancer treatment, antibodies that target immune cells to boost or otherwise modulate an immune response have also been developed. For example, an anti-CD40 antibody that is a CD40 agonist can be used to activate dendritic cells to enhance the immune response.

Cluster of Differentiation 40 (CD40) is a member of the Tumor Necrosis Factor Receptor (TNF-R) family. CD40 can be a 50 kDa cell surface glycoprotein that can be constitutively expressed in normal cells, such as monocytes, macrophages, B lymphocytes, dendritic cells, endothelial cells, smooth muscle cells, fibroblasts and epithelium, and in tumor cells, including B-cell lymphomas and many types of solid tumors. Expression of CD40 can be increased in antigen presenting cells in response to IL-1βp, IFN-γ, GM-CSF, and LPS induced signaling events.

Humoral and cellular immune responses can be regulated, in part, by CD40. For example, in the absence of CD40 activation by its cognate binding partner, CD40 Ligand (CD40L), antigen presentation can result in tolerance. However, CD40 activation can ameliorate tolerance. In addition, CD40 activation can positively impact immune responses by enhancing antigen presentation by antigen presenting cells (APCS), increasing cytokine and chemokine secretion, stimulating expression of and signaling by co-stimulatory molecules, and activating the cytolytic activity of different types of immune cells. Accordingly, the interaction between CD40 and CD40L can be essential to maintain proper humoral and cellular immune responses.

The intracellular effects of CD40 and CD40L interaction can include association of the CD40 cytoplasmic domain with TRAFs (TNF-R associated factors), which can lead to the activation of NFκB and Jun/AP1 pathways. While the response to activation of NFκB and Jun/AP1 pathways can be cell type-specific, often such activation can lead to increased production and secretion of cytokines, including IL-6, IL-8, IL-12, IL-15; increased production and secretion of chemokines, including MIP1α and β and RANTES; and increased expression of cellular adhesion molecules, including ICAM. While the effects of cytokines, chemokines and cellular adhesion molecules can be widespread, such effects can include enhanced survival and activation of T cells.

In addition to the enhanced immune responses induced by CD40 activation, CD40 activation can also be involved in chemokine- and cytokine-mediated cellular migration and differentiation; activation of immune cells, including monocytes; activation of and increased cytolytic activity of immune cells, including cytolytic T lymphocytes and natural killer cells; induction of CD40-positive tumor cell apoptosis and enhanced immunogenicity of CD40-positive tumors. In addition, CD40 can initiate and enhance immune responses by many different mechanisms, including, inducing antigen-presenting cell maturation and increased expression of costimulatory molecules, increasing production of and secretion of cytokines, and enhancing effector functions.

CD40 activation can be effective for inducing immune-mediated antitumor responses. For example, CD40 activation reverses host immune tolerance to tumor-specific antigens which leads to enhanced antitumor responses by T cells. Such antitumor activity can also occur in the absence of immune cells. Similarly, antitumor effects can occur in response to anti-CD40 antibody-mediated activation of CD40 and can be independent of or can involve antibody-dependent cellular cytotoxicity (ADCC). In addition to other CD40-mediated mechanisms of antitumor effects, CD40L-stimulation can cause dendritic cell maturation and stimulation. CD40L-stimulated dendritic cells can contribute to the antitumor response. Furthermore, vaccination strategies including CD40 can result in regression of CD40-positive and CD40-negative tumors.

CD40 activating antibodies (e.g., anti-CD40 activating monoclonal antibodies) can be useful for treatment of tumors. This can occur through one or more mechanisms, including cell activation, antigen presentation, production of cytokines and chemokines, amongst others. For example, CD40 antibodies activate dendritic cells, leading to processing and presentation of tumor antigens as well as enhanced immunogenicity of CD40-positive tumor cells. Not only can enhanced immunogenicity result in activation of CD40-positive tumor specific CD4⁺ and CD8⁺ T cells, but further antitumor activity can include, recruitment and activation monocytes, enhanced cytolytic activity of cytotoxic lymphocytes and natural killer cells as well as induction of apoptosis or by stimulation of a humoral response so as to directly target tumor cells. In addition, tumor cell debris, including tumor-specific antigens, can be presented to other cells of the immune system by CD40-activated antigen presenting cells.

Since CD40 can be important in an immune response, there is a need for enhanced CD40 meditated signaling events to provide reliable and rapid treatment options to patients suffering from diseases which may be ameliorated by treatment with CD40-targeted therapeutic strategies.

The CD40 mediated immune response can be further enhanced by targeting CD40 activation to the localized tumor site(s) through pairing with a tumor antigen binding domain. Such targeted CD40 activation and recruitment of immune cells to tumor cells may provide the advantage of maintaining therapeutic effectiveness with a lower dosage of a CD40 activating antibody construct or conjugate. A lower dosage may help mitigate any side effects of systemic CD40 activation such as cytokine release syndrome, which has been observed in some subjects treated with the agonistic CD40 monoclonal antibodies such as CP-870,893, dacetuzumab, Chi Lob 7/4, SEA-CD40, ADC-1013, 3C3, or 3G5. Systemic CD40 activation may also pose a risk of autoimmunity by causing APCs to break tolerance of autoantigens. For example, autoreactive T cells that manage to evade thymic selection may persist in the periphery in a state of tolerance against autoantigens, but CD40 activation can cause them to break tolerance and exhibit an autoimmune response. Accordingly, there is an important need for therapeutic, clinically relevant targeted CD40 activation that is enhanced at the localized tumor site relative to systemic activation. The presently described conjugates can be utilized to enhance the immune response. A conjugate can comprise an antigen binding domain, or a pair of antigen binding domains, and a CD40 binding domain, wherein the antigen binding domain(s) specifically binds a tumor antigen and the CD40 binding domain comprises a CD40 agonist. This combination of a tumor antigen binding domain(s) and a CD40 agonist can provide enhanced CD40 activation and recruitment of immune cells to the localized tumor site.

In addition to targeting immune cells to boost the immune response using anti-CD40 antibodies, antibodies that target other antigens expressed by immune cells can be used. For example, an anti-DEC205 antibody, an anti-CD36 mannose scavenger receptor 1 antibody, an anti-CLEC9A antibody, CLEC12A, an anti-DC-SIGN antibody, an anti-BDCA-2 antibody, an anti-OX40L antibody, an anti-41BBL antibody, an anti-CD204 antibody, an anti-MARCO antibody, an anti-CLEC5A antibody, an anti-Dectin 1 antibody, an anti-Dectin 2 antibody, an anti-CLEC10A antibody, an anti-CD206 antibody, an anti-CD64 antibody, an anti-CD32A antibody, an anti-CD16A antibody, an anti-HVEM antibody, an anti-PD-L1 antibody, an anti-CD32B antibody or an anti-CD47 antibody can be used to target, respectively, surface DEC-205, CD36 mannose scavenger receptor 1, CLEC9A, CLEC12A, DC-SIGN, BDCA-2, OX40L, 41BBL, CD204, MARCO, CLEC5A, Dectin 1, Dectin 2, CLEC10A, CD206, CD64, CD32A, CD16A, HVEM, PD-L1, or CD32B molecules expressed by antigen presenting cells or CD47 molecules expressed by T cells.

Cluster of Differentiation 205 (CD205 or DEC-205) is a member of the C-type multilectin family of endocytic receptors, which can include the macrophage mannose receptor (MMR) and the phospholipase A2 receptor (PLA₂R). DEC-205 can be a 205 kDa endocytic receptor highly expressed in cortical thymic epithelial cells, thymic medullary dendritic cells (CD11c⁺ CD8⁺), subpopulations of peripheral dendritic cells (CD11c⁺ CD8⁺). The DEC-205⁺ CD11c⁺ CD8⁺ dendritic cells (DCs) can function in cross-presentation of antigens derived from apoptotic cells. Additionally, DEC-205 can be significantly upregulated during DC maturation. DEC-205 can also be expressed at moderate levels in B cells and low levels in macrophages and T cells.

After antigen binding to DEC-205, the receptor-antigen complex can be internalized whereupon the antigen can be processed and be presented on the DC surface by a major histocompatibility complex class II (MHC II) or MHC class I. DEC-205 can deliver antigen to DCs for antigen presentation on MHC class II and cross-presentation on MHC class I. DEC-205 mediated antigen delivery for antigen presentation in DCs without an inflammatory stimulus can result in tolerance. Conversely, DEC-205 mediated antigen delivery in DCs in the presence of a maturational stimulus (e.g. a CD40 agonist) can result in long-term immunity via activation of antigen-specific CD4⁺ and CD8⁺ T cells.

CD36 mannose scavenger receptor 1 is an oxidized LDL receptor with two transmembrane domains located in the caveolae of the plasma membrane. It can be classified as a Class B scavenger receptor, which can be characterized by involvement in the removal of foreign substances and waste materials. This receptor can also be involved in cell adhesion, phagocytosis of apoptotic cells, and metabolism of long-chain fatty acids.

CLEC9A is a group V C-type lectin receptor. This receptor can be expressed as on myeloid lineage cells, and can be characterized as an activation receptor.

CLEC12A is a member of the C-type lectin/C-type lectin like domain super family that can be a negative regulator of granulocyte and monocyte function. It can also be involved in cell adhesion, cell-cell signaling, and glycoprotein turnover, and can play a role in the inflammatory response.

Dendritic cell-specific inter cellular adhesion molecule-3-grabbing non-integrin (DC-SIGN) or CD209, is a C-type lectin receptor that can be expressed on the surface of macrophages and dendritic cells. This receptor can recognize and bind to mannose type carbohydrates and be involved in activating phagocytosis, can mediate dendritic cell rolling, and can be involved in CD4+ T cell activation.

BDCA-2 is a C-type lectin that is a membrane protein of plasmacytoid dendritic cells. It can be involved in plasmacytoid dendritic cell function, such as ligand internalization and presentation.

OX40L, which can also be referred to as CD252, is the ligand for CD134 that can be expressed on dendritic cells. It can be involved in T cell activation.

41BBL, which can also be referred to as CD137L, is a member of the TNF superfamily, and can be expressed on B cells, dendritic cells, activated T cells, and macrophages. It can provide co-stimulatory signal for T cell activation and expansion.

CD204, which can also be referred to as macrophage scavenger receptor 1, is a macrophage scavenger receptor receptor. The gene for CD204 can encode three different class A macrophage scavenger receptor isoforms. The type 1 and type 2 isoforms can be involved in binding, internalizing, and processing negatively charged macromolecules, such as low density lipoproteins. The type 3 isoform can undergo altered intracellular processing in which it can be retained within the endoplasmic reticulum, and has been shown to have a dominant negative effect on the type 1 and type 2 isoforms.

Macrophage receptor with collagenous structure (MARCO), which can also be referred to as SCARA2, is a class A scavenger receptor with collagen-like and cysteine-rich domains. It can be expressed in macrophages, and can bind to modified low density lipoproteins. It can be involved in the removal of foreign substances and waste materials.

C-type lectin domain family 5 member A (CLEC5A) is a C-type lectin. It can be involved in the myeloid lineage activating pathway.

Dendritic cell-associated c-type lectin-1 (Dectin 1), which can also be referred to as CLEC7A, is member of the C-type lectin/C-type lectin-like super family. It can be expressed by myeloid dendritic cells, monocytes, macrophages, and B cells, and can be involved in antifungal immunity.

Dendritic cell-associated c-type lectin-2 (Dectin 2), which can also be referred to as CLEC6A, is member of the C-type lectin/C-type lectin-like super family. It can be expressed by dendritic cells, macrophages, monocytes and neutrophils. It can be involved in antifungal immunity.

CLEC10A, which can also be referred to as CD301, is member of the C-type lectin/C-type lectin-like super family. It can be expressed by dendritic cells, monocytes, and CD33+ myeloid cells, and can be involved in macrophage adhesion and migration.

CD206, which can also be referred to as macrophage mannose receptor, is a C-type lectin type I membrane glycoprotein. It can be expressed on dendritic cells, macrophages and endothelial cells, and can act as a pattern recognition receptor and bind high-mannose structures of viruses, bacteria, and fungi.

CD64, which can also be referred to as FcγRI, is a high affinity Fc receptor for IgG. It can be expressed by monocytes and macrophages. It can be involved in mediating phagocytosis, antigen capture, and antibody dependent cell-mediated cytoxicity.

CD32A, which can also be referred to as FcγRIIa, is a low affinity Fc receptor. It can be expressed on monocytes, granulocytes, B cells, and eosinophils. It can be involved in phagocytosis, antigen capture, and antibody dependent cell-mediated cytoxicity.

CD16A, which can also be referred to as FcγRIIIa, is low affinity Fc receptor. It can be expressed on NK cells, and can be involved in phagocytosis and antibody dependent cell-mediated cytotoxicity.

Herpesvirus entry mediator (HVEM), which can also be referred to as CD270, is a member of the TNF-receptor superfamily. It can be expressed on B cells, dendritic cells, T cells, NK cells, CD33+ myeloid cells, and monocytes. It can be involved in activating the immune response.

CD32B, which can also be referred to as FcγRIIb, is a low affinity Fc receptor. It can be expressed on B cells and myeloid dendritic cells. It can be involved in inhibiting maturation and cell activation of dendritic cells.

The HER2/neu (human epidermal growth factor receptor 2/receptor tyrosine-protein kinase erbB-2) is part of the human epidermal growth factor family. Overexpression of this protein can be shown to play an important role in the progression of cancer, for example, breast cancer. The HER2/neu protein can function as a receptor tyrosine kinase and autophosphorylates upon dimerization with binding partners. HER2/neu can activate several signaling pathways including, for example, mitogen-activated protein kinase, phosphoinositide 3-kinase, phospholipase Cy, protein kinase C, and signal transducer and activator of transcription (STAT). Examples of antibodies that can target and inhibit HER2/neu can include trastuzumab and pertuzumab.

EGFR (epidermal growth factor receptor) encodes a member of the human epidermal growth factor family. Mutations that can lead to EGFR overexpression or over activity can be associated with a number of cancers, including squamous cell carcinoma and glioblastomas. EGFR can function as a receptor tyrosine kinase and ligand binding can trigger dimerization with binding partners and autophosphorylation. The phosphorylated EGFR can then activate several downstream signaling pathways including mitogen-activated protein kinase, phosphoinositide 3-kinase, phospholipase Cy, protein kinase C, and signal transducer and activator of transcription (STAT). Examples of antibodies that can target and inhibit EGFR can include cetuximab, panutumumab, nimotuzumab, and zalutumumab.

One mutant variant of EGFR is EGFRvIII (epidermal growth factor receptor variant III). EGFRvIII can be the result of an EGFR gene rearrangement in which exons 2-7 of the extracellular domain are deleted. This mutation can result in a mutant receptor incapable of binding to any known ligand. The resulting receptor can engage in a constitutive low-level signaling and can be implicated in tumor progression. Examples of antibodies that can target EGFRvIII can include AMG595 and ABT806.

C-Met (hepatocyte growth factor receptor) encodes a member of the receptor tyrosine kinase family of proteins. C-Met overexpression and over activity can be implicated in various cancers including lung adenocarcinomas, and high c-Met levels can be associated with poor patient outcome. Binding of hepatocyte growth factor can induce dimerization and autophosphorylation of c-Met. The c-Met receptor can activate various downstream signaling pathways including mitogen-activated protein kinase, phosphoinositide 3-kinase, and protein kinase C pathways. The antibody onartuzumab can target and inhibit c-Met.

HER3 (human epidermal growth factor receptor 3) encodes a member of the human epidermal growth factor receptor family. Ligand binding can induce dimerization and autophosphorylation of cytoplasmic tyrosine residues that then can recruit signaling proteins for downstream signaling pathway activation including mitogen-activated protein kinase and phosphoinoside 3-kinase pathways. HER3 can play an active role in cell proliferation and survival, and can be overexpressed, overactive, and/or mutated in various cancers. For example, HER3 can be overexpressed in breast, ovarian, prostate, colon, pancreas, stomach, oral, and lung cancers. The antibody patritumab can target and inhibit HER3.

MUC1 (mucin 1, cell surface associated) encodes a member of the mucin family of glycosylated proteins that can play an important role in cell adhesion and forming protective mucosal layers on epithelial surfaces. MUC1 can be proteolytically cleaved into alpha and beta subunits that form a heterodimeric complex with the N-terminal alpha subunit providing cell-adhesion functionality and the C-terminal beta subunit modulating cell signaling pathways including the mitogen activated map kinase pathway. MUC1 can play a role in cancer progression, for example, by regulating TP53-mediated transcription. MUC1 overexpression, aberrant intracellular localization, and glycosylation changes can all be associated with carcinomas including pancreatic cancer cells. The antibody clivatuzumab can target MUC1.

MUC16 (mucin 16, cell surface associated) encodes the largest member of the mucin family of glycosylated proteins that can play an important role in cell adhesion and forming protective mucosal layers on epithelial surfaces. MUC16 can be a highly glycosylated 2.5MDa transmembrane protein that can provide a hydrophilic lubricating barrier on epithelial cells. The cytoplasmic tail of MUC16 can be involved with various signaling pathways including the JAK2-STAT3 and Src kinase pathways. A peptide epitope of MUC16 can be used as biomarker for detecting ovarian cancer. Elevated expression of MUC16 can be present in advanced ovarian cancers and pancreatic cancers. The antibody sofituzumab can target MUC16.

EPCAM (epithelial cell adhesion molecule) encodes a transmembrane glycoprotein that can be frequently and highly expressed in carcinomas and tumor-initiating cells. EPCAM can also be a pluripotent stem cell marker. EPCAM can modulate a variety of pathways including cell-cell adhesion, cellular proliferation, migration, invasion, maintenance of a pluripotent state, and differentiation in the context of tumor cells. The antibodies edrecolomab and adecatumumab can target EPCAM.

MSLN (mesothelin) encodes a 40 kDa cell GPI-anchored membrane surface protein believed to function in cell adhesion. MSLN is overexpressed in mesothelioma and certain types of pancreatic, lung, and ovarian cancers. MSLN-related peptides that circulate in serum of patients suffering from pleural mesothelioma are used as biomarkers for monitoring the disease. MSLN may promote metastasis by inducing matrix metalloproteinase 7 and 9 expression. The monoclonal antibody anetumab has been developed to target MSLN.

CA6 (carbonic anhydrase VI) encodes one of several isozymes of carbonic anhydrase. CA6 is found in salivary glands and may play a role in the reversible hydration of carbon dioxide. CA6 is expressed in human serous ovarian adenocarcinomas. The monoclonal antibody huDS6 has been developed to target CA6.

NAPI2B (sodium/phosphate cotransporter 2B) encodes a type II sodium-phosphate cotransporter. NAPI2B is highly expressed on the tumor surface in lung, ovarian, and thyroid cancers as well as in normal lung pneumocytes. The monoclonal antibody lifastuzumab has been developed to target NAPI2B.

TROP2 (trophoblast antigen 2) encodes a transmembrane glycoprotein that acts as an intracellular calcium signal transducer. TROP2 binds to multiple factors such as IGF-1, claudin-1, claudin-7, cyclin D1, and PKC. TROP2 including intracellular calcium signaling and the mitogen activated protein kinase pathway. TROP2 plays a role in cell self-renewal, proliferation, invasion, and survival. Discovered first in trophoblast cells that have the ability to invade uterine decidua during placental implantation, TROP2 overexpression has been shown to be capable of stimulating cancer growth. TROP2 overexpression has been observed in breast, cervix, colorectal, esophagus, lung, non-Hodgkin's lymphoma, chronic lymphocytic lymphoma, Raji Burkitt lymphoma, oral squamous cell, ovarian, pancreatic, prostate, stomach, thyroid, urinary bladder, and uterine carcinomas. The monoclonal antibody sactuzumab has been developed to target TROP2.

CEA (carcinoembryonic antigen) encodes a family of related glycoproteins involved in cell adhesion. CEA is a biomarker for gastrointestinal cancers and may promote tumor development by means of its cell adhesion function. CEA levels have been found to be elevated in serum of individuals with colorectal carcinoma. CEA levels have also been found to be elevated in gastric carcinoma, pancreatic carcinoma, lung carcinoma, breast carcinoma, and medullary thyroid carcinoma. The monoclonal antibodies PR1A3 and Ab2-3 have been developed to target CEA.

CLDN18.2 (claudin 18) encodes a member of the claudin family of integral membrane proteins. CLDN18.2 is a component of tight junctions that create a physical barrier to prevent diffusion of solutes and water through the paracellular space between epithelial cells. CLDN18.2 is overexpressed in infiltrating ductal adenocarcinomas, but is reduced in some gastric carcinomas. The monoclonal antibody claudiximab has been developed to target CLDN18.2.

FAP (fibroblast activation protein, alpha) encodes a homodimeric integral membrane protein from a family of serine proteases. FAP is believed to play a role in many processes including tissue remodeling, fibrosis, wound healing, inflammation, and tumor growth. FAP enhances tumor growth and invasion by promoting angiogenesis, collagen fiber degradation and apoptosis, and by downregulating the immune response. FAP is selectively expressed on fibroblasts within the tumor stroma. The monoclonal antibody sibrotuzumab has been developed to target FAP.

EphA2 (EPH Receptor A2) encodes a member of the ephrin receptor subfamily of the protein-tyrosine kinase family. EphA2 binds to ephrin-A ligands. Activation of EphA2 receptor upon ligand binding can result in modulation of migration, integrin-mediated adhesion, proliferation, and differentiation. EphA2 is overexpressed in various cancers including breast, prostate, urinary bladder, skin, lung, ovarian, and brain cancers. High EphA2 expression is also correlated with poor prognosis. The monoclonal antibodies DS-8895a opt1, DS-8895 opt2, and MEDI-547 have been developed to target EphA2.

RON (macrophage stimulating 1 receptor) encodes a cell surface receptor for macrophage stimulating protein (MSP) with tyrosine kinase activity and belongs to the MET proto-oncogene family. RON has significant structural similarity and sequence identity with the cancer-related gene C-MET. RON plays a significant role in KRAS oncogene addiction and has also been shown to be overexpressed in pancreatic cancers. Altered Ron expression and activation has been associated with decreased survival and cancer progression in various cancers including gastric, colon, breast, bladder, renal cell, ovarian, and hepatocellular cancers. The monoclonal antibody narnatumab has been developed to target RON.

LY6E (lymphocyte antigen 6 complex, locus E) encodes an interferon alpha-inducible GPI-anchored cell membrane protein. LY6E is overexpressed in numerous cancers including lung, gastric, ovarian, breast, kidney, pancreatic, and head and neck carcinomas. The monoclonal antibody RG7841 has been developed to target LY6E.

FRA (folate receptor alpha) encodes a GPI-anchored cell surface glycoprotein. FRA binds folic acid, a molecule needed for cell growth and DNA synthesis, and mediates its internalization via receptor-mediated endocytosis. FRA is overexpressed in various cancers including prostate, breast, ovarian, pancreatic, mesothelioma, non-small cell lung carcinoma, and head and neck cancer. FRA expression has also been found to enhance tumor cell proliferation. The monoclonal antibodies farletuzumab and mirvetuximab have been developed to target FRA.

PSMA (prostate specific membrane antigen) is a type II transmembrane glycoprotein belonging to the M28 peptidase family that is expressed in all types of prostate tissues. PSMA is upregulated in cancer cells within the prostate and is used as a marker for prostate cancer. PSMA expression may also serve as a predictor of disease recurrence in prostate cancer patients. The monoclonal antibodies J591 variant 1 and J591 variant 2 have been developed to target PSMA.

DLL3 (delta-like 3) encodes a ligand in the Notch signaling pathway that is associated with neuroendocrine cancer. DLL3 is most highly expressed in the fetal brain and is involved in somitogenesis in the paraxial mesoderm. DLL3 is expressed on tumor cell surfaces but not in normal tissues. The monoclonal antibody rovalpituzumab has been developed to target DLL3.

PTK7 (tyrosine protein kinase-like 7) encodes a receptor tyrosine kinase that lacks catalytic tyrosine kinase activity but is nevertheless capable of signal transduction. PTK7 interacts with the WNT signaling pathway, which itself has important roles in epithelial mesenchymal transition and various cancers such as breast cancer. PTK7 overexpression has been associated with patient prognosis depending on the cancer type. The monoclonal antibodies PF-06647020 and the anti-PTK7 antibody described by SEQ ID NO 341 and 346 have been developed to target PTK7.

LIV1 (LIV-1 protein, estrogen regulated) encodes a member of the LIV-1 subfamily of ZIP (Zrt-, Irt-like proteins) zinc transporters. LIV1 is an estrogen regulated protein that transports zinc and/or other ions across the cell membrane. Elevated levels of LIV1 have been shown in estrogen receptor positive breast cancers, and LIV1 is used as a marker of ER-positive cancers. LIV1 has also been implicated as a downstream target of the STAT3 transcription factor and as playing an essential role in the nuclear localization of the Snail transcription factor that modulates epithelial-to-mesenchymal transition. The monoclonal antibody of SGN-LIV1A, ladiratuzumab, has been developed to target LIV1.

ROR1 (receptor tyrosine kinase-like orphan receptor 1) encodes a member of the ROR family of orphan receptors. ROR1 has been found to bind Wnt5a, a non-canonical Wnt via a Frizzled domain (FZD), and plays an important role in skeletal, cardiorespiratory, and neurological development. ROR1 expression is predominantly restricted to embryonic development and is absent in most mature tissues. In contrast, ROR1 expression is upregulated in B-Cell chronic lymphocytic leukemia, acute lymphocytic leukemia, non-Hodgkin lymphoma, and myeloid malignancies. The monoclonal antibody cirmtuzumab has been developed to target ROR1.

MAGE-A3 (melanoma-associated antigen 3) encodes a member of the melanoma-associated antigen gene family. The function of MAGE-A3 is not known, but its elevated expression has been observed in various cancers including melanoma, non-small cell lung cancer, and in putative cancer stem cell populations in bladder cancer. The monoclonal antibody described by SEQ ID NO 371 and 376 has been developed to target MAGE-A3.

NY-ESO-1 (New York esophageal squamous cell carcinoma 1) encodes a member of the cancer-testis family of proteins. Cancer-testis antigen expression is normally restricted to testicular germ cells in adult tissues, but has been found to be aberrantly expressed in various tumors including soft tissue sarcomas, melanoma, epithelial cancers, and myxoid and round cell liposarcomas. The monoclonal antibody described by SEQ ID NO 381 and 386 has been developed to target NY-ESO-1.

### Conjugates

The presently described immune modulatory conjugates (also referred to as antibody conjugates) can be utilized as strategy to enhance or otherwise modulate immune responses. A conjugate typically comprises an antibody construct and at least one immune-modulatory agent, optionally each immune-modulatory agent is attached to the antibody construct via a linker. An antibody contruct can comprise at least one binding domain (e.g., a first binding domain), typically at least two antigen binding domains, and optionally additional antigen binding domains (e.g., a third antigen binding domain, and a fourth antigen binding domain) and an Fc domain. In some embodiments, an antibody construct comprises a first antigen binding domain attached to an Fc domain. In some embodiments, an antibody construct can comprise a first antigen binding domain, a second antigen binding domain and an Fc domain, where each antigen binding domain is attached to the Fc domain. In some embodiments, a conjugate can comprise an antibody construct having a first antigen binding domain, a second antigen binding domain and an Fc domain, the first and second antigen binding domains attached to the Fc domain, and at least one immune-modulatory agent, optionally each immune-modulatory agent attached to the antibody construct via a linker.

In some embodiments, an antibody construct can comprise a first antigen binding domain, a second antigen binding domain, a third antigen binding domain and an Fc domain. The first antigen binding domain and the second antigen binding domain are attached to the Fc domain, and the third antigen binding domain is attached to a C-terminal end of a light chain of the first binding domain, to the C-terminal end of a light chain of the second binding domain or the C-terminal end of the Fc domain.

In some embodiments, the linker that attaches an immune-modulatory agent to an antibody construct has a protease-cleavage site that is cleavable by proteases present in the microenvironment of the disease. In some embodiments, the Fc domain of the antibody construct has an altered binding affinity for at least one Fc receptor, such as an Fc gamma receptor(s) or FcRn, allowing selective delivery of an immune-modulatory agent to certain immune cells.

These and other embodiments are described herein.

### Binding Domains

An antibody construct of a conjugate can contain one or more antigen binding domains (also referred to as binding domains). In some embodiments, an antibody construct has a first binding domain. In some embodiments, an antibody construct has a first and a second binding domain that bind to the same antigen. In some embodiments, an antibody construct has a first and a second binding domain that bind to different antigens. In some embodiments, an antibody construct has a first and a second binding domain, and optionally a third binding domain.

A binding domain typically recognizes a single antigen. An antibody contruct of a conjugate can have binding domains that can recognize, for example, two, three, four, five, six, seven, eight, nine, ten, or more antigens. An antibody construct can comprise two binding domains in which each binding domain can recognize the same antigen. An antibody construct can comprise two binding domains in which each binding domain can recognize a different antigen. An antibody construct can comprise three binding domains in which each binding domain can recognize a different antigen. An antibody construct can comprise three binding domains in which two of the binding domains can recognize the same antigen and the third binding domain recognizes a different antigen. In some embodiments, an antibody construct is bivalent and mono-specific (i.e., having two binding domains that specifically bind to the same antigen). In embodiments in which an antibody construct is trivalent or greater, the antibody construct is typically bi-specific or greater. Antibody constructs having a third binding domain attached to the C-terminal end of a light chain and/or the C-terminal end of an Fc domain can be bi-specific, tri-specific or multi-specific.

In some embodiments, an antibody construct can be a fusion protein, such as an Fc fusion protein, having a first binding domain and optionally a second binding domain. In some embodiments, two antigen binding domains and an Fc domain can be expressed as a fusion protein, optionally formed by expression of separate polypeptide chains.

A binding domain can specifically bind to an antigen on a cell surface or to a fragment thereof. A binding domain can specifically bind an antigen on a cell surface, for example, on a tumor cell, on an antigen presenting cell such as a dendritic cell or macrophage, or on another immune cell such as a T cell. In some embodiments, a binding domain can specifically bind to an antigen on a cell surface of a tumor cell or an antigen presenting cell (such as a dendritic cell or macrophage), but not on other immune cells such as T cells. In some embodiments, a binding domain can specifically bind to a tumor antigen. In some embodiments, a binding domain can specifically bind to an antigen on an antigen presenting cell. In some embodiments, a binding domain can be a cell surface receptor agonist, such as an agonistic antibody.

A binding domain can be an antigen-binding portion of an antibody (an antigen bind domain) or an antigen binding antibody fragment. A binding domain can be one or more fragments of an antibody that can retain the ability to specifically bind to an antigen. A binding domain can be in a scaffold, in which a scaffold is a supporting framework for the binding domain. A binding domain, such as an antigen binding fragment of an antibody, can be in an antibody scaffold or antibody-like scaffold. A binding domain can be in a non-antibody scaffold.

Antibody constructs can comprise a binding domain(s) that can specifically bind to a tumor antigen. A tumor antigen can be a tumor specific antigen and/or a tumor associated antigen. As described herein, a “tumor antigen” refers to a molecular marker that can be expressed on a neoplastic tumor cell and/or within a tumor microenvironment. The molecular marker can be a cell surface receptor. For example, a tumor antigen antigen can be an antigen expressed on a cell associated with a tumor, such as a neoplastic cell, stromal cell, endothelial cell, fibroblast, or tumor-infiltrating immune cell. For example, the tumor antigen antigen Her2/Neu can be overexpressed by certain types of breast and ovarian cancer. A tumor antigen can also be ectopically expressed by a tumor and contribute to deregulation of the cell cycle, reduced apoptosis, metastasis, and/or escape from immune surveillance. Tumor antigens are generally proteins or polypeptides derived therefrom, but can be glycans, lipids, or other small organic molecules. Additionally, a tumor antigen can arise through increases or decreases in post-translational processing exhibited by a cancer cell compared to a normal cell, for example, protein glycosylation, protein lipidation, protein phosphorylation, or protein acetylation.

In certain embodiments, a binding domain can specifically bind to a tumor antigen, such as, for example, GD2, GD3, GM2, Le^{y}, sLe, polysialic acid, fucosyl GM1, Tn, STn, BM3, or GloboH. In certain embodiments, a binding domain specifically can bind to a tumor antigen having an amino acid sequence having at least 80%, 90%, 95%, 97%, 98%, 99% or 100% identity to the amino acid sequence of CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, βvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, STN1, TNC, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), VISTA, or a fragment thereof.

In certain embodiments, an antigen binding domain specifically binds to a tumor antigen, such as those selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, αvβ3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In certain embodiments, an antigen binding domain specifically binds to an antigen associated with fibrotic or inflammatory disease, such as those selected from Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, MMP14, GPX8, and F2RL2.

A binding domain of an antibody construct can be selected from any domain that specifically binds to an antigen, including a binding domain of an antibody or a non-antibody binding domain. A binding domain of an antibody can be a monoclonal antibody, a polyclonal antibody, a recombinant antibody, or an antigen binding fragment thereof, for example, a heavy chain variable domain (VH) and a light chain variable domain (VL). A binding domain of a non-antibody scaffold can be a lipocalin, an anticalin, ‘T-body’, a peptide (e.g., a Bicycle™ peptide), an affibody, a peptibody, a DARPin, an affimer, an avimer, a knottin, a monobody, an affinity clamp, an ectodomain, a receptor ectodomain, a receptor, a cytokine, a ligand, an immunocytokine, a centryin, a T-cell receptor, or a recombinant T-cell receptor. In some embodiments, a binding domain of a non-antibody scaffold can be a lipocalin, an anticalin, ‘T-body’, an affibody, a peptide (e.g., a Bicycle™ peptide) a peptibody, a DARPin, an affimer, an avimer, a knottin, a monobody, a centryin or an affinity clamp.

In some embodiments, an antigen binding domain is other than an antibody or antigen binding fragment thereof, such as a bicyclic peptide (e.g., a Bicycle®), as described in Published International Application No. WO2014/140342, WO2013/050615, WO2013/050616, and WO2013/050617 (the disclosures of which are incorporated by reference herein).

In some embodiments, a binding domain of an antibody construct is an antigen binding domain from a monoclonal antibody and can comprise a light chain and a heavy chain. In an aspect, the monoclonal antibody binds to a tumor antigen comprises the light chain of a tumor antigen antibody and the heavy chain of a tumor antigen antibody, which specifically bind to the tumor antigen. In another aspect, the monoclonal antibody binds to an antigen present on the surface of an immune cell (immune cell antigen) and comprises the light chain of an anti-immune cell antigen antibody and the heavy chain of an anti-immune cell antigen antibody, which specifically bind to an immune cell antigen. In another aspect, the monoclonal antibody specifically binds to an antigen present on the surface of an antigen presenting cell (APC antigen) and comprises the light chain of an anti-APC antigen antibody and the heavy chain of an anti-APC antigen antibody, which bind an APC antigen.

In some embodiments, an antibody construct can comprise an antibody, such as a bivalent, mono-specific antibody. An antibody can consist of two identical light protein chains (light chains) and two identical heavy protein chains (heavy chains), all held together covalently by interchain disulfide linkages. The N-terminal regions of the light and heavy chains together can form the antigen recognition site of the antibody. Structurally, various functions of an antibody can be confined to discrete protein domains or regions. The portions that can recognize and can specifically bind to an antigen consist of three complementarity determining regions (CDRs) that lie within the variable heavy chain regions and variable light chain regions at the N-terminal ends of the heavy and light chains. The constant domains provide the general framework of the antibody and may not be involved directly in binding the antibody to an antigen, but can be involved in various effector functions, such as participation of the antibody in antibody-dependent cellular cytotoxicity (ADCC).

In some embodiments, an antibody construct comprises an antigen binding domain of an antibody that includes the light chain (LC) CDRs (LCDRs) and (HC) CDRs (HCDRs) of the antibody. For example, an antigen binding domain of an antibody can comprise one or more of the following: a light chain complementary determining region 1 (LCDR1), a light chain complementary determining region 2 (LCDR2), or a light chain complementary determining region 3 (LCDR3). As another example, an antibody binding domain can comprise one or more of the following: a heavy chain complementary determining region 1 (HCDR1), a heavy chain complementary determining region 2 (HCDR2), or a heavy chain complementary determining region 3 (HCDR3). In some embodiments an antigen binding domain comprises all of the following: LCDR1, LCDR2, LCDR3, HCDR1, HCDR2 and HCDR3. Unless stated otherwise, the CDRs described herein are defined according to the IMGT (the international ImMunoGeneTics information system) or the Kabat numbering system. In some embodiments, an antigen binding domain can comprise only the heavy chain of an antibody (e.g., does not include the light chain(s)). In some embodiments, an antigen binding domain can comprise only the light chain of an antibody (e.g., does not include the heavy chain(s)). In some embodiments, an antigen binding domain can comprise only the variable region of the heavy chain of an antibody. In some embodiments, an antigen binding domain can comprise only the variable region of the light chain of an antibody.

An antibody construct can also comprise any antigen binding fragment of an antibody or recombinant form thereof, including but not limited to an scFv, Fab, Fab′, Fab2, variable Fv fragment (Fv), domain antibody, and any other fragment thereof that can specifically bind to an antigen.

An antibody used herein can be chimeric or “humanized.” Humanized forms of non-human (e.g., murine) antibodies can be chimeric immunoglobulins, immunoglobulin chains or fragments thereof (such as Fv, Fab, Fab′, F(ab′)2 scFv, variable Fv fragment (Fv), domain antibody or other antigen-binding subdomains of antibodies), that contain minimal sequences derived from non-human immunoglobulin (e.g., the CDRs). In general, a humanized antibody can comprise substantially all of at least one, and typically two, variable domains, in which all or substantially all of the CDRs correspond to those of a non-human immunoglobulin and all or substantially all of the framework regions (FR) are those of a human immunoglobulin sequence. The humanized antibody can also comprise at least a portion of an immunoglobulin constant region (Fc), typically that of a human immunoglobulin consensus sequence.

An antibody also can be a human antibody. As used herein, “human antibodies” include antibodies having, for example, the amino acid sequence of a human immunoglobulin and include antibodies isolated from human immunoglobulin libraries or from animals transgenic for one or more human immunoglobulins that do not express endogenous immunoglobulins. Human antibodies can be produced using transgenic mice which are incapable of expressing functional endogenous immunoglobulins, but which can express human immunoglobulin genes.

Completely human antibodies that recognize a selected epitope can be generated using guided selection. In this approach, a selected non-human monoclonal antibody, e.g., a mouse antibody, is used to guide the selection of a completely human antibody recognizing the same epitope

An antibody can be a bispecific antibody or a dual variable domain antibody (DVD). Bispecific and DVD antibodies are monoclonal, often human or humanized, antibodies that have binding specificities for at least two different antigens (e.g., bi-specific).

An antibody described herein can be a derivatized antibody. For example, derivatized antibodies can be modified by glycosylation, acetylation, pegylation, phosphorylation, amidation, derivatization by known protecting/blocking groups, proteolytic cleavage, or the like. An antibody can also be modified, such as by defucosylation or deglycosylation.

A binding domain of an antibody construct can bind to tumor cells, such as an antibody against a cell surface receptor or a tumor antigen.

In some embodiments, an antibody construct can comprise a first binding domain or a first and second binding domain that specifically bind(s) to a tumor antigen. In some embodiments, a binding domain specifically can bind to a tumor antigen such as GD2, GD3, GM2, Le^{y}, sLe, polysialic acid, fucosyl GM1, Tn, STn, BM3, or GloboH. In some embodiments, a binding domain specifically can bind to a tumor antigen having an amino acid sequence comprising at least 80%, 90%, 95%, 97%, 98%, 99% or 100% identity to the amino acid sequence of CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), STN1, TNC, CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), VISTA, or a fragment thereof.

In certain embodiments, an antigen binding domain specifically binds to a tumor antigen, such as those selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In some embodiments, a first binding domain, or a first and second binding domain, can specifically bind a tumor antigen, wherein the tumor antigen is selected from the group consisting of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, and a fragment thereof. A first binding domain, or a first and second binding domain, can specifically bind a tumor antigen, wherein the tumor antigen is selected from the group consisting of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4) and STRA6 and a fragment thereof.

In certain embodiments, a first or a first and second antigen binding domain can specifically bind to an antigen associated with fibrotic or inflammatory disease, such as those selected from Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, MMP14, GPX8, and F2RL2,

An antibody construct of a conjugate can comprise a first binding domain, or a first and second binding domain, that specifically binds a tumor antigen. A conjugate or antibody construct can comprise a first binding domain, or a first and second binding domain, comprising one or more CDRs. A first binding domain, or a first and second binding domain, can comprise at least 80% sequence identity (or at least 90%, 95%, or 100% sequence identity) to any protein sequence in TABLE 1. A first binding domain, or a first and second binding domain, can comprise at set of six CDRs selected from the sets of CDRs set forth in TABLE 1.

TABLE 1
Tumor Antibody CDRs

| | | SEQ | Sequence |
|---|---|---|---|
| | | ID | |
| Antibody | Region | NO: | |
| Pertuzumab | HCDR1 | 13 | GFTFTDYT |
| | HCDR2 | 14 | VNPNSGGS |
| | HCDR3 | 15 | ARNLGPSFYFDY |
| | LCDR1 | 18 | QDVSIG |
| | LCDR2 | 19 | SAS |
| | LCDR3 | 20 | QQYYIYPYT |
| Cetuximab | HCDR1 | 23 | GFSLTNYG |
| | HCDR2 | 24 | IWSGGNT |
| | HCDR3 | 25 | ARALTYYDYEFAY |
| | LCDR1 | 28 | QSIGTN |
| | LCDR2 | 29 | YAS |
| | LCDR3 | 30 | QQNNNWPTT |
| Panitumumab | HCDR1 | 33 | GGSVSSGDYY |
| | HCDR2 | 34 | IYYSGNT |
| | HCDR3 | 35 | VRDRVTGAFDI |
| | LCDR1 | 38 | QDISNY |
| | LCDR2 | 39 | DAS |
| | LCDR3 | 40 | QHFDHLPLA |
| Nimotuzumab | HCDR1 | 43 | GYTFTNYY |
| | HCDR2 | 44 | INPTSGGS |
| | HCDR3 | 45 | ARQGLWFDSDGRGFDF |
| | LCDR1 | 48 | QNIVHSNGNTY |
| | LCDR2 | 49 | KVS |
| | LCDR3 | 50 | FQYSHVPWT |
| Zalutumumab | HCDR1 | 53 | GFTFSTYG |
| | HCDR2 | 54 | IWDDGSYK |
| | HCDR3 | 55 | ARDGITMVRGVMKDYFDY |
| | LCDR1 | 58 | QDISSA |
| | LCDR2 | 59 | DAS |
| | LCDR3 | 60 | QQFNSYPLT |
| Onartuzumab | HCDR1 | 63 | GYTFTSYW |
| | HCDR2 | 64 | IDPSNSDT |
| | HCDR3 | 65 | ATYRSYVTPLDY |
| | LCDR1 | 68 | QSLLYTSSQKNY |
| | LCDR2 | 69 | WAS |
| | LCDR3 | 70 | QQYYAYPWT |
| Patritumab | HCDR1 | 73 | GGSFSGYY |
| | HCDR2 | 74 | INHSGST |
| | HCDR3 | 75 | ARDKWTWYFDL |
| | LCDR1 | 78 | QSVLYSSSNRNY |
| | LCDR2 | 79 | WAS |
| | LCDR3 | 80 | QQYYSTPRT |
| Clivatuzumab | HCDR1 | 83 | GYTFPSYV |
| | HCDR2 | 84 | INPYNDGT |
| | HCDR3 | 85 | ARGFGGSYGFAY |
| | LCDR1 | 88 | SSVSSSY |
| | LCDR2 | 89 | STS |
| | LCDR3 | 90 | HQWNRYPYT |
| Sofituzumab | HCDR1 | 93 | GYSITNDYA |
| | HCDR2 | 94 | ISYSGYT |
| | HCDR3 | 95 | ARWTSGLDY |
| | LCDR1 | 98 | DLIHNW |
| | LCDR2 | 99 | GAT |
| | LCDR3 | 100 | QQYWTTPFT |
| Edrecolomab | HCDR1 | 103 | GYAFTNYL |
| | HCDR2 | 104 | INPGSGGT |
| | HCDR3 | 105 | ARDGPWFAY |
| | LCDR1 | 108 | ENVVTY |
| | LCDR2 | 109 | GAS |
| | LCDR3 | 110 | GQGYSYPYT |
| Adecatumumab | HCDR1 | 113 | GFTFSSYG |
| | HCDR2 | 114 | ISYDGSNK |
| | HCDR3 | 115 | AKDMGWGSGWRPYYYYGMDV |
| | LCDR1 | 118 | QSISSY |
| | LCDR2 | 119 | WAS |
| | LCDR3 | 120 | QQSYDIPYT |
| Anetumab | HCDR1 | 123 | GYSFTSYW |
| | HCDR2 | 124 | IDPGDSRT |
| | HCDR3 | 125 | ARGQLYGGTYMDG |
| | LCDR1 | 128 | SSDIGGYNS |
| | LCDR2 | 129 | GVN |
| | LCDR3 | 130 | SSYDIESATPV |
| huDS6 | HCDR1 | 133 | GYTFTSYN |
| | HCDR2 | 134 | IYPGNGAT |
| | HCDR3 | 135 | ARGDSVPFAY |
| | LCDR1 | 138 | SSVSF |
| | LCDR2 | 139 | STS |
| | LCDR3 | 140 | QQRSSFPLT |
| Lifastuzumab | HCDR1 | 143 | GFSFSDFA |
| | HCDR2 | 144 | IGRVAFHT |
| | HCDR3 | 145 | ARHRGFDVGHFDF |
| | LCDR1 | 148 | ETLVHSSGNTY |
| | LCDR2 | 149 | RVS |
| | LCDR3 | 150 | FQGSFNPLT |
| Sacituzumab | HCDR1 | 153 | GYTFTNYG |
| | HCDR2 | 154 | INTYTGEP |
| | HCDR3 | 155 | ARGGFGSSYWYFDV |
| | LCDR1 | 158 | QDVSIA |
| | LCDR2 | 159 | SAS |
| | LCDR3 | 160 | QQHYITPLT |
| PR1A3 | HCDR1 | 163 | GYTFTEFG |
| | HCDR2 | 164 | INTKTGEA |
| | HCDR3 | 165 | ARWDFYDYVEAMDY |
| | LCDR1 | 168 | QNVGTN |
| | LCDR2 | 169 | SAS |
| | LCDR3 | 170 | HQYYTYPLFT |
| Humanized | HCDR1 | 173 | GYTFTEFG |
| PR1A3 | HCDR2 | 174 | INTKTGEA |
| | HCDR3 | 175 | ARWDFAYYVEAMDY |
| | LCDR1 | 178 | AAVGTY |
| | LCDR2 | 179 | SAS |
| | LCDR3 | 180 | HQYYTYPLFT |
| Humanized Ab2-3 | HCDR1 | 183 | GFVFSSYD |
| | HCDR2 | 184 | YISSGGGIT |
| | HCDR3 | 185 | AAHYFGSSGPFAY |
| | LCDR1 | 188 | ENIFSY |
| | LCDR2 | 189 | NTR |
| | LCDR3 | 190 | QHHYGTPFT |
| IMAB362, | HCDR1 | 193 | GYTFTSYW |
| CLAUDIXIMAB | HCDR2 | 194 | IYPSDSYT |
| | HCDR3 | 195 | TRSWRGNSFDY |
| | LCDR1 | 198 | QSLLNSGNQKNY |
| | LCDR2 | 199 | WAS |
| | LCDR3 | 200 | QNDYSYPFT |
| AMG595 | HCDR1 | 203 | GFTFRNYG |
| | HCDR2 | 204 | IWYDGSDK |
| | HCDR3 | 205 | ARDGYDILTGNPRDFDY |
| | LCDR1 | 208 | QSLVHSDGNTY |
| | LCDR2 | 209 | RIS |
| | LCDR3 | 210 | MQSTHVPRT |
| ABT806 | HCDR1 | 213 | GYSISRDFA |
| | HCDR2 | 214 | ISYNGNT |
| | HCDR3 | 215 | VTASRGFPY |
| | LCDR1 | 218 | QDINSN |
| | LCDR2 | 219 | HGT |
| | LCDR3 | 220 | VQYAQFPWT |
| Sibrotuzumab | HCDR1 | 223 | RYTFTEYT |
| | HCDR2 | 224 | INPNNGIP |
| | HCDR3 | 225 | ARRRIAYGYDEGHAMDY |
| | LCDR1 | 228 | QSLLYSRNQKNY |
| | LCDR2 | 229 | WAS |
| | LCDR3 | 230 | QQYFSYPLT |
| DS-8895a variant | HCDR1 | 233 | GYTFIDYS |
| 1 | HCDR2 | 234 | INTYTGEP |
| | HCDR3 | 235 | ATYYRYERDFDY |
| | LCDR1 | 238 | QSIVHSSGITY |
| | LCDR2 | 239 | KVS |
| | LCDR3 | 240 | FQGSHVPYT |
| DS-8895a variant | HCDR1 | 243 | GYTFIDYS |
| 2 | HCDR2 | 244 | INTYTGEP |
| | HCDR3 | 245 | ATYYRYERDFDY |
| | LCDR1 | 248 | QSIVHSSGITY |
| | LCDR2 | 249 | KVS |
| | LCDR3 | 250 | FQGSHVPYT |
| MEDI-547 | HCDR1 | 253 | GFTFSHYM |
| | HCDR2 | 254 | IGPSGGPT |
| | HCDR3 | 255 | AGYDSGYDYVAVAGPAEYFQH |
| | LCDR1 | 258 | QSISTW |
| | LCDR2 | 259 | KAS |
| | LCDR3 | 260 | QQYNSYSRT |
| Namatumab | HCDR1 | 263 | GFTFSSYL |
| | HCDR2 | 264 | IKQDGSEK |
| | HCDR3 | 265 | TRDGYSSGRHYGMDV |
| | LCDR1 | 268 | QSVSRY |
| | LCDR2 | 269 | DAS |
| | LCDR3 | 270 | QQRSNWPRT |
| RG7841 | HCDR1 | 273 | GFSLTGYS |
| | HCDR2 | 274 | IWGDGST |
| | HCDR3 | 275 | ARDYYFNYASWFAY |
| | LCDR1 | 278 | QGISNYL |
| | LCDR2 | 279 | YTS |
| | LCDR3 | 280 | QQYSELPWT |
| Farletuzumab | HCDR1 | 283 | GFTFSGYG |
| | HCDR2 | 284 | ISSGGSYT |
| | HCDR3 | 285 | ARHGDDPAWFAY |
| | LCDR1 | 288 | SSISSNN |
| | LCDR2 | 289 | GTS |
| | LCDR3 | 290 | QQWSSYPYMYT |
| Mirvetuximab | HCDR1 | 293 | GYTFTGYF |
| | HCDR2 | 294 | IHPYDGDT |
| | HCDR3 | 295 | TRYDGSRAMDY |
| | LCDR1 | 298 | QSVSFAGTSL |
| | LCDR2 | 299 | RAS |
| | LCDR3 | 300 | QQSREYPYT |
| J591 variant 1 | HCDR1 | 303 | GYTFTEXT |
| | HCDR2 | 304 | INPNNGGT |
| | HCDR3 | 305 | AAGWNFDY |
| | LCDR1 | 308 | QDVGTA |
| | LCDR2 | 309 | WAS |
| | LCDR3 | 310 | QQYNSYPLT |
| J591 variant 2 | HCDR1 | 313 | GYTFTEYT |
| | HCDR2 | 314 | INPNNGGT |
| | HCDR3 | 315 | AAGWNFDY |
| | LCDR1 | 318 | ENVVTY |
| | LCDR2 | 319 | GAS |
| | LCDR3 | 320 | GQGYSYPYT |
| Rovalpituzumab | HCDR1 | 323 | GYTFTNYG |
| | HCDR2 | 324 | INTYTGEP |
| | HCDR3 | 325 | ARIGDSSPSDY |
| | LCDR1 | 328 | QSVSND |
| | LCDR2 | 329 | YAS |
| | LCDR3 | 330 | QQDYTSPWT |
| PF-06647020 | HCDR1 | 333 | GYTFTDYA |
| | HCDR2 | 334 | ISTYNDYT |
| | HCDR3 | 335 | ARGNSYFYALDY |
| | LCDR1 | 338 | ESVDSYGKSF |
| | LCDR2 | 339 | RAS |
| | LCDR3 | 340 | QQSNEDPWT |
| Antibody to PTK7 | HCDR1 | 343 | GFTFSSYA |
| | HCDR2 | 344 | ISYDGSIK |
| | HCDR3 | 345 | ARTYYFDY |
| | LCDR1 | 348 | QSIGSS |
| | LCDR2 | 349 | YAS |
| | LCDR3 | 350 | HQSSSLPIT |
| Ladiratuzumab of | HCDR1 | 353 | GLTIEDYY |
| SGN-LIV1A | HCDR2 | 354 | IDPENGDT |
| | HCDR3 | 355 | AVHNAHYGTWFAY |
| | LCDR1 | 358 | QSLLHSSGNTY |
| | LCDR2 | 359 | KIS |
| | LCDR3 | 360 | FQGSHVPYT |
| Cirmtuzumab | HCDR1 | 363 | GYAFTAYN |
| | HCDR2 | 364 | FDPYDGGS |
| | HCDR3 | 365 | ARGWYYFDY |
| | LCDR1 | 368 | KSISKY |
| | LCDR2 | 369 | SGS |
| | LCDR3 | 370 | QQHDESPYT |
| Antibody to | HCDR1 | 373 | GFRFRSHG |
| MAGE-A3 | HCDR2 | 374 | SYDGNNK |
| | HCDR3 | 375 | ASPYTSDWQYFQY |
| | LCDR1 | 378 | QNISTT |
| | LCDR2 | 379 | DTS |
| | LCDR3 | 380 | QQSNSWPLT |
| Antibody to NY- | HCDR1 | 383 | GFSFIDYG |
| ESO-1 | HCDR2 | 384 | MNWSGDKK |
| | HCDR3 | 385 | ARGEYSNRFDP |
| | LCDR1 | 388 | QSLVFTDGNTY |
| | LCDR2 | 389 | KVS |
| | LCDR3 | 390 | MQGTHWPPI |
| Trastuzumab | HCDR1 | 393 | GFNIKDTY |
| | HCDR2 | 394 | IYPTNGYT |
| | HCDR3 | 395 | SRWGGDGFYAMDY |
| | LCDR1 | 398 | QDVNTA |
| | LCDR2 | 399 | SAS |
| | LCDR3 | 400 | QQHYTTPPT |

A conjugate can comprise a first binding domain that binds a tumor antigen, wherein the first binding domain comprises at least 80% sequence identity to: a) HCDR1 comprising an amino acid sequence of SEQ ID NO: 13, HCDR2 comprising an amino acid sequence of SEQ ID NO: 14, HCDR3 comprising an amino acid sequence of SEQ ID NO: 15, LCDR1 comprising an amino acid sequence of SEQ ID NO: 18, LCDR2 comprising an amino acid sequence of SEQ ID NO: 19, and LCDR3 comprising an amino acid sequence of SEQ ID NO: 20; b) HCDR1 comprising an amino acid sequence of SEQ ID NO: 23, HCDR2 comprising an amino acid sequence of SEQ ID NO: 24, HCDR3 comprising an amino acid sequence of SEQ ID NO: 25, LCDR1 comprising an amino acid sequence of SEQ ID NO: 28, LCDR2 comprising an amino acid sequence of SEQ ID NO: 29, and LCDR3 comprising an amino acid sequence of SEQ ID NO: 30; c) HCDR1 comprising an amino acid sequence of SEQ ID NO: 33, HCDR2 comprising an amino acid sequence of SEQ ID NO: 34, HCDR3 comprising an amino acid sequence of SEQ ID NO: 35, LCDR1 comprising an amino acid sequence of SEQ ID NO: 38, LCDR2 comprising an amino acid sequence of SEQ ID NO: 39, and LCDR3 comprising an amino acid sequence of SEQ ID NO: 40; d) HCDR1 comprising an amino acid sequence of SEQ ID NO: 43, HCDR2 comprising an amino acid sequence of SEQ ID NO: 44, HCDR3 comprising an amino acid sequence of SEQ ID NO: 45, LCDR1 comprising an amino acid sequence of SEQ ID NO: 48, LCDR2 comprising an amino acid sequence of SEQ ID NO: 49, and LCDR3 comprising an amino acid sequence of SEQ ID NO: 50; e) HCDR1 comprising an amino acid sequence of SEQ ID NO: 53, HCDR2 comprising an amino acid sequence of SEQ ID NO: 54, HCDR3 comprising an amino acid sequence of SEQ ID NO: 55, LCDR1 comprising an amino acid sequence of SEQ ID NO: 58, LCDR2 comprising an amino acid sequence of SEQ ID NO: 59, and LCDR3 comprising an amino acid sequence of SEQ ID NO: 60; f) HCDR1 comprising an amino acid sequence of SEQ ID NO: 63, HCDR2 comprising an amino acid sequence of SEQ ID NO: 64, HCDR3 comprising an amino acid sequence of SEQ ID NO: 65, LCDR1 comprising an amino acid sequence of SEQ ID NO: 68, LCDR2 comprising an amino acid sequence of SEQ ID NO: 69, and LCDR3 comprising an amino acid sequence of SEQ ID NO: 70; g) HCDR1 comprising an amino acid sequence of SEQ ID NO: 73, HCDR2 comprising an amino acid sequence of SEQ ID NO: 74, HCDR3 comprising an amino acid sequence of SEQ ID NO: 75, LCDR1 comprising an amino acid sequence of SEQ ID NO: 78, LCDR2 comprising an amino acid sequence of SEQ ID NO: 79, and LCDR3 comprising an amino acid sequence of SEQ ID NO: 80; h) HCDR1 comprising an amino acid sequence of SEQ ID NO: 83, HCDR2 comprising an amino acid sequence of SEQ ID NO: 84, HCDR3 comprising an amino acid sequence of SEQ ID NO: 85, LCDR1 comprising an amino acid sequence of SEQ ID NO: 88, LCDR2 comprising an amino acid sequence of SEQ ID NO: 89, and LCDR3 comprising an amino acid sequence of SEQ ID NO: 90; i) HCDR1 comprising an amino acid sequence of SEQ ID NO: 93, HCDR2 comprising an amino acid sequence of SEQ ID NO: 94, HCDR3 comprising an amino acid sequence of SEQ ID NO: 95, LCDR1 comprising an amino acid sequence of SEQ ID NO: 98, LCDR2 comprising an amino acid sequence of SEQ ID NO: 99, and LCDR3 comprising an amino acid sequence of SEQ ID NO: 100; j) HCDR1 comprising an amino acid sequence of SEQ ID NO: 103, HCDR2 comprising an amino acid sequence of SEQ ID NO: 104, HCDR3 comprising an amino acid sequence of SEQ ID NO: 105, LCDR1 comprising an amino acid sequence of SEQ ID NO: 108, LCDR2 comprising an amino acid sequence of SEQ ID NO: 109, and LCDR3 comprising an amino acid sequence of SEQ ID NO: 110; k) HCDR1 comprising an amino acid sequence of SEQ ID NO: 113, HCDR2 comprising an amino acid sequence of SEQ ID NO: 114, HCDR3 comprising an amino acid sequence of SEQ ID NO: 115, LCDR1 comprising an amino acid sequence of SEQ ID NO: 118, LCDR2 comprising an amino acid sequence of SEQ ID NO: 119, and LCDR3 comprising an amino acid sequence of SEQ ID NO: 120; l) HCDR1 comprising an amino acid sequence of SEQ ID NO: 123, HCDR2 comprising an amino acid sequence of SEQ ID NO: 124, HCDR3 comprising an amino acid sequence of SEQ ID NO: 125, LCDR1 comprising an amino acid sequence of SEQ ID NO: 128, LCDR2 comprising an amino acid sequence of SEQ ID NO: 129, and LCDR3 comprising an amino acid sequence of SEQ ID NO: 130; m) HCDR1 comprising an amino acid sequence of SEQ ID NO: 133, HCDR2 comprising an amino acid sequence of SEQ ID NO: 134, HCDR3 comprising an amino acid sequence of SEQ ID NO: 135, LCDR1 comprising an amino acid sequence of SEQ ID NO: 138, LCDR2 comprising an amino acid sequence of SEQ ID NO: 139, and LCDR3 comprising an amino acid sequence of SEQ ID NO: 140; n) HCDR1 comprising an amino acid sequence of SEQ ID NO: 143, HCDR2 comprising an amino acid sequence of SEQ ID NO: 144, HCDR3 comprising an amino acid sequence of SEQ ID NO: 145, LCDR1 comprising an amino acid sequence of SEQ ID NO: 148, LCDR2 comprising an amino acid sequence of SEQ ID NO: 149, and LCDR3 comprising an amino acid sequence of SEQ ID NO: 150; o) HCDR1 comprising an amino acid sequence of SEQ ID NO: 153, HCDR2 comprising an amino acid sequence of SEQ ID NO: 154, HCDR3 comprising an amino acid sequence of SEQ ID NO: 155, LCDR1 comprising an amino acid sequence of SEQ ID NO: 158, LCDR2 comprising an amino acid sequence of SEQ ID NO: 159, and LCDR3 comprising an amino acid sequence of SEQ ID NO: 160; p) HCDR1 comprising an amino acid sequence of SEQ ID NO: 163, HCDR2 comprising an amino acid sequence of SEQ ID NO: 164, HCDR3 comprising an amino acid sequence of SEQ ID NO: 165, LCDR1 comprising an amino acid sequence of SEQ ID NO: 168, LCDR2 comprising an amino acid sequence of SEQ ID NO: 169, and LCDR3 comprising an amino acid sequence of SEQ ID NO: 170; q) HCDR1 comprising an amino acid sequence of SEQ ID NO: 173, HCDR2 comprising an amino acid sequence of SEQ ID NO: 174, HCDR3 comprising an amino acid sequence of SEQ ID NO: 175, LCDR1 comprising an amino acid sequence of SEQ ID NO: 178, LCDR2 comprising an amino acid sequence of SEQ ID NO: 179, and LCDR3 comprising an amino acid sequence of SEQ ID NO: 180; r) HCDR1 comprising an amino acid sequence of SEQ ID NO: 183, HCDR2 comprising an amino acid sequence of SEQ ID NO: 184, HCDR3 comprising an amino acid sequence of SEQ ID NO: 185, LCDR1 comprising an amino acid sequence of SEQ ID NO: 188, LCDR2 comprising an amino acid sequence of SEQ ID NO: 189, and LCDR3 comprising an amino acid sequence of SEQ ID NO: 190; s) HCDR1 comprising an amino acid sequence of SEQ ID NO: 193, HCDR2 comprising an amino acid sequence of SEQ ID NO: 194, HCDR3 comprising an amino acid sequence of SEQ ID NO: 195, LCDR1 comprising an amino acid sequence of SEQ ID NO: 198, LCDR2 comprising an amino acid sequence of SEQ ID NO: 199, and LCDR3 comprising an amino acid sequence of SEQ ID NO: 200; t) HCDR1 comprising an amino acid sequence of SEQ ID NO: 203, HCDR2 comprising an amino acid sequence of SEQ ID NO: 204, HCDR3 comprising an amino acid sequence of SEQ ID NO: 205, LCDR1 comprising an amino acid sequence of SEQ ID NO: 208, LCDR2 comprising an amino acid sequence of SEQ ID NO: 209, and LCDR3 comprising an amino acid sequence of SEQ ID NO: 210; u) HCDR1 comprising an amino acid sequence of SEQ ID NO: 213, HCDR2 comprising an amino acid sequence of SEQ ID NO: 214, HCDR3 comprising an amino acid sequence of SEQ ID NO: 215, LCDR1 comprising an amino acid sequence of SEQ ID NO: 218, LCDR2 comprising an amino acid sequence of SEQ ID NO: 219, and LCDR3 comprising an amino acid sequence of SEQ ID NO: 220; v) HCDR1 comprising an amino acid sequence of SEQ ID NO: 223, HCDR2 comprising an amino acid sequence of SEQ ID NO: 224, HCDR3 comprising an amino acid sequence of SEQ ID NO: 225, LCDR1 comprising an amino acid sequence of SEQ ID NO: 228, LCDR2 comprising an amino acid sequence of SEQ ID NO: 229, and LCDR3 comprising an amino acid sequence of SEQ ID NO: 230; w) HCDR1 comprising an amino acid sequence of SEQ ID NO: 233, HCDR2 comprising an amino acid sequence of SEQ ID NO: 234, HCDR3 comprising an amino acid sequence of SEQ ID NO: 235, LCDR1 comprising an amino acid sequence of SEQ ID NO: 238, LCDR2 comprising an amino acid sequence of SEQ ID NO: 239, and LCDR3 comprising an amino acid sequence of SEQ ID NO: 240; x) HCDR1 comprising an amino acid sequence of SEQ ID NO: 243, HCDR2 comprising an amino acid sequence of SEQ ID NO: 244, HCDR3 comprising an amino acid sequence of SEQ ID NO: 245, LCDR1 comprising an amino acid sequence of SEQ ID NO: 248, LCDR2 comprising an amino acid sequence of SEQ ID NO: 249, and LCDR3 comprising an amino acid sequence of SEQ ID NO: 250; y) HCDR1 comprising an amino acid sequence of SEQ ID NO: 253, HCDR2 comprising an amino acid sequence of SEQ ID NO: 254, HCDR3 comprising an amino acid sequence of SEQ ID NO: 255, LCDR1 comprising an amino acid sequence of SEQ ID NO: 258, LCDR2 comprising an amino acid sequence of SEQ ID NO: 259, and LCDR3 comprising an amino acid sequence of SEQ ID NO: 260; z) HCDR1 comprising an amino acid sequence of SEQ ID NO: 263, HCDR2 comprising an amino acid sequence of SEQ ID NO: 264, HCDR3 comprising an amino acid sequence of SEQ ID NO: 265, LCDR1 comprising an amino acid sequence of SEQ ID NO: 268, LCDR2 comprising an amino acid sequence of SEQ ID NO: 269, and LCDR3 comprising an amino acid sequence of SEQ ID NO: 270; aa) HCDR1 comprising an amino acid sequence of SEQ ID NO: 273, HCDR2 comprising an amino acid sequence of SEQ ID NO: 274, HCDR3 comprising an amino acid sequence of SEQ ID NO: 275, LCDR1 comprising an amino acid sequence of SEQ ID NO: 278, LCDR2 comprising an amino acid sequence of SEQ ID NO: 279, and LCDR3 comprising an amino acid sequence of SEQ ID NO: 280; bb) HCDR1 comprising an amino acid sequence of SEQ ID NO: 283, HCDR2 comprising an amino acid sequence of SEQ ID NO: 284, HCDR3 comprising an amino acid sequence of SEQ ID NO: 285, LCDR1 comprising an amino acid sequence of SEQ ID NO: 288, LCDR2 comprising an amino acid sequence of SEQ ID NO: 289, and LCDR3 comprising an amino acid sequence of SEQ ID NO: 290; cc) HCDR1 comprising an amino acid sequence of SEQ ID NO: 293, HCDR2 comprising an amino acid sequence of SEQ ID NO: 294, HCDR3 comprising an amino acid sequence of SEQ ID NO: 295, LCDR1 comprising an amino acid sequence of SEQ ID NO: 298, LCDR2 comprising an amino acid sequence of SEQ ID NO: 299, and LCDR3 comprising an amino acid sequence of SEQ ID NO: 300; dd) HCDR1 comprising an amino acid sequence of SEQ ID NO: 303, HCDR2 comprising an amino acid sequence of SEQ ID NO: 304, HCDR3 comprising an amino acid sequence of SEQ ID NO: 305, LCDR1 comprising an amino acid sequence of SEQ ID NO: 308, LCDR2 comprising an amino acid sequence of SEQ ID NO: 309, and LCDR3 comprising an amino acid sequence of SEQ ID NO: 310; ee) HCDR1 comprising an amino acid sequence of SEQ ID NO: 313, HCDR2 comprising an amino acid sequence of SEQ ID NO: 314, HCDR3 comprising an amino acid sequence of SEQ ID NO: 315, LCDR1 comprising an amino acid sequence of SEQ ID NO: 318, LCDR2 comprising an amino acid sequence of SEQ ID NO: 319, and LCDR3 comprising an amino acid sequence of SEQ ID NO: 320; ff) HCDR1 comprising an amino acid sequence of SEQ ID NO: 323, HCDR2 comprising an amino acid sequence of SEQ ID NO: 324, HCDR3 comprising an amino acid sequence of SEQ ID NO: 325, LCDR1 comprising an amino acid sequence of SEQ ID NO: 328, LCDR2 comprising an amino acid sequence of SEQ ID NO: 329, and LCDR3 comprising an amino acid sequence of SEQ ID NO: 330; gg) HCDR1 comprising an amino acid sequence of SEQ ID NO: 333, HCDR2 comprising an amino acid sequence of SEQ ID NO: 334, HCDR3 comprising an amino acid sequence of SEQ ID NO: 335, LCDR1 comprising an amino acid sequence of SEQ ID NO: 338, LCDR2 comprising an amino acid sequence of SEQ ID NO: 339, and LCDR3 comprising an amino acid sequence of SEQ ID NO: 340; hh) HCDR1 comprising an amino acid sequence of SEQ ID NO: 343, HCDR2 comprising an amino acid sequence of SEQ ID NO: 344, HCDR3 comprising an amino acid sequence of SEQ ID NO: 345, LCDR1 comprising an amino acid sequence of SEQ ID NO: 348, LCDR2 comprising an amino acid sequence of SEQ ID NO: 349, and LCDR3 comprising an amino acid sequence of SEQ ID NO: 350; ii) HCDR1 comprising an amino acid sequence of SEQ ID NO: 353, HCDR2 comprising an amino acid sequence of SEQ ID NO: 354, HCDR3 comprising an amino acid sequence of SEQ ID NO: 355, LCDR1 comprising an amino acid sequence of SEQ ID NO: 358, LCDR2 comprising an amino acid sequence of SEQ ID NO: 359, and LCDR3 comprising an amino acid sequence of SEQ ID NO: 360; jj) HCDR1 comprising an amino acid sequence of SEQ ID NO: 363, HCDR2 comprising an amino acid sequence of SEQ ID NO: 364, HCDR3 comprising an amino acid sequence of SEQ ID NO: 365, LCDR1 comprising an amino acid sequence of SEQ ID NO: 368, LCDR2 comprising an amino acid sequence of SEQ ID NO: 369, and LCDR3 comprising an amino acid sequence of SEQ ID NO: 370; kk) HCDR1 comprising an amino acid sequence of SEQ ID NO: 373, HCDR2 comprising an amino acid sequence of SEQ ID NO: 374, HCDR3 comprising an amino acid sequence of SEQ ID NO: 375, LCDR1 comprising an amino acid sequence of SEQ ID NO: 378, LCDR2 comprising an amino acid sequence of SEQ ID NO: 379, and LCDR3 comprising an amino acid sequence of SEQ ID NO: 380; ll) HCDR1 comprising an amino acid sequence of SEQ ID NO: 383, HCDR2 comprising an amino acid sequence of SEQ ID NO: 384, HCDR3 comprising an amino acid sequence of SEQ ID NO: 385, LCDR1 comprising an amino acid sequence of SEQ ID NO: 388, LCDR2 comprising an amino acid sequence of SEQ ID NO: 389, and LCDR3 comprising an amino acid sequence of SEQ ID NO: 390; or mm) HCDR1 comprising an amino acid sequence of SEQ ID NO: 393, HCDR2 comprising an amino acid sequence of SEQ ID NO: 394, HCDR3 comprising an amino acid sequence of SEQ ID NO: 395, LCDR1 comprising an amino acid sequence of SEQ ID NO: 398, LCDR2 comprising an amino acid sequence of SEQ ID NO: 399, and LCDR3 comprising an amino acid sequence of SEQ ID NO: 400.

An antibody construct of a conjugate can comprise a first binding domain, or a first and second binding domain, that specifically binds a tumor antigen, each binding domain comprising one or more variable regions. For example, a binding domain can comprise a light chain variable domain (VL domain). A binding domain can comprise a VL sequence set forth in TABLE 2. A binding domain can comprise a sequence having at least 80% sequence identity (or at least 90%, 95%, or 100% sequence identity) to a VL sequence in TABLE 2. A binding domain can comprise a heavy chain variable domain (VH domain). A binding domain can comprise VH sequence in TABLE 2. A binding domain can comprising a sequence having at least 80% sequence identity (or at least 90%, 95%, or 100% sequence identity) to any VH sequence in Table 4. A binding domain can comprise a pair of VL and VH sequences set forth in TABLE 2.

TABLE 2
Tumor Antibody V_{H} sequences and V_{L} sequences

| Antibody | Region | SEQ ID NO: | Sequence |
|---|---|---|---|
| Pertuzumab | V_{H} | 12 | EVQLVESGGGLVQPGGSLRLSCAASGFTFTDYTMDWV |
| | | | RQAPGKGLEWVADVNPNSGGSIYNQRFKGRFTLSVDRS |
| | | | KNTLYLQMNSLRAEDTAVYYCARNLGPSFYFDYWGQG |
| | | | TLVTVSS |
| | V_{L} | 17 | DIQMTQSPSSLSASVGDRVTITCKASQDVSIGVAWYQQ |
| | | | KPGKAPKLLIYSASYRYTGVPSRFSGSGSGTDFTLTISSL |
| | | | QPEDFATYYCQQYYIYPYTFGQGTKVEIK |
| Cetuximab | V_{H} | 22 | QVQLKQSGPGLVQPSQSLSITCTVSGFSLTNYGVHWVR |
| (EGFR) | | | QSPGKGLEWLGVIWSGGNTDYNTPFTSRLSINKDNSKS |
| | | | QVFFKMNSLQSNDTAIYYCARALTYYDYEFAYWGQGT |
| | | | LVTVSA |
| | V_{L} | 27 | DILLTQSPVILSVSPGERVSFSCRASQSIGTNIHWYQQRT |
| | | | NGSPRLLIKYASESISGIPSRFSGSGSGTDFTLSINSVESED |
| | | | IADYYCQQNNNWPTTFGAGTKLELK |
| Panitumumab | V_{H} | 32 | QVQLQESGPGLVKPSETLSLTCTVSGGSVSSGDYYWTW |
| (EGFR) | | | IRQSPGKGLEWIGHIYYSGNTNYNPSLKSRLTISIDTSKT |
| | | | QFSLKLSSVTAADTAIYYCVRDRVTGAFDIWGQGTMVT |
| | | | VSS |
| | V_{L} | 37 | DIQMTQSPSSLSASVGDRVTITCQASQDISNYLNWYQQK |
| | | | PGKAPKLLIYDASNLETGVPSRFSGSGSGTDFTFTISSLQP |
| | | | EDIATYFCQHFDHLPLAFGGGTKVEIK |
| Nimotuzumab | V_{H} | 42 | QVQLQQSGAEVKKPGSSVKVSCKASGYTFTNYYIYWV |
| (EGFR) | | | RQAPGQGLEWIGGINPTSGGSNFNEKFKTRVTITVDEST |
| | | | NTAYMELSSLRSEDTAFYFCARQGLWFDSDGRGFDFW |
| | | | GQGSTVTVSS |
| | V_{L} | 47 | DIQMTQSPSSLSASVGDRVTITCRSSQNIVHSNGNTYLD |
| | | | WYQQTPGKAPKLLIYKVSNRFSGVPSRFSGSGSGTDFTF |
| | | | TISSLQPEDIATYYCFQYSHVPWTFGQGTKLEIK |
| Zalutumumab | V_{H} | 52 | QVQLVESGGGVVQPGRSLRLSCAASGFTFSTYGMHWV |
| (EGFR) | | | RQAPGKGLEWVAVIWDDGSYKYYGDSVKGRFTISRDN |
| | | | SKNTLYLQMNSLRAEDTAVYYCARDGITMVRGVMKD |
| | | | YFDYWGQGTLVTVSS |
| | V_{L} | 57 | AIQLTQSPSSLSASVGDRVTITCRASQDISSALVWYQQKP |
| | | | GKAPKLLIYDASSLESGVPSRFSGSESGTDFTLTISSLQPE |
| | | | DFATYYCQQFNSYPLTFGGGTKVEIK |
| Onartuzumab | V_{H} | 62 | EVQLVESGGGLVQPGGSLRLSCAASGYTFTSYWLHWV |
| | | | RQAPGKGLEWVGMIDPSNSDTRFNPNFKDRFTISADTSK |
| | | | NTAYLQMNSLRAEDTAVYYCATYRSYVTPLDYWGQGT |
| | | | LVTVSS |
| | V_{L} | 67 | DIQMTQSPSSLSASVGDRVTITCKSSQSLLYTSSQKNYLA |
| | | | WYQQKPGKAPKLLIYWASTRESGVPSRFSGSGSGTDFT |
| | | | LTISSLQPEDFATYYCQQYYAYPWTFGQGTKVEIK |
| Patritumab | V_{H} | 72 | QVQLQQWGAGLLKPSETLSLTCAVYGGSFSGYYWSWI |
| | | | RQPPGKGLEWIGEINHSGSTNYNPSLKSRVTISVETSKNQ |
| | | | FSLKLSSVTAADTAVYYCARDKWTWYFDLWGRGTLVT |
| | | | VSS |
| | V_{L} | 77 | DIEMTQSPDSLAVSLGERATINCRSSQSVLYSSSNRNYL |
| | | | AWYQQNPGQPPKLLIYWASTRESGVPDRFSGSGSGTDF |
| | | | TLTISSLQAEDVAVYYCQQYYSTPRTFGQGTKVEIK |
| Clivatuzumab | V_{H} | 82 | QVQLQQSGAEVKKFGASVKVSCEASGYTFPSYVLHWV |
| | | | KQAPGQGLEWIGYINPYNDGTQTNKKFKGKATLTRDTS |
| | | | INTAYMELSRLRSDDTAVYYCARGFGGSYGFAYNGQG |
| | | | TLVTVSS |
| | V_{L} | 87 | DIQLTQSPSSLSASVGDRVTMTCSASSSVSSSYLYWYQQ |
| | | | KPGKAPKLWIYSTSNLASGVPARFSGSGSGTDFTLTISSL |
| | | | QPEDSASYFCHQWNRYPYTFGGGTRLEIK |
| Sofituzumab | V_{H} | 92 | EVQLVESGGGLVQPGGSLRLSCAASGYSITNDYAWNW |
| (MUC16) | | | VRQAPGKGLEWVGYISYSGYTTYNPSLKSRFTISRDTSK |
| | | | NTLYLQMNSLRAEDTAVYYCARWTSGLDYWGQGTLV |
| | | | TVSS |
| | V_{L} | 97 | DIQMTQSPSSLSASVGDRVTITCKASDLIHNWLAWYQQ |
| | | | KPGKAPKLLIYGATSLETGVPSRFSGSGSGTDFTLTISSL |
| | | | QPEDFATYYCQQYWTTPFTFGQGTKVEIK |
| Edrecolomab | V_{H} | 102 | QVQLQQSGAELVRPGTSVKVSCKASGYAFTNYLIEWVK |
| | | | QRPGQGLEWIGVINPGSGGTNYNEKFKGKATLTADKSS |
| | | | STAYMQLSSLTSDDSAVYFCARDGPWFAYWGQGTLVT |
| | | | VSA |
| | V_{L} | 107 | NIVMTQSPKSMSMSVGERVTLTCKASENVVTYVSWYQ |
| | | | QKPEQSPKLLIYGASNRYTGVPDRFTGSGSATDFTLTISS |
| | | | VQAEDLADYHCGQGYSYPYTFGGGTKLEIK |
| Adecatumumab | V_{H} | 112 | EVQLLESGGGVVQPGRSLRLSCAASGFTFSSYGMHWVR |
| | | | QAPGKGLEWVAVISYDGSNKYYADSVKGRFTISRDNSK |
| | | | NTLYLQMNSLRAEDTAVYYCAKDMGWGSGWRPYYYY |
| | | | GMDVWGQGTTVTVSS |
| | V_{L} | 117 | ELQMTQSPSSLSASVGDRVTITCRTSQSISSYLNWYQQK |
| | | | PGQPPKLLIYWASTRESGVPDRFSGSGSGTDFTLTISSLQ |
| | | | PEDSATYYCQQSYDIPYTFGQGTKLEIK |
| Anetumab | V_{H} | 122 | QVELVQSGAEVKKPGESLKISCKGSGYSFTSYWIGWVR |
| (MSLN) | | | QAPGKGLEWMGIIDPGDSRTRYSPSFQGQVTISADKSIST |
| | | | AYLQWSSLKASDTAMYYCARGQLYGGTYMDGWGQG |
| | | | TLVTVSS |
| | V_{L} | 127 | DIALTQPASVSGSPGQSITISCTGTSSDIGGYNSVSWYQQ |
| | | | HPGKAPKLMIYGVNNRPSGVSNRFSGSKSGNTASLTISG |
| | | | LQAEDEADYYCSSYDIESATPVFGGGTKLTVL |
| huDS6 | V_{H} | 132 | QAQLVQSGAEVVKPGASVKMSCKASGYTFTSYNMHW |
| | | | VKQTPGQGLEWIGYIYPGNGATNYNQKFQGKATLTADP |
| | | | SSSTAYMQISSLTSEDSAVYFCARGDSVPFAYWGQGTL |
| | | | VTVSA |
| | V_{L} | 137 | EIVLTQSPATMSASPGERVTITCSAHSSVSFMHWFQQKP |
| | | | GTSPKLWIYSTSSLASGVPARFGGSGSGTSYSLTISSMEA |
| | | | EDAATYYCQQRSSFPLTFGAGTKLELK |
| Lifastuzumab | V_{H} | 142 | EVQLVESGGGLVQPGGSLRLSCAASGFSFSDFAMSWVR |
| | | | QAPGKGLEWVATIGRVAFHTYYPDSMKGRFTISRDNSK |
| | | | NTLYLQMNSLRAEDTAVYYCARHRGFDVGHFDFWGQ |
| | | | GTLVTVSS |
| | V_{L} | 147 | DIQMTQSPSSLSASVGDRVTITCRSSETLVHSSGNTYLE |
| | | | WYQQKPGKAPKLLIYRVSNRFSGVPSRFSGSGSGTDFTL |
| | | | TISSLQPEDFATYYCFQGSFNPLTFGQGTKVEIK |
| Sacituzumab | V_{H} | 152 | QVQLQQSGSELKKPGASVKVSCKASGYTFTNYGMNWV |
| (TROP2) | | | KQAPGQGLKWMGWINTYTGEPTYTDDFKGRFAFSLDT |
| | | | SVSTAYLQISSLKADDTAVYFCARGGFGSSYWYFDVWG |
| | | | QGSLVTVSS |
| | V_{L} | 157 | DIQLTQSPSSLSASVGDRVSITCKASQDVSIAVAWYQQK |
| | | | PGKAPKLLIYSASYRYTGVPDRFSGSGSGTDFTLTISSLQ |
| | | | PEDFAVYYCQQHYITPLTFGAGTKVEIK |
| PR1A3 | V_{H} | 162 | QVKLQQSGPELKKPGETVKISCKASGYTFTEFGMNWVK |
| (CEA) | | | QAPGKGLKWMGWINTKTGEATYVEEFKGRFAFSLETS |
| | | | ATTAYLQINNLKNEDTAKYFCARWDFYDYVEAMDYW |
| | | | GQGTTVTVSS |
| | V_{L} | 167 | DIVMTQSQRFMSTSVGDRVSVTCKASQNVGTNVAWYQ |
| | | | QKPGQSPKALIYSASYRYSGVPDRFTGSGSGTDFTLTISN |
| | | | VQSEDLAEYFCHQYYTYPLFTFGSGTKLEMK |
| Humanized | V_{H} | 172 | QVQLVQSGAEVKKPGASVKVSCKASGYTFTEFGMNWV |
| PR1A3 | | | RQAPGQGLEWMGWINTKTGEATYVEEFKGRVTFTTDT |
| (CEA) | | | STSTAYMELRSLRSDDTAVYYCARWDFAYYVEAMDY |
| | | | WGQGTTVTVSS |
| | V_{L} | 177 | DIQMTQSPSSLSASVGDRVTITCKASAAVGTYVAWYQQ |
| | | | KPGKAPKLLIYSASYRKRGVPSRFSGSGSGTDFTLTISSL |
| | | | QPEDFATYYCHQYYTYPLFTFGQGTKLEIK |
| Humanized Ab2-3 | V_{H} | 182 | EVQLQESGPGLVKPGGSLSLSCAASGFVFSSYDMSWVR |
| | | | QTPERGLEWVAYISSGGGITYAPSTVKGRFTVSRDNAK |
| | | | NTLYLQMNSLTSEDTAVYYCAAHYFGSSGPFAYWGQG |
| | | | TLVTVSS |
| | V_{L} | 187 | DIQMTQSPASLSASVGDRVTITCRASENIFSYLAWYQQK |
| | | | PGKSPKLLVYNTRTLAEGVPSRFSGSGSGTDFSLTISSLQ |
| | | | PEDFATYYCQHHYGTPFTFGSGTKLEIK |
| IMAB362, | V_{H} | 192 | QVQLQQPGAELVRPGASVKLSCKASGYTFTSYWINWV |
| CLAUDIXIMAB | | | KQRPGQGLEWIGNIYPSDSYTNYNQKFKDKATLTVDKS |
| (CLDN18.2) | | | SSTAYMQLSSPTSEDSAVYYCTRSWRGNSFDYWGQGTT |
| | | | LTVSS |
| | V_{L} | 197 | DIVMTQSPSSLTVTAGEKVTMSCKSSQSLLNSGNQKNY |
| | | | LTWYQQKPGQPPKLLIYWASTRESGVPDRFTGSGSGTD |
| | | | FTLTISSVQAEDLAVYYCQNDYSYPFTFGSGTKLEIK |
| AMG595 | V_{H} | 202 | QVQLVESGGGVVQSGRSLRLSCAASGFTFRNYGMHWV |
| (EGFRvIII) | | | RQAPGKGLEWVAVIWYDGSDKYYADSVRGRFTISRDN |
| | | | SKNTLYLQMNSLRAEDTAVYYCARDGYDILTGNPRDFD |
| | | | YWGQGTLVTVSS |
| | V_{L} | 207 | DTVMTQTPLSSHVTLGQPASISCRSSQSLVHSDGNTYLS |
| | | | WLQQRPGQPPRLLIYRISRRFSGVPDRFSGSGAGTDFTLE |
| | | | ISRVEAEDVGVYYCMQSTHVPRTFGQGTKVEIK |
| ABT806 | V_{H} | 212 | EVQLQESGPGLVKPSQTLSLTCTVSGYSISRDFAWNWIR |
| | | | QPPGKGLEWMGYISYNGNTRYQPSLKSRITISRDTSKNQ |
| | | | FFLKLNSVTAADTATYYCVTASRGFPYWGQGTLVTVSS |
| | V_{L} | 217 | DIQMTQSPSSMSVSVGDRVTITCHSSQDINSNIGWLQQK |
| | | | PGKSFKGLIYHGTNLDDGVPSRFSGSGSGTDYTLTISSLQ |
| | | | PEDFATYYCVQYAQFPWTFGGGTKLEIK |
| Sibrotuzuma(FAP) | V_{H} | 222 | QVQLVQSGAEVKKPGASVKVSCKTSRYTFTEYTIHWVR |
| | | | QAPGQRLEWIGGINPNNGIPNYNQKFKGRVTITVDTSAS |
| | | | TAYMELSSLRSEDTAVYYCARRRIAYGYDEGHAMDYW |
| | | | GQGTLVTVSS |
| | V_{L} | 227 | DIVMTQSPDSLAVSLGERATINCKSSQSLLYSRNQKNYL |
| | | | AWYQQKPGQPPKLLIFWASTRESGVPDRFSGSGFGTDFT |
| | | | LTISSLQAEDVAVYYCQQYFSYPLTFGQGTKVEIK |
| DS-8895a variant | V_{H} | 232 | QVQLVQSGAEVKKPGASVKVSCKASGYTFIDYSMHWV |
| 1 | | | RQAPGQGLEWMGWINTYTGEPTYSDDFKGRVTITADTS |
| | | | TSTAYLELSSLRSEDTAVYYCATYYRYERDFDYWGQGT |
| | | | LVTVSS |
| | V_{L} | 237 | DIVMTQSPLSLPVTPGEPASISCRSSQSIVHSSGITYLEWY |
| | | | LQKPGQSPQLLIYKVSNRFSGVPDRFSGSGSGTDFTLKIS |
| | | | RVEAEDVGVYYCFQGSHVPYTFGQGTKVEIK |
| DS-8895a variant | V_{H} | 242 | QIQLVQSGAEVKKPGASVKVSCKASGYTFIDYSMHWVR |
| 2 | | | QAPGQGLKWMGWINTYTGEPTYSDDFKGRFAFSLDTST |
| | | | STAYLELSSLRSEDTAVYYCATYYRYERDFDYWGQGTL |
| | | | VTVSS |
| | V_{L} | 247 | DVLMTQSPLSLPVTPGEPASISCRSSQSIVHSSGITYLEW |
| | | | YLQKPGQSPQLLIYKVSNRFSGVPDRFSGSGSGTDFTLKI |
| | | | SRVEAEDVGVYYCFQGSHVPYTFGQGTKVEIK |
| MEDI-547 | V_{H} | 252 | EVQLLESGGGLVQPGGSLRLSCAASGFTFSHYMMAWV |
| | | | RQAPGKGLEWVSRIGPSGGPTHYADSVKGRFTISRDNSK |
| | | | NTLYLQMNSLRAEDTAVYYCAGYDSGYDYVAVAGPA |
| | | | EYFQHWGQGTLVTVSS |
| | V_{L} | 257 | DIQMTQSPSSLSASVGDRVTITCRASQSISTWLAWYQQK |
| | | | PGKAPKLLIYKASNLHTGVPSRFSGSGSGTEFSLTISGLQ |
| | | | PDDFATYYCQQYNSYSRTFGQGTKVEIK |
| Narnatumab | V_{H} | 262 | EVQLVESGGGLVQPGGSLRLSCAASGFTFSSYLMTWVR |
| | | | QAPGKGLEWVANIKQDGSEKYYVDSVKGRFTISRDNA |
| | | | KNSLNLQMNSLRAEDTAVYYCTRDGYSSGRHYGMDV |
| | | | WGQGTTVIVSS |
| | V_{L} | 267 | EIVLTQSPATLSLSPGERATLSCRASQSVSRYLAWYQQK |
| | | | PGQAPRLLIYDASNRATGIPARFSGSGSGTDFTLTISSLEP |
| | | | EDFAVYYCQQRSNWPRTFGQGTKVEIK |
| RG7841 | V_{H} | 272 | EVQLVESGPALVKPTQTLTLTCTVSGFSLTGYSVNWIRQ |
| | | | PPGKALEWLGMIWGDGSTDYNSALKSRLTISKDTSKNQ |
| | | | VVLTMTNMDPVDTATYYCARDYYFNYASWFAYWGQG |
| | | | TLVTVSS |
| | V_{L} | 277 | DIQMTQSPSSLSASVGDRVTITCSASQGISNYLNWYQQK |
| | | | PGKTVKLLIYYTSNLHSGVPSRFSGSGSGTDYTLTISSLQ |
| | | | PEDFATYYCQQYSELPWTFGQGTKVEIK |
| Farletuzumab | V_{H} | 282 | EVQLVESGGGVVQPGRSLRLSCSASGFTFSGYGLSWVR |
| | | | QAPGKGLEWVAMISSGGSYTYYADSVKGRFAISRDNAK |
| | | | NTLFLQMDSLRPEDTGVYFCARHGDDPAWFAYWGQGT |
| | | | PVTVSS |
| | V_{L} | 287 | DIQLTQSPSSLSASVGDRVTITCSVSSSISSNNLHWYQQK |
| | | | PGKAPKPWIYGTSNLASGVPSRFSGSGSGTDYTFTISSLQ |
| | | | PEDIATYYCQQWSSYPYMYTFGQGTKVEIK |
| Mirvetuximab | V_{H} | 292 | QVQLVQSGAEVVKPGASVKISCKASGYTFTGYFMNWV |
| | | | KQSPGQSLEWIGRIHPYDGDTFYNQKFQGKATLTVDKS |
| | | | SNTAHMELLSLTSEDFAVYYCTRYDGSRAMDYWGQGT |
| | | | TVTVSS |
| | V_{L} | 297 | DIVLTQSPLSLAVSLGQPAIISCKASQSVSFAGTSLMHWY |
| | | | HQKPGQQPRLLIYRASNLEAGVPDRFSGSGSKTDFTLTIS |
| | | | PVEAEDAATYYCQQSREYPYTFGGGTKLEIK |
| J591 variant 1 | V_{H} | 302 | EVQLQQSGPELKKPGTSVRISCKTSGYTFTEYTIHWVKQ |
| | | | SHGKSLEWIGNINPNNGGTTYNQKFEDKATLTVDKSSST |
| | | | AYMELRSLTSEDSAVYYCAAGWNFDYWGQGTTLTVSS |
| | V_{L} | 307 | DIVMTQSHKFMSTSVGDRVSIICKASQDVGTAVDWYQQ |
| | | | KPGQSPKLLIYWASTRHTGVPDRFTGSGSGTDFTLTITN |
| | | | VQSEDLADYFCQQYNSYPLTFGAGTMLDLK |
| J591 variant 2 | V_{H} | 312 | EVQLQQSGPELVKPGTSVRISCKTSGYTFTEYTIHWVKQ |
| | | | SHGKSLEWIGNINPNNGGTTYNQKFEDKATLTVDKSSST |
| | | | AYMELRSLTSEDSAVYYCAAGWNFDYWGQGTTLTVSS |
| | V_{L} | 317 | NIVMTQSPKSMSMSVGERVTLTCKASENVVTYVSWYQ |
| | | | QKPEQSPKLLIYGASNRYTGVPDRFTGSGSATDFTLTISS |
| | | | VQAEDLADYHCGQGYSYPYTFGGGTKLEIK |
| Rovalpituzumab | V_{H} | 322 | QVQLVQSGAEVKKPGASVKVSCKASGYTFTNYGMNW |
| | | | VRQAPGQGLEWMGWINTYTGEPTYADDFKGRVTMTT |
| | | | DTSTSTAYMELRSLRSDDTAVYYCARIGDSSPSDYWGQ |
| | | | GTLVTVSS |
| | V_{L} | 327 | EIVMTQSPATLSVSPGERATLSCKASQSVSNDVVWYQQ |
| | | | KPGQAPRLLIYYASNRYTGIPARFSGSGSGTEFTLTISSLQ |
| | | | SEDFAVYYCQQDYTSPWTFGQGTKLEIK |
| PF-06647020 | V_{H} | 332 | QVQLVQSGPEVKKPGASVKVSCKASGYTFTDYAVHWV |
| | | | RQAPGKRLEWIGVISTYNDYTYNNQDFKGRVTMTRDTS |
| | | | ASTAYMELSRLRSEDTAVYYCARGNSYFYALDYWGQG |
| | | | TSVTVSS |
| | V_{L} | 337 | EIVLTQSPATLSLSPGERATLSCRASESVDSYGKSFMHW |
| | | | YQQKPGQAPRLLIYRASNLESGIPARFSGSGSGTDFTLTI |
| | | | SSLEPEDFAVYYCQQSNEDPWTFGGGTKLEIK |
| Antibody to PTK7 | V_{H} | 342 | QVQLVESGGGVVQPGRSLRLSCAASGFTFSSYAFHWVR |
| | | | QAPGKGLEWVAVISYDGSIKYYADSVKGRFTISRDNSK |
| | | | NTLYLQMNSLRAEDTAVYYCARTYYFDYWGQGTLVT |
| | | | VSS |
| | V_{L} | 347 | EIVLTQSPDFQSVTPKEKVTITCRASQSIGSSLHWYQQKP |
| | | | DQSPKLLIKYASQSFSGVPSRFSGSGSGTDFTLTINSLEAE |
| | | | DAAAYYCHQSSSLPITFGQGTRLEIK |
| Ladiratuzumab of | V_{H} | 352 | QVQLVQSGAEVKKPGASVKVSCKASGLTIEDYYMHWV |
| SGN-LIV1A | | | RQAPGQGLEWMGWIDPENGDTEYGPKFQGRVTMTRDT |
| | | | SINTAYMELSRLRSDDTAVYYCAVHNAHYGTWFAYWG |
| | | | QGTLVTVSS |
| | V_{L} | 357 | DVVMTQSPLSLPVTLGQPASISCRSSQSLLHSSGNTYLEY |
| | | | FQQRPGQSPRPLIYKISTRFSGVPDRFSGSGSGTDFTLKIS |
| | | | RVEAEDVGVYYCFQGSHVPYTFGGGTKVEIK |
| Cirmtuzumab | V_{H} | 362 | QVQLQESGPGLVKPSQTLSLTCTVSGYAFTAYNIHWVR |
| | | | QAPGQGLEWMGSFDPYDGGSSYNQKFKDRLTISKDTSK |
| | | | NQVVLTMTNMDPVDTATYYCARGWYYFDYWGHGTL |
| | | | VTVSS |
| | V_{L} | 367 | DIVMTQTPLSLPVTPGEPASISCRASKSISKYLAWYQQKP |
| | | | GQAPRLLIYSGSTLQSGIPPRFSGSGYGTDFTLTINNIESE |
| | | | DAAYYFCQQHDESPYTFGEGTKVEIK |
| Antibody to | V_{H} | 372 | QVQLVESGGGVVQPGRSLRLSCTASGFRFRSHGMHWV |
| MAGE-A3 | | | RQAPGKGLEWVAVISYDGNNKLYADSVKGRITISRDNS |
| | | | KNTLFLQMNNVRAEDTAVYYCASPYTSDWQYFQYWG |
| | | | QGTLVIVSS |
| | V_{L} | 377 | EIVMTQSPATLSVSPGERATFSCRASQNISTTLAWYQQK |
| | | | PGQAPRLLIYDTSTRATGIPARFSGSGSGTEFTLTISSLQS |
| | | | EDLAVYYCQQSNSWPLTFGGGTKVEIK |
| Antibody to NY- | V_{H} | 382 | QVQLVQSGGGVVRPGGSLRLSCAASGFSFIDYGMSWVR |
| ESO-1 | | | QVPGKGLEWVAGMNWSGDKKGHAESVKGRFIISRDNA |
| | | | KNTLYLEMSSLRVEDTALYFCARGEYSNRFDPRGRGTL |
| | | | VTVSS |
| | V_{L} | 387 | DIVMTQTPLSLPVTLGQPASLSCRSSQSLVFTDGNTYLN |
| | | | WFQQRPGQSPRRLIYKVSSRDPGVPDRFSGTGSGTDFTL |
| | | | EISRVEAEDIGVYYCMQGTHWPPIFGQGTKVEIK |
| Trastuzumab | V_{H} | 392 | EVQLVESGGGLVQPGGSLRLSCAASGFNIKDTYIHWVR |
| | | | QAPGKGLEWVARIYPTNGYTRYADSVKGRFTISADTSK |
| | | | NTAYLQMNSLRAEDTAVYYCSRWGGDGFYAMDYWG |
| | | | QGTLVTVSS |
| | V_{L} | 397 | DIQMTQSPSSLSASVGDRVTITCRASQDVNTAVAWYQQ |
| | | | KPGKAPKLLIYSASFLYSGVPSRFSGSRSGTDFTLTISSLQ |
| | | | PEDFATYYCQQHYTTPPTFGQGTKVEIK |
| 4H11 | V_{H} | 494 | EVKLQESGGGFVKPGGSLKVSCAASGFTFSSYAMSWVR |
| (MUC16) | | | LSPEMRLEWVATISSAGGYIFYSDSVQGRFTISRDNAKN |
| | | | TLHLQMGSLRSGDTAMYYCARQGFGNYGDYYAMDY |
| | | | WGQGTTVTVSS |
| | V_{L} | 495 | DIELTQSPSSLAVSAGEKVTMSCKSSQSLLNSRTRKNQL |
| | | | AWYQQKPGQSPELLIYWASTRQSGVPDRFTGSGSGTDF |
| | | | TLTISSVQAEDLAVYYCQQSYNLLTFGPGTKLEVK |
| 4A5 | V_{H} | 496 | EVKLEESGGGFVKPGGSLKISCAASGFTFRNYAMSWVR |
| (MUC16) | | | LSPEMRLEWVATISSAGGYIFYSDSVQGRFTISRDNAKN |
| | | | TLHLQMGSLRSGDTAMYYCARQGFGNYGDYYAMDY |
| | | | WGQGTTVTVSS |
| | V_{L} | 497 | DIELTQSPSSLAVSAGEKVTMSCKSSQSLLNSRTRKNQL |
| | | | AWYQQKTGQSPELLIYWASTRQSGVPDRFTGSGSGTDF |
| | | | TLTISSVQAEDLAVYYCQQSYNLLTFGPGTKLEIK |
| Arcitumomab | V_{H} | 498 | EVKLVESGGGLVQPGGSLRLSCATSGFTFTDYYMNWV |
| (CEA) | | | RQPPGKALEWLGFIGNKANGYTTEYSASVKGRFTISRD |
| | | | KSQSILYLQMNTLRAEDSATYYCTRDRGLRFYFDYWGQ |
| | | | GTTLTVSS |
| | V_{L} | 499 | QTVLSQSPAILSASPGEKVTMTCRASSSVTYIHWYQQKP |
| | | | GSSPKSWIYATSNLASGVPARFSGSGSGTSYSLTISRVEA |
| | | | EDAATYYCQHWSSKPPTFGGGTKLEIK |
| MORab-009 | V_{H} | 500 | QVQLQQSGPELEKPGASVKISCKASGYSFTGYTMNWVK |
| | | | QSHGKSLEWIGLITPYNGASSYNQKFRGKATLTVDKSSS |
| | | | TAYMDLLSLTSEDSAVYFCARGGYDGRGFDYWGSGTP |
| | | | VTVSS |
| | V_{L} | 501 | DIELTQSPAIMSASPGEKVTMTCSASSSVSYMHWYQQK |
| | | | SGTSPKRWIYDTSKLASGVPGRFSGSGSGNSYSLTISSVE |
| | | | AEDDATYYCQQWSKHPLTFGSGTKVEIK |
| RG7787 | V_{H} | 502 | MQVQLVQSGAEVKKPGASVKVSCKASGYSFTGYTMN |
| | | | WVRQAPGQGLEWMGLITPYNGASSYNQKFRGKATMT |
| | | | VDTSTSTVYMELSSLRSEDTAVYYCARGGYDGRGFDY |
| | | | WGQGTLVTVSS |
| | V_{L} | 503 | MDIQMTQSPSSLSASVGDRVTITCSASSSVSYMHWYQQ |
| | | | KSGKAPKLLIYDTSKLASGVPSRFSGSGSGTDFTLTISSL |
| | | | QPEDFATYYCQQWSKHPLTFGQGTKLEIK |
| Atezolizumab | V_{H} | 504 | EVQLVESGGGLVQPGGSLRLSCAASGFTFSDSWIHWVR |
| | | | QAPGKGLEWVAWISPYGGSTYYADSVKGRFTISADTSK |
| | | | NTAYLQMNSLRAEDTAVYYCARRHWPGGFDYWGQGT |
| | | | LVTVSS |
| | V_{L} | 505 | DIQMTQSPSSLSASVGDRVTITCRASQDVSTAVAWYQQ |
| | | | KPGKAPKLLIYSASFLYSGVPSRFSGSGSGTDFTLTISSLQ |
| | | | PEDFATYYCQQYLYHPATFGQGTKVEIK |
| MDX-1105 | V_{H} | 506 | QVQLVQSGAEVKKPGSSVKVSCKTSGDTFSTYAISWVR |
| | | | QAPGQGLEWMGGIIPIFGKAHYAQKFQGRVTITADESTS |
| | | | TAYMELSSLRSEDTAVYFCARKFHFVSGSPFGMDVWG |
| | | | QGTTVTVSS |
| | V_{L} | 507 | EIVLTQSPATLSLSPGERATLSCRASQSVSSYLAWYQQK |
| | | | PGQAPRLLIYDASNRATGIPARFSGSGSGTDFTLTISSLEP |
| | | | EDFAVYYCQQRSNWPTFGQGTKVEIK |
| Antibody to FAP | V_{H} | 508 | QVQLQESGPGLVKPSQTLSLTCAISGDSVSSNSVTWNWI |
| variant 1 | | | RQSPSRGLEWLGRTYYRSKWYNDYAVSVKGRITINPDT |
| | | | SKNQFYLQLKSVTPEDAAVYYCARDSSILYGDYWGQG |
| | | | TLVTVSS |
| | V_{H} | 509 | QVQLQQSGPGLVKPSQTLSLTCAISGDSVSSNSVTWNWI |
| | | | RQSPSRGLEWLGRTYYRSKWYNDYAVSVKGRITINPDT |
| | | | SKNQFYLQLKSVTPEDAAVYYCARDSSILYGDYWGQG |
| | | | TLVTVS |
| | V_{L} | 510 | QAVLTQPSSLSASPGASASLTCTLPSGINVGTYRIFWFQQ |
| | | | KPGSPPQYLLSYKSDSDNHQGSGVPSRFSGSKDASANA |
| | | | GILLISGLQSEDEADYYCMIWHSSAWVFGGGTKLTVL |
| Antibody to FAP | V_{H} | 511 | QVQLVQSGAEVKKPGASVKVSCKTSGYTFTDYYIHWV |
| variant 2 | | | RQAPGQGLEWMGWINPNRGGTNYAQKFQGRVTMTRD |
| | | | TSIATAYMELSRLRSDDTAVYYCATASLKIAAVGTFDC |
| | | | WGQGTLVTVSS |
| | V_{L} | 512 | SYELTQPPSVSVSPGQTARITCSGDALSKQYAFWFQQKP |
| | | | GQAPILVIYQDTKRPSGIPGRFSGSSSGTTVTLTISGAQA |
| | | | DDEADYYCQSADSSGTYVFGTGTKVTVL |
| Antibody to FAP | V_{H} | 513 | EVQLVETGGGVVQPGRSLRLSCAASGFSFSTHGMYWV |
| variant 3 | | | RQPPGKGLEWVAVISYDGSDKKYADSVKGRFTISRDNS |
| | | | KNTVYLEMSSVRAEDTALYYCFCRRDAFDLWGQGTMV |
| | | | TVSS |
| | V_{L} | 514 | SYVLTQPPSVSVSPGQTARITCSGDALPKKYAYWYQQK |
| | | | SGQAPVLVIYEDTKRPSGIPERFSGSSSGTMATLTISGAQ |
| | | | VEDEADYYCYSTDSSGNYWVFGGGTEVTVL |
| Antibody to FAP | V_{H} | 515 | EVQLVESGGGLVEPGGSLRLSCAASGFTFSDAWMNWV |
| variant 4 | | | RQAPGKGLEWVGRIKTKSDGGTTDYAAPVRGRFSISRD |
| | | | DSKNTLFLEMNSLKTEDTAIYYCFITVIVVSSESPLDHW |
| | | | GQGTLVTVSS |
| | V_{L} | 516 | SYELTQPPSVSVSPGQTARITCSGDELPKQYAYWYQQKP |
| | | | GQAPVLVIYKDRQRPSGIPERFSGSSSGTTVTLTISGVQA |
| | | | EDEADYYCQSAYSINTYVIFGGGTKLTVL |
| Antibody to FAP | V_{H} | 517 | EVQLVESGGGLVKPGGSLRLSCAASGFTFSDYYMSWIR |
| variant 5 | | | QAPGKGLEWISYISSGSSYTNYADSVKGRFTISRDNAKK |
| | | | SVYLEVNGLTVEDTAVYYCARVRYGDREMATIGGFDF |
| | | | WGQGTLVTVSS |
| | V_{L} | 518 | SYELTQPPSVSVSPGQTARITCSGDALPKQYAYWYQQSP |
| | | | GQAPVLVIYKDSERPSGIPERFSGSSSGTTVTLTISGVQA |
| | | | EDEADYYCQSADSGGTSRIFGGGTKLTVL |
| Antibody to FAP | V_{H} | 519 | QVQLQESGPGLVRSTETLSLTCLVSGDSINSHYWSWLR |
| variant 6 | | | QSPGRGLEWIGYIYYTGPTNYNPSLKSRVSISLGTSKDQF |
| | | | SLKLSSVTAADTARYYCARNKVFWRGSDFYYYMDVW |
| | | | GKGTTVTVSS |
| | V_{L} | 520 | EIVLTQSPGTLSLSLGERATLSCRASQSLANNYLAWYQQ |
| | | | KPGQAPRLLMYDASTRATGIPDRFSGSGSGTDFTLTISRL |
| | | | EPEDFAVYYCQQFVTSHHMYIFGQGTKVEIK |
| Antibody to FAP | V_{H} | 521 | HVQLQESGPGLVKPSETLSLTCTVSGGSISSNNYYWGWI |
| variant 7 | | | RQTPGKGLEWIGSIYYSGSTNYNPSLKSRVTISVDTSKN |
| | | | QFSLKLSSVTAADTAVYYCARGARWQARPATRIDGVAF |
| | | | DIWGQGTMVTVSS |
| | V_{H} | 522 | QVQLQESGPGLVKPSETLSLTCTVSGGSISSNNYYWGWI |
| | | | RQTPGKGLEWIGSIYYSGSTNYNPSLKSRVTISVDTSKN |
| | | | QFSLKLSSVTAADTAVYYCARGARWQARPATRIDGVAF |
| | | | DIWGQGTMVTVSS |
| | V_{H} | 523 | EVQLVQSGAEVKKPGASVKVSCKASGYTFTSYGISWVR |
| | | | QAPGQGLEWMGWISAYNGNTNYAQKLQGRVTMTTDT |
| | | | STSTAYMELRSLRSDDTAVYYCARDWSRSGYYLPDYW |
| | | | GQGTLVTVSS |
| | V_{L} | 524 | ETTLTQSPGTLSLSPGERATLSCRASQTVTRNYLAWYQQ |
| | | | KPGQAPRLLMYGASNRAAGVPDRFSGSGSGTDFTLTISR |
| | | | LEPEDFAVYYCQQFGSPYTFGQGTKVEIK |
| | V_{L} | 525 | DVVMTQSPLSLPVTLGQPASISCRSSQSLLHSNGYNYLD |
| | | | WYLQRPGQSPHLLIFLGSNRASGVPDRFSGSGSGTDFTL |
| | | | KISRVEAEDVGIYYCMQALQTPPTFGQGTKVEIK |
| Antibody huM25 | V_{H} | 526 | EVQLVQSGAEVKKPGASVKVSCKASGYKFSSYWIEWV |
| to LRRC15 | | | KQAPGQGLEWIGEILPGSDTTNYNEKFKDRATFTSDTSI |
| | | | NTAYMELSRLRSDDTAVYYCARDRGNYRAWFGYWGQ |
| | | | GTLVTVSS |
| | V_{L} | 527 | DIQMTQSPSSLSASVGDRVTITCRASQDISNYLNWYQQK |
| | | | PGGAVKFLIYYTSRLHSGVPSRFSGSGSGTDYTLTISSLQ |
| | | | PEDFATYFCQQGEALPWTFGGGTKVEIK |
| Antibody | V_{H} | 528 | EVQLVQSGAEVKKPGSSVKVSCKASGFTFTDYYIHWVK |
| huAD208.4.1 to | | | QAPGQGLEWIGLVYPYIGGTNYNQKFKGKATLTVDTST |
| LRRC15 | | | TTAYMEMSSLRSEDTAVYYCARGDNKYDAMDYWGQG |
| | | | TTVTVSS |
| | V_{L} | 529 | DIVLTQSPDSLAVSLGERATINCRASQSVSTSSYSYMHW |
| | | | YQQKPGQPPKLLIKYASSLESGVPDRFSGSGSGTDFTLTI |
| | | | SSLQ AEDVAVYYCEQSWEIRTFGGGTKVEIK |
| Antibody | V_{H} | 530 | EVQLVQSGAEVKKPGSSVKVSCKASGYTFTNYWMHW |
| huAD208.12.1 to | | | VKQAPGQGLEWIGMIHPNSGSTKHNEKFRGKATLTVDE |
| LRRC15 | | | STTTAYMELSSLRSEDTAVYYCARSDFGNYRWYFDVW |
| | | | GQGTTVTVSS |
| | V_{L} | 531 | EIVLTQSPATLSLSPGERATLSCRASQSSSNNLHWYQQK |
| | | | PGQAPRVLIKYVSQSISGIPARFSGSGSGTDFTLTISSLEPE |
| | | | DFA VYFCQQSNSWPFTFGQGTKLEIK |
| Antibody | V_{H} | 532 | EVQLVQSGAEVKKPGSSVKVSCKASGFTFTDYYIHWVK |
| huAD208.14.1 to | | | QAPGQGLEWIGLVYPYIGGSSYNQQFKGKATLTVDTST |
| LRRC15 | | | STAYMELSSLRSEDTAVYYCARGDNNYDAMDYWGQG |
| | | | TTVTVSS |
| | V_{L} | 533 | DIVLTQSPDSLAVSLGERATISCRASQSVSTSTYNYMHW |
| | | | YQQKPGQPPKLLVKYASNLESGVPDRFSGSGSGTDFTLT |
| | | | ISSL QAEDVAVYYCHHTWEIRTFGGGTKVEIK |
| Antibody | V_{H} | 534 | EVQLVESGGGLVQPGGSLRLSCAVSGFSLTSYGVHWVR |
| hu139.10 to | | | QATGKGLEWLGVIWAGGSTNYNSALMSRLTISKENAKS |
| LRRC15 | | | SVYLQMNSLRAGDTAMYYCATHMITEDYYGMDYWGQ |
| | | | GTTVTVSS |
| | V_{L} | 535 | DIVMTQSPDSLAVSLGERATINCKSSQSLLNSRTRKNYL |
| | | | AWYQQKPGQSPKLLIYWASTRESGVPDRFSGSGSGTDF |
| | | | TLTISS LQAEDVAVYYCKQSYNLPTFGGGTKVEIK |
| h1D11 Vh1.9 | V_{H} | 536 | EVQLVQSGAEVKKPGASVKVSCKASGVTFTSYWIGWV |
| VAR C2 | | | RQAPGQGLEWIGDIYPGGGYTNYNEKFKGRVTITRDTS |
| (VTCN1) | | | TSTAYLELSSLASEDTAVYYCARLAGSSYRGAMDSWG |
| | | | QGTLVTVSS |
| | V_{L} | 537 | DIQMTQSPSSLSASVGDRVTITCKASQGFNKYVAWYQQ |
| | | | KPGKAPKLLIYYTSTLQPGVPSRFSGSGSGRDYTLTISSL |
| | | | QPEDFATYYCLQYGDLLYAFGQGTKVEIKR |
| BNC101 | V_{H} | 538 | EVQLVQSGAEVKKPGESLRISCKGSGYSFTAYWIEWVR |
| (LRG5) | | | QAPGKGLEWIGEILPGSDSTNYNEKFKGHVTISADKSIST |
| | | | AYLQWSSLKASDTAVYYCARSGYYGSSQYWGQGTLVT |
| | | | VSS |
| | V_{L} | 539 | DIVLTQSPASLAVSPGQRATITCRASESVDSYGNSFMHW |
| | | | YQQKPGQPPKLLIYLTSNLESGVPDRFSGSGSGTDFTLTI |
| | | | NPVEANDAATYYCQQNAEDPRTFGGGTKLEIK |
| T2-6C | V_{H} | 540 | QMQLVESGGGLVQPGRSLRLSCAASGFTFDDYAIHWVR |
| (TMPRSS4) | | | QAPGKGLEWVSGISWNSEIVGYGDSVKGRFTISRDNAK |
| | | | NSLDLQMNSLRAEDTAVYYCARGSSGRAFDIWGQGTM |
| | | | VTVSS |
| | V_{L} | 541 | SGVGSDIQMTQSPSSVSASVGDRITITCRASQSISTYLNW |
| | | | YQQKPGKAPKLLIYGATSLQSGVPSKFSGSGSGTOFTLTI |
| | | | RGLQPDDFGTYYCQQSYNL1PRTFGQGTKLDIKR |
| T2-6G | V_{H} | 542 | QVQLVESGGGVVQPGRSLRLSCVGSFTFSNYGMHWVR |
| (TMPRSS4) | | | QAPGKGLQWVAVXSYDGSLKKYYADSVKGRFTISRDN |
| | | | SKNTLYLQMNSLRSEDTAVYYCARGTTMDVWGQGKG |
| | | | TTVTVSS |
| | V_{L} | 543 | SGVGSQSALTQPPSASGTPGQRVTISCSGSNSNIGSNTVN |
| | | | WYQQFPGKAPQLLIFGHNQRPSGVPDRFSGSKSGTSASL |
| | | | SISGLQSEDEAHYYCASWDDTVSGPKWVFGGGTKVDIK |
| | | | R |
| Labetuzumab | V_{H} | 544 | EVQLVESGGGVVQPGRSLRLSCSSSGFDFTTYWMSWVR |
| (CEACAM5) | | | QAPGKGLEWVAEIHPDSSTINYAPSLKDRFTISRDNSKN |
| | | | TLFLQMDSLRPEDTGVYFCASLYFGFPWFAYWGQGTPV |
| | | | TVSS |
| | V_{L} | 545 | DIQLTQSPSSLSASVGDRVTITCKASQDVGTSVAWYQQ |
| | | | KPGKAPKLLIYWTSTRHTGVPSRFSGSGSGTDFTFTISSL |
| | | | QPEDIATYYCQQYSLYRSFGQGTKVEIK |
| Ab to EPHA2 | V_{H} | 546 | EVQLLESGGGLVQPGGSLRLSCAASGFTFSHYMMAWV |
| | | | RQAPGKGLEWVSRIGPSGGPTHYADSVKGRFTISRDNSK |
| | | | NTLYLQMNSLRAEDTAVYYCAGYDSGYDYVAVAGPA |
| | | | EYFQHWGQGTLVTVSS |
| | V_{L} | 547 | DIQMTQSPSSLSASVGDRVTITCRASQSISTWLAWYQQK |
| | | | PGKAPKLLIYKASNLHTGVPSRFSGSGSGTEFSLTISGLQ |
| | | | PDDFATYYCQQYNSYSRTFGQGTKVEIK |

An antibody construct can comprise a first binding domain, or a first and second binding domain, that specifically binds a tumor antigen, wherein the first binding domain, or first and second binding domain, comprises: a) a VH sequence having at least 80% sequence identity to an amino acid sequence of SEQ ID NO: 12, and a VL sequence having at least 80% sequence identity to an amino acid sequence of SEQ ID NO: 17; b) a VH sequence having at least 80% sequence identity to an amino acid sequence of SEQ ID NO: 22, and a VL sequence having at least 80% sequence identity to an amino acid sequence of SEQ ID NO: 27; c) a VH sequence having at least 80% sequence identity to an amino acid sequence of SEQ ID NO: 32, and a VL sequence having at least 80% sequence identity to an amino acid sequence of SEQ ID NO: 37; d) a VH sequence having at least 80% sequence identity to an amino acid sequence of SEQ ID NO: 42, and a VL sequence having at least 80% sequence identity to an amino acid sequence of SEQ ID NO: 47; e) a VH sequence having at least 80% sequence identity to an amino acid sequence of SEQ ID NO: 52, and a VL sequence having at least 80% sequence identity to an amino acid sequence of SEQ ID NO: 57; f) a VH sequence having at least 80% sequence identity to an amino acid sequence of SEQ ID NO: 62, and a VL sequence having at least 80% sequence identity to an amino acid sequence of SEQ ID NO: 67; g) a VH sequence having at least 80% sequence identity to an amino acid sequence of SEQ ID NO: 72, and a VL sequence having at least 80% sequence identity to an amino acid sequence of SEQ ID NO: 77; h) a VH sequence having at least 80% sequence identity to an amino acid sequence of SEQ ID NO: 82, and a VL sequence having at least 80% sequence identity to an amino acid sequence of SEQ ID NO: 87; i) a VH sequence having at least 80% sequence identity to an amino acid sequence of SEQ ID NO: 92, and a VL sequence having at least 80% sequence identity to an amino acid sequence of SEQ ID NO: 97; j) a VH sequence having at least 80% sequence identity to an amino acid sequence of SEQ ID NO: 102, and a VL sequence having at least 80% sequence identity to an amino acid sequence of SEQ ID NO: 107; k) a VH sequence having at least 80% sequence identity to an amino acid sequence of SEQ ID NO: 112, and a VL sequence having at least 80% sequence identity to an amino acid sequence of SEQ ID NO: 117; l) a VH sequence having at least 80% sequence identity to an amino acid sequence of SEQ ID NO: 122, and a VL sequence having at least 80% sequence identity to an amino acid sequence of SEQ ID NO: 127; m) a VH sequence having at least 80% sequence identity to an amino acid sequence of SEQ ID NO: 132, and a VL sequence having at least 80% sequence identity to an amino acid sequence of SEQ ID NO: 137; n) a VH sequence having at least 80% sequence identity to an amino acid sequence of SEQ ID NO: 142, and a VL sequence having at least 80% sequence identity to an amino acid sequence of SEQ ID NO: 147; o) a VH sequence having at least 80% sequence identity to an amino acid sequence of SEQ ID NO: 152, and a VL sequence having at least 80% sequence identity to an amino acid sequence of SEQ ID NO: 157; p) a VH sequence having at least 80% sequence identity to an amino acid sequence of SEQ ID NO: 162, and a VL sequence having at least 80% sequence identity to an amino acid sequence of SEQ ID NO: 167; q) a VH sequence having at least 80% sequence identity to an amino acid sequence of SEQ ID NO: 172, and a VL sequence having at least 80% sequence identity to an amino acid sequence of SEQ ID NO: 177; r) a VH sequence having at least 80% sequence identity to an amino acid sequence of SEQ ID NO: 182, and a VL sequence having at least 80% sequence identity to an amino acid sequence of SEQ ID NO: 187; s) a VH sequence having at least 80% sequence identity to an amino acid sequence of SEQ ID NO: 192, and a VL sequence having at least 80% sequence identity to an amino acid sequence of SEQ ID NO: 197; t) a VH sequence having at least 80% sequence identity to an amino acid sequence of SEQ ID NO: 202, and a VL sequence having at least 80% sequence identity to an amino acid sequence of SEQ ID NO: 207; u) a VH sequence having at least 80% sequence identity to an amino acid sequence of SEQ ID NO: 212, and a VL sequence having at least 80% sequence identity to an amino acid sequence of SEQ ID NO: 217; v) a VH sequence having at least 80% sequence identity to an amino acid sequence of SEQ ID NO: 222, and a VL sequence having at least 80% sequence identity to an amino acid sequence of SEQ ID NO: 227; w) a VH sequence having at least 80% sequence identity to an amino acid sequence of SEQ ID NO: 232, and a VL sequence having at least 80% sequence identity to an amino acid sequence of SEQ ID NO: 237; x) a VH sequence having at least 80% sequence identity to an amino acid sequence of SEQ ID NO: 242, and a VL sequence having at least 80% sequence identity to an amino acid sequence of SEQ ID NO: 247; y) a VH sequence having at least 80% sequence identity to an amino acid sequence of SEQ ID NO: 252, and a VL sequence having at least 80% sequence identity to an amino acid sequence of SEQ ID NO: 257; z) a VH sequence having at least 80% sequence identity to an amino acid sequence of SEQ ID NO: 262, and a VL sequence having at least 80% sequence identity to an amino acid sequence of SEQ ID NO: 267; aa) a VH sequence having at least 80% sequence identity to an amino acid sequence of SEQ ID NO: 272, and a VL sequence having at least 80% sequence identity to an amino acid sequence of SEQ ID NO: 277; bb) a VH sequence having at least 80% sequence identity to an amino acid sequence of SEQ ID NO: 282, and a VL sequence having at least 80% sequence identity to an amino acid sequence of SEQ ID NO: 287; cc) a VH sequence having at least 80% sequence identity to an amino acid sequence of SEQ ID NO: 292, and a VL sequence having at least 80% sequence identity to an amino acid sequence of SEQ ID NO: 297; dd) a VH sequence having at least 80% sequence identity to an amino acid sequence of SEQ ID NO: 302, and a VL sequence having at least 80% sequence identity to an amino acid sequence of SEQ ID NO: 307; ee) a VH sequence having at least 80% sequence identity to an amino acid sequence of SEQ ID NO: 312, and a VL sequence having at least 80% sequence identity to an amino acid sequence of SEQ ID NO: 317; ff) a VH sequence having at least 80% sequence identity to an amino acid sequence of SEQ ID NO: 322, and a VL sequence having at least 80% sequence identity to an amino acid sequence of SEQ ID NO: 327; gg) a VH sequence having at least 80% sequence identity to an amino acid sequence of SEQ ID NO: 332, and a VL sequence having at least 80% sequence identity to an amino acid sequence of SEQ ID NO: 337; hh) a VH sequence having at least 80% sequence identity to an amino acid sequence of SEQ ID NO: 342, and a VL sequence having at least 80% sequence identity to an amino acid sequence of SEQ ID NO: 347; ii) a VH sequence having at least 80% sequence identity to an amino acid sequence of SEQ ID NO: 352, and a VL sequence having at least 80% sequence identity to an amino acid sequence of SEQ ID NO: 357; jj) a VH sequence having at least 80% sequence identity to an amino acid sequence of SEQ ID NO: 362, and a VL sequence having at least 80% sequence identity to an amino acid sequence of SEQ ID NO: 367; kk) a VH sequence having at least 80% sequence identity to an amino acid sequence of SEQ ID NO: 372, and a VL sequence having at least 80% sequence identity to an amino acid sequence of SEQ ID NO: 377; ll) a VH sequence having at least 80% sequence identity to an amino acid sequence of SEQ ID NO: 382, and a VL sequence having at least 80% sequence identity to an amino acid sequence of SEQ ID NO: 387; or mm) a VH sequence having at least 80% sequence identity to an amino acid sequence of SEQ ID NO: 392, and a VL sequence having at least 80% sequence identity to an amino acid sequence of SEQ ID NO: 397.

An antibody construct can comprise a first binding domain, or a first and second binding domain, that specifically binds a tumor antigen, wherein the first binding domain, or first and second binding domain, comprising a) a VH sequence having at least 80% sequence identity to an amino acid sequence of SEQ ID NO: 494, and a VL sequence having at least 80% sequence identity to an amino acid sequence of SEQ ID NO: 495; b) a VH sequence having at least 80% sequence identity to an amino acid sequence of SEQ ID NO: 496, and a VL sequence having at least 80% sequence identity to an amino acid sequence of SEQ ID NO: 497; c) a VH sequence having at least 80% sequence identity to an amino acid sequence of SEQ ID NO: 498, and a VL sequence having at least 80% sequence identity to an amino acid sequence of SEQ ID NO: 499; d) a VH sequence having at least 80% sequence identity to an amino acid sequence of SEQ ID NO: 500, and a VL sequence having at least 80% sequence identity to an amino acid sequence of SEQ ID NO: 501; e) a VH sequence having at least 80% sequence identity to an amino acid sequence of SEQ ID NO: 502, and a VL sequence having at least 80% sequence identity to an amino acid sequence of SEQ ID NO: 503; f) a VH sequence having at least 80% sequence identity to an amino acid sequence of SEQ ID NO: 504, and a VL sequence having at least 80% sequence identity to an amino acid sequence of SEQ ID NO: 505; g) a VH sequence having at least 80% sequence identity to an amino acid sequence of SEQ ID NO: 506, and a VL sequence having at least 80% sequence identity to an amino acid sequence of SEQ ID NO: 507; h) a VH sequence having at least 80% sequence identity to an amino acid sequence of SEQ ID NO: 508, and a VL sequence having at least 80% sequence identity to an amino acid sequence of SEQ ID NO: 510; i) a VH sequence having at least 80% sequence identity to an amino acid sequence of SEQ ID NO: 509, and a VL sequence having at least 80% sequence identity to an amino acid sequence of SEQ ID NO: 510; j) a VH sequence having at least 80% sequence identity to an amino acid sequence of SEQ ID NO: 511, and a VL sequence having at least 80% sequence identity to an amino acid sequence of SEQ ID NO: 512; k) a VH sequence having at least 80% sequence identity to an amino acid sequence of SEQ ID NO: 513, and a VL sequence having at least 80% sequence identity to an amino acid sequence of SEQ ID NO: 514; l) a VH sequence having at least 80% sequence identity to an amino acid sequence of SEQ ID NO: 515, and a VL sequence having at least 80% sequence identity to an amino acid sequence of SEQ ID NO: 516; m) a VH sequence having at least 80% sequence identity to an amino acid sequence of SEQ ID NO: 517, and a VL sequence having at least 80% sequence identity to an amino acid sequence of SEQ ID NO: 518; n) a VH sequence having at least 80% sequence identity to an amino acid sequence of SEQ ID NO: 519, and a VL sequence having at least 80% sequence identity to an amino acid sequence of SEQ ID NO: 520; o) a VH sequence having at least 80% sequence identity to an amino acid sequence of SEQ ID NO: 521, 522, or 523, and a VL sequence having at least 80% sequence identity to an amino acid sequence of SEQ ID NO: 524 or 525; p) a VH sequence having at least 80% sequence identity to an amino acid sequence of SEQ ID NO: 526, and a VL sequence having at least 80% sequence identity to an amino acid sequence of SEQ ID NO: 527; q) a VH sequence having at least 80% sequence identity to an amino acid sequence of SEQ ID NO: 528, and a VL sequence having at least 80% sequence identity to an amino acid sequence of SEQ ID NO: 529; r) a VH sequence having at least 80% sequence identity to an amino acid sequence of SEQ ID NO: 530, and a VL sequence having at least 80% sequence identity to an amino acid sequence of SEQ ID NO: 531; s) a VH sequence having at least 80% sequence identity to an amino acid sequence of SEQ ID NO: 532, and a VL sequence having at least 80% sequence identity to an amino acid sequence of SEQ ID NO: 533; t) a VH sequence having at least 80% sequence identity to an amino acid sequence of SEQ ID NO: 534, and a VL sequence having at least 80% sequence identity to an amino acid sequence of SEQ ID NO: 535; u) a VH sequence having at least 80% sequence identity to an amino acid sequence of SEQ ID NO: 536, and a VL sequence having at least 80% sequence identity to an amino acid sequence of SEQ ID NO: 537; v) a VH sequence having at least 80% sequence identity to an amino acid sequence of SEQ ID NO: 538, and a VL sequence having at least 80% sequence identity to an amino acid sequence of SEQ ID NO: 539; w) a VH sequence having at least 80% sequence identity to an amino acid sequence of SEQ ID NO: 540, and a VL sequence having at least 80% sequence identity to an amino acid sequence of SEQ ID NO: 541; x) a VH sequence having at least 80% sequence identity to an amino acid sequence of SEQ ID NO: 542, and a VL sequence having at least 80% sequence identity to an amino acid sequence of SEQ ID NO: 543; y) a VH sequence having at least 80% sequence identity to an amino acid sequence of SEQ ID NO: 544, and a VL sequence having at least 80% sequence identity to an amino acid sequence of SEQ ID NO: 545; or z) a VH sequence having at least 80% sequence identity to an amino acid sequence of SEQ ID NO: 546, and a VL sequence having at least 80% sequence identity to an amino acid sequence of SEQ ID NO: 547.

An antibody construct of a conjugate can comprise a first binding domain, a second binding domain and an Fc domain, wherein the first and second binding domains and the Fc domain comprise an antibody. The first binding domain and the second binding domain can bind to a tumor antigen. Such an antibody construct can comprise an antibody light chain. An antibody construct can comprise a light chain comprising a light chain sequence in TABLE 3. An antibody construct can comprise a light chain comprising a sequence having at least 80% sequence identity to a light chain sequence in TABLE 3. An antibody construct can comprise an antibody heavy chain. An antibody construct can comprise a heavy chain comprising a heavy chain sequence in TABLE 3. An antibody construct can comprise a heavy chain comprising a sequence having at least 80% sequence identity (or at least 90%, 95%, or 100% sequence identity) to any heavy chain sequence in Table 3. An antibody construct can comprise a pair of heavy and light chains having sequences having at least 80% sequence identity (or at least 90%, 95%, or 100% sequence identity) to any pair of sequences in TABLE 3. An antibody construct can comprise a pair of heavy and light chains having sequences set forth in TABLE 3.

TABLE 3
Tumor Antibody Heavy Chain and Light Chain sequences

| Antibody | Region | SEQ ID NO: | Sequence |
|---|---|---|---|
| Pertuzumab | Heavy | 11 | EVQLVESGGGLVQPGGSLRLSCAASGFTFTDYTMDWV |
| | Chain | | RQAPGKGLEWVADVNPNSGGSIYNQRFKGRFTLSVDRS |
| | | | KNTLYLQMNSLRAEDTAVYYCARNLGPSFYFDYWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKKVEPKSCDKTHTCP |
| | | | PCPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 16 | DIQMTQSPSSLSASVGDRVTITCKASQDVSIGVAWYQQ |
| | Chain | | KPGKAPKLLIYSASYRYTGVPSRFSGSGSGTDFTLTISSL |
| | | | QPEDFATYYCQQYYIYPYTFGQGTKVEIKRTVAAPSVFI |
| | | | FPPSDEQLKSGTASVVCLLNNFYPREAKVQWKVDNALQ |
| | | | SGNSQESVTEQDSKDSTYSLSSTLTLSKADYEKHKVYA |
| | | | CEVTHQGLSSPVTKSFNRGEC |
| Cetuximab | Heavy | 21 | QVQLKQSGPGLVQPSQSLSITCTVSGFSLTNYGVHWVR |
| | Chain | | QSPGKGLEWLGVIWSGGNTDYNTPFTSRLSINKDNSKS |
| | | | QVFFKMNSLQSNDTAIYYCARALTYYDYEFAYWGQGT |
| | | | LVTVSAASTKGPSVFPLAPSSKSTSGGTAALGCLVKDYF |
| | | | PEPVTVSWNSGALTSGVHTFPAVLQSSGLYSLSSVVTVP |
| | | | SSSLGTQTYICNVNHKPSNTKVDKKVEPKSCDKTHTCPP |
| | | | CPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSRDELTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 26 | DILLTQSPVILSVSPGERVSFSCRASQSIGTNIHWYQQRT |
| | Chain | | NGSPRLLIKYASESISGIPSRFSGSGSGTDFTLSINSVESED |
| | | | IADYYCQQNNNWPTTFGAGTKLELKRTVAAPSVFIFPPS |
| | | | DEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| Panitumumab | Heavy | 31 | QVQLQESGPGLVKPSETLSLTCTVSGGSVSSGDYYWTW |
| | Chain | | IRQSPGKGLEWIGHIYYSGNTNYNPSLKSRLTISIDTSKT |
| | | | QFSLKLSSVTAADTAIYYCVRDRVTGAFDIWGQGTMVT |
| | | | VSSASTKGPSVFPLAPCSRSTSESTAALGCLVKDYFPEPV |
| | | | TVSWNSGALTSGVHTFPAVLQSSGLYSLSSVVTVPSSNF |
| | | | GTQTYTCNVDHKPSNTKVDKTVERKCCVECPPCPAPPV |
| | | | AGPSVFLFPPKPKDTLMISRTPEVTCVVVDVSHEDPEVQ |
| | | | FNWYVDGVEVHNAKTKPREEQFNSTFRVVSVLTVVHQ |
| | | | DWLNGKEYKCKVSNKGLPAPIEKTISKTKGQPREPQVY |
| | | | TLPPSREEMTKNQVSLTCLVKGFYPSDIAVEWESNGQPE |
| | | | NNYKTTPPMLDSDGSFFLYSKLTVDKSRWQQGNVFSCS |
| | | | VMHEALHNHYTQKSLSLSPGK |
| | Light | 36 | DIQMTQSPSSLSASVGDRVTITCQASQDISNYLNWYQQK |
| | Chain | | PGKAPKLLIYDASNLETGVPSRFSGSGSGTDFTFTISSLQP |
| | | | EDIATYFCQHFDHLPLAFGGGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACE |
| | | | VTHQGLSSPVTKSFNRGEC |
| Nimotuzumab | Heavy | 41 | QVQLQQSGAEVKKPGSSVKVSCKASGYTFTNYYIYWV |
| | Chain | | RQAPGQGLEWIGGINPTSGGSNFNEKFKTRVTITVDEST |
| | | | NTAYMELSSLRSEDTAFYFCARQGLWFDSDGRGFDFW |
| | | | GQGSTVTVSSASTKGPSVFPLAPSSKSTSGGTAALGCLV |
| | | | KDYFPEPVTVSWNSGALTSGVHTFPAVLQSSGLYSLSSV |
| | | | VTVPSSSLGTQTYICNVNHKPSNTKVDKKVEPKSCDKT |
| | | | HTCPPCPAPELLGGPSVFLFPPKPKDTLMISRTPEVTCVV |
| | | | VDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| | Light | 46 | DIQMTQSPSSLSASVGDRVTITCRSSQNIVHSNGNTYLD |
| | Chain | | WYQQTPGKAPKLLIYKVSNRFSGVPSRFSGSGSGTDFTF |
| | | | TISSLQPEDIATYYCFQYSHVPWTFGQGTKLEIKRTVAA |
| | | | PSVFIFPPSDEQLKSGTASVVCLLNNFYPREAKVQWKVD |
| | | | NALQSGNSQESVTEQDSKDSTYSLSSTLTLSKADYEKHK |
| | | | VYACEVTHQGLSSPVTKSFNRGEC |
| Zalutumumab | Heavy | 51 | QVQLVESGGGVVQPGRSLRLSCAASGFTFSTYGMHWV |
| | Chain | | RQAPGKGLEWVAVIWDDGSYKYYGDSVKGRFTISRDN |
| | | | SKNTLYLQMNSLRAEDTAVYYCARDGITMVRGVMKD |
| | | | YFDYWGQGTLVTVSSASTKGPSVFPLAPSSKSTSGGTAA |
| | | | LGCLVKDYFPEPVTVSWNSGALTSGVHTFPAVLQSSGL |
| | | | YSLSSVVTVPSSSLGTQTYICNVNHKPSNTKVDKKVEPK |
| | | | SCDKTHTCPPCPAPELLGGPSVFLFPPKPKDTLMISRTPE |
| | | | VTCVVVDVSHEDPEVKFNWYVDGVEVHNAKTKPREEQ |
| | | | YNSTYRVVSVLTVLHQDWLNGKEYKCKVSNKALPAPIE |
| | | | KTISKAKGQPREPQVYTLPPSREEMTKNQVSLTCLVKGF |
| | | | YPSDIAVEWESNGQPENNYKTTPPVLDSDGSFFLYSKLT |
| | | | VDKSRWQQGNVFSCSVMHEALHNHYTQKSLSLSPGK |
| | Light | 56 | AIQLTQSPSSLSASVGDRVTITCRASQDISSALVWYQQKP |
| | Chain | | GKAPKLLIYDASSLESGVPSRFSGSESGTDFTLTISSLQPE |
| | | | DFATYYCQQFNSYPLTFGGGTKVEIKRTVAAPSVFIFPPS |
| | | | DEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| Onartuzumab | Heavy | 61 | EVQLVESGGGLVQPGGSLRLSCAASGYTFTSYWLHWV |
| | Chain | | RQAPGKGLEWVGMIDPSNSDTRFNPNFKDRFTISADTSK |
| | | | NTAYLQMNSLRAEDTAVYYCATYRSYVTPLDYWGQGT |
| | | | LVTVSSASTKGPSVFPLAPSSKSTSGGTAALGCLVKDYF |
| | | | PEPVTVSWNSGALTSGVHTFPAVLQSSGLYSLSSVVTVP |
| | | | SSSLGTQTYICNVNHKPSNTKVDKKVEPKSCDKTHTCPP |
| | | | CPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 66 | DIQMTQSPSSLSASVGDRVTITCKSSQSLLYTSSQKNYLA |
| | Chain | | WYQQKPGKAPKLLIYWASTRESGVPSRFSGSGSGTDFT |
| | | | LTISSLQPEDFATYYCQQYYAYPWTFGQGTKVEIKRTV |
| | | | AAPSVFIFPPSDEQLKSGTASVVCLLNNFYPREAKVQWK |
| | | | VDNALQSGNSQESVTEQDSKDSTYSLSSTLTLSKADYEK |
| | | | HKVYACEVTHQGLSSPVTKSFNRGEC |
| Patritumab | Heavy | 71 | QVQLQQWGAGLLKPSETLSLTCAVYGGSFSGYYWSWI |
| | Chain | | RQPPGKGLEWIGEINHSGSTNYNPSLKSRVTISVETSKNQ |
| | | | FSLKLSSVTAADTAVYYCARDKWTWYFDLWGRGTLVT |
| | | | VSSASTKGPSVFPLAPSSKSTSGGTAALGCLVKDYFPEP |
| | | | VTVSWNSGALTSGVHTFPAVLQSSGLYSLSSVVTVPSSS |
| | | | LGTQTYICNVNHKPSNTKVDKKVEPKSCDKTHTCPPCP |
| | | | APELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 76 | DIEMTQSPDSLAVSLGERATINCRSSQSVLYSSSNRNYL |
| | Chain | | AWYQQNPGQPPKLLIYWASTRESGVPDRFSGSGSGTDF |
| | | | TLTISSLQAEDVAVYYCQQYYSTPRTFGQGTKVEIKRTV |
| | | | AAPSVFIFPPSDEQLKSGTASVVCLLNNFYPREAKVQWK |
| | | | VDNALQSGNSQESVTEQDSKDSTYSLSSTLTLSKADYEK |
| | | | HKVYACEVTHQGLSSPVTKSFNRGEC |
| Clivatuzumab | Heavy | 81 | QVQLQQSGAEVKKPGASVKVSCEASGYTFPSYVLHWV |
| | Chain | | KQAPGQGLEWIGYINPYNDGTQYNEKFKGKATLTRDTS |
| | | | INTAYMELSRLRSDDTAVYYCARGFGGSYGFAYWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKKVEPKSCDKTHTCP |
| | | | PCPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 86 | DIQLTQSPSSLSASVGDRVTMTCSASSSVSSSYLYWYQQ |
| | Chain | | KPGKAPKLWIYSTSNLASGVPARFSGSGSGTDFTLTISSL |
| | | | QPEDSASYFCHQWNRYPYTFGGGTRLEIKRTVAAPSVFI |
| | | | FPPSDEQLKSGTASVVCLLNNFYPREAKVQWKVDNALQ |
| | | | SGNSQESVTEQDSKDSTYSLSSTLTLSKADYEKHKVYA |
| | | | CEVTHQGLSSPVTKSFNRGEC |
| Sofituzumab | Heavy | 91 | EVQLVESGGGLVQPGGSLRLSCAASGYSITNDYAWNW |
| | Chain | | VRQAPGKGLEWVGYISYSGYTTYNPSLKSRFTISRDTSK |
| | | | NTLYLQMNSLRAEDTAVYYCARWTSGLDYWGQGTLV |
| | | | TVSSASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPCP |
| | | | APELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 96 | DIQMTQSPSSLSASVGDRVTITCKASDLIHNWLAWYQQ |
| | Chain | | KPGKAPKLLIYGATSLETGVPSRFSGSGSGTDFTLTISSL |
| | | | QPEDFATYYCQQYWTTPFTFGQGTKVEIKRTVAAPSVFI |
| | | | FPPSDEQLKSGTASVVCLLNNFYPREAKVQWKVDNALQ |
| | | | SGNSQESVTEQDSKDSTYSLSSTLTLSKADYEKHKVYA |
| | | | CEVTHQGLSSPVTKSFNRGEC |
| Edrecolomab | Heavy | 101 | QVQLQQSGAELVRPGTSVKVSCKASGYAFTNYLIEWVK |
| | Chain | | QRPGQGLEWIGVINPGSGGTNYNEKFKGKATLTADKSS |
| | | | STAYMQLSSLTSDDSAVYFCARDGPWFAYWGQGTLVT |
| | | | VSAASTKGPSVFPLAPSSKSTSGGTAALGCLVKDYFPEP |
| | | | VTVSWNSGALTSGVHTFPAVLQSSGLYSLSSVVTVPSSS |
| | | | LGTQTYICNVNHKPSNTKVDKKVEPKSCDKTHTCPPCP |
| | | | APELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 106 | NIVMTQSPKSMSMSVGERVTLTCKASENVVTYVSWYQ |
| | Chain | | QKPEQSPKLLIYGASNRYTGVPDRFTGSGSATDFTLTISS |
| | | | VQAEDLADYHCGQGYSYPYTFGGGTKLEIKRTVAAPSV |
| | | | FIFPPSDEQLKSGTASVVCLLNNFYPREAKVQWKVDNA |
| | | | LQSGNSQESVIEQDSKDSTYSLSSTLTLSKADYEKHKVY |
| | | | ACEVTHQGLSSPVTKSFNRGEC |
| Adecatumumab | Heavy | 111 | EVQLLESGGGVVQPGRSLRLSCAASGFTFSSYGMHWVR |
| | Chain | | QAPGKGLEWVAVISYDGSNKYYADSVKGRFTISRDNSK |
| | | | NTLYLQMNSLRAEDTAVYYCAKDMGWGSGWRPYYYY |
| | | | GMDVWGQGTTVTVSSASTKGPSVFPLAPSSKSTSGGTA |
| | | | ALGCLVKDYFPEPVTVSWNSGALTSGVHTFPAVLQSSG |
| | | | LYSLSSVVTVPSSSLGTQTYICNVNHKPSNTKVDKKVEP |
| | | | KSCDKTHTCPPCPAPELLGGPSVFLFPPKPKDTLMISRTP |
| | | | EVTCVVVDVSHEDPEVKFNWYVDGVEVHNAKTKPREE |
| | | | QYNSTYRVVSVLTVLHQDWLNGKEYKCKVSNKALPAP |
| | | | IEKTISKAKGQPREPQVYTLPPSRDELTKNQVSLTCLVK |
| | | | GFYPSDIAVEWESNGQPENNYKTTPPVLDSDGSFFLYSK |
| | | | LTVDKSRWQQGNVFSCSVMHEALHNHYTQKSLSLSPG |
| | | | K |
| | Light | 116 | ELQMTQSPSS LSASVGDRVT ITCRTSQSIS |
| | Chain | | SYLNWYQQKP GQPPKLLIYW ASTRESGVPD |
| | | | RFSGSGSGTD FTLTISSLQP EDSATYYCQQ |
| | | | SYDIPYTFGQ GTKLEIKRTV AAPSVFIFPP |
| | | | SDEQLKSGTA SVVCLLNNFY PREAKVQWKV |
| | | | DNALQSGNSQ ESVTEQDSKD STYSLSSTLT |
| | | | LSKADYEKHK VYACEVTHQG LSSPVTKSFN RGEC |
| Anetumab | Heavy | 121 | QVELVQSGAEVKKPGESLKISCKGSGYSFTSYWIGWVR |
| | Chain | | QAPGKGLEWMGIIDPGDSRTRYSPSFQGQVTISADKSIST |
| | | | AYLQWSSLKASDTAMYYCARGQLYGGTYMDGWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKKVEPKSCDKTHTCP |
| | | | PCPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSRDELTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 126 | DIALTQPASVSGSPGQSITISCTGTSSDIGGYNSVSWYQQ |
| | Chain | | HPGKAPKLMIYGVNNRPSGVSNRFSGSKSGNTASLTISG |
| | | | LQAEDEADYYCSSYDIESATPVFGGGTKLTVLGQPKAA |
| | | | PSVTLFPPSSEELQANKATLVCLISDFYPGAVTVAWKGD |
| | | | SSPVKAGVETTTPSKQSNNKYAASSYLSLTPEQWKSHRS |
| | | | YSCQVTHEGSTVEKT VAPTECS |
| huDS6 | Heavy | 131 | QAQLVQSGAEVVKPGASVKMSCKASGYTFTSYNMHW |
| | Chain | | VKQTPGQGLEWIGYIYPGNGATNYNQKFQGKATLTADP |
| | | | SSSTAYMQISSLTSEDSAVYFCARGDSVPFAYWGQGTL |
| | | | VTVSAASTKGPSVFPLAPSSKSTSGGTAALGCLVKDYFP |
| | | | EPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCWVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSRDELTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 136 | EIVLTQSPATMSASPGERVTITCSAHSSVSFMHWFQQKP |
| | Chain | | GTSPKLWIYSTSSLASGVPARFGGSGSGTSYSLTISSMEA |
| | | | EDAATYYCQQRSSFPLTFGAGTKLELKRTVAAPSVFIFP |
| | | | PSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| Lifastuzumab | Heavy | 141 | EVQLVESGGGLVQPGGSLRLSCAASGFSFSDFAMSWVR |
| | Chain | | QAPGKGLEWVATIGRVAFHTYYPDSMKGRFTISRDNSK |
| | | | NTLYLQMNSLRAEDTAVYYCARHRGFDVGHFDFWGQ |
| | | | GTLVTVSSASTKGPSVFPLAPSSKSTSGGTAALGCLVKD |
| | | | YFPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVVVD |
| | | | VSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| | Light | 146 | DIQMTQSPSSLSASVGDRVTITCRSSETLVHSSGNTYLE |
| | Chain | | WYQQKPGKAPKLLIYRVSNRFSGVPSRFSGSGSGTDFTL |
| | | | TISSLQPEDFATYYCFQGSFNPLTFGQGTKVEIKRTVAAP |
| | | | SVFIFPPSDEQLKSGTASVVCLLNNFYPREAKVQWKVD |
| | | | NALQSGNSQESVTEQDSKDSTYSLSSTLTLSKADYEKHK |
| | | | VYACEVTHQGLSSPVTKSFNRGEC |
| Sacituzumab | Heavy | 151 | QVQLQQSGSELKKPGASVKVSCKASGYTFTNYGMNWV |
| | Chain | | KQAPGQGLKWMGWINTYTGEPTYTDDFKGRFAFSLDT |
| | | | SVSTAYLQISSLKADDTAVYFCARGGFGSSYWYFDVWG |
| | | | QGSLVTVSSASTKGPSVFPLAPSSKSTSGGTAALGCLVK |
| | | | DYFPEPVTVSWNSGALTSGVHTFPAVLQSSGLYSLSSVV |
| | | | TVPSSSLGTQTYICNVNHKPSNTKVDKRVEPKSCDKTHT |
| | | | CPPCPAPELLGGPSVFLFPPKPKDTLMISRTPEVTCVVVD |
| | | | VSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| | Light | 156 | DIQLTQSPSS LSASVGDRVS ITCKASQDVS |
| | Chain | | IAVAWYQQKP GKAPKLLIYS ASYRYTGVPD |
| | | | RFSGSGSGTD FTLTISSLQP EDFAVYYCQQ |
| | | | HYITPLTFGA GTKVEIKRTV AAPSVFIFPP |
| | | | SDEQLKSGTA SVVCLLNNFY PREAKVQWKV |
| | | | DNALQSGNSQ ESVTEQDSKD STYSLSSTLT |
| | | | LSKADYEKHK VYACEVTHQG LSSPVTKSFN RGEC |
| PR1A3 | Heavy | 161 | QVKLQQSGPELKKPGETVKISCKASGYTFTEFGMNWVK |
| | Chain | | QAPGKGLKWMGWINTKTGEATYVEEFKGRFAFSLETS |
| | | | ATTAYLQINNLKNEDTAKYFCARWDFYDYVEAMDYW |
| | | | GQGTTVTVSSASTKGPSVFPLAPSSKSTSGGTAALGCLV |
| | | | KDYFPEPVTVSWNSGALTSGVHTFPAVLQSSGLYSLSSV |
| | | | VTVPSSSLGTQTYICNVNHKPSNTKVDKKVEPKSCDKT |
| | | | HTCPPCPAPELLGGPSVFLFPPKPKDTLMISRTPEVTCVV |
| | | | VDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSRDELTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| | Light | 166 | DIVMTQSQRFMSTSVGDRVSVTCKASQNVGTNVAWYQ |
| | Chain | | QKPGQSPKALIYSASYRYSGVPDRFTGSGSGTDFTLTISN |
| | | | VQSEDLAEYFCHQYYTYPLFTFGSGTKLEMKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHKV |
| | | | YACEVTHQGLSSPVTKSFNRGEC |
| Humanized | Heavy | 171 | QVQLVQSGAEVKKPGASVKVSCKASGYTFTEFGMNWV |
| PR1A3 | Chain | | RQAPGQGLEWMGWINTKTGEATYVEEFKGRVTFTTDT |
| | | | STSTAYMELRSLRSDDTAVYYCARWDFAYYVEAMDY |
| | | | WGQGTTVTVSSASTKGPSVFPLAPSSKSTSGGTAALGCL |
| | | | VKDYFPEPVTVSWNSGALTSGVHTFPAVLQSSGLYSLSS |
| | | | VVTVPSSSLGTQTYICNVNHKPSNTKVDKKVEPKSCDK |
| | | | THTCPPCPAPELLGGPSVFLFPPKPKDTLMISRTPEVTCV |
| | | | VVDVSHEDPEVKFNWYVDGVEVHNAKTKPREEQYNST |
| | | | YRVVSVLTVLHQDWLNGKEYKCKVSNKALPAPIEKTIS |
| | | | KAKGQPREPQVYTLPPSREEMTKNQVSLTCLVKGFYPS |
| | | | DIAVEWESNGQPENNYKTTPPVLDSDGSFFLYSKLTVD |
| | | | KSRWQQGNVFSCSVMHEALHNHYTQKSLSLSPGK |
| | Light | 176 | DIQMTQSPSSLSASVGDRVTITCKASAAVGTYVAWYQQ |
| | Chain | | KPGKAPKLLIYSASYRKRGVPSRFSGSGSGTDFTLTISSL |
| | | | QPEDFATYYCHQYYTYPLFTFGQGTKLEIKRTVAAPSVF |
| | | | IFPPSDEQLKSGTASVVCLLNNFYPREAKVQWKVDNAL |
| | | | QSGNSQESVTEQDSKDSTYSLSSTLTLSKADYEKHKVY |
| | | | ACEVTHQGLSSPVTKSFNRGEC |
| Humanized Ab2-3 | Heavy | 181 | EVQLQESGPGLVKPGGSLSLSCAASGFVFSSYDMSWVR |
| | Chain | | QTPERGLEWVAYISSGGGITYAPSTVKGRFTVSRDNAK |
| | | | NTLYLQMNSLTSEDTAVYYCAAHYFGSSGPFAYWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKKVEPKSCDKTHTCP |
| | | | PCPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 186 | DIQMTQSPASLSASVGDRVTITCRASENIFSYLAWYQQK |
| | Chain | | PGKSPKLLVYNTRTLAEGVPSRFSGSGSGTDFSLTISSLQ |
| | | | PEDFATYYCQHHYGTPFTFGSGTKLEIKRTVAAPSVFIFP |
| | | | PSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| IMAB362, | Heavy | 191 | QVQLQQPGAELVRPGASVKLSCKASGYTFTSYWINWV |
| CLAUDIXIMAB | Chain | | KQRPGQGLEWIGNIYPSDSYTNYNQKFKDKATLTVDKS |
| | | | SSTAYMQLSSPTSEDSAVYYCTRSWRGNSFDYWGQGTT |
| | | | LTVSSASTKGPSVFPLAPSSKSTSGGTAALGCLVKDYFP |
| | | | EPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 196 | DIVMTQSPSSLTVTAGEKVTMSCKSSQSLLNSGNQKNY |
| | Chain | | LTWYQQKPGQPPKLLIYWASTRESGVPDRFTGSGSGTD |
| | | | FTLTISSVQAEDLAVYYCQNDYSYPFTFGSGTKLEIKRT |
| | | | VAAPSVFIFPPSDEQLKSGTASVVCLLNNFYPREAKVQW |
| | | | KVDNALQSGNSQESVTEQDSKDSTYSLSSTLTLSKADYE |
| | | | KHKVYACEVTHQGLSSPVTKSFNRGEC |
| AMG595 | Heavy | 201 | QVQLVESGGGVVQSGRSLRLSCAASGFTFRNYGMHWV |
| | Chain | | RQAPGKGLEWVAVIWYDGSDKYYADSVRGRFTISRDN |
| | | | SKNTLYLQMNSLRAEDTAVYYCARDGYDILTGNPRDFD |
| | | | YWGQGTLVTVSSASTKGPSVFPLAPSSKSTSGGTAALGC |
| | | | LVKDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEVTC |
| | | | VVVDVSHEDPEVKFNWYVDGVEVHNAKTKPREEQYNS |
| | | | TYRVVSVLTVLHQDWLNGKEYKCKVSNKALPAPIEKTI |
| | | | SKAKGQPREPQVYTLPPSREEMTKNQVSLTCLVKGFYP |
| | | | SDIAVEWESNGQPENNYKTTPPVLDSDGSFFLYSKLTVD |
| | | | KSRWQQGNVFSCSVMHEALHNHYTQKSLSLSPGK |
| | Light | 206 | DTVMTQTPLSSHVTLGQPASISCRSSQSLVHSDGNTYLS |
| | Chain | | WLQQRPGQPPRLLIYRISRRFSGVPDRFSGSGAGTDFTLE |
| | | | ISRVEAEDVGVYYCMQSTHVPRTFGQGTKVEIKRTVAA |
| | | | PSVFIFPPSDEQLKSGTASVVCLLNNFYPREAKVQWKVD |
| | | | NALQSGNSQESVTEQDSKDSTYSLSSTLTLSKADYEKHK |
| | | | VYACEVTHQGLSSPVTKSFNRGEC |
| ABT806 | Heavy | 211 | EVQLQESGPGLVKPSQTLSLTCTVSGYSISRDFAWNWIR |
| | Chain | | QPPGKGLEWMGYISYNGNTRYQPSLKSRITISRDTSKNQ |
| | | | FFLKLNSVTAADTATYYCVTASRGFPYWGQGTLVTVSS |
| | | | ASTKGPSVFPLAPSSKSTSGGTAALGCLVKDYFPEPVTV |
| | | | SWNSGALTSGVHTFPAVLQSSGLYSLSSVVTVPSSSLGT |
| | | | QTYICNVNHKPSNTKVDKKVEPKSCDKTHTCPPCPAPEL |
| | | | LGGPSVFLFPPKPKDTLMISRTPEVTCVVVDVSHEDPEV |
| | | | KFNWYVDGVEVHNAKTKPREEQYNSTYRVVSVLTVLH |
| | | | QDWLNGKEYKCKVSNKALPAPIEKTISKAKGQPREPQV |
| | | | YTLPPSREEMTKNQVSLTCLVKGFYPSDIAVEWESNGQP |
| | | | ENNYKTTPPVLDSDGSFFLYSKLTVDKSRWQQGNVFSC |
| | | | SVMHEALHNHYTQKSLSLSPGK |
| | Light | 216 | DIQMTQSPSSMSVSVGDRVTITCHSSQDINSNIGWLQQK |
| | Chain | | PGKSFKGLIYHGTNLDDGVPSRFSGSGSGTDYTLTISSLQ |
| | | | PEDFATYYCVQYAQFPWTFGGGTKLEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| Sibrotuzumab | Heavy | 221 | QVQLVQSGAEVKKPGASVKVSCKTSRYTFILYTIHWVR |
| | Chain | | QAPGQRLEWIGGINPNNGIPNYNQKFKGRVTITVDTSAS |
| | | | TAYMELSSLRSEDTAVYYCARRRIAYGYDEGHAMDYW |
| | | | GQGTLVTVSSASTKGPSVFPLAPSSKSTSGGTAALGCLV |
| | | | KDYFPEPVTVSWNSGALTSGVHTFPAVLQSSGLYSLSSV |
| | | | VTVPSSSLGTQTYICNVNHKPSNTKVDKKVEPKSCDKT |
| | | | HTCPPCPAPELLGGPSVFLFPPKPKDTLMISRTPEVTCVV |
| | | | VDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| | Light | 226 | DIVMTQSPDSLAVSLGERATINCKSSQSLLYSRNQKNYL |
| | Chain | | AWYQQKPGQPPKLLIFWASTRESGVPDRFSGSGFGTDFT |
| | | | LTISSLQAEDVAVYYCQQYFSYPLTFGQGTKVEIKRTVA |
| | | | APSVFIFPPSDEQLKSGTASVVCLLNNFYPREAKVQWKV |
| | | | DNALQSGNSQESVTEQDSKDSTYSLSSTLTLSKADYEKH |
| | | | KVYACEVTHQGLSSPVTKSFNRGEC |
| DS-8895a variant | Heavy | 231 | QVQLVQSGAEVKKPGASVKVSCKASGYTFIDYSMHWV |
| 1 | Chain | | RQAPGQGLEWMGWINTYTGEPTYSDDFKGRVTITADTS |
| | | | TSTAYLELSSLRSEDTAVYYCATYYRYERDFDYWGQGT |
| | | | LVTVSSASTKGPSVFPLAPSSKSTSGGTAALGCLVKDYF |
| | | | PEPVTVSWNSGALTSGVHTFPAVLQSSGLYSLSSVVTVP |
| | | | SSSLGTQTYICNVNHKPSNTKVDKKVEPKSCDKTHTCPP |
| | | | CPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 236 | DIVMTQSPLSLPVTPGEPASISCRSSQSIVHSSGITYLEWY |
| | Chain | | LQKPGQSPQLLIYKVSNRFSGVPDRFSGSGSGTDFTLKIS |
| | | | RVEAEDVGVYYCFQGSHVPYTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHKV |
| | | | YACEVTHQGLSSPVTKSFNRGEC |
| DS-8895a variant | Heavy | 241 | QIQLVQSGAEVKKPGASVKVSCKASGYTFIDYSMHWVR |
| 2 | Chain | | QAPGQGLKWMGWINTYTGEPTYSDDFKGRFAFSLDTST |
| | | | STAYLELSSLRSEDTAVYYCATYYRYERDFDYWGQGTL |
| | | | VTVSSASTKGPSVFPLAPSSKSTSGGTAALGCLVKDYFP |
| | | | EPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 246 | DVLMTQSPLSLPVTPGEPASISCRSSQSIVHSSGITYLEW |
| | Chain | | YLQKPGQSPQLLIYKVSNRFSGVPDRFSGSGSGTDFTLKI |
| | | | SRVEAEDVGVYYCFQGSHVPYTFGQGTKVEIKRTVAAP |
| | | | SVFIFPPSDEQLKSGTASVVCLLNNFYPREAKVQWKVD |
| | | | NALQSGNSQESVTEQDSKDSTYSLSSTLTLSKADYEKHK |
| | | | VYACEVTHQGLSSPVTKSFNRGEC |
| MEDI-547 | Heavy | 251 | EVQLLESGGGLVQPGGSLRLSCAASGFTFSHYMMAWV |
| | Chain | | RQAPGKGLEWVSRIGPSGGPTHYADSVKGRFTISRDNSK |
| | | | NTLYLQMNSLRAEDTAVYYCAGYDSGYDYVAVAGPA |
| | | | EYFQHWGQGTLVTVSSASTKGPSVFPLAPSSKSTSGGTA |
| | | | ALGCLVKDYFPEPVTVSWNSGALTSGVHTFPAVLQSSG |
| | | | LYSLSSVVTVPSSSLGTQTYICNVNHKPSNTKVDKKVEP |
| | | | KSCDKTHTCPPCPAPELLGGPSVFLFPPKPKDTLMISRTP |
| | | | EVTCVVVDVSHEDPEVKFNWYVDGVEVHNAKTKPREE |
| | | | QYNSTYRVVSVLTVLHQDWLNGKEYKCKVSNKALPAP |
| | | | IEKTISKAKGQPREPQVYTLPPSREEMTKNQVSLTCLVK |
| | | | GFYPSDIAVEWESNGQPENNYKTTPPVLDSDGSFFLYSK |
| | | | LTVDKSRWQQGNVFSCSVMHEALHNHYTQKSLSLSPG |
| | | | K |
| | Light | 256 | DIQMTQSPSSLSASVGDRVTITCRASQSISTWLAWYQQK |
| | Chain | | PGKAPKLLIYKASNLHTGVPSRFSGSGSGTEFSLTISGLQ |
| | | | PDDFATYYCQQYNSYSRTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| Narnatumab | Heavy | 261 | EVQLVESGGGLVQPGGSLRLSCAASGFTFSSYLMTWVR |
| | Chain | | QAPGKGLEWVANIKQDGSEKYYVDSVKGRFTISRDNA |
| | | | KNSLNLQMNSLRAEDTAVYYCTRDGYSSGRHYGMDV |
| | | | WGQGTTVIVSSASTKGPSVFPLAPSSKSTSGGTAALGCL |
| | | | VKDYFPEPVTVSWNSGALTSGVHTFPAVLQSSGLYSLSS |
| | | | VVTVPSSSLGTQTYICNVNHKPSNTKVDKRVEPKSCDK |
| | | | THTCPPCPAPELLGGPSVFLFPPKPKDTLMISRTPEVTCV |
| | | | VVDVSHEDPEVKFNWYVDGVEVHNAKTKPREEQYNST |
| | | | YRVVSVLTVLHQDWLNGKEYKCKVSNKALPAPIEKTIS |
| | | | KAKGQPREPQVYTLPPSREEMTKNQVSLTCLVKGFYPS |
| | | | DIAVEWESNGQPENNYKTTPPVLDSDGSFFLYSKLTVD |
| | | | KSRWQQGNVFSCSVMHEALHNHYTQKSLSLSPGK |
| | Light | 266 | EIVLTQSPATLSLSPGERATLSCRASQSVSRYLAWYQQK |
| | Chain | | PGQAPRLLIYDASNRATGIPARFSGSGSGTDFTLTISSLEP |
| | | | EDFAVYYCQQRSNWPRTFGQGTKVEIKRTVAAPSVFIFP |
| | | | PSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| RG7841 | Heavy | 271 | EVQLVESGPALVKPTQTLTLTCTVSGFSLTGYSVNWIRQ |
| | Chain | | PPGKALEWLGMIWGDGSTDYNSALKSRLTISKDTSKNQ |
| | | | VVLTMTNMDPVDTATYYCARDYYFNYASWFAYWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKKVEPKSCDKTHTCP |
| | | | PCPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 276 | DIQMTQSPSSLSASVGDRVTITCSASQGISNYLNWYQQK |
| | Chain | | PGKTVKLLIYYTSNLHSGVPSRFSGSGSGTDYTLTISSLQ |
| | | | PEDFATYYCQQYSELPWTFGQGTKVEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| Farletuzumab | Heavy | 281 | EVQLVESGGGVVQPGRSLRLSCSASGFTFSGYGLSWVR |
| | Chain | | QAPGKGLEWVAMISSGGSYTYYADSVKGRFAISRDNAK |
| | | | NTLFLQMDSLRPEDTGVYFCARHGDDPAWFAYWGQGT |
| | | | PVTVSSASTKGPSVFPLAPSSKSTSGGTAALGCLVKDYF |
| | | | PEPVTVSWNSGALTSGVHTFPAVLQSSGLYSLSSVVTVP |
| | | | SSSLGTQTYICNVNHKPSNTKVDKKVEPKSCDKTHTCPP |
| | | | CPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSRDELTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 286 | DIQLTQSPSSLSASVGDRVTITCSVSSSISSNNLHWYQQK |
| | Chain | | PGKAPKPWIYGTSNLASGVPSRFSGSGSGTDYTFTISSLQ |
| | | | PEDIATYYCQQWSSYPYMYTFGQGTKVEIKRTVAAPSV |
| | | | FIFPPSDEQLKSGTASVVCLLNNFYPREAKVQWKVDNA |
| | | | LQSGNSQESV1EQDSKDSTYSLSSTLTLSKADYEKHKVY |
| | | | ACEVTHQGLSSPVTKSFNRGEC |
| Mirvetuximab | Heavy | 291 | QVQLVQSGAEVVKPGASVKISCKASGYTFTGYFMNWV |
| | Chain | | KQSPGQSLEWIGRIHPYDGDTFYNQKFQGKATLTVDKS |
| | | | SNTAHMELLSLTSEDFAVYYCTRYDGSRAMDYWGQGT |
| | | | TVTVSSAS |
| | | | TKGPSVFPLAPSSKSTSGGTAALGCLVKDYFPEPVTVSW |
| | | | NSGALTSGVHTFPAVLQSSGLYSLSSVVTVPSSSLGTQT |
| | | | YICNVNHKPSNTKVDKKVEPKSCDKTHTCPPCPAPELLG |
| | | | GPSVFLFPPKPKDTLMISRTPEVTCVVVDVSHEDPEVKF |
| | | | NWYVDGVEVHNAKTKPREEQYNSTYRVVSVLTVLHQD |
| | | | WLNGKEYKCKVSNKALPAPIEKTISKAKGQPREPQVYT |
| | | | LPPSRDELTKNQVSLTCLVKGFYPSDIAVEWESNGQPEN |
| | | | NYKTTPPVLDSDGSFFLYSKLTVDKSRWQQGNVFSCSV |
| | | | MHEALHNHYTQKSLSLSPG |
| | Light | 296 | DIVLTQSPLSLAVSLGQPAIISCKASQSVSFAGTSLMHWY |
| | Chain | | HQKPGQQPRLLIYRASNLEAGVPDRFSGSGSKTDFTLTIS |
| | | | PVEAEDAATYYCQQSREYPYTFGGGTKLEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHKV |
| | | | YACEVTHQGLSSPVTKSFNRGEC |
| J591 variant 1 | Heavy | 301 | EVQLQQSGPELKKPGTSVRISCKTSGYTFTEYTIHWVKQ |
| | Chain | | SHGKSLEWIGNINPNNGGTTYNQKFEDKATLTVDKSSST |
| | | | AYMELRSLTSEDSAVYYCAAGWNFDYWGQGTTLTVSS |
| | | | ASTKGPSVFPLAPSSKSTSGGTAALGCLVKDYFPEPVTV |
| | | | SWNSGALTSGVHTFPAVLQSSGLYSLSSVVTVPSSSLGT |
| | | | QTYICNVNHKPSNTKVDKKVEPKSCDKTHTCPPCPAPEL |
| | | | LGGPSVFLFPPKPKDTLMISRTPEVTCVVVDVSHEDPEV |
| | | | KFNWYVDGVEVHNAKTKPREEQYNSTYRVVSVLTVLH |
| | | | QDWLNGKEYKCKVSNKALPAPIEKTISKAKGQPREPQV |
| | | | YTLPPSREEMTKNQVSLTCLVKGFYPSDIAVEWESNGQP |
| | | | ENNYKTTPPVLDSDGSFFLYSKLTVDKSRWQQGNVFSC |
| | | | SVMHEALHNHYTQKSLSLSPGK |
| | Light | 306 | DIVMTQSHKFMSTSVGDRVSIICKASQDVGTAVDWYQQ |
| | Chain | | KPGQSPKLLIYWASTRHTGVPDRFTGSGSGTDFTLTITN |
| | | | VQSEDLADYFCQQYNSYPLTFGAGTMLDLKRTVAAPSV |
| | | | FIFPPSDEQLKSGTASVVCLLNNFYPREAKVQWKVDNA |
| | | | LQSGNSQESV1EQDSKDSTYSLSSTLTLSKADYEKHKVY |
| | | | ACEVTHQGLSSPVTKSFNRGEC |
| J591 variant 2 | Heavy | 311 | EVQLQQSGPELVKPGTSVRISCKTSGYTFTEYTIHWVKQ |
| | Chain | | SHGKSLEWIGNINPNNGGTTYNQKFEDKATLTVDKSSST |
| | | | AYMELRSLTSEDSAVYYCAAGWNFDYWGQGTTLTVSS |
| | | | ASTKGPSVFPLAPSSKSTSGGTAALGCLVKDYFPEPVTV |
| | | | SWNSGALTSGVHTFPAVLQSSGLYSLSSVVTVPSSSLGT |
| | | | QTYICNVNHKPSNTKVDKKVEPKSCDKTHTCPPCPAPEL |
| | | | LGGPSVFLFPPKPKDTLMISRTPEVTCVVVDVSHEDPEV |
| | | | KFNWYVDGVEVHNAKTKPREEQYNSTYRVVSVLTVLH |
| | | | QDWLNGKEYKCKVSNKALPAPIEKTISKAKGQPREPQV |
| | | | YTLPPSREEMTKNQVSLTCLVKGFYPSDIAVEWESNGQP |
| | | | ENNYKTTPPVLDSDGSFFLYSKLTVDKSRWQQGNVFSC |
| | | | SVMHEALHNHYTQKSLSLSPGK |
| | Light | 316 | NIVMTQSPKSMSMSVGERVTLTCKASENVVTYVSWYQ |
| | Chain | | QKPEQSPKLLIYGASNRYTGVPDRFTGSGSATDFTLTISS |
| | | | VQAEDLADYHCGQGYSYPYTFGGGTKLEIKRTVAAPSV |
| | | | FIFPPSDEQLKSGTASVVCLLNNFYPREAKVQWKVDNA |
| | | | LQSGNSQESVTEQDSKDSTYSLSSTLTLSKADYEKHKVY |
| | | | ACEVTHQGLSSPVTKSFNRGEC |
| Rovalpituzumab | Heavy | 321 | QVQLVQSGAEVKKPGASVKVSCKASGYTFTNYGMNW |
| | Chain | | VRQAPGQGLEWMGWINTYTGEPTYADDFKGRVTMTT |
| | | | DTSTSTAYMELRSLRSDDTAVYYCARIGDSSPSDYWGQ |
| | | | GTLVTVSSASTKGPSVFPLAPSSKSTSGGTAALGCLVKD |
| | | | YFPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVVVD |
| | | | VSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSRDELTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPG |
| | Light | 326 | EIVMTQSPATLSVSPGERATLSCKASQSVSNDVVWYQQ |
| | Chain | | KPGQAPRLLIYYASNRYTGIPARFSGSGSGIEFTLTISSLQ |
| | | | SEDFAVYYCQQDYTSPWTFGQGTKLEIKRTVAAPSVFIF |
| | | | PPSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| PF-06647020 | Heavy | 331 | QVQLVQSGPEVKKPGASVKVSCKASGYTFTDYAVHWV |
| | Chain | | RQAPGKRLEWIGVISTYNDYTYNNQDFKGRVTMTRDTS |
| | | | ASTAYMELSRLRSEDTAVYYCARGNSYFYALDYWGQG |
| | | | TSVTVSSASTKGPSVFPLAPSSKSTSGGTAALGCLVKDY |
| | | | FPEPVTVSWNSGALTSGVHTFPAVLQSSGLYSLSSVVTV |
| | | | PSSSLGTQTYICNVNHKPSNTKVDKKVEPKSCDKTHTCP |
| | | | PCPAPELLGGPSVFLFPPKPKDTLMISRTPEVTCVVVDVS |
| | | | HEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| | Light | 336 | EIVLTQSPATLSLSPGERATLSCRASESVDSYGKSFMHW |
| | Chain | | YQQKPGQAPRLLIYRASNLESGIPARFSGSGSGTDFTLTI |
| | | | SSLEPEDFAVYYCQQSNEDPWTFGGGTKLEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHKV |
| | | | YACEVTHQGLSSPVTKSFNRGEC |
| Antibody to PTK7 | Heavy | 341 | QVQLVESGGGVVQPGRSLRLSCAASGFTFSSYAFHWVR |
| | Chain | | QAPGKGLEWVAVISYDGSIKYYADSVKGRFTISRDNSK |
| | | | NTLYLQMNSLRAEDTAVYYCARTYYFDYWGQGTLVT |
| | | | VSSASTKGPSVFPLAPSSKSTSGGTAALGCLVKDYFPEP |
| | | | VTVSWNSGALTSGVHTFPAVLQSSGLYSLSSVVTVPSSS |
| | | | LGTQTYICNVNHKPSNTKVDKKVEPKSCDKTHTCPPCP |
| | | | APELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 346 | EIVLTQSPDFQSVTPKEKVTITCRASQSIGSSLHWYQQKP |
| | Chain | | DQSPKLLIKYASQSFSGVPSRFSGSGSGTDFTLTINSLEAE |
| | | | DAAAYYCHQSSSLPITFGQGTRLEIKRTVAAPSVFIFPPS |
| | | | DEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| Ladiratuzumab of | Heavy | 351 | QVQLVQSGAEVKKPGASVKVSCKASGLTIEDYYMHWV |
| SGN-LIV1A | Chain | | RQAPGQGLEWMGWIDPENGDTEYGPKFQGRVTMTRDT |
| | | | SINTAYMELSRLRSDDTAVYYCAVHNAHYGTWFAYWG |
| | | | QGTLVTVSSASTKGPSVFPLAPSSKSTSGGTAALGCLVK |
| | | | DYFPEPVTVSWNSGALTSGVHTFPAVLQSSGLYSLSSVV |
| | | | TVPSSSLGTQTYICNVNHKPSNTKVDKKVEPKSCDKTHT |
| | | | CPPCPAPELLGGPSVFLFPPKPKDTLMISRTPEVTCVVVD |
| | | | VSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| | Light | 356 | DVVMTQSPLSLPVTLGQPASISCRSSQSLLHSSGNTYLEY |
| | Chain | | FQQRPGQSPRPLIYKISTRFSGVPDRFSGSGSGTDFTLKIS |
| | | | RVEAEDVGVYYCFQGSHVPYTFGGGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHKV |
| | | | YACEVTHQGLSSPVTKSFNRGEC |
| Cirmtuzumab | Heavy | 361 | QVQLQESGPGLVKPSQTLSLTCTVSGYAFTAYNIHWVR |
| | Chain | | QAPGQGLEWMGSFDPYDGGSSYNQKFKDRLTISKDTSK |
| | | | NQVVLTMTNMDPVDTATYYCARGWYYFDYWGHGTL |
| | | | VTVSSASTKGPSVFPLAPSSKSTSGGTAALGCLVKDYFP |
| | | | EPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 366 | DIVMTQTPLSLPVTPGEPASISCRASKSISKYLAWYQQKP |
| | Chain | | GQAPRLLIYSGSTLQSGIPPRFSGSGYGTDFTLTINNIESE |
| | | | DAAYYFCQQHDESPYTFGEGTKVEIKRTVAAPSVFIFPP |
| | | | SDEQLKSGTASVVCLLNNFYPREAKVQWKVDNALQSG |
| | | | NSQESVTEQDSKDSTYSLSSTLTLSKADYEKHKVYACE |
| | | | VTHQGLSSPVTKSFNRGEC |
| Antibody to | Heavy | 371 | QVQLVESGGGVVQPGRSLRLSCTASGFRFRSHGMHWV |
| MAGE-A3 | Chain | | RQAPGKGLEWVAVISYDGNNKLYADSVKGRITISRDNS |
| | | | KNTLFLQMNNVRAEDTAVYYCASPYTSDWQYFQYWG |
| | | | QGTLVIVSSASTKGPSVFPLAPSSKSTSGGTAALGCLVK |
| | | | DYFPEPVTVSWNSGALTSGVHTFPAVLQSSGLYSLSSVV |
| | | | TVPSSSLGTQTYICNVNHKPSNTKVDKKVEPKSCDKTHT |
| | | | CPPCPAPELLGGPSVFLFPPKPKDTLMISRTPEVTCVVVD |
| | | | VSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| | Light | 376 | EIVMTQSPATLSVSPGERATFSCRASQNISTTLAWYQQK |
| | Chain | | PGQAPRLLIYDTSTRATGIPARFSGSGSGTEFTLTISSLQS |
| | | | EDLAVYYCQQSNSWPLTFGGGTKVEIKRTVAAPSVFIFP |
| | | | PSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |
| Antibody to NY- | Heavy | 381 | QVQLVQSGGGVVRPGGSLRLSCAASGFSFIDYGMSWVR |
| ESO-1 | Chain | | QVPGKGLEWVAGMNWSGDKKGHAESVKGRFIISRDNA |
| | | | KNTLYLEMSSLRVEDTALYFCARGEYSNRFDPRGRGTL |
| | | | VTVSSASTKGPSVFPLAPSSKSTSGGTAALGCLVKDYFP |
| | | | EPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPSDIAVEWES |
| | | | NGQPENNYKTTPPVLDSDGSFFLYSKLTVDKSRWQQGN |
| | | | VFSCSVMHEALHNHYTQKSLSLSPGK |
| | Light | 386 | DIVMTQTPLSLPVTLGQPASLSCRSSQSLVFTDGNTYLN |
| | Chain | | WFQQRPGQSPRRLIYKVSSRDPGVPDRFSGTGSGTDFTL |
| | | | EISRVEAEDIGVYYCMQGTHWPPIFGQGTKVEIKRTVAA |
| | | | PSVFIFPPSDEQLKSGTASVVCLLNNFYPREAKVQWKVD |
| | | | NALQSGNSQESVTEQDSKDSTYSLSSTLTLSKADYEKHK |
| | | | VYACEVTHQGLSSPVTKSFNRGEC |
| Trastuzumab | Heavy | 391 | EVQLVESGGGLVQPGGSLRLSCAASGFNIKDTYIHWVR |
| | Chain | | QAPGKGLEWVARIYPTNGYTRYADSVKGRFTISADTSK |
| | | | NTAYLQMNSLRAEDTAVYYCSRWGGDGFYAMDYWG |
| | | | QGTLVTVSSASTKGPSVFPLAPSSKSTSGGTAALGCLVK |
| | | | DYFPEPVTVSWNSGALTSGVHTFPAVLQSSGLYSLSSVV |
| | | | TVPSSSLGTQTYICNVNHKPSNTKVDKKVEPKSCDKTHT |
| | | | CPPCPAPELLGGPSVFLFPPKPKDTLMISRTPEVTCVVVD |
| | | | VSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| | Light | 396 | DIQMTQSPSSLSASVGDRVTITCRASQDVNTAVAWYQQ |
| | Chain | | KPGKAPKLLIYSASFLYSGVPSRFSGSRSGTDFTLTISSLQ |
| | | | PEDFATYYCQQHYTTPPTFGQGTKVEIKRTVAAPSVFIFP |
| | | | PSDEQLKSGTASVVCLLNNFYPREAKVQWKVDNALQS |
| | | | GNSQESVTEQDSKDSTYSLSSTLTLSKADYEKHKVYAC |
| | | | EVTHQGLSSPVTKSFNRGEC |

An antibody construct can comprise an anti-tumor antibody, wherein the antibody comprises: a) a heavy chain sequence having at least 80% sequence identity to an amino acid sequence of SEQ ID NO: 11, and a light chain sequence having at least 80% sequence identity to an amino acid sequence of SEQ ID NO: 16; b) a heavy chain sequence having at least 80% sequence identity to an amino acid sequence of SEQ ID NO: 21, and a light chain sequence having at least 80% sequence identity to an amino acid sequence of SEQ ID NO: 26; c) a heavy chain sequence having at least 80% sequence identity to an amino acid sequence of SEQ ID NO: 31, and a light chain sequence having at least 80% sequence identity to an amino acid sequence of SEQ ID NO: 36; d) a heavy chain sequence having at least 80% sequence identity to an amino acid sequence of SEQ ID NO: 41, and a light chain sequence having at least 80% sequence identity to an amino acid sequence of SEQ ID NO: 46; e) a heavy chain sequence having at least 80% sequence identity to an amino acid sequence of SEQ ID NO: 51, and a light chain sequence having at least 80% sequence identity to an amino acid sequence of SEQ ID NO: 56; f) a heavy chain sequence having at least 80% sequence identity to an amino acid sequence of SEQ ID NO: 61, and a light chain sequence having at least 80% sequence identity to an amino acid sequence of SEQ ID NO: 66; g) a heavy chain sequence having at least 80% sequence identity to an amino acid sequence of SEQ ID NO: 71, and a light chain sequence having at least 80% sequence identity to an amino acid sequence of SEQ ID NO: 76; h) a heavy chain sequence having at least 80% sequence identity to an amino acid sequence of SEQ ID NO: 81, and a light chain sequence having at least 80% sequence identity to an amino acid sequence of SEQ ID NO: 86; i) a heavy chain sequence having at least 80% sequence identity to an amino acid sequence of SEQ ID NO: 91, and a light chain sequence having at least 80% sequence identity to an amino acid sequence of SEQ ID NO: 96; j) a heavy chain sequence having at least 80% sequence identity to an amino acid sequence of SEQ ID NO: 101, and a light chain sequence having at least 80% sequence identity to an amino acid sequence of SEQ ID NO: 106; k) a heavy chain sequence having at least 80% sequence identity to an amino acid sequence of SEQ ID NO: 111, and a light chain sequence having at least 80% sequence identity to an amino acid sequence of SEQ ID NO: 116; 1) a heavy chain sequence having at least 80% sequence identity to an amino acid sequence of SEQ ID NO: 121, and a light chain sequence having at least 80% sequence identity to an amino acid sequence of SEQ ID NO: 126; m) a heavy chain sequence having at least 80% sequence identity to an amino acid sequence of SEQ ID NO: 131, and a light chain sequence having at least 80% sequence identity to an amino acid sequence of SEQ ID NO: 136; n) a heavy chain sequence having at least 80% sequence identity to an amino acid sequence of SEQ ID NO: 141, and a light chain sequence having at least 80% sequence identity to an amino acid sequence of SEQ ID NO: 146; o) a heavy chain sequence having at least 80% sequence identity to an amino acid sequence of SEQ ID NO: 151, and a light chain sequence having at least 80% sequence identity to an amino acid sequence of SEQ ID NO: 156; p) a heavy chain sequence having at least 80% sequence identity to an amino acid sequence of SEQ ID NO: 161, and a light chain sequence having at least 80% sequence identity to an amino acid sequence of SEQ ID NO: 166; q) a heavy chain sequence having at least 80% sequence identity to an amino acid sequence of SEQ ID NO: 171, and a light chain sequence having at least 80% sequence identity to an amino acid sequence of SEQ ID NO: 176; r) a heavy chain sequence having at least 80% sequence identity to an amino acid sequence of SEQ ID NO: 181, and a light chain sequence having at least 80% sequence identity to an amino acid sequence of SEQ ID NO: 186; s) a heavy chain sequence having at least 80% sequence identity to an amino acid sequence of SEQ ID NO: 191, and a light chain sequence having at least 80% sequence identity to an amino acid sequence of SEQ ID NO: 196; t) a heavy chain sequence having at least 80% sequence identity to an amino acid sequence of SEQ ID NO: 201, and a light chain sequence having at least 80% sequence identity to an amino acid sequence of SEQ ID NO: 206; u) a heavy chain sequence having at least 80% sequence identity to an amino acid sequence of SEQ ID NO: 211, and a light chain sequence having at least 80% sequence identity to an amino acid sequence of SEQ ID NO: 216; v) a heavy chain sequence having at least 80% sequence identity to an amino acid sequence of SEQ ID NO: 221, and a light chain sequence having at least 80% sequence identity to an amino acid sequence of SEQ ID NO: 226; w) a heavy chain sequence having at least 80% sequence identity to an amino acid sequence of SEQ ID NO: 231, and a light chain sequence having at least 80% sequence identity to an amino acid sequence of SEQ ID NO: 236; x) a heavy chain sequence having at least 80% sequence identity to an amino acid sequence of SEQ ID NO: 241, and a light chain sequence having at least 80% sequence identity to an amino acid sequence of SEQ ID NO: 246; y) a heavy chain sequence having at least 80% sequence identity to an amino acid sequence of SEQ ID NO: 251, and a light chain sequence having at least 80% sequence identity to an amino acid sequence of SEQ ID NO: 256; z) a heavy chain sequence having at least 80% sequence identity to an amino acid sequence of SEQ ID NO: 261, and a light chain sequence having at least 80% sequence identity to an amino acid sequence of SEQ ID NO: 266; aa) a heavy chain sequence having at least 80% sequence identity to an amino acid sequence of SEQ ID NO: 271, and a light chain sequence having at least 80% sequence identity to an amino acid sequence of SEQ ID NO: 276; bb) a heavy chain sequence having at least 80% sequence identity to an amino acid sequence of SEQ ID NO: 281, and a light chain sequence having at least 80% sequence identity to an amino acid sequence of SEQ ID NO: 286; cc) a heavy chain sequence having at least 80% sequence identity to an amino acid sequence of SEQ ID NO: 291, and a light chain sequence having at least 80% sequence identity to an amino acid sequence of SEQ ID NO: 296; dd) a heavy chain sequence having at least 80% sequence identity to an amino acid sequence of SEQ ID NO: 301, and a light chain sequence having at least 80% sequence identity to an amino acid sequence of SEQ ID NO: 306; ee) a heavy chain sequence having at least 80% sequence identity to an amino acid sequence of SEQ ID NO: 311, and a light chain sequence having at least 80% sequence identity to an amino acid sequence of SEQ ID NO: 316; ff) a heavy chain sequence having at least 80% sequence identity to an amino acid sequence of SEQ ID NO: 321, and a light chain sequence having at least 80% sequence identity to an amino acid sequence of SEQ ID NO: 326; gg) a heavy chain sequence having at least 80% sequence identity to an amino acid sequence of SEQ ID NO: 331, and a light chain sequence having at least 80% sequence identity to an amino acid sequence of SEQ ID NO: 336; hh) a heavy chain sequence having at least 80% sequence identity to an amino acid sequence of SEQ ID NO: 341, and a light chain sequence having at least 80% sequence identity to an amino acid sequence of SEQ ID NO: 346; ii) a heavy chain sequence having at least 80% sequence identity to an amino acid sequence of SEQ ID NO: 351, and a light chain sequence having at least 80% sequence identity to an amino acid sequence of SEQ ID NO: 356; jj) a heavy chain sequence having at least 80% sequence identity to an amino acid sequence of SEQ ID NO: 361, and a light chain sequence having at least 80% sequence identity to an amino acid sequence of SEQ ID NO: 366; kk) a heavy chain sequence having at least 80% sequence identity to an amino acid sequence of SEQ ID NO: 371, and a light chain sequence having at least 80% sequence identity to an amino acid sequence of SEQ ID NO: 376; ll) a heavy chain sequence having at least 80% sequence identity to an amino acid sequence of SEQ ID NO: 381, and a light chain sequence having at least 80% sequence identity to an amino acid sequence of SEQ ID NO: 386; or mm) a heavy chain sequence having at least 80% sequence identity to an amino acid sequence of SEQ ID NO: 391, and a light chain sequence having at least 80% sequence identity to an amino acid sequence of SEQ ID NO: 396.

An antibody construct can comprise a first binding domain and a second binding domain or a third binding domain that specifically binds to a different antigen than the first binding domain. An antibody construct can comprise a second binding domain or a third binding domain that specifically binds to an antigen on an immune cell. An immune cell can be a T cell, B cell, dendritic cell, macrophage, NK cell, or NKT cell. An immune cell can be an antigen presenting cell, such as a dendritic cell, a macrophage or a monocyte. An antibody construct can comprise a first binding domain and a second binding domain or third binding domain that specifically binds to an antigen on an immune cell, such as an antigen presenting cell. An antigen presenting cell can be a dendritic cell, a monocyte or a macrophage. An antigen presenting cell can be a dendritic cell or a macrophage.

A second binding domain or a third binding domain can specifically bind to an antigen on an immune cell, wherein the antigen comprises a sequence having at least 80% identity to an amino acid sequence of a group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, CD32B, PD-L1 and CD47. A second binding domain or a third binding domain can specifically bind to an antigen on an immune cell, wherein the antigen is selected from the group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, CD32B, PD-L1 and CD47. A second binding domain or a third binding domain can specifically bind to an antigen on an antigen presenting cell, wherein the antigen comprises a sequence having at least 80% identity to an amino acid sequence selected from the group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, and CD32B. A second binding domain or a third binding domain can specifically bind to an antigen on an antigen presenting cell, wherein the antigen is selected from the group consisting of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1, and CD32B. TABLE 2 shows exemplary amino acid sequences of antigens on immune cells.

An antibody construct of a conjugate can comprise a second binding domain or third binding domain comprising one or more CDRs that specifically binds to an antigen on an antigen presenting cell. A second binding domain can comprise at least 80% sequence identity (or at least 90%, 95%, or 100% sequence identity) to a sequence in TABLE 4. A second binding domain or third binding domain can comprise a set of six CDR sequences set forth in TABLE 4.

TABLE 4
APC Antibody CDRs

| | | SEQ ID | |
|---|---|---|---|
| Antibody | Region | NO: | Sequence |
| CP-8709893 | HCDR1 | 3 | GYTFTGYY |
| | HCDR2 | 4 | INPDSGGT |
| | HCDR3 | 5 | ARDQPLGYCTNGVCSYFDY |
| | LCDR1 | 8 | QGIYSW |
| | LCDR2 | 9 | TAS |
| | LCDR3 | 10 | QQANIFPLT |
| SBT-040 | HCDR1 | 3 | GYTFTGYY |
| (G1/G2) | HCDR2 | 4 | INPDSGGT |
| | HCDR3 | 5 | ARDQPLGYCTNGVCSYFDY |
| | LCDR1 | 8 | QGIYSW |
| | LCDR2 | 9 | TAS |
| | LCDR3 | 10 | QQANIFPLT |
| Dacetuzumab | HCDR1 | 406 | GYSFTGYY |
| | HCDR2 | 407 | VIPNAGGT |
| | HCDR3 | 408 | AREGIYW |
| | LCDR1 | 411 | QSLVHSNGNTF |
| | LCDR2 | 412 | TVS |
| | LCDR3 | 413 | SQTTHVPWT |
| Bleselumab | HCDR1 | 416 | GGSISSPGYY |
| | HCDR2 | 417 | IYKSGST |
| | HCDR3 | 418 | TRPVVRYFGWFDP |
| | LCDR1 | 421 | QGISSA |
| | LCDR2 | 422 | DAS |
| | LCDR3 | 423 | QQFNSYPT |
| lucatumumab | HCDR1 | 426 | GFTFSSYG |
| | HCDR2 | 427 | ISYEESNR |
| | HCDR3 | 428 | ARDGGIAAPGPDY |
| | LCDR1 | 431 | QSLLYSNGYNY |
| | LCDR2 | 432 | LGS |
| | LCDR3 | 433 | MQARQTPFT |
| ADC-1013 | HCDR1 | 436 | GFTFSTYG |
| | HCDR2 | 437 | ISGGSSYI |
| | HCDR3 | 438 | ARILRGGSGMDL |
| | LCDR1 | 441 | SSNIGAGYN |
| | LCDR2 | 442 | GNI |
| | LCDR3 | 443 | AAWDKSISGLV |
| APX005 | HCDR1 | 446 | GFSFSSTY |
| | HCDR2 | 447 | IYTGDGTN |
| | HCDR3 | 448 | ARPDITYGFAINFW |
| | LCDR1 | 451 | QSISSR |
| | LCDR2 | 452 | RAS |
| | LCDR3 | 453 | QCTGYGISWP |
| Chi Lob 7/4 | HCDR1 | 456 | GYTFTEYI |
| | HCDR2 | 457 | IIPNNGGT |
| | HCDR3 | 458 | TRREVYGRNYYALDY |
| | LCDR1 | 461 | QGINNY |
| | LCDR2 | 462 | YTS |
| | LCDR3 | 463 | QQYSNLPYT |
| DEC-205 | HCDR1 | 466 | GFTFSNYG |
| variant 1 | HCDR2 | 467 | IWYDGSNK |
| | HCDR3 | 468 | ARDLWGWYFDY |
| | LCDR1 | 471 | QSVSSY |
| | LCDR2 | 472 | DAS |
| | LCDR3 | 473 | QQRRNWPLT |
| DEC-205 | HCDR1 | 476 | GDSFTTYW |
| variant 2 | HCDR2 | 477 | IYPGDSDT |
| | HCDR3 | 478 | TRGDRGVDY |
| | LCDR1 | 481 | QGISRW |
| | LCDR2 | 482 | AAS |
| | LCDR3 | 483 | QQYNSYPRT |
| DC-SIGN | HCDR1 | 552 | QHFWNTPWT |
| variant 1 | HCDR2 | 553 | QQGHTLPYT |
| | HCDR3 | 554 | SNDGYYS |
| | LCDR1 | 555 | RYYLGVD |
| | LCDR2 | 556 | DDSGRFP |
| | LCDR3 | 557 | YGYAVDY |
| DC-SIGN | HCDR1 | 558 | YYGIYVDY |
| variant 2 | HCDR2 | 559 | FLVY |
| | HCDR3 | 560 | NFGILGY |
| | LCDR1 | 561 | YPNALDY |
| | LCDR2 | 562 | GLKSFYAMDH |
| | LCDR3 | 563 | QQGKTLPWT |
| DC-SIGN | HCDR1 | 564 | QQGNTLPPT |
| variant 3 | HCDR2 | 565 | QQHYITPLT |
| | HCDR3 | 566 | QQYGNLPYT |
| | LCDR1 | 567 | QQYYSTPRT |
| | LCDR2 | 568 | GQSYNYPPT |
| | LCDR3 | 569 | WQDTHFPHV |

An antibody construct of a conjugate can comprise a second binding domain or a third binding domain that specifically binds to CD40. An antibody construct can comprise a second binding domain or a third antigen binding domain that is a CD40 agonist. An antibody construct can comprise a second binding domain or a third binding domain that specifically binds to CD40, wherein the second binding domain or third binding domain comprises at least 80% sequence identity to: a) HCDR1 comprising an amino acid sequence of SEQ ID NO: 3, HCDR2 comprising an amino acid sequence of SEQ ID NO: 4, HCDR3 comprising an amino acid sequence of SEQ ID NO: 5, LCDR1 comprising an amino acid sequence of SEQ ID NO: 8, LCDR2 comprising an amino acid sequence of SEQ ID NO: 9, and LCDR3 comprising an amino acid sequence of SEQ ID NO: 10; b) HCDR1 comprising an amino acid sequence of SEQ ID NO: 406, HCDR2 comprising an amino acid sequence of SEQ ID NO: 407, HCDR3 comprising an amino acid sequence of SEQ ID NO: 408, LCDR1 comprising an amino acid sequence of SEQ ID NO: 411, LCDR2 comprising an amino acid sequence of SEQ ID NO: 412, and LCDR3 comprising an amino acid sequence of SEQ ID NO: 413; c) HCDR1 comprising an amino acid sequence of SEQ ID NO: 416, HCDR2 comprising an amino acid sequence of SEQ ID NO: 417, HCDR3 comprising an amino acid sequence of SEQ ID NO: 418, LCDR1 comprising an amino acid sequence of SEQ ID NO: 421, LCDR2 comprising an amino acid sequence of SEQ ID NO: 422, and LCDR3 comprising an amino acid sequence of SEQ ID NO: 423; d) HCDR1 comprising an amino acid sequence of SEQ ID NO: 426, HCDR2 comprising an amino acid sequence of SEQ ID NO: 427, HCDR3 comprising an amino acid sequence of SEQ ID NO: 428, LCDR1 comprising an amino acid sequence of SEQ ID NO: 431, LCDR2 comprising an amino acid sequence of SEQ ID NO: 432, and LCDR3 comprising an amino acid sequence of SEQ ID NO: 433; e) HCDR1 comprising an amino acid sequence of SEQ ID NO: 436, HCDR2 comprising an amino acid sequence of SEQ ID NO: 437, HCDR3 comprising an amino acid sequence of SEQ ID NO: 438, LCDR1 comprising an amino acid sequence of SEQ ID NO: 441, LCDR2 comprising an amino acid sequence of SEQ ID NO: 442, and LCDR3 comprising an amino acid sequence of SEQ ID NO: 443; f) HCDR1 comprising an amino acid sequence of SEQ ID NO: 446, HCDR2 comprising an amino acid sequence of SEQ ID NO: 447, HCDR3 comprising an amino acid sequence of SEQ ID NO: 448, LCDR1 comprising an amino acid sequence of SEQ ID NO: 451, LCDR2 comprising an amino acid sequence of SEQ ID NO: 452, and LCDR3 comprising an amino acid sequence of SEQ ID NO: 453; or g) HCDR1 comprising an amino acid sequence of SEQ ID NO: 456, HCDR2 comprising an amino acid sequence of SEQ ID NO: 457, HCDR3 comprising an amino acid sequence of SEQ ID NO: 458, LCDR1 comprising an amino acid sequence of SEQ ID NO: 461, LCDR2 comprising an amino acid sequence of SEQ ID NO: 462, and LCDR3 comprising an amino acid sequence of SEQ ID NO: 463.

A conjugate or antibody construct can comprise a second binding domain or third binding domain that specifically binds to DC-SIGN. A conjugate or antibody construct can comprise a second binding domain or third binding domain that binds DC-SIGN, wherein the second binding domain or third binding domain comprises at least 80% sequence identity to: a) HCDR1 comprising an amino acid sequence of SEQ ID NO: 552, HCDR2 comprising an amino acid sequence of SEQ ID NO: 553, HCDR3 comprising an amino acid sequence of SEQ ID NO: 554, LCDR1 comprising an amino acid sequence of SEQ ID NO: 555, LCDR2 comprising an amino acid sequence of SEQ ID NO: 556, and LCDR3 comprising an amino acid sequence of SEQ ID NO: 557; b) HCDR1 comprising an amino acid sequence of SEQ ID NO: 558, HCDR2 comprising an amino acid sequence of SEQ ID NO: 559, HCDR3 comprising an amino acid sequence of SEQ ID NO: 560, LCDR1 comprising an amino acid sequence of SEQ ID NO: 561, LCDR2 comprising an amino acid sequence of SEQ ID NO: 562, and LCDR3 comprising an amino acid sequence of SEQ ID NO: 563; or c) HCDR1 comprising an amino acid sequence of SEQ ID NO: 564, HCDR2 comprising an amino acid sequence of SEQ ID NO: 565, HCDR3 comprising an amino acid sequence of SEQ ID NO: 566, LCDR1 comprising an amino acid sequence of SEQ ID NO: 567, LCDR2 comprising an amino acid sequence of SEQ ID NO: 568, and LCDR3 comprising an amino acid sequence of SEQ ID NO: 569.

An antibody construct of a conjugate can comprise a second binding domain or a third binding domain that specifically binds to DEC-205. An antibody construct comprising a second binding domain or a third binding domain that specifically binds to DEC-205 can comprise at least 80% sequence identity to: a) HCDR1 comprising an amino acid sequence of SEQ ID NO: 466, HCDR2 comprising an amino acid sequence of SEQ ID NO: 467, HCDR3 comprising an amino acid sequence of SEQ ID NO: 468, LCDR1 comprising an amino acid sequence of SEQ ID NO: 471, LCDR2 comprising an amino acid sequence of SEQ ID NO: 472, and LCDR3 comprising an amino acid sequence of SEQ ID NO: 473; orb) HCDR1 comprising an amino acid sequence of SEQ ID NO: 476, HCDR2 comprising an amino acid sequence of SEQ ID NO: 477, HCDR3 comprising an amino acid sequence of SEQ ID NO: 478, LCDR1 comprising an amino acid sequence of SEQ ID NO: 481, LCDR2 comprising an amino acid sequence of SEQ ID NO: 482, and LCDR3 comprising an amino acid sequence of SEQ ID NO: 483.

An antibody construct of a conjugate can comprise a second binding domain or a third binding domain comprising one or more variable domains that specifically bind to an antigen on an antigen presenting cell. An antibody construct can comprise a second binding domain or a third binding domain comprising a light chain variable domain (VL domain). A second binding domain or a third binding domain can comprise a sequence having at least 80% sequence identity (or at least 90%, 95%, or 100% sequence identity) to any VL sequence in TABLE 5. An antibody construct can comprise a second binding domain or a third binding domain comprising a heavy chain variable domain. A second binding domain or a third antigen binding domain can comprise a sequence having at least 80% sequence identity (or at least 90%, 95%, or 100% sequence identity) to any VH sequence in TABLE 5. A second binding domain or a third binding domain can comprise at least 80% sequence identity (or at least 90%, 95%, or 100% sequence identity) to any sequence in TABLE 5. A second binding domain or a third binding domain can comprise a pair of VH and VL sequences set forth in TABLE 5.

TABLE 5
APC Antibody V_{H} sequences and V_{L} sequences

| Antibody | Region | SEQ ID NO: | Sequence |
|---|---|---|---|
| CP-8709893 | V_{H} | 2 | QVQLVQSGAEVKKPGASVKVSCKASGYTFTGYY |
| | | | MHWVRQAPGQGLEWMGWINPDSGGTNYAQKFQ |
| | | | GRVTMTRDTSISTAYMELNRLRSDDTAVYYCAR |
| | | | DQPLGYCTNGVCSYFDYWGQGTLVTVSS |
| | V_{L} | 7 | DIQMTQSPSSVSASVGDRVTITCRASQGIYSWLA |
| | | | WYQQKPGKAPNLLIYTASTLQSGVPSRFSGSGSGT |
| | | | DFTLTISSLQPEDFATYYCQQANIFPLTFGGGTKVE |
| | | | IK |
| SBT-040 | V_{H} | 2 | QVQLVQSGAEVKKPGASVKVSCKASGYTFTGYY |
| | | | MHWVRQAPGQGLEWMGWINPDSGGTNYAQKFQ |
| | | | GRVTMTRDTSISTAYMELNRLRSDDTAVYYCAR |
| | | | DQPLGYCTNGVCSYFDYWGQGTLVTVSS |
| | V_{L} | 7 | DIQMTQSPSSVSASVGDRVTITCRASQGIYSWLA |
| | | | WYQQKPGKAPNLLIYTASTLQSGVPSRFSGSGSGT |
| | | | DFTLTISSLQPEDFATYYCQQANIFPLTFGGGTKVE |
| | | | IK |
| Dacetuzumab | V_{H} | 405 | EVQLVESGGGLVQPGGSLRLSCAASGYSFTGYYI |
| | | | HWVRQAPGKGLEWVARVIPNAGGTSYNQKFKGR |
| | | | FTLSVDNSKNTAYLQMNSLRAEDTAVYYCAREGI |
| | | | YWWGQGTLVTVSS |
| | V_{L} | 410 | DIQMTQSPSSLSASVGDRVTITCRSSQSLVHSNGN |
| | | | TFLHWYQQKPGKAPKLLIYTVSNRFSGVPSRFSGS |
| | | | GSGTDFTLTISSLQPEDFATYFCSQTTHVPWTFGQ |
| | | | GTKVEIK |
| Bleselumab | V_{H} | 415 | QLQLQESGPGLLKPSETLSLTCTVSGGSISSPGYYG |
| | | | GWIRQPPGKGLEWIGSIYKSGSTYHNPSLKSRVTIS |
| | | | VDTSKNQFSLKLSSVTAADTAVYYCTRPVVRYFG |
| | | | WFDPWGQGTLVTVSS |
| | V_{L} | 420 | AIQLTQSPSSLSASVGDRVTITCRASQGISSALAWY |
| | | | QQKPGKAPKLLIYDASNLESGVPSRFSGSGSGTDF |
| | | | TLTISSLQPEDFATYYCQQFNSYPTFGQGTKVEIK |
| Lucatumumab | V_{H} | 425 | QVQLVESGGGVVQPGRSLRLSCAASGFTFSSYGM |
| | | | HWVRQAPGKGLEWVAVISYEESNRYHADSVKGR |
| | | | FTISRDNSKITLYLQMNSLRTEDTAVYYCARDGGI |
| | | | AAPGPDYWGQGTLVTVSS |
| | V_{L} | 430 | DIVMTQSPLSLTVTPGEPASISCRSSQSLLYSNGYN |
| | | | YLDWYLQKPGQSPQVLISLGSNRASGVPDRFSGS |
| | | | GS GTDFTLKISRVEAEDVGVYYCMQARQTPFTFG |
| | | | PGTKVDIR |
| ADC-1013 | V_{H} | 435 | EVQLLESGGGLVQPGGSLRLSCAASGFTFSTYGM |
| | | | HWVRQAPGKGLEWLSYISGGSSYIFYADSVRGRF |
| | | | TISRDNSENALYLQMNSLRAEDTAVYYCARILRG |
| | | | GSGMDLWGQGTLVTVSS |
| | V_{L} | 440 | QSVLTQPPSASGTPGQRVTISCTGSSSNIGAGYNV |
| | | | YWYQQLPGTAPKLLIYGNINRPSGVPDRFSGSKSG |
| | | | TSASLAISGLRSEDEADYYCAAWDKSISGLVFGG |
| | | | GTKLTVL |
| APX005 | V_{H} | 445 | QVQLVESGGGVVQPGRSLRLSCAASGFSFSSTYV |
| | | | CWVRQAPGKGLEWIACIYTGDGTNYSASWAKGR |
| | | | FTISKDSSKNTVYLQMNSLRAEDTAVYFCARPDIT |
| | | | YGFAINFWGPGTLVTVSS |
| | V_{L} | 450 | DIQMTQSPSSLSASVGDRVTIKCQASQSISSRLAW |
| | | | YQQKPGKPPKLLIYRASTLASGVPSRFSGSGSGTD |
| | | | FTLTISSLQPEDVATYYCQCTGYGISWPIGGGTKV |
| | | | EIK |
| Chi Lob 7/4 | V_{H} | 455 | EVQLQQSGPDLVKPGASVKISCKTSGYTFTEYIMH |
| | | | WVKQSHGKSLEWIGGIIPNNGGTSYNQKFKDKAT |
| | | | MTVDKSSSTGYMELRSLTSEDSAVYYCTRREVYG |
| | | | RNYYALDYWGQGTLVTVSS |
| | V_{L} | 460 | DIQMTQTTSSLSASLGDRVTITCSASQGINNYLNW |
| | | | YQQKPDGTVKLLIYYTSSLHSGVPSRFSGSGSGTD |
| | | | YSLTISNLEPEDIATYYCQQYSNLPYTFGGGTKLEI |
| | | | K |
| DEC-205 | V_{H} | 465 | QVQLVESGGGVVQPGRSLRLSCAASGFTFSNYGM |
| variant 1 | | | YWVRQAPGKGLEWVAVIWYDGSNKYYADSVKG |
| | | | RFTISRDNSKNTLYLQMNSLRAEDTAVYYCARDL |
| | | | WGWYFDYWGQGTLVTVSS |
| | V_{L} | 470 | EIVLTQSPATLSLSPGERATLSCRASQSVSSYLAW |
| | | | YQQKPGQAPRLLIYDASNRATGIPARFSGSGSGTD |
| | | | FTLTISSLEPEDFAVYYCQQRRNWPLTFGGGTKVE |
| | | | IK |
| DEC-205 | V_{H} | 475 | EVQLVQSGAEVKKPGESLRISCKGSGDSFTTYWIG |
| variant 2 | | | WVRQMPGKGLEWMGIIYPGDSDTIYSPSFQGQVT |
| | | | ISADKSISTAYLQWSSLKASDTAMYYCTRGDRGV |
| | | | DYWGQGTLVTVSS |
| | V_{L} | 480 | DIQMTQSPSSLSASVGDRVTITCRASQGISRWLAW |
| | | | YQQKPEKAPKSLIYAASSLQSGVPSRFSGSGSGTD |
| | | | FTLTISGLQPEDFATYYCQQYNSYPRTFGQGTKVE |
| | | | IK |
| CD36 mannose | V_{H} | 548 | DIQMTQSPSSLSASVGDRVTITCRASQGISRWLAW |
| Scavenger | | | YQQKPEKAPKSLIYAASSLQSGVPSRFSGSGSGTD |
| Receptor | | | FTLTISGLQPEDFATYYCQQYNSYPRTFGQGTKVE |
| | | | IK |
| | V_{L} | 549 | EVQLVQSGAEVKKPGESLRISCKGSGDSFTTYWIG |
| | | | WVRQMPGKGLEWMGIIYPGDSDTIYSPSFQGQVT |
| | | | ISADKSISTAYLQWSSLKASDTAMYYCTRGDRGV |
| | | | DYWGQGTLVTVSS |
| CLEC9A | V_{H} | 550 | QIVESGGGLVQPKESLKISCTASGFTFSNAAIYWV |
| | | | RQTPGKGLEWVGRIRTRPSKYATDYADSVRGRFT |
| | | | ISRDDSKSMVYLQMDNLRTEDTAMYYCTPRATE |
| | | | DVPFYWGQGVMVTVSS |
| | V_{L} | 551 | DIVMTQTP SSQAVSAGEKVTMNCKSSQSVLYDEN |
| | | | KKNYLAWYQQKSGQSPKLLIYWASTGESGVPDR |
| | | | FIGSGSGTDFTLTISSVQAEDLAVYYCQQYYDFPP |
| | | | TFGGGTK |

An antibody construct of a conjugate can comprise a second binding domain or a third binding domain that specifically binds to CD40. An antibody construct can comprise a second binding domain or a third binding domain that is a CD40 agonist. An antibody construct can comprise a second binding domain or a third binding domain that specifically binds to CD40, wherein the second binding domain or the third binding domain comprises: a) a VH sequence having at least 80% sequence identity to an amino acid sequence of SEQ ID NO: 2, and a VL sequence having at least 80% sequence identity to an amino acid sequence of SEQ ID NO: 7; b) a VH sequence having at least 80% sequence identity to an amino acid sequence of SEQ ID NO: 405, and a VL sequence having at least 80% sequence identity to an amino acid sequence of SEQ ID NO: 410; c) a VH sequence having at least 80% sequence identity to an amino acid sequence of SEQ ID NO: 415, and a VL sequence having at least 80% sequence identity to an amino acid sequence of SEQ ID NO: 420; d) a VH sequence having at least 80% sequence identity to an amino acid sequence of SEQ ID NO: 425, and a VL sequence having at least 80% sequence identity to an amino acid sequence of SEQ ID NO: 430; e) a VH sequence having at least 80% sequence identity to an amino acid sequence of SEQ ID NO: 435, and a VL sequence having at least 80% sequence identity to an amino acid sequence of SEQ ID NO: 440; f) a VH sequence having at least 80% sequence identity to an amino acid sequence of SEQ ID NO: 445, and a VL sequence having at least 80% sequence identity to an amino acid sequence of SEQ ID NO: 450; g) a VH sequence having at least 80% sequence identity to an amino acid sequence of SEQ ID NO: 455, and a VL sequence having at least 80% sequence identity to an amino acid sequence of SEQ ID NO: 460.

An antibody construct of a conjugate can comprise a second binding domain or a third binding domain that specifically binds to DEC-205. An antibody construct of a conjugate can comprise a second binding domain or a third binding domain that specifically binds to DEC-205, wherein the second binding domain or the third binding domain comprises: a) a VH sequence having at least 80% sequence identity to an amino acid sequence of SEQ ID NO: 465, and a VL sequence having at least 80% sequence identity to an amino acid sequence of SEQ ID NO: 470; or b) a VH sequence having at least 80% sequence identity to an amino acid sequence of SEQ ID NO: 475, and a VL sequence having at least 80% sequence identity to an amino acid sequence of SEQ ID NO: 480.

An antibody construct of a conjugate can comprise a second binding domain or a third binding domain that specifically binds to CD36 mannose scavenger receptor 1. An antibody construct can comprise a second binding domain or a third binding domain that specifically binds to CD36 mannose scavenger receptor 1, wherein the second binding domain or the third binding domain comprises a VH sequence having at least 80% sequence identity to an amino acid sequence of SEQ ID NO: 548, and a VL sequence having at least 80% sequence identity to an amino acid sequence of SEQ ID NO: 549.

An antibody construct of a conjugate can comprise a second binding domain or a third binding domain that specifically binds to CLEC9A. An antibody construct can comprise a second binding domain or a third binding domain that specifically binds to CLEC9A, wherein the second binding domain or the third binding domain comprises a VH sequence having at least 80% sequence identity to an amino acid sequence of SEQ ID NO: 550, and a VL sequence having at least 80% sequence identity to an amino acid sequence of SEQ ID NO: 551.

An antibody construct can comprise a first binding domain, a second binding domain and an Fc domain, wherein the second binding domain and the Fc domain comprise an antibody that specifically binds to an antigen on an antigen presenting cell. An antibody construct can comprise a first binding domain, second binding domain, a third binding domain and an Fc domain, wherein the second binding domain, the third binding domain and the Fc domain comprise an antibody that specifically binds to an antigen on an antigen presenting cell. An antibody construct can comprise a heavy chain and a light chain that target an antigen expressed by an immune cell such as an antigen presenting cell. An antibody construct can comprise an antibody light chain. An antibody construct can comprise a light chain comprising a sequence having at least 80% sequence identity (or at least 90%, 95%, or 100% sequence identity) to any light chain sequence in TABLE 6. An antibody construct can comprise an antibody heavy chain. An antibody construct can comprise a heavy chain comprising a sequence having at least 80% sequence identity (or at least 90%, 95%, or 100% sequence identity) to any heavy chain sequence in TABLE 6. An antibody construct can comprise at least 80% sequence identity (or at least 90%, 95%, or 100% sequence identity) to any sequence in TABLE 6. An antibody construct can comprise a pair of heavy and light chain sequences set forth in TABLE 6.

TABLE 6
APC Antibody Heavy Chain and Light Chain Sequences

| Antibody | Region | SEQ ID NO: | Sequence |
|---|---|---|---|
| CP-8709893 | Heavy | 1 | QVQLVQSGAEVKKPGASVKVSCKASGYTFTGYYMHWV |
| | Chain | | RQAPGQGLEWMGWINPDSGGTNYAQKFQGRVTMTRDT |
| | | | SISTAYMELNRLRSDDTAVYYCARDQPLGYCTNGVCSYF |
| | | | DYWGQGTLVTVSSASTKGPSVFPLAPCSRSTSESTAALG |
| | | | CLVKDYFPEPVTVSWNSGALTSGVHTFPAVLQSSGLYSL |
| | | | SSVVTVPSSNFGTQTYTCNVDHKPSNTKVDKTVERKCCV |
| | | | ECPPCPAPPVAGPSVFLFPPKPKDTLMISRTPEVTCVVVD |
| | | | VSHEDPEVQFNWYVDGVEVHNAKTKPREEQFNSTFRVV |
| | | | SVLTVVHQDWLNGKEYKCKVSNKGLPAPIEKTISKTKGQ |
| | | | PREPQVYTLPPSREEMTKNQVSLTCLVKGFYPSDIAVEW |
| | | | ESNGQPENNYKTTPPMLDSDGSFFLYSKLTVDKSRWQQ |
| | | | GNVFSCSVMHEALHNHYTQKSLSLSPGK |
| | Light | 6 | DIQMTQSPSSVSASVGDRVTITCRASQGIYSWLAWYQQK |
| | Chain | | PGKAPNLLIYTASTLQSGVPSRFSGSGSGTDFTLTISSLQP |
| | | | EDFATYYCQQANIFPLTFGGGTKVEIKRTVAAPSVFIFPPS |
| | | | DEQLKSGTASVVCLLNNFYPREAKVQWKVDNALQSGNS |
| | | | QESVTEQDSKDSTYSLSSTLTLSKADYEKHKVYACEVTH |
| | | | QGLSSPVTKSFNRGEC |
| SBT-040 | Heavy | 401 | MDWTWRILFLVAAATGAHSQVQLVQSGAEVKKPGASV |
| | Chain | | KVSCKASGYTFTGYYMHWVRQAPGQGLEWMGWINPDS |
| | (IgG1) | | GGTNYAQKFQGRVTMTRDTSISTAYMELNRLRSDDTAV |
| | | | YYCARDQPLGYCTNGVCSYFDYWGQGTLVTVSSASTKG |
| | | | PSVFPLAPCSRSTSESTAALGCLVKDYFPEPVTVSWNSGA |
| | | | LTSGVHTFPAVLQSSGLYSLSSVVTVPSSNFGTQTYTCNV |
| | | | DHKPSNTKVDKTVEPKSCDKTHTCPPCPAPELLGGPSVFL |
| | | | FPPKPKDTLMISRTPEVTCVVVDVSHEDPEVKFNWYVDG |
| | | | VEVHNAKTKPREEQYNSTYRVVSVLTVLHQDWLNGKE |
| | | | YKCKVSNKALPAPIEKTISKAKGQPREPQVYTLPPSREEM |
| | | | TKNQVSLTCLVKGFYPSDIAVEWESNGQPENNYKTTPPV |
| | | | LDSDGSFFLYSKLTVDKSRWQQGNVFSCSVMHEALHNH |
| | | | YTQKSLSLSPGK |
| | Heavy | 402 | MDWTWRILFLVAAATGAHSQVQLVQSGAEVKKPGASV |
| | Chain | | KVSCKASGYTFTGYYMHWVRQAPGQGLEWMGWINPDS |
| | (IgG2) | | GGTNYAQKFQGRVTMTRDTSISTAYMELNRLRSDDTAV |
| | | | YYCARDQPLGYCTNGVCSYFDYWGQGTLVTVSSASTKG |
| | | | PSVFPLAPCSRSTSESTAALGCLVKDYFPEPVTVSWNSGA |
| | | | LTSGVHTFPAVLQSSGLYSLSSVVTVPSSNFGTQTYTCNV |
| | | | DHKPSNTKVDKTVERKCCVECPPCPAPPVAGPSVFLFPPK |
| | | | PKDTLMISRTPEVTCVVVDVSHEDPEVQFNWYVDGVEV |
| | | | HNAKTKPREEQFNSTFRVVSVLTVVHQDWLNGKEYKCK |
| | | | VSNKGLPAPIEKTISKTKGQPREPQVYTLPPSREEMTKNQ |
| | | | VSLTCLVKGFYPSDIAVEWESNGQPENNYKTTPPMLDSD |
| | | | GSFFLYSKLTVDKSRWQQGNVFSCSVMHEALHNHYTQK |
| | | | SLSLSPGK |
| | Light | 403 | MRLPAQLLGLLLLWFPGSRCDIQMTQSPSSVSASVGDRV |
| | Chain | | TITCRASQGIYSWLAWYQQKPGKAPNLLIYTASTLQSGV |
| | | | PSRFSGSGSGTDFTLTISSLQPEDFATYYCQQANIFPLTFG |
| | | | GGTKVEIKRTVAAPSVFIFPPSDEQLKSGTASVVCLLNNF |
| | | | YPREAKVQWKVDNALQSGNSQESVTEQDSKDSTYSLSS |
| | | | TLTLSKADYEKHKVYACEVTHQGLSSPVTKSFNRGEC |
| Dacetuzumab | Heavy | 404 | EVQLVESGGGLVQPGGSLRLSCAASGYSFTGYYIHWVRQ |
| | Chain | | APGKGLEWVARVIPNAGGTSYNQKFKGRFTLSVDNSKN |
| | | | TAYLQMNSLRAEDTAVYYCAREGIYWWGQGTLVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKKVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYNSTYRVVSVLTVLHQDW |
| | | | LNGKEYKCKVSNKALPAPIEKTISKAKGQPREPQVYTLPP |
| | | | SREEMTKNQVSLTCLVKGFYPSDIAVEWESNGQPENNYK |
| | | | TTPPVLDSDGSFFLYSKLTVDKSRWQQGNVFSCSVMHEA |
| | | | LHNHYTQKSLSLSPGK |
| | Light | 409 | DIQMTQSPSSLSASVGDRVTITCRSSQSLVHSNGNTFLHW |
| | Chain | | YQQKPGKAPKLLIYTVSNRFSGVPSRFSGSGSGTDFTLTIS |
| | | | SLQPEDFATYFCSQTTHVPWTFGQGTKVEIKRTVAAPSV |
| | | | FIFPPSDEQLKSGTASVVCLLNNFYPREAKVQWKVDNAL |
| | | | QSGNSQESVTEQDSKDSTYSLSSTLTLSKADYEKHKVYA |
| | | | CEVTHQGLSSPVTKSFNRGEC |
| Bleselumab | Heavy | 414 | QLQLQESGPGLLKPSETLSLTCTVSGGSISSPGYYGGWIR |
| | Chain | | QPPGKGLEWIGSIYKSGSTYHNPSLKSRVTISVDTSKNQF |
| | | | SLKLSSVTAADTAVYYCTRPVVRYFGWFDPWGQGTLVT |
| | | | VSSASTKGPSVFPLAPCSRSTSESTAALGCLVKDYFPEPV |
| | | | TVSWNSGALTSGVHTFPAVLQSSGLYSLSSVVTVPSSSLG |
| | | | TKTYTCNVDHKPSNTKVDKRVESKYGPPCPPCPAPEFEG |
| | | | GPSVFLFPPKPKDTLMISRTPEVTCVVVDVSQEDPEVQFN |
| | | | WYVDGVEVHNAKTKPREEQFNSTYRVVSVLTVLHQDW |
| | | | LNGKEYKCKVSNKGLPSSIEKTISKAKGQPREPQVYTLPP |
| | | | SQEEMTKNQVSLTCLVKGFYPSDIAVEWESNGQPENNY |
| | | | KTTPPVLDSDGSFFLYSRLTVDKSRWQEGNVFSCSVMHE |
| | | | ALHNHYTQKSLSLSLGK |
| | Light | 419 | AIQLTQSPSSLSASVGDRVTITCRASQGISSALAWYQQKP |
| | Chain | | GKAPKLLIYDASNLESGVPSRFSGSGSGTDFTLTISSLQPE |
| | | | DFATYYCQQFNSYPTFGQGTKVEIKRTVAAPSVFIFPPSD |
| | | | EQLKSGTASVVCLLNNFYPREAKVQWKVDNALQSGNSQ |
| | | | ESVTEQDSKDSTYSLSSTLTLSKADYEKHKVYACEVTHQ |
| | | | GLSSPVTKSFNRGEC |
| Lucatumumab | Heavy | 424 | QVQLVESGGGVVQPGRSLRLSCAASGFTFSSYGMHWVR |
| | Chain | | QAPGKGLEWVAVISYEESNRYHADSVKGRFTISRDNSKI |
| | | | TLYLQMNSLRTEDTAVYYCARDGGIAAPGPDYWGQGTL |
| | | | VTVSSASTKGPSVFPLAPASKSTSGGTAALGCLVKDYFPE |
| | | | PVTVSWNSGALTSGVHTFPAVLQSSGLYSLSSVVTVPSSS |
| | | | LGTQTYICNVNHKPSNTKVDKRVEPKSCDKTHTCPPCPA |
| | | | PELLGGPSVFLFPPKPKDTLMISRTPEVTCVVVDVSHEDP |
| | | | EVKFNWYVDGVEVHNAKTKPREEQYNSTYRVVSVLTVL |
| | | | HQDWLNGKEYKCKVSNKALPAPIEKTISKAKGQPREPQV |
| | | | YTLPPSREEMTKNQVSLTCLVKGFYPSDIAVEWESNGQP |
| | | | ENNYKTTPPVLDSDGSFFLYSKLTVDKSRWQQGNVFSCS |
| | | | VMHEALHNHYTQKSLSLSPGK |
| | Light | 429 | DIVMTQSPLSLTVTPGEPASISCRSSQSLLYSNGYNYLDW |
| | Chain | | YLQKPGQSPQVLISLGSNRASGVPDRFSGSGSGTDFTLKIS |
| | | | RVEAEDVGVYYCMQARQTPFTFGPGTKVDIRRTVAAPS |
| | | | VFIFPPSDEQLKSGTASVVCLLNNFYPREAKVQWKVDNA |
| | | | LQSGNSQESVTEQDSKDSTYSLSSTLTLSKADYEKHKVY |
| | | | ACEVTHQGLSSPVTKSFNRGEC |
| ADC-1013 | Heavy | 434 | EVQLLESGGGLVQPGGSLRLSCAASGFTFSTYGMHWVR |
| | Chain | | QAPGKGLEWLSYISGGSSYIFYADSVRGRFTISRDNSENA |
| | | | LYLQMNSLRAEDTAVYYCARILRGGSGMDLWGQGTLV |
| | | | TVSSASTKGPSVFPLAPSSKSTSGGTAALGCLVKDYFPEP |
| | | | VTVSWNSGALTSGVHTFPAVLQSSGLYSLSSWTVPSSSL |
| | | | GTQTYICNVNHKPSNTKVDKKVEPKSCDKTHTCPPCPAP |
| | | | ELLGGPSVFLFPPKPKDTLMISRTPEVTCNAVDVSHEDPE |
| | | | VKFNWYVDGVEVHNAKTKPREEQYNSTYRWSVLTVLH |
| | | | QDWLNGKEYKCKVSNKALPAPIEKTISKAKGQPREPQVY |
| | | | TLPPSRDELTKNQVSLTCLVKGFYPSDIAVEWESNGQPEN |
| | | | NYKTTPPVLDSDGSFFLYSKLTVDKSRWQQGNVFSCSV |
| | | | MHEALHNHYTQKSLSLSPGK |
| | Light | 439 | QSVLTQPPSASGTPGQRVTISCTGSSSNIGAGYNVYWYQ |
| | Chain | | QLPGTAPKLLIYGNINRPSGVPDRFSGSKSGTSASLATSGL |
| | | | RSEDEADYYCAAWDKSISGLVFGGGTKLTVLGQPKAAP |
| | | | SVTLFPPSSEELQANKATLVCLISDFYPGAVTVAWKADSS |
| | | | PVKAGVETTTPSKQSNNKYAASSYLSLTPEQWKSHRSYS |
| | | | CQVTHEGSTVEKTVAPTECS |
| APX005 | Heavy | 444 | QVQLVESGGGVVQPGRSLRLSCAASGFSFSSTYVCWVRQ |
| | Chain | | APGKGLEWIACIYTGDGTNYSASWAKGRFTISKDSSKNT |
| | | | VYLQMNSLRAEDTAVYFCARPDITYGFAINFWGPGTLVT |
| | | | VSSASTKGPSVFPLAPSSKSTSGGTAALGCLVKDYFPEPV |
| | | | TVSWNSGALTSGVHTFPAVLQSSGLYSLSSVVTVPSSSLG |
| | | | TQTYICNVNHKPSNTKVDKKVEPKSCDKTHTCPPCPAPE |
| | | | LLGGPSVFLFPPKPKDTLMISRTPEVTCVVVDVSHEDPEV |
| | | | KFNWYVDGVEVHNAKTKPREEQYNSTYRVVSVLTVLH |
| | | | QDWLNGKEYKCKVSNKALPAPIEKTISKAKGQPREPQVY |
| | | | TLPPSREEMTKNQVSLTCLVKGFYPSDIAVEWESNGQPE |
| | | | NNYKTTPPVLDSDGSFFLYSKLTVDKSRWQQGNVFSCSV |
| | | | MHEALHNHYTQKSLSLSPGK |
| | Light | 449 | DIQMTQSPSSLSASVGDRVTIKCQASQSISSRLAWYQQKP |
| | Chain | | GKPPKLLIYRASTLASGVPSRFSGSGSGTDFTLTISSLQPE |
| | | | DVATYYCQCTGYGISWPIGGGTKVEIKRTVAAPSVFIFPP |
| | | | SDEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| Chi Lob 7/4 | Heavy | 454 | EVQLQQSGPDLVKPGASVKISCKTSGYTFTEYIMHWVKQ |
| | Chain | | SHGKSLEWIGGIIPNNGGTSYNQKFKDKATMTVDKSSST |
| | | | GYMELRSLTSEDSAVYYCTRREVYGRNYYALDYWGQG |
| | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVLT |
| | | | VLHQDWLNGKEYKCKVSNKALPAPIEKTISKAKGQPREP |
| | | | QVYTLPPSREEMTKNQVSLTCLVKGFYPSDIAVEWESNG |
| | | | QPENNYKTTPPVLDSDGSFFLYSKLTVDKSRWQQGNVFS |
| | | | CSVMHEALHNHYTQKSLSLSPGK |
| | Light | 459 | DIQMTQTTSSLSASLGDRVTITCSASQGINNYLNWYQQK |
| | Chain | | PDGTVKLLIYYTSSLHSGVPSRFSGSGSGTDYSLTISNLEP |
| | | | EDIATYYCQQYSNLPYTFGGGTKLEIKRTVAAPSVFIFPPS |
| | | | DEQLKSGTASVVCLLNNFYPREAKVQWKVDNALQSGNS |
| | | | QESVTEQDSKDSTYSLSSTLTLSKADYEKHKVYACEVTH |
| | | | QGLSSPVTKSFNRGEC |
| DEC-205 | Heavy | 464 | QVQLVESGGGVVQPGRSLRLSCAASGFTFSNYGMYWVR |
| (variant 1) | Chain | | QAPGKGLEWVAVIWYDGSNKYYADSVKGRFTISRDNSK |
| | | | NTLYLQMNSLRAEDTAVYYCARDLWGWYFDYWGQGT |
| | | | LVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKKVEPKSCDKTHTCPPCP |
| | | | APELLGGPSVFLFPPKPKDTLMISRTPEVTCVVVDVSHED |
| | | | PEVKFNWYVDGVEVHNAKTKPREEQYNSTYRVVSVLTV |
| | | | LHQDWLNGKEYKCKVSNKALPAPIEKTISKAKGQPREPQ |
| | | | VYTLPPSREEMTKNQVSLTCLVKGFYPSDIAVEWESNGQ |
| | | | PENNYKTTPPVLDSDGSFFLYSKLTVDKSRWQQGNVFSC |
| | | | SVMHEALHNHYTQKSLSLSPGK |
| | Light | 469 | EIVLTQSPATLSLSPGERATLSCRASQSVSSYLAWYQQKP |
| | Chain | | GQAPRLLIYDASNRATGIPARFSGSGSGTDFTLTISSLEPE |
| | | | DFAVYYCQQRRNWPLTFGGGTKVEIKRTVAAPSVFIFPPS |
| | | | DEQLKSGTASVVCLLNNFYPREAKVQWKVDNALQSGNS |
| | | | QESVTEQDSKDSTYSLSSTLTLSKADYEKHKVYACEVTH |
| | | | QGLSSPVTKSFNRGEC |
| DEC-205 | Heavy | 474 | EVQLVQSGAEVKKPGESLRISCKGSGDSFTTYWIGWVRQ |
| (variant 2) | Chain | | MPGKGLEWMGIIYPGDSDTIYSPSFQGQVTISADKSISTA |
| | | | YLQWSSLKASDTAMYYCTRGDRGVDYWGQGTLVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKKVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYNSTYRVVSVLTVLHQDW |
| | | | LNGKEYKCKVSNKALPAPIEKTISKAKGQPREPQVYTLPP |
| | | | SREEMTKNQVSLTCLVKGFYPSDIAVEWESNGQPENNYK |
| | | | TTPPVLDSDGSFFLYSKLTVDKSRWQQGNVFSCSVMHEA |
| | | | LHNHYTQKSLSLSPGK |
| | Light | 479 | DIQMTQSPSSLSASVGDRVTITCRASQGISRWLAWYQQK |
| | Chain | | PEKAPKSLIYAASSLQSGVPSRFSGSGSGTDFTLTISGLQP |
| | | | EDFATYYCQQYNSYPRTFGQGTKVEIKRTVAAPSVFIFPP |
| | | | SDEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| CLEC12A | Heavy | 484 | QVQLQESGPGLVKPSETLSLTCVVSGGSISSSNWWSWVR |
| | Chain | | QPPGKGLEWIGEIYHSGSPDYNPSLKSRVTISVDKSRNQF |
| | variant | | SLKLSSVTAADTAVYYCAKVSTGGFFDYWGQGTLVTVS |
| | 1 | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELL |
| | | | GGPSVFLFPPKPKDTLMISRTPEVTCVVVDVSHEDPEVKF |
| | | | NWYVDGVEVHNAKTKPREEQYNSTYRVVSVLTVLHQD |
| | | | WLNGKEYKCKVSNKALPAPIEKTISKAKGQPREPQVYTL |
| | | | PPSREEMTKNQVSLTCLVKGFYPSDIAVEWESNGQPENN |
| | | | YKTTPPVLDSDGSFFLYSKLTVDKSRWQQGNVFSCSVM |
| | | | HEALHNHYTQKSLSLSPGK |
| | Heavy | 485 | QVQLQESGPGLVKPSETLSLTCVVSGGSISSSNWWSWVR |
| | Chain | | QPPGKGLEWIGEIYHSGSPNYNPSLKSRVTISVDKSKNQF |
| | variant | | SLKLSSVTAADTAVYYCARSSSGGFFDYWGQGTLVTVSS |
| | 2 | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKRVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYNSTYRVVSVLTVLHQDW |
| | | | LNGKEYKCKVSNKALPAPIEKTISKAKGQPREPQVYTLPP |
| | | | SREEMTKNQVSLTCLVKGFYPSDIAVEWESNGQPENNYK |
| | | | TTPPVLDSDGSFFLYSKLTVDKSRWQQGNVFSCSVMHEA |
| | | | LHNHYTQKSLSLSPGK |
| | Heavy | 486 | QVQLQESGPGLVKPSETLSLTCVVSGGSISSSNWWSWVR |
| | Chain | | QPPGKGLEWIGEIYHSGSPNYNPSLKSRVTISVDKSKNQF |
| | variant | | SLKLSSVTAADTAVYYCARQTTAGSFDYWGQGTLVTVS |
| | 3 | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELL |
| | | | GGPSVFLFPPKPKDTLMISRTPEVTCVVVDVSHEDPEVKF |
| | | | NWYVDGVEVHNAKTKPREEQYNSTYRVVSVLTVLHQD |
| | | | WLNGKEYKCKVSNKALPAPIEKTISKAKGQPREPQVYTL |
| | | | PPSREEMTKNQVSLTCLVKGFYPSDIAVEWESNGQPENN |
| | | | YKTTPPVLDSDGSFFLYSKLTVDKSRWQQGNVFSCSVM |
| | | | HEALHNHYTQKSLSLSPGK |
| | Light | 487 | DIQMTQSPSSLSASVGDRVTITCRASQSISSYLNWYQQKP |
| | Chain | | GKAPKLLIYAASSLQSGVPSRFSGSGSGTDFTLTISSLQPE |
| | | | DFATYYCQQSYSTPPTFGQGTKVEIKRTVAAPSVFIFPPSD |
| | | | EQLKSGTASVVCLLNNFYPREAKVQWKVDNALQSGNSQ |
| | | | ESVTEQDSKDSTYSLSSTLTLSKADYEKHKVYACEVTHQ |
| | | | GLSSPVTKSFNRGEC |
| BDCA-2 | Heavy | 488 | QVQLVESGGGVVQPGRSLRLSCAASGFTLSSYGMHWVR |
| Variant 1 | Chain | | QAPGKGLEWVAVIWYDGNDKYYADSVKGRFTISRDNSK |
| | | | NTLYLQVNSLRAEDTAVYYCARGTGTPYWYFDLWGRG |
| | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVLT |
| | | | VLHQDWLNGKEYKCKVSNKALPAPIEKTISKAKGQPREP |
| | | | QVYTLPPSRDELTKNQVSLTCLVKGFYPSDIAVEWESNG |
| | | | QPENNYKTTPPVLDSDGSFFLYSKLTVDKSRWQQGNVFS |
| | | | CSVMHEALHNHYTQKSLSLSPGK |
| | Light | 489 | EIVLTQSPATLSLSPGERATLSCRASQSVNNYLAWYQQK |
| | Chain | | PGQAPRLLIYDASNRATGIPARFSGSGSGTDFTLTISSLEPE |
| | | | DFAVYYCQQRSTWPPYTFGQGTKLEIKRTVAAPSVFIFPP |
| | | | SDEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| BDCA-2 | Heavy | 490 | EVQLVESGGGLVQPGGSLRLSCAASGFTFSNYLMNWVR |
| Variant 2 | Chain | | QAPGKGLEWVANIEQDGSEKYYVDSVKGRFTISRDNAK |
| | | | NSLYLQMNSLRAEDTAVYFCARDGDTAMITFDFWGQGT |
| | | | LVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKKVEPKSCDKTHTCPPCP |
| | | | APELLGGPSVFLFPPKPKDTLMISRTPEVTCVVVDVSHED |
| | | | PEVKFNWYVDGVEVHNAKTKPREEQYNSTYRVVSVLTV |
| | | | LHQDWLNGKEYKCKVSNKALPAPIEKTISKAKGQPREPQ |
| | | | VYTLPPSRDELTKNQVSLTCLVKGFYPSDIAVEWESNGQ |
| | | | PENNYKTTPPVLDSDGSFFLYSKLTVDKSRWQQGNVFSC |
| | | | SVMHEALHNHYTQKSLSLSPGK |
| | Light | 491 | DIQMTQSPSSVSASVGDRVTITCRASQGIRRWLAWYQQK |
| | Chain | | PGKAPKLLIYAASSLQRGVPSRFSGSGSGTDFTLTISSLQP |
| | | | EDFATYYCQQANSFPWTFGQGTKVEIKRTVAAPSVFIFPP |
| | | | SDEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| BDCA-2 | Heavy | 492 | QVQLQESGPGLVKPSQTLSLTCTVSGGSISSGGYYWNWI |
| Variant 3 | Chain | | RQHPGKGLEWIGYIYYSGNTYYNPSLKSRVTISVDTSKN |
| | | | QFSLKLSSVTAADAAVYHCARGYGDYGGGYFDYWGQG |
| | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVLT |
| | | | VLHQDWLNGKEYKCKVSNKALPAPIEKTISKAKGQPREP |
| | | | QVYTLPPSRDELTKNQVSLTCLVKGFYPSDIAVEWESNG |
| | | | QPENNYKTTPPVLDSDGSFFLYSKLTVDKSRWQQGNVFS |
| | | | CSVMHEALHNHYTQKSLSLSPGK |
| | Light | 493 | DIQMTQSPSSLSASVGDRVTITCQASQDISNYLNWYQQK |
| | Chain | | PGKAPKFLIYDVSNLETGVPSRFSGSGSGTDFTFTISSLQP |
| | | | EDIATYYCQQYDNLPYTFGQGTKLEIKRTVAAPSVFIFPP |
| | | | SDEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |

An antibody construct of a conjugate can comprise a heavy chain and a light chain that specifically bind to an antigen expressed by an immune cell such as an antigen presenting cell. An antibody construct can comprise a first binding domain and an Fc domain, wherein the first binding domain and the Fc domain comprise an antibody. An antibody construct can comprise a first binding domain, a second binding domain and an Fc domain, wherein the first binding domain, the second binding domain and the Fc domain comprise an antibody. An antibody construct of a conjugate can comprise an anti-CD40 antibody, the antibody construct comprising: a) a heavy chain sequence having at least 80% sequence identity to an amino acid sequence of SEQ ID NO: 1 and a light chain sequence having at least 80% sequence identity to an amino acid sequence of SEQ ID NO: 6; b) a heavy chain sequence having at least 80% sequence identity to an amino acid sequence of SEQ ID NO: 401 or SEQ ID NO: 402, and a light chain sequence having at least 80% sequence identity to an amino acid sequence of SEQ ID NO: 403; c) a heavy chain sequence having at least 80% sequence identity to an amino acid sequence of SEQ ID NO: 404, and a light chain sequence having at least 80% sequence identity to an amino acid sequence of SEQ ID NO: 409; d) a heavy chain sequence having at least 80% sequence identity to an amino acid sequence of SEQ ID NO: 414, and a light chain sequence having at least 80% sequence identity to an amino acid sequence of SEQ ID NO: 419; e) a heavy chain sequence having at least 80% sequence identity to an amino acid sequence of SEQ ID NO: 424, and a light chain sequence having at least 80% sequence identity to an amino acid sequence of SEQ ID NO: 429; f) a heavy chain sequence having at least 80% sequence identity to an amino acid sequence of SEQ ID NO: 434, and a light chain sequence having at least 80% sequence identity to an amino acid sequence of SEQ ID NO: 439; g) a heavy chain sequence having at least 80% sequence identity to an amino acid sequence of SEQ ID NO: 444, and a light chain sequence having at least 80% sequence identity to an amino acid sequence of SEQ ID NO: 449; or h) a heavy chain sequence having at least 80% sequence identity to an amino acid sequence of SEQ ID NO: 454, and a light chain sequence having at least 80% sequence identity to an amino acid sequence of SEQ ID NO: 459.

An antibody construct of a conjugate can comprise a first binding domain and an Fc domain, wherein the first binding domain and the Fc domain comprise an antibody. An antibody construct of a conjugate can comprise a first binding domain, a second binding domain and an Fc domain, wherein the first binding domain, the second binding domain and the Fc domain comprise an antibody. An antibody construct of a conjugate can comprise an anti-DEC-205 antibody, the antibody construct comprising: a) a heavy chain sequence having at least 80% sequence identity to an amino acid sequence of SEQ ID NO: 464, and a light chain sequence having at least 80% sequence identity to an amino acid sequence of SEQ ID NO: 469; or b) a heavy chain sequence having at least 80% sequence identity to an amino acid sequence of SEQ ID NO: 474, and a light chain sequence having at least 80% sequence identity to an amino acid sequence of SEQ ID NO: 479.

An antibody construct of a conjugate can comprise a first binding domain and an Fc domain, wherein the first binding domain and the Fc domain comprise an antibody. An antibody construct of a conjugate can comprise a first binding domain, a second binding domain and an Fc domain, wherein the first binding domain, the second binding domain and the Fc domain comprise an antibody. An antibody construct of a conjugate can comprise an anti-CLEC12A antibody, the antibody construct comprising: a) a heavy chain sequence having at least 80% sequence identity to an amino acid sequence of SEQ ID NO: 484, and a light chain sequence having at least 80% sequence identity to an amino acid sequence of SEQ ID NO: 487; b) a heavy chain sequence having at least 80% sequence identity to an amino acid sequence of SEQ ID NO: 485, and a light chain sequence having at least 80% sequence identity to an amino acid sequence of SEQ ID NO: 487; or c) a heavy chain sequence having at least 80% sequence identity to an amino acid sequence of SEQ ID NO: 486, and a light chain sequence having at least 80% sequence identity to an amino acid sequence of SEQ ID NO: 487.

An antibody construct of a conjugate can comprise a first binding domain and an Fc domain, wherein the first binding domain and the Fc domain comprise an antibody. An antibody construct of a conjugate can comprise a first binding domain, a second binding domain and an Fc domain, wherein the first binding domain, the second binding domain and the Fc domain comprise an antibody. An antibody construct of a conjugate can comprise an anti-BDCA-2 antibody, the antibody construct comprising: a) a heavy chain sequence having at least 80% sequence identity to an amino acid sequence of SEQ ID NO: 488, and a light chain sequence having at least 80% sequence identity to an amino acid sequence of SEQ ID NO: 489; b) a heavy chain sequence having at least 80% sequence identity to an amino acid sequence of SEQ ID NO: 490, and a light chain sequence having at least 80% sequence identity to an amino acid sequence of SEQ ID NO: 491; or c) a heavy chain sequence having at least 80% sequence identity to an amino acid sequence of SEQ ID NO: 492, and a light chain sequence having at least 80% sequence identity to an amino acid sequence of SEQ ID NO: 493.

### Antibody-ScFv Fusion Protein Products

An antibody construct of a conjugate can comprise a first binding domain, a second binding domain, and optionally a third binding, and an Fc domain, where one or more of the binding domains are attached to another binding domain or the Fc domain as a fusion protein. In some embodiments, a first binding domain and a second binding domain can be attached to the Fc domain as a fusion protein. In some embodiments, a first binding domain can be attached to the Fc domain at an N-terminal end of the Fc domain, wherein a second binding domain can be attached to the Fc domain at a C-terminal end. In some embodiments, a first binding domain can be attached to the Fc domain at an N-terminal end of the Fc domain, and a second binding domain can be attached to the Fc domain at a C-terminal end via a polypeptide linker ranging from 10 to 25 amino acids comprising the sequence [G4S]n where n=2 to 5. In some embodiments, a first binding domain can be attached to the Fc domain at a C-terminal end of the Fc domain, and a second binding domain can be attached to the Fc domain at an N-terminal end. In some embodiments, a first binding domain can be attached to the Fc domain at a C-terminal end of the Fc domain via a polypeptide linker ranging from 10 to 25 amino acids comprising the sequence [G4S]n where n=2 to 5, and a second binding domain can be attached to the Fc domain at an N-terminal end.

In some embodiments, a first binding domain, a second binding domain and an Fc domain can comprise an antibody. In some embodiments, a second binding domain and an Fc domain can comprise an antibody and a first binding domain can comprise a single chain variable fragment (scFv). In some embodiments, a second binding domain, a third binding domain and an Fc domain can comprise an antibody and a first binding domain can comprise a single chain variable fragment (scFv).

A single chain variable fragment typically comprises a heavy chain variable domain and a light chain variable domain of an antibody. In some embodiments, a first binding domain of the fusion protein can be attached to the second binding domain at a heavy chain variable domain of the single chain variable fragment of the first binding domain (HL orientation). In some embodiments, a first binding domain of the fusion protein can be attached to the second binding domain at a light chain variable domain of the single chain variable fragment of the first binding domain (LH orientation). In either orientation, the first binding domain and the second binding domain can be attached via a polypeptide linker varying in length from 15 to 25 amino acids, wherein the linker comprises the sequence [G4S]n where n=3 to 5.

In other embodiments, a first binding domain and an Fc domain can comprise an antibody and the second binding domain can comprise a single chain variable fragment (scFv). The second binding domain of the fusion protein can be attached to the first binding domain at a heavy chain variable domain of the single chain variable fragment of the first binding domain (HL orientation). Alternatively, the second binding domain of the fusion protein can be attached to the first binding domain at a light chain variable domain of the single chain variable fragment of the first binding domain (LH orientation). In either orientation, the first binding domain or second binding domain can be attached via a polypeptide linker varying in length from 15 to 25 amino acids, wherein the linker comprises the sequence [G4S]n where n=3 to 5.

In other embodiments, a first binding domain, a second binding domain and an Fc domain can comprise an antibody and a third binding domain can comprise a single chain variable fragment (scFv). The third binding domain of the fusion protein can be attached to the first binding domain at a heavy chain variable domain of the single chain variable fragment of the first binding domain (HL orientation). Alternatively, the third binding domain of the fusion protein can be attached to the first binding domain at a light chain variable domain of the single chain variable fragment of the first binding domain (LH orientation). In either orientation, the third binding domain can be attached via a polypeptide linker varying in length from 15 to 25 amino acids, wherein the linker comprises the sequence [G4S]n where n=3 to 5.

A conjugate or an antibody construct can comprise a first binding domain and a second binding domain, wherein the second binding domain can be attached to the first binding domain. The conjugate or antibody construct can comprise an antibody comprising a light chain and a heavy chain. The first binding domain can comprise a Fab fragment of the light and heavy chains. The second binding domain can be attached to the light chain at a C-terminus or C-terminal end of the light chain as a fusion protein. The second binding domain can comprise a single chain variable fragment (scFv).

A conjugate or antibody construct can comprise a first binding domain, a second binding domain, and an Fc domain, wherein the first binding domain and the second binding domain are attached to the Fc domain as a fusion protein. The second binding domain of the fusion protein can specifically target an antigen with at least 80% identity to CD40. The second binding domain of the fusion protein can be a CD40 agonist. The first binding domain of the fusion protein can target a tumor antigen. The conjugate or antibody construct can comprise a fusion protein comprising a heavy chain (HC) attached to a single chain variable fragment.

In certain embodiments, a first binding domain or a first and second binding domain can specifically bind to a tumor antigen, such as, for example, GD2, GD3, GM2, Le^{y}, sLe, polysialic acid, fucosyl GM1, Tn, STn, BM3, or GloboH. In certain embodiments, a first binding domain or a first and second binding domain can specifically can bind to a tumor antigen having an amino acid sequence having at least 80%, 90%, 95%, 97%, 98%, 99% or 100% identity to the amino acid sequence of CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, STN1, TNC, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), VISTA, or a fragment thereof.

In certain embodiments, a first binding domain or a first and second binding domain specifically binds to a tumor antigen, such as those selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, αvβ3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In some embodiments, a first binding domain of the fusion protein can target an antigen with at least 80% or 100% identity to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, or NY-ESO-1. In some embodiments, a first binding domain of the fusion protein also can target an antigen with at least 80% or 100% identity to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4), or STRA6. In some embodiments, a first binding domain can target an antigen with at least 80% or 100% identity to the amino acid sequence of TROP2, CEA, MUC16, LRRC15, CLDN6, CLDN16, UPK1B, VTCN1 (B7-H4) or STRA6.

In certain embodiments, a first or first and second antigen binding domain can specifically bind to an antigen associated with fibrotic or inflammatory disease, such as those selected from Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, MMP14, GPX8, and F2RL2.

In some embodiments, a first binding domain of a fusion protein binds to a tumor antigen, as set forth above. A second binding domain or a third binding domain of the fusion protein can specifically target an antigen of an immune cell such as an antigen presenting cell (APC). A second binding domain or a third binding domain of the fusion protein can specifically bind to target an antigen with at least 80% identity to CD40. A second binding domain or a third binding domain of the fusion protein can be a CD40 agonist. A second binding domain or a third binding domain of the fusion protein can specifically bind to an antigen with at least 80% identity to DEC-205. A second binding domain or a third binding domain of the fusion protein can specifically bind to an antigen with at least 80% identity to DC-SIGN. A second binding domain or a third binding domain of the fusion protein can specifically bind to an antigen with at least 80% identity to CD36 mannose scavenger receptor. A second binding domain or a third binding domain of the fusion protein can specifically bind to an antigen with at least 80% identity to CLEC12A. A second binding domain or a third binding domain of the fusion protein can specifically bind to an antigen with at least 80% identity to BDCA-2. A second binding domain or a third binding domain of the fusion protein can specifically bind to an antigen with at least 80% or 100% identity to the amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1 or CD32B. A first binding domain, or a first and second binding domain of the fusion protein can target a tumor antigen.

Alternatively, the second binding domain of the fusion protein can specifically bind to a tumor antigen. The second binding domain of the fusion protein can specifically bind to an antigen with at least 80% or 100% identity to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, or NY-ESO-1. The second binding domain of the fusion protein also can specifically bind to an antigen with at least 80% or 100% identity to HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4), or STRA6. A first binding domain of the fusion protein can specifically bind to an antigen with at least 80% or 100% identity to the amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1 or CD32B.

In some embodiments, the first binding domain can specifically bind to an antigen with at least 80% or 100% identity to the amino acid sequence of TROP2, CEA, MUC16, LRRC15, CLDN6, CLDN16, UPK1B, VTCN1 (B7-H4) and STRA6 and a second binding domain can specifically bind to an antigen with at least 80% or 100% identity to the amino acid sequence of CD40 or PD-L1.

In some embodiments, a third binding domain of the fusion protein can specifically bind to an antigen of an immune cell such as an antigen presenting cell (APC). A third binding domain of the fusion protein can specifically bind to an antigen with at least 80% identity to the amino acid sequence of CD40. A third binding domain of the fusion protein can specifically bind to an antigen with at least 80% identity to the amino acid sequence of CD40, DEC-205, CD36 mannose scavenger receptor 1, CLEC9A, DC-SIGN, CLEC12A, BDCA-2, OX40L, 41BBL, CD204, MARCO, CLEC5A, Dectin 1, Dectin 2, CLEC10A, CD206, CD64, CD32A, CD16A, HVEM, PD-L1 or CD32B.

In some embodiments, a third binding domain can specifically bind to a tumor antigen. In some embodiments, a third binding domain can specifically bind to a tumor antigen, such as, for example, GD2, GD3, GM2, Le^{y}, sLe, polysialic acid, fucosyl GM1, Tn, STn, BM3, or GloboH. In some embodiments, a third binding domain specifically can bind to a tumor antigen having an amino acid sequence having at least 80%, 90%, 95%, 97%, 98%, 99% or 100% identity to the amino acid sequence of CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, STN1, TNC, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), VISTA, or a fragment thereof.

In some embodiments, a third binding domain of the fusion protein can specifically bind to an antigen with at least 80% or 100% identity to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, or NY-ESO-1. In some embodiments, a third binding domain of the fusion protein can specifically bind to an antigen with at least 80% or 100% identity to the amino acid sequence of HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, LRRC15, GLP-3, CLDN6, CLDN16, UPK1B, VTCN1 (B7-H4), or STRA6.

### Other Characteristics of Antibody Constructs

Conjugates comprising antibody constructs described herein, including those containing the sequences referenced in TABLES 1-6 can have a dissociation constant (Kd) that is less than 10 nM for the antigen of the first binding domain. The conjugates of the antibody constructs described herein, including those containing the sequences referenced in TABLES 1-6 can have a dissociation constant (Kd) that is less than 10 nM for the antigen of the second binding domain. The conjugates of the antibody constructs described herein, including those containing the sequences referenced in TABLES 1-6 can have a dissociation constant (Kd) that is less than 10 nM for the antigen of the third binding domain. The conjugates or antibody constructs can have a dissociation constant (Kd) for the antigen of the first binding domain that is less than 1 nM, less than 100 pM, less than 10 pM, less than 1 pM, or less than 0.1 pM. The conjugates or antibody constructs can have a dissociation constant (Kd) for the antigen of the second binding domain that is less than 1 nM, less than 100 pM, less than 10 pM, less than 1 pM, or less than 0.1 pM. The conjugates or antibody constructs can have a dissociation constant (Kd) for the antigen of the third binding domain that is less than 1 nM, less than 100 pM, less than 10 pM, less than 1 pM, or less than 0.1 pM.

### Fc Domains

An antibody construct of a conjugate includes an Fc domain. An Fc domain is a structure that can bind to one or more Fc receptors (FcRs). An Fc domain can be from an antibody. An Fc domain can contain an Fc region. An Fc region can be an Fc domain.

An Fc region of an antibody construct can be of any suitable type, which can be selected from the classes of immunoglobins, IgA, IgD, IgE, IgG, and IgM. Several different classes can be further divided into isotypes, e.g., IgG1, IgG2, IgG3, IgG4, IgA1, and IgA2. The heavy-chain constant regions (Fc) that corresponds to the different classes of immunoglobulins can be α, δ, ε, γ, and μ, respectively. The light chains can be one of either kappa or κ and lambda or λ. The Fc region comprises a number of Fc domains, C_{H}1, C_{H}2, C_{H}3 and C_{H}4, according to the type of antibody.

In some embodiments, an Fc domain can have an IgG1 isotype. In some embodiments, an Fc domain can have an IgG2 isotype. In some embodiments, an Fc domain can have an IgG3 isotype. In some embodiments, an Fc domain can have an IgG4 isotype. In some embodiments, an Fc domain can have a hybrid isotype comprising constant regions from two or more isotypes.

An Fc receptor can bind to an Fc domain. Fc domains are portions of the Fc region of an antibody. FcRs can bind to an Fc domain of an antibody. FcRs can bind to an Fc domain of an antibody construct (e.g., an antibody) bound to an antigen. FcRs are organized into classes (e.g., gamma (γ), alpha (α) and epsilon (ε)) based on the class of antibody that the FcR recognizes. The FcαR class can bind to IgA and includes several isoforms, FcαRI (CD89) and FcαμR. The FcγR class can bind to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant. An FcγRIIIA (CD16a) can be an FcγRIIIA (CD16a) V158 variant. Each FcγR isoform can differ in affinity to the Fc region or Fc domain of an IgG antibody. For example, FcγRI can bind to IgG with greater affinity than FcγRII or FcγRIII. The affinity of a particular FcγR isoform to IgG can be controlled, in part, by a glycan (e.g., oligosacccharaide) at position CH2 84.4 of the IgG antibody. For example, fucose containing CH2 84.4 glycans can reduce IgG affinity for FcγRIIIA In addition, G0 glucans can have increased affinity for FcγRIIIA due to the lack of galactose and terminal GlcNAc moiety.

Binding of an Fc domain to an FcR can enhance an immune response. FcR-mediated signaling that can result from an Fc region binding to an FcR can lead to the maturation of immune cells. FcR-mediated signaling that can result from an Fc domain binding to an FcR can lead to the maturation of dendritic cells. FcR-mediated signaling that can result from an Fc domain binding to an FcR can lead to antibody dependent cellular cytotoxicity. FcR-mediated signaling that can result from an Fc domain binding to an FcR can lead to more efficient immune cell antigen uptake and processing. FcR-mediated signaling that can result from an Fc region binding to an FcR can lead to more efficient dendritic cell antigen uptake and processing. FcR-mediated signaling that can result from an Fc region binding to an FcR can increase antigen presentation. FcR-mediated signaling that can result from an Fc region binding to an FcR can increase antigen presentation by immune cells. FcR-mediated signaling that can result from an Fc region binding to an FcR can increase antigen presentation by antigen presenting cells. FcR-mediated signaling that can result from an Fc domain binding to an FcR can increase antigen presentation by dendritic cells. FcR-mediated signaling that can result from an Fc domain binding to an FcR can promote the expansion and activation of T cells. FcR-mediated signaling that can result from an Fc domain binding to an FcR can promote the expansion and activation of CD8+ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a TH1 cell response).

The profile of FcRs on a DC can impact the ability of the DC to respond upon stimulation. For example, most DC can express both CD32A and CD32B, which can have opposing effects on IgG-mediated maturation and function of DCs: binding of IgG to CD32A can mature and activate DCs in contrast with CD32B, which can mediate inhibition due to phosphorylation of immunoreceptor tyrosine-based inhibition motif (ITIM), after CD32B binding of IgG. Therefore, the activity of these two receptors can establish a threshold of DC activation. Furthermore, difference in functional avidity of these receptors for IgG can shift their functional balance. Hence, altering the Fc domain binding to FcRs can also shift their functional balance, allowing for manipulation (either enhanced activity or enhanced inhibition) of the DC immune response.

An Fc domain can have a sequence that has been modified, as compared to a wild type sequence, to alter at least one constant region-mediated biological effector function relative to the corresponding wild type sequence. For example, in some embodiments, an Fc domain can be modified to reduce or increase at least one constant region-mediated biological effector function relative to an unmodified Fc domain, e.g., reduced or increased binding to an Fc receptor (FcR). FcR binding can be reduced or increased by, for example, modifying the immunoglobulin constant region segment of the antibody construct at particular regions involved in (e.g., necessary for or affecting) FcR interactions.

In some embodiments, an antibody construct described herein can have an Fc domain that is modified to acquire or improve at least one constant region-mediated biological effector function relative to an unmodified Fc domain, e.g., to enhance FcγR interactions. For example, an antibody construct with an Fc domain that binds to FcγRIIA, FcγRIIB and/or FcγRIIIA with greater affinity than the corresponding wild type Fc domain can be produced according to the methods known to the skilled artisan.

A modification in the amino acid sequence Fc domain can alter the recognition of an FcR for the Fc domain. However, such modifications can still allow for FcR-mediated signaling. A modification can be a substitution of an amino acid at a residue (e.g., wildtype) for a different amino acid at that residue. A modification can permit binding of an FcR to a site on the Fc region that the FcR may not otherwise bind to. A modification can increase binding affinity of an FcR to the Fc domain that the FcR may have reduced affinity for binding. A modification can decrease binding affinity of an FcR to a site on the Fc domain that the FcR may have increased affinity for binding. A modification can increase the subsequent FcR-mediated signaling after Fc binding to an FcR.

In some embodiments, a modification in the amino acid sequence Fc domain can alter the recognition of an FcR, or multiple FcRs, for the Fc domain. Such a modification(s) can alter the ability of a conjugate to interact with immune cells. Such a modification or series of modifications to an Fc domain, can permit selective binding of an Fc domain to FcRs on immune cells, such as APCs, and selective delivery of the immune-modulatory agent to the immune cells (APCs). For example, modifications to the Fc domain an reduce binding by the FC domain to Fc gamma receptors, but retain the ability of the Fc domain to bind to FcRn.

An Fc region can have at least one amino acid change as compared to the sequence of the wild-type Fc region. An Fc domain with at least one amino acid change as compared to the sequence of the wild-type Fc domain. An amino acid change in an Fc region can allow the antibody construct or conjugate to bind to at least one Fc receptor with greater affinity compared to a wild-type Fc region. An amino acid change in an Fc domain can allow the antibody to bind to at least one Fc receptor with greater affinity compared to a wild-type Fc domain. An amino acid change in an Fc region can allow the antibody construct or conjugate to bind to at least one Fc receptor with decreased affinity compared to a wild-type Fc region. An amino acid change in an Fc domain can allow the antibody to bind to at least one Fc receptor with decreased affinity compared to a wild-type Fc domain.

An Fc region can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc domain can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence.

An antibody construct can comprise an Fc domain or Fc region comprising a sequence of the IgG1 isoform that has been modified from the wild type IgG1 sequence. A modification can comprise a substitution at one or more one amino acid residues of an Fc domain such as at 5 different amino acid residues including L235V/F243L/R292P/Y300L/P396L (IgG1VLPLL), according to the EU index of Kabat. A modification can comprise a substitution at one or more amino acid residues such as at 2 different amino acid residues of an Fc domain including S239D/I332E (IgG1DE), according to the EU index of Kabat. A modification can comprise a substitution at one or more amino acid residues such as at 3 different amino acid residues of an Fc domain including S298A/E333A/K334A (IgG1AAA), according to the EU index of Kabat.

In some embodiments, an Fc domain or region can exhibit reduced binding affinity to one or more Fc receptors. In some embodiments, an Fc domain or region can exhibit reduced binding affinity to one or more Fcgamma receptors. In some embodiments, an Fc domain or region can exhibit reduced binding affinity to FcRn receptors. In some embodiments, an Fc domain or region can exhibit reduced binding affinity to Fcgamma and FcRn receptors. In some embodiments, an Fc domain is an Fc null domain or region. As used herein, an “Fc null” refers to a domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

The Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more amino acid substitutions that reduces the binding affinity of the Fc domain or region to an Fc receptor.

In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Kabat numbering.

In some embodiments, the Fc domain or region can comprise a sequence of an IgG isoform that has been modified from the wild-type IgG sequence. In some embodiments, the Fc domain or region can comprise a sequence of the IgG1 isoform that has been modified from the wild-type IgG1 sequence. In some embodiments, the modification comprises substitution of one or more amino acids that reduce binding affinity of an IgG Fc domain or region to all Fcγ receptors. A modification can be substitution of E233, L234 and L235, such as E233P/L234V/L235A or E233P/L234V/L235A/ΔG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain or region comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain or region comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain or region to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain or region to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain or region to FcγRII receptor. A modification can be a substitution of R255, such as R255A, according to the EU index of Kabat. A modification can be a substitution of E258, such as E258A, according to the EU index of Kabat. A modification can be a substitution of 5267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of 5337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc domain or region to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain or region to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain or region to FcγRII receptor and increases the binding affinity to FcγRIIIA receptor. A modification can be a substitution of S298, such as S298A, according to the EU index of Kabat. A modification can be substitution of S239, 1332 and A330, such as S239D/1332E/A330L. A modification can be substitution of S239 and 1332, such as S239D/I332E.

In some embodiments, the modification comprises substitution of one or more amino acids that decreases binding affinity of an IgG Fc domain or region to FcγRIIIA receptor. A modification can be substitution of F241 and F243, such as F241S/F243S or F241I/F2431, according to the EU index of Kabat.

In some embodiments, the modification comprises substitution of one or more amino acids that decreases binding affinity of an IgG Fc domain or region to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain or region to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and 1332, such as S239D/I332E.

In some embodiments, the modification comprises substitution of one or more amino acids that increases binding affinity of an IgG Fc domain or region to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc domain that affect its interaction with one or more Fcγ receptors are disclosed in U.S. Pat. Nos. 7,317,091 and 8,969,526 (the disclosures of which are incorporated by reference herein).

In some embodiments, an IgG Fc domain or region comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at I253, such as I253A according to the EU index of Kabat. A modification can comprise a substitution at H310, such as H310A according to the EU index of Kabat. A modification can comprise substitutions at I253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P2571, V279E, V279Q, V279Y, A281S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

### Immune-Modulatory Agents

The conjugates described herein further comprise an immune-modulatory agent(s) attached to the antibody construct. In certain embodiments, an immune-modulatory agent can be coupled to the Fc domain of an antibody construct. An immune-modulatory agent can be an immune-stimulatory compound (or molecule) or an immune-suppressive compound (or molecule). An immune-modulatory agent can provide a direct, indirect or adjuvant effect. An immune-stimulatory compound can be a compound or molecule that directly or indirectly stimulates an immune response, such as anti-tumor immune response, after administration. For example, an immune-stimulatory compound can directly stimulate an immune response by causing the release of cytokines by an immune cell expressing a receptor of the immune-stimulatory compound, which results in the activation of immune cells that can, for example, prevent tumor growth or kill tumor cells. As another example, an immune-stimulatory compound can indirectly stimulate an immune response by supressing IL-10 production and secretion by the target cell and/or by supressing the activity of regulatory T cells, resulting in, for example, an increased anti-tumor response by immune cells. An immune-inhibitory compound can be a compound or molecule that directly or indirectly inhibits an immune response, such as an inflammatory response, after administration.

In certain embodiments, an immune-modulatory agent can target a pattern recognition receptor (PRR). These receptors can be transmembrane or intra-endosomal proteins which can prime activation of the immune system in response to infectious agents such as pathogens. PRRs can recognize pathogen-associated molecular patterns (PAMPs) molecules and damage-associated molecular patterns (DAMPs) molecules. A PRR can be membrane bound. A PRR can be cytosolic. Membrane-bound PRRs include toll-like receptors and C-type lectin receptors, such as mannose receptors and asialoglycoprotein receptors. Cytoplastic PRRs include NOD-like receptors, and RIG-I-like receptors.

In certain embodiments, an immune-modulatory agent can be a Damage-Associated Molecular Pattern (DAMP) molecule or a Pathogen-Associated Molecular Pattern (PAMP) molecule, such as a DAMP agonist or a PAMP agonist. DAMP molecules and PAMP molecules can be recognized by receptors of the innate immune system, such as Toll-like receptors (TLRs), Nod-like receptors, C-type lectins, and RIG-I-like receptors. In certain embodiments, an immune-modulatory agent is a Toll-like receptor agonist, a STING agonist, or a RIG-I agonist.

Examples of DAMP molecules can include proteins such as chromatin-associated protein high-mobility group box 1 (HMGB1), S100 molecules of the calcium modulated family of proteins and glycans, such as hyaluronan fragments, and glycan conjugates. DAMP molecules can also be nucleic acids, such as DNA, when released from tumor cells following apoptosis or necrosis. Examples of additional DAMP nucleic acids can include RNA and purine metabolites, such as ATP, adenosine and uric acid, present outside of the nucleus or mitochondria.

In some embodiments, an immune-modulatory agent can be a cytosolic DNA and bacterial nucleic acids called cyclic dinucleotides, that are recognized by Interferon Regulatory Factor (IRF) or stimulator of interferon genes (STING), which can act a cytosolic DNA sensor. Compounds recognized by Interferon Regulatory Factor (IRF) can play a role in immunoregulation by TLRs and other pattern recognition receptors.

An immune-modulatory agent can be a toll-like receptor (TLR) agonist. An immune-modulatory agent can be RIG-I-like receptor ligand. An immune-modulatory agent can be a C-type lectin receptor ligand. An immune-modulatory agent can be a NOD-like receptor ligand.

In some embodiments, an immune-modulatory agent can be a TLR agonist. In some embodiments, an immune-modulatory agent can be selected from a TLR1, TLR2, TLR3, TLR4, TLR5, TLR6, TLR7, TLR8, TLR9, TLR10, TLR11, TLR12 or TLR13 agonist, according the animal species.

In some embodiments, an immune-stimulatory compound is a ligand of TLR2 selected from the group consisting of: (a) a heat killed bacteria product, preferably HKAL, HKEB, HKHP, HKLM, HKLP, HKLR, HKMF, HKPA, HKPG, or HKSA, HKSP, and (b) a cell-wall components product, preferably LAM, LM, LPS, LIA, LIA, PGN, FSL, Pam2CSK4, Pam3CSK4, or Zymosan.

In some embodiments, an immune-stimulatory compound is a ligand of TLR3 selected from the group consisting of: rintatolimod, poly-ICLC, RIBOXXON®, Apoxxim, RIBOXXIM®, IPH-33, MCT-465, MCT-475, and ND-1.1.

In some embodiments, an immune-stimulatory compound is a ligand of TLR4 selected from the group consisting of LPS, MPLA or a pyrimido[5,4-b]indole such as those described in WO 2014/052828 (U of Cal), AZ126 (N-(2-(cyclopentylamino)-2-oxo-1-(pyridin-4-yl)ethyl)-N-(4-methoxyphenyl)-3-methyl-5-phenyl-1H-pyrrole-2-carboxamide) or AZ368 ((E)-3-(4-(2-(cyclopentylamino)-1-(N-(4-isopropylphenyl)-1,5-diphenyl-1H-pyrazole-3-carboxamido)-2-oxoethyl)phenyl)acrylic acid).

In some embodiments, an immune-stimulatory compound is a ligand of TLR5 selected from the group consisting of: FLA and Flagellin;

In some embodiments, an immune-stimulatory compound is a ligand of TLR6.

In certain embodiments, an immune-stimulatory compound is a TLR7 agonist and/or a TLR8 agonist. In certain embodiments, an immune-stimulatory compound is a TLR7 agonist. In certain embodiments, an immune-stimulatory compound is a TLR8 agonist. In some embodiments, an immune-stimulatory compound selectively agonizes TLR7 and not TLR8. In other embodiments, an immune-stimulatory compound selectively agonizes TLR8 and not TLR7.

In certain embodiments, an immune-stimulatory compound is a TLR7 agonist. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine, but is other than a guanosine analog, an adenosine analog, a thymidine homopolymer, ssRNA, CpG-A, PolyG10, and PolyG3. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences). In some embodiments, a TLR7 agonist has an EC50 value of 500 nM or less by PBMC assay measuring TNFalpha or IFNalpha production. In some embodiments, a TLR7 agonist has an EC50 value of 100 nM or less by PBMC assay measuring TNFalpha or IFNalpha production. In some embodiments, a TLR7 agonist has an EC50 value of 50 nM or less by PBMC assay measuring TNFalpha or IFNalpha production. In some embodiments, a TLR7 agonist has an EC50 value of 10 nM or less by PBMC assay measuring TNFalpha or IFNalpha production.

In certain embodiments, an immune-stimulatory compound is a TLR8 agonist. In certain embodiments, the TLR8 agonist is selected from the group consisting of a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In certain embodiments, a TLR8 agonist is selected from the group consisting of a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine and is other a ssRNA. In some embodiments, an immune-modulatory agent is a TLR8 agonist, other than a naturally occurring TLR8 agonist or a benzazepine agonist of TLR8. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463, and the compounds disclosed in US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics

In some embodiments, the immune-stimulatory compound is a compound of Formula (IA), (IB), or (IC):

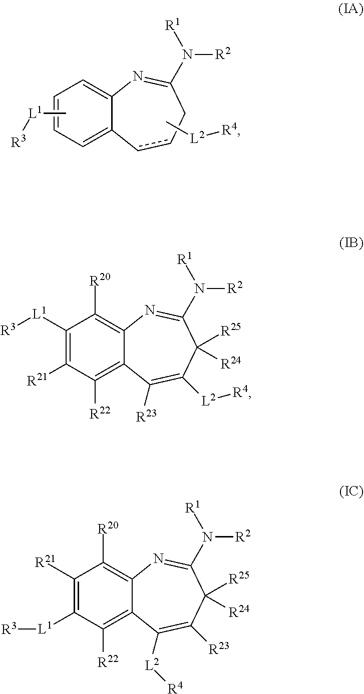

or a pharmaceutically acceptable salt thereof:

L¹ is selected from —N(R¹⁰)C(O)—*, —S(O)₂N(R¹⁰)—*, —CR¹⁰2N(R¹⁰)C(O)—*and —X²—C₁₋₆ alkylene-X²—C₁₋₆ alkylene-, wherein * represents where L¹ is bound to R³;

L² is —C(O)—;

X² at each occurrence is independently selected from —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰)—, —N(R¹⁰)C(O)—, —N(R¹⁰)C(O)N(R¹⁰)—, and —N(R¹⁰)C(O)O—;

R¹ and R² are independently selected from hydrogen; and C₁₋₁₀ alkyl optionally substituted with one or more substituents selected from halogen, —OR¹⁰, —SR¹⁰, —C(O)N(R¹⁰)₂, —N(R¹⁰)₂, —NO₂, ═O, ═S, ═N(R¹⁰), and —CN;

R³ is selected from an optionally substituted phenyl, heteroaryl and 9- or 10-membered bicyclic carbocycle wherein substituents are independently selected at each occurrence from: halogen, —OR¹⁰, —SR¹⁰, —C(O)N(R¹⁰)₂, —NO₂, ═O, ═S, ═N(R¹⁰), and —CN; C₁₋₁₀ alkyl optionally substituted at each occurrence with one or more substituents selected from halogen, —OR¹⁰, —SR¹⁰, —N(R¹⁰)₂, —C(O)R¹⁰, —NO₂, ═O, ═S, ═N(R¹⁰), —CN, C₃₋₁₂ carbocycle, and 3- to 12-membered heterocycle;

R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl optionally substituted at each occurrence with one or more substituents selected from halogen, —OR¹⁰, —SR¹⁰, —N(R¹⁰)₂, —C(O)R¹⁰, —NO₂, ═O, ═S, ═N(R¹⁰), —CN, C₃₋₁₂ carbocycle, and 3- to 12-membered heterocycle;

R¹⁰ is independently selected at each occurrence from: hydrogen, —NH₂, —C(O)OCH₂C₆H; and C₁₋₁₀ alkyl, C₂₋₁₀ alkenyl, C₂₋₁₀ alkynyl, C₃₋₁₂ carbocycle, and 3- to 12-membered heterocycle, each of which is independently optionally substituted at each occurrence with one or more substituents selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl; and wherein any substitutable carbon on the benzazepine core of Formula (IA) is optionally substituted with one or more R¹², wherein R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle; or for a compound of Formula (IB) or (IC), R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁰, R²¹, R²², and R²³ are each hydrogen; and R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁴ and R²⁵ are each hydrogen.

In some embodiments, the immune-stimulatory compound is a compound of Formula (IIA), (IIB) or (IIC):

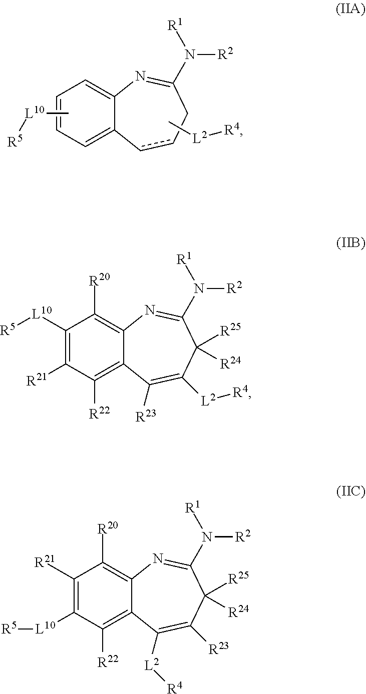

or a pharmaceutically acceptable salt thereof, wherein:

L¹⁰ is selected from —C(O)N(R¹⁰)—* and —C(O)—, wherein * represents where Lo is bound to R⁵;

L² is —C(O)—;

R¹ and R² are each hydrogen;

R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰;

R⁵ is selected from optionally substituted fused 5-5, fused 5-6, and fused 6-6 bicyclic heterocycle wherein substituents are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;

R¹⁰ is independently selected at each occurrence from hydrogen, —NH₂, —C(O)OCH₂C₆H₅; and C₁₋₁₀ alkyl optionally substituted with one or more substituents independently selected from halogen, —OH, —CN, —NO₂, —NH₂, ═O, ═S, —C(O)OCH₂C₆H₅, —NHC(O)OCH₂C₆H₅, C₃₋₁₂ carbocycle, and 3- to 12-membered heterocycle; and

wherein any substitutable carbon on the benzazepine core of Formula (IIA) is optionally substituted by a substituent independently selected from R¹², wherein R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, or

for a compound of Formula (IIB) or (IIC), R²⁰, R²¹, R²², and R²³ are independently selected from hydrogen, halogen, —OR¹⁰, —SR¹⁰, —N(R¹⁰)₂, —S(O)R¹⁰, —S(O)₂R′, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁰, R²¹, R²², and R²³ are each hydrogen; and

R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁴ and R²⁵ are each hydrogen.

In some embodiments, the immune-stimulatory compound is a compound of Formula (IIB) or (IIC):

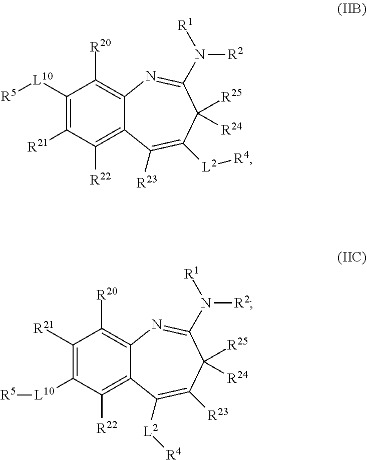

or a pharmaceutically acceptable salt thereof, wherein:

L¹⁰ is selected from —C(O)N(R¹⁰)—*, such as —C(O)NH—*, wherein * represents where L¹⁰ is bound to R⁵;

L² is —C(O)—;

R¹ and R² are each hydrogen;

R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰, such as R⁴ is —N(R¹⁰)₂;

R⁵ is selected from optionally substituted optionally substituted fused 5-5, fused 5-6, and fused 6-6 bicyclic heterocycle, such as R⁵ is selected from a tetrahydroquinoline, tetrahydroisoquinaline, dihydroindene, wherein substituents on R5 are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and C₁₋₁₀ alkyl optionally substituted at each occurrence with one or more substituents selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle;

R¹⁰ is independently selected at each occurrence from hydrogen, —NH₂, —C(O)OCH₂C₆H₅; and C₁₋₁₀ alkyl optionally substituted with one or more substituents independently selected from halogen, —OH, —CN, —NO₂, —NH₂, ═O, ═S, —C(O)OCH₂C₆H₅, —NHC(O)OCH₂C₆H₅, C₃₋₁₂ carbocycle, and 3- to 12-membered heterocycle;

R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C2-1o alkenyl, and C2-1o alkynyl, such as R²⁰, R²¹, R²², and R²³ are each hydrogen; and

R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁴ and R²⁵ are each hydrogen.

In certain embodiments, the immune-stimulatory compound is a compound of Formula (IIIA), (IIIB) or (IIIC):

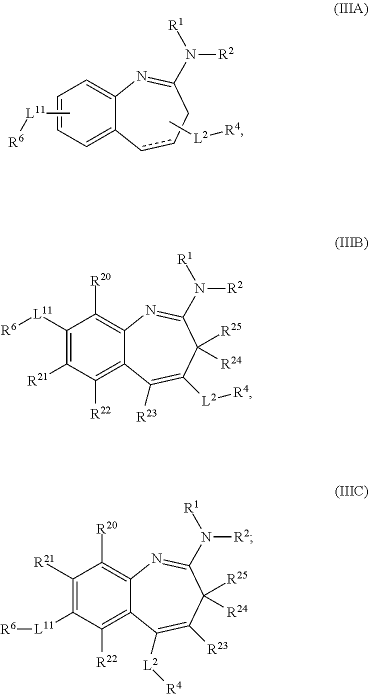

or a pharmaceutically acceptable salt thereof, wherein:

L¹¹ is —C(O)N(R¹⁰)—*, wherein * represents where L¹¹ is bound to R⁶;

L² is —C(O)—;

R¹ and R² are each hydrogen;

R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰;

R⁶ is selected from phenyl and 5- or 6-membered heteroaryl, any one of which is substituted with one or more substituents selected from R⁷;

R⁷ is selected from —C(O)NHNH₂, —C(O)NH—C₁₋₃alkylene-NH(R¹⁰), —C(O)CH₃, —C₁₋₃alkylene-NHC(O)OR¹⁰, —C₁₋₃alkylene-NHC(O)R¹⁰, —C₁₋₃alkylene-NHC(O)NHR¹⁰, and —C₁₋₃alkylene-NHC(O)—C₁₋₃alkylene-(R¹⁰)₂;

R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is independently optionally substituted at each occurrence with one or more substituents selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;

R¹¹ is selected from C₃₋₁₂ carbocycle and 3- to 12-membered heterocycle, each of which is independently optionally substituted with one or more substituents selected from R¹²; and

wherein any substitutable carbon on the benzazepine core or Formula (IIIA) is optionally substituted by a substituent independently selected from R¹², wherein R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R, —NO₂, ═O, ═S, ═N(R¹⁰), and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle, or

for a compound of Formula (IIIB) or (IIIC), R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁰, R²¹, R²², and R²³ are each hydrogen; and

R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁴ and R²⁵ are each hydrogen.

In certain embodiments, for a compound or salt of Formula (IIIB) or (IIIC):

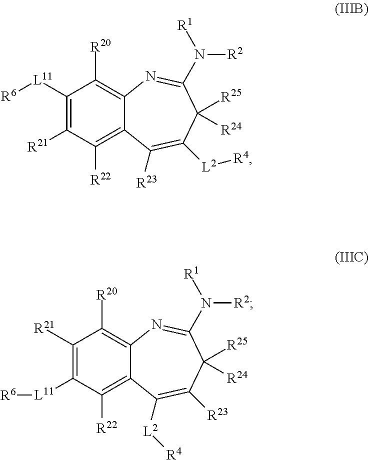

or a pharmaceutically acceptable salt thereof, wherein:

L¹¹ is —C(O)N(R¹⁰)—*, such as L¹¹ is —C(O)NH—, wherein * represents where L¹¹ is bound to R⁶;

L² is —C(O)—;

R¹ and R² are each hydrogen;

R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰;

R⁶ is selected from phenyl and 5- or 6-membered heteroaryl, e.g., pyridyl, any one of which is substituted with one or more substituents selected from R⁷;

R⁷ is selected from —C(O)NHNH₂, —C(O)NH—C₁₋₃alkylene-NH(R¹⁰), —C(O)CH₃, —C₁₋₃alkylene-NHC(O)OR¹¹, —C₁₋₃alkylene-NHC(O)NHR¹⁰, and —C₁₋₃alkylene-NHC(O)—C₁₋₃alkylene-(R¹⁰)₂; and a 3- to 12-membered heterocycle optionally substituted with one or more substituents independently selected from R¹²;

R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is independently optionally substituted at each occurrence with one or more substituents selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;

R¹¹ is selected from C3-12 carbocycle and 3- to 12-membered heterocycle, each of which is independently optionally substituted with one or more substituents selected from R¹² wherein R¹² is independently selected at each occurrence from halogen, —ORI, —SRI, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle;

R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁰, R²¹, R²², and R²³ are each hydrogen; and R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl, such as R²⁴ and R²⁵ are each hydrogen.

In certain embodiments for a compound or salt of Formula (IA), (IB), (IC), (IIA), (IIB), (IIC), (IIIA), (IIIB), or (IIIC), the compound may further comprise a linker (L³), as described elsewhere herein. The linker may be covalently bound to any position, valence permitting, on a compound or salt of Formula (IA), (IB), (IC), (IIA), (IIB), (IIC), (IIIA), (IIIB), or (IIIC). In certain embodiments, the linker is bound to a nitrogen or oxygen atom of a compound or salt of Formula (IA), (IB), (IC), (IIA), (IIB), (IIC), (IIIA), (IIIB), or (IIIC). The linker may comprise a reactive moiety, e.g., an electrophile that can react to form a covalent bond with a moiety of an antibody, e.g., a lysine, serine, threonine, cysteine, tyrosine, aspartic acid, glutamine, a non-natural amino acid residue, or glutamic acid residue of an antibody. In some embodiments, a compound or salt of Formula (IA), (IB), (IC), (IIA), (IIB), (IIC), (IIIA), (IIIB), or (IIIC), may be covalently bound through the linker to an antibody.

In certain embodiments, an immune-modulatory agent can be a TLR7 or TLR8 agonist, such as S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, SB9200, SB11285, 8-substituted imidazo[1,5-a]pyridine, or CL663.

In some embodiments, an immune-stimulatory compound is a ligand of TLR9 selected from the group consisting of. ODN1585, ODN1668, ODN1826, PF-3512676 (ODN2006), ODN2007, ODN2216, ODN2336, ODN2395, BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbGl0.

In some embodiments, an immune-stimulatory compound is a ligand of TLR10.

In other embodiments, an immune-modulatory agent selectively agonizes TLR9, TLR3, TLR4, TLR2, TLR5, RIG-I, STING, cGAS, NOD1, NOD2, NOD1/NOD2, NRLP3, ALPK1, MDAS AIM2, IRE1 and PERK

In some embodiments, an immune-modulatory agent is a ligand of nucleotide-oligomerization domain (NOD)-like selected from the group consisting of: NOD1 agonist (C12-iE-DAP, iE-DAP, Tri-DAP), NOD2 agonist (L18-MDP, MDP, M-TriLYS, M-TriLYS-D-ASN, Murabutide, N-Glycolyl-MDP), and NOD1/NOD2 agonists (M-TriDAP, PGN).

In some embodiments, an immune-modulatory agent is a ligand of one or more RIG-I-Like receptors (RLR) selected from the group consisting of: S′ppp-dsRNA, Poly (dA:dT), Poly(dG:dC), and Poly (I:C).

In some embodiments, an immune-modulatory agent is a ligand of one or more C-type lectin receptors (CLR) selected from the group consisting of: Cnrdlan AL, HKCA, HKSC, WGP, Zymosan, and Trehalose-6,6-dibehenate.

In some embodiments, an immune-modulatory agent is a ligand of one or more Cytosolic DNA Sensors (CDS) selected from the group consisting of: ADU-S100, c-GMP, c-G-AMP, c-G-GMP, c-A-AMP, c-di-AMP, c-di-IMP, c-di-GMP, c-di-UMP, HSV-60, ISD, pCpG, Poly (dA:dT), Poly(dG:dC), Poly (dA), VACV-70 and a-mangostin and the compounds disclosed in WO2018156625 (U of Texas), WO 2018152453 (Eisai), WO 2018138685 (Janssen), WO2018100558 (Takeda), WO2018098203 (Janssen), WO2018065360 (Biolog Life Sciences), WO2018060323 (Boehringer Ingelheim), WO2018045204 (IFM Therapeutics), WO2018009466 (Aduro), WO 2017161349 (Immune Sensor), WO2017123669, WO2017123657, WO2017027646 (Merck), WO2017027645 (Merck), WO2016120305 (GSK), WO2016096174 (InvivoGen), and US20140341976 (Aduro).

In some embodiments, an immune-modulatory agent can be a TLR7 agonist, such as TLR7 agonist R848. In some embodiments, an immune-modulatory agent is an imidazoquinoline, an imidazoquinoline amine, a thiazoquinoline, a guanosine analog, an adenosine analog, a thymidine homopolymer, ssRNA, CpG-A, PolyG10, or PolyG3. In some embodiments, an immune-modulatory agent is selected from the group consisting of gardiquimod, imiquimod, resiquimod, GS-9620, and imidazoquinoline 852A.

In certain embodiments, an immune-modulatory agent is a benzazepine, an imidazoquinoline, a thiazoloquinolone, an aminoquinoline or a ssRNA. In certain embodiments, a an immune-modulatory agent is selected from the group consisting of an imidazoquinoline, a thiazoloquinolone, an aminoquinoline and is other a ssRNA or a benzazepine. In certain embodiments, the TLR8 agonist is selected from VTX-2337, VTX-294 and resiquimod.

An immune-modulatory agent can be an inhibitor of TGFβ, TGFβRI, TGFβRII, the Beta-Catenin pathway, PI3K-beta, STAT3, IL-10, IDO or TDO. An immune-modulatory agent can be an inhibitor of TNIK or Tankyrase. In certain embodiments, an immune-modulatory agent is a kinase inhibitor. In certain embodiments, the kinase inhibitor can be an inhibitor of CDK4/6, such as, for example, abemaciclib or palbociclib.

An immune-modulatory agent can be LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, 8-substituted 2-amino-3H-benzo[b]azepine-4-carboxamide, or INCB023843.

An immune-modulatory agent can be an inhibitor of the β-catenin pathway, such as those disclosed in U.S. Pat. No. 9,505,749, US Published Application Nos. 2015/0044368 and 20150225396, Chinese application 107226808, WO 2017/076484, and WO 2018/046933, the disclosure of which is incorporated by reference herein.

### Linkers

The conjugates described herein include a linker or linkers that attach at least one immune-modulatory agent to an antibody construct. The linker may be a cleavable linker, e.g., a peptide linker, or a non-cleavable linker. A conjugate can comprise multiple linkers. These linkers can be the same linkers or different linkers.

As will be appreciated by skilled artisans, the linkers attach an immune-modulatory agent(s) to an antibody construct by forming a covalent linkage to the immune-modulatory agent at one location and a covalent linkage to the antibody construct of the conjugate at another. Covalent linkages can be formed by reaction between functional groups on the linker and functional groups on the agents and antibody construct of the conjugate. As used herein, the expression “linker” can include (i) unconjugated forms of the linker that can include a functional group capable of covalently attaching the linker to an immune-modulatory agent and a functional group capable of covalently attaching the linker to an antibody construct; (ii) partially conjugated forms of the linker that can include a functional group capable of covalently attaching the linker to an antibody construct of the conjugate and that can be covalently attached to an immune-modulatory agent, or vice versa; and (iii) fully conjugated forms of the linker that can be covalently attached to both an immune-modulatory agent and an antibody construct of the conjugate. In some embodiments, in the immune-modulatory conjugates described herein, moieties comprising the functional groups on the linker and covalent linkages formed between the linker and antibody construct of the conjugate can be specifically illustrated as Rx and Rx respectively.

One embodiment pertains to a conjugate formed by contacting an antibody construct that binds a cell surface receptor, such as a tumor antigen expressed on a tumor cell, with a linker attached to an immune-modulatory agent under conditions in which the linker covalently attaches to the antibody construct. One embodiment pertains to a method of making a conjugate formed by contacting a linker attached to an immune-modulatory agent under conditions in which the linker covalently links to an antibody construct.

As further described herein, a linker can be short, flexible, rigid, hydrophilic or hydrophobic. A linker can contain segments that have different characteristics, such as segments of flexibility or segments of rigidity. A linker can be chemically stable to extracellular environments, for example, chemically stable in the blood stream, or may include linkages that are not stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases.

### Selectively Protease Cleavable Linkers for Selective Release of Immune-Modulatory Agents

In one embodiment, the cleavable linkers are specifically cleaved by a protease that is present in the extracellular portion of the disease microenvironment. A cleavable linker can contain protease cleavage site for a protease that is over-expressed in the disease microenvironment. Cleavage of the protease cleavage site allows selective release of the immune-modulatory agent in the disease environment, while sparing normal tissues from the immune-modulatory agent.

In some embodiments, the cleavable linker contains a protease cleavage site for a protease selected from the group consisting of legumain, plasmin, TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA. In some embodiments, the cleavable linker contains a protease cleavage site for a protease selected from the group consisting of TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA.

Exemplary protease cleavage sites include the following: Pro-urokinase, PRFKIIGG or PRFRIIGG; TGFbeta, SSRHRRALD; Plasminogen, RKSSIIIRMRDVVL; MMP cleavable sequences: Gelatinase, A PLGLWA; Collagenase cleavable sequences: Calf skin collagen (alpha1(I) chain), GPQGIAGQ, Calf skin collagen (alpha2(I) chain), GPQGLLGA; Bovine cartilage collagen (alpha(II) chain), GIAGQ, Human liver collagen (alphal(III) chain), GPLGIAGI; Human alpha2M, GPEGLRVG; Human PZP, YGAGLGVV, AGLGVVER or AGLGISST; and Human fibroblast collagenase, DVAQFVLT (autolytic cleavages), VAQFVLTE, AQFVLTEG or PVQPIGPQ.

In some embodiments, the protease cleavage site is a substrate for at least one MMP. In some embodiments, the protease cleavage site is a substrate for a protease selected from the group consisting of MMP9, MMP14, MMP1, MMP3, MMP13, MMP17, MMP11, and MMP19. In some embodiments the protease cleavage site is a substrate for MMP9.

In some embodiments, the protease cleavage site is a substrate for MMP14. In some embodiments, the protease cleavage site is a substrate that includes the sequence (SEQ ID NO: 570); SARGPSRW (SEQ ID NO: 571); TARGPSFK (SEQ ID NO: 572); LSGRSDNH (SEQ ID NO: 573); GGWHTGRN (SEQ ID NO: 574); HTGRSGAL (SEQ ID NO: 575); PLTGRSGG (SEQ ID NO: 576); AARGPAIH (SEQ ID NO: 577) RGPAFNPM (SEQ ID NO: 578); SSRGPA YL (SEQ ID NO: 579); RGPATPIM (SEQ ID NO: 580); RGPA (SEQ ID NO: 581); GGQPSGMWGW (SEQ ID NO: 582); FPRPLGITGL (SEQ ID NO: 583); VHMPLGFLGP (SEQ ID NO: 584); SPLTGRSG (SEQ ID NO: 585); SAGFSLPA (SEQ ID NO: 586); LAPLGLQRR (SEQ ID NO: 587); SGGPLGVR (SEQ ID NO: 588); PLGL (SEQ ID NO: 589); LSGRSGNH (SEQ ID NO: 590); SGRSANPRG (SEQ ID NO: 591); LSGRSDDH (SEQ ID NO: 592); LSGRSDIH (SEQ ID NO: 593); LSGRSDQH (SEQ ID NO: 594); LSGRSDTH (SEQ ID NO: 595); LSGRSDYH (SEQ ID NO: 596); LSGRSDNP (SEQ ID NO: 597); LSGRSANP (SEQ ID NO: 598); LSGRSANI (SEQ ID NO: 599); and/or LSGRSDNI (SEQ ID NO: 600).

In some embodiments, the protease cleavage site comprises the amino acid sequence LSGRSDNH (SEQ ID NO: 601). In some embodiments, the protease cleavage site comprises the amino acid sequence TGRGPSWV (SEQ ID NO: 602). In some embodiments, the protease cleavage site comprises the amino acid sequence PLTGRSGG (SEQ ID NO: 603). In some embodiments, the protease cleavage site comprises the amino acid sequence GGQPSGMWGW (SEQ ID NO: 604). In some embodiments, the protease cleavage site comprises the amino acid sequence FPRPLGITGL (SEQ ID NO: 605). In some embodiments, the protease cleavage site comprises the amino acid sequence VHMPLGFLGP (SEQ ID NO: 606). In some embodiments, the protease cleavage site comprises the amino acid sequence PLGL (SEQ ID NO: 607). In some embodiments, the protease cleavage site comprises the amino acid sequence SARGPSRW (SEQ ID NO: 608). In some embodiments, the protease cleavage site comprises the amino acid sequence TARGPSFK (SEQ ID NO: 609). In some embodiments, the protease cleavage site comprises the amino acid sequence GGWHTGRN (SEQ ID NO: 610). In some embodiments, the protease cleavage site comprises the amino acid sequence HTGRSGAL (SEQ ID NO: 611). In some embodiments, the protease cleavage site comprises the amino acid sequence AARGPAIH (SEQ ID NO: 612). In some embodiments, the protease cleavage site comprises the amino acid sequence RGPAFNPM (SEQ ID NO: 613). In some embodiments, the protease cleavage site comprises the amino acid sequence SSRGPAYL (SEQ ID NO: 614). In some embodiments, the protease cleavage site comprises the amino acid sequence RGPATPIM (SEQ ID NO: 615). In some embodiments, the protease cleavage site comprises the amino acid sequence RGPA (SEQ ID NO: 616). In some embodiments, the protease cleavage site comprises the amino acid sequence LSGRSGNH (SEQ ID NO: 617). In some embodiments, the protease cleavage site comprises the amino acid sequence SGRSANPRG (SEQ ID NO: 618). In some embodiments, the protease cleavage site comprises the amino acid sequence LSGRSDDH (SEQ ID NO: 619). In some embodiments, the protease cleavage site comprises the amino acid sequence LSGRSDIH (SEQ ID NO: 620). In some embodiments, the protease cleavage site comprises the amino acid sequence LSGRSDQH (SEQ ID NO: 621). In some embodiments, the protease cleavage site comprises the amino acid sequence LSGRSDTH (SEQ ID NO: 622). In some embodiments, the protease cleavage site comprises the amino acid sequence LSGRSDYH (SEQ ID NO: 623). In some embodiments, the protease cleavage site comprises the amino acid sequence LSGRSDNP (SEQ ID NO: 624). In some embodiments, the protease cleavage site comprises the amino acid sequence LSGRSANP (SEQ ID NO: 625). In some embodiments, the protease cleavage site comprises the amino acid sequence LSGRSANI (SEQ ID NO: 626). In some embodiments, the protease cleavage site comprises the amino acid sequence LSGRSDNI (SEQ ID NO: 627).

In some embodiments, the protease cleavage site is a substrate for an MMP and includes the sequence ISSGLSS (SEQ ID NO: 628); QNQALRMA (SEQ ID NO: 629); AQNLLGMV (SEQ ID NO: 630); STFPFGMF (SEQ ID NO: 631); PVGYTSSL (SEQ ID NO: 632); DWL YWPGI (SEQ ID NO: 633), ISSGLLSS (SEQ ID NO: 634), LKAAPRWA (SEQ ID NO: 635); GPSHLVLT (SEQ ID NO: 636); LPGGLSPW (SEQ ID NO: 637); MGLFSEAG (SEQ ID NO: 638); SPLPLRVP (SEQ ID NO: 639); RMHLRSLG (SEQ ID NO: 640); LAAPLGLL (SEQ ID NO: 641); AVGLLAPP (SEQ ID NO: 642); LLAPSHRA (SEQ ID NO: 643); PAGLWLDP (SEQ ID NO: 644); MIAPVA YR (SEQ ID NO: 645); RPSPMWAY (SEQ ID NO: 646); WATPRPMR (SEQ ID NO: 647); FRLLDWQW (SEQ ID NO: 648); ISSGL (SEQ ID NO: 649); ISSGLLS (SEQ ID NO: 650); and/or ISSGLL (SEQ ID NO: 651).

In some embodiments, the protease cleavage site comprises the amino acid sequence ISSGLSS (SEQ ID NO: 652). In some embodiments, the protease cleavage site comprises the amino acid sequence QNQALRMA (SEQ ID NO: 653). In some embodiments, the protease cleavage site comprises the amino acid sequence AQNLLGMV (SEQ ID NO: 654). In some embodiments, the protease cleavage site comprises the amino acid sequence STFPFGMF (SEQ ID NO: 655). In some embodiments, the protease cleavage site comprises the amino acid sequence PVGYTSSL (SEQ ID NO: 656). In some embodiments, the protease cleavage site comprises the amino acid sequence DWL YWPGI (SEQ ID NO: 657). In some embodiments, the protease cleavage site comprises the amino acid sequence ISSGLLSS (SEQ ID NO: 658). In some embodiments, the protease cleavage site comprises the amino acid sequence LKAAPRWA (SEQ ID NO: 659). In some embodiments, the protease cleavage site comprises the amino acid sequence GPSHLVLT (SEQ ID NO: 660). In some embodiments, the protease cleavage site comprises the amino acid sequence LPGGLSPW (SEQ ID NO: 661). In some embodiments, the protease cleavage site comprises the amino acid sequence MGLFSEAG (SEQ ID NO: 662). In some embodiments, the protease cleavage site comprises the amino acid sequence SPLPLRVP (SEQ ID NO: 663). In some embodiments, the protease cleavage site comprises the amino acid sequence RMHLRSLG (SEQ ID NO: 664). In some embodiments, the protease cleavage site comprises the amino acid sequence LAAPLGLL (SEQ ID NO: 665). In some embodiments, the protease cleavage site comprises the amino acid sequence A VGLLAPP (SEQ ID NO: 666). In some embodiments, the protease cleavage site comprises the amino acid sequence LLAPSHRA (SEQ ID NO: 667). In some embodiments, the protease cleavage site comprises the amino acid sequence PAGLWLDP (SEQ ID NO: 668). In some embodiments, the protease cleavage site comprises the amino acid sequence MIAPV A YR (SEQ ID NO: 669). In some embodiments, the protease cleavage site comprises the amino acid sequence RPSPMWAY (SEQ ID NO: 670). In some embodiments, the protease cleavage site comprises the amino acid sequence WATPRPMR (SEQ ID NO: 671). In some embodiments, the protease cleavage site comprises the amino acid sequence FRLLDWQW (SEQ ID NO: 672). In some embodiments, the protease cleavage site comprises the amino acid sequence ISSGL (SEQ ID NO: 673). In some embodiments, the protease cleavage site comprises the amino acid sequence ISSGLLS (SEQ ID NO: 674). In some embodiments, the protease cleavage site comprises the amino acid sequence ISSGLL (SEQ ID NO: 675).

In some embodiments, the protease cleavage site is a substrate for thrombin. In some embodiments, the protease cleavage site is a substrate for thrombin and includes the sequence GPRSFGL (SEQ ID NO: 676) or GPRSFG (SEQ ID NO: 677). In some embodiments, the protease cleavage site comprises the amino acid sequence GPRSFGL (SEQ ID NO: 678).

In some embodiments, the protease cleavage site comprises the amino acid sequence GPRSFG (SEQ ID NO: 679). In some embodiments, the protease cleavage site comprises an amino acid sequence selected from the group consisting of NTLSGRSENHSG (SEQ ID NO: 680); NTLSGRSGNHGS (SEQ ID NO: 681); TSTSGRSANPRG (SEQ ID NO: 682); TSGRSANP (SEQ ID NO: 683); VAGRSMRP (SEQ ID NO: 684); VVPEGRRS (SEQ ID NO: 685); ILPRSPAF (SEQ ID NO: 686); MVLGRSLL (SEQ ID NO: 687); QGRAITFI (SEQ ID NO: 688); SPRSIMLA (SEQ ID NO: 689); and SMLRSMPL (SEQ ID NO: 690).

In some embodiments, the protease cleavage site comprises the amino acid sequence NTLSGRSENHSG (SEQ ID NO: 691). In some embodiments, the protease cleavage site comprises the amino acid sequence NTLSGRSGNHGS (SEQ ID NO: 692). In some embodiments, the protease cleavage site comprises the amino acid sequence TSTSGRSANPRG (SEQ ID NO: 693). In some embodiments, the protease cleavage site comprises the amino acid sequence TSGRSANP (SEQ ID NO: 694). In some embodiments, the protease cleavage site comprises the amino acid sequence VAGRSMRP (SEQ ID NO: 695). In some embodiments, the protease cleavage site comprises the amino acid sequence VVPEGRRS (SEQ ID NO: 696). In some embodiments, the protease cleavage site comprises the amino acid sequence ILPRSP AF (SEQ ID NO: 697). In some embodiments, the protease cleavage site comprises the amino acid sequence MVLGRSLL (SEQ ID NO: 698). In some embodiments, the protease cleavage site comprises the amino acid sequence QGRAITFI (SEQ ID NO: 699). In some embodiments, the protease cleavage site comprises the amino acid sequence SPRSIMLA (SEQ ID NO: 700). In some embodiments, the protease cleavage site comprises the amino acid sequence SMLRSMPL (SEQ ID NO: 701).

In some embodiments, the protease cleavage site is a substrate for a neutrophil elastase. In some embodiments, the protease cleavage site is a substrate for a serine protease. In some embodiments, the protease cleavage site is a substrate for uPA. In some embodiments, the protease cleavage site is a substrate for legumain. In some embodiments, the protease cleavage site is a substrate for matriptase. In some embodiments, the protease cleavage site is a substrate for a cysteine protease. In some embodiments, the protease cleavage site is a substrate for a cysteine protease, such as a cathepsin.

In some embodiments, the protease cleavage site comprises an amino acid sequence selected from the following: LSGRSDNH; (SEQ ID NO: 702) TGRGPSWV; (SEQ ID NO: 703) PLTGRSGG; (SEQ ID NO: 704) TARGPSFK; (SEQ ID NO: 705) NTLSGRSENHSG; (SEQ ID NO: 706) NTLSGRSGNHGS; (SEQ ID NO: 707) TSTSGRSANPRG; (SEQ ID NO: 708) TSGRSANP; (SEQ ID NO: 709) VHMPLGFLGP; (SEQ ID NO: 710) AVGLLAPP; (SEQ ID NO: 711) AQNLLGMV; (SEQ ID NO: 712) QNQALRMA; SEQ ID NO: 713) LAAPLGLL; (SEQ ID NO: 714) STFPFGMF; (SEQ ID NO: 715) ISSGLLSS; (SEQ ID NO: 716) PAGLWLDP; (SEQ ID NO: 717) VAGRSMRP; (SEQ ID NO: 718) VVPEGRRS; (SEQ ID NO: 719) ILPRSPAF; (SEQ ID NO: 720) MVLGRSLL; (SEQ ID NO: 721) QGRAITFI; (SEQ ID NO: 722) SPRSIMLA; (SEQ ID NO: 723) SMLRSMPL; (SEQ ID NO: 724) SARGPSRW; (SEQ ID NO: 725) GWHTGRN; (SEQ ID NO: 726) HTGRSGAL; (SEQ ID NO: 727) AARGPAIH; (SEQ ID NO: 728) RGPAFNPM; (SEQ ID NO: 729) SSRGPAYL; (SEQ ID NO: 730) RGPATPIM; (SEQ ID NO: 731) RGPA; (SEQ ID NO: 732) GGQPSGMWGW; (SEQ ID NO: 733) FPRPLGITGL; (SEQ ID NO: 734) SPLTGRSG; (SEQ ID NO: 735) SAGFSLPA; (SEQ ID NO: 736) LAPLGLQRR; (SEQ ID NO: 737) SGGPLGVR; (SEQ ID NO: 738) PLGL; (SEQ ID NO: 739) ISSGLSS; (SEQ ID NO: 740) PVGYTSSL; (SEQ ID NO: 741) DWLYWPGI; (SEQ ID NO: 742) LKAAPRWA; (SEQ ID NO: 743) GPSHLVLT; (SEQ ID NO: 744) LPGGLSPW; (SEQ ID NO: 745) MGLFSEAG; (SEQ ID NO: 746) SPLPLRVP; (SEQ ID NO: 747) RMHLRSLG; (SEQ ID NO: 748) LLAPSHRA; (SEQ ID NO: 749) GPRSFGL; (SEQ ID NO: 750) GPRSFG; (SEQ ID NO: 751) LSGRSGNH; (SEQ ID NO: 752) SGRSANPRG; (SEQ ID NO: 753) LSGRSDDH; (SEQ ID NO: 754) LSGRSDIH; (SEQ ID NO: 755) LSGRSDQH; (SEQ ID NO: 756) LSGRSDTH; (SEQ ID NO: 757) LSGRSDYH; (SEQ ID NO: 758) LSGRSDNP; (SEQ ID NO: 759) LSGRSANP; (SEQ ID NO: 760) LSGRSANI; (SEQ ID NO: 761) LSGRSDNI; (SEQ ID NO: 762) MIAPVAYR; (SEQ ID NO: 763) RPSPMWAY; (SEQ ID NO: 764) WATPRPMR; (SEQ ID NO: 765) FRLLDWQW; (SEQ ID NO: 766) ISSGL; (SEQ ID NO: 767) ISSGLLS; (SEQ ID NO: 768) ISSGLL; (SEQ ID NO: 769) ISSGLLSGRSDNH; (SEQ ID NO: 770) AVGLLAPPGGLSGRSDNH; (SEQ ID NO: 771) ISSGLLSSGGSGGSLSGRSDNH; (SEQ ID NO: 772) ISSGLLSSGGSGGSLSGRSDNH; (SEQ ID NO: 773) AVGLLAPPGGTSTSGRSANPRG; (SEQ ID NO: 774) TSTSGRSANPRGGGAVGLLAPP; (SEQ ID NO: 775) VHMPLGFLGPGGTSTSGRSANPRG (SEQ ID NO: 776) TSTSGRSANPRGGGVHMPLGFLGP (SEQ ID NO: 777) LSGRSDNHGGAVGLLAPP (SEQ ID NO: 778) VHMPLGFLGPGGLSGRSDNH (SEQ ID NO: 779) LSGRSDNHGGVHMPLGFLGP (SEQ ID NO: 780) SGRSDNHGGSGGSISSGLLSS (SEQ ID NO: 781) LSGRSGNHGGSGGSISSGLLSS (SEQ ID NO: 782) ISSGLLSSGGSGGSLSGRSGNH (SEQ ID NO: 783) LSGRSDNHGGSGGSQNQALRMA (SEQ ID NO: 784) QNQALRMAGGSGGSLSGRSDNH (SEQ ID NO: 785) LSGRSGNHGGSGGSQNQALRMA (SEQ ID NO: 786) QNQALRMAGGSGGSLSGRSGNH (SEQ ID NO: 787) ISSGLLSGRSGNH (SEQ ID NO: 788) ISSGLLSSGGSGGSLSGRNH (SEQ ID NO: 789) ISSGLLSGRSANPRG (SEQ ID NO: 790) AVGLLAPPTSGRSANPRG (SEQ ID NO: 791) AVGLLAPPSGRSANPRG (SEQ ID NO: 792) ISSGLLSGRSDDH (SEQ ID NO: 793) ISSGLLSGRSDIH SEQ ID NO: 794) ISSGLLSGRSDQH (SEQ ID NO: 795) ISSGLLSGRSDTH (SEQ ID NO: 796) ISSGLLSGRSDYH (SEQ ID NO: 797) ISSGLLSGRSDNP (SEQ ID NO: 798) ISSGLLSGRSANP (SEQ ID NO: 799) ISSGLLSGRSANI (SEQ ID NO: 800) AVGLLAPPGGLSGRSDDH (SEQ ID NO: 801) AVGLLAPPGGLSGRSDIH (SEQ ID NO: 802) AVGLLAPPGGLSGRSDQH (SEQ ID NO: 803) AVGLLAPPGGLSGRSDTH (SEQ ID NO: 804) AVGLLAPPGGLSGRSDYH (SEQ ID NO: 805) AVGLLAPPGGLSGRSDNP (SEQ ID NO: 806) AVGLLAPPGGLSGRSANP (SEQ ID NO: 807) AVGLLAPPGGLSGRSANI (SEQ ID NO: 808) ISSGLLSGRSDNI (SEQ ID NO: 809) or AVGLLAPPGGLSGRSDNI (SEQ ID NO: 810).

A linker containing such a protease cleavage site can further contain a pentafluorophenyl group, a succimide or a maleimide group. A linker containing such a protease cleavage site can further contain a para aminobenzoic acid (PABA) group, a para aminobenzoic acid (PABA) group and a maleimide, or a PABA group and a pentafluorophenyl group. A linker containing such a protease cleavage site can further contain a PABA group and a succinimide group or a PABA group and a maleimide group. A linker can be a (maleimidocaproyl)-(protease cleavage site)-(para-aminobenzyloxycarbonyl) linker.

A cleavable linker can also contain segments of alkylene, alkenylene, alkynylene, polyether, polyester, polyamide, polyamino acids, polypeptides, or aminobenzylcarbamates. A cleavable linker can contain a maleimide at one end and a selectively cleavage protease cleavage siet at the other end. A linker can contain a lysine with an N-terminal amine acetylated, and a protease cleavage site. A linker can include a segment created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 811) to an N-terminal GGG motif to regenerate a native amide bond. The linker created can therefore link a moiety attached to the LXPTG recognition motif (SEQ ID NO: 811) with a moiety attached to the N-terminal GGG motif. A linker can be a link created between an unnatural amino acid on one moiety reacting with oxime bond that was formed by modifying a ketone group with an alkoxyamine on another moiety. A moiety can be a portion of an antibody construct, such as an antibody. A moiety can be an immune-modulatory agent. A moiety can be a binding domain. A linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

The cleavable linkers are specifically cleaved by a protease that is present in the extracellular portion of the disease microenvironment. For example, in some embodiments, a linker contains a protease cleavage site for a protease selected from the group consisting of legumain, plasmin, TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA. In some embodiments, a linker contains a protease cleavage site for a protease selected from the group consisting of TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA. In some embodiments, a linker contains a protease cleavage site for a matrix metalloprotease selected from the group consisting of MMP1, MMP2, MMP-3, MMP-9, MMP-8, MMP-14, and MT1-MMP. In some embodiments, a linker contains a protease cleavage site for a caspace protease selected from the group consisting of Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and Caspase-14. In some embodiments, a linker contains a protease cleavage site for a protease selected from the group consisting of TMPRSS3, TMPRSS4, and TMPRSS6. In some embodiments, the linker contains a protease cleavage site for a protease selected from the group consisting of TACE, human neutrophil elastase, beta-secretase, uPA, fibroblast associated protein, matriptase, PSMA and PSA.

The direct attachment of an immune-modulatory agent to a peptide linker can result in proteolytic release of an amino acid adduct of the immune-modulatory agent. Such adducts can be active in vivo. Alternatively, the direct attachment of an immune-modulatory agent to a peptide linker can result in proteolytic release of an amino acid adduct of the immune-modulatory agent, thereby impairing its activity. In some embodiments, selectively protease cleavable linkers can include a self-immolative spacer to spatially separate an immune-modulatory agent from the site of proteolytic cleavage. The use of a self-immolative spacer can allow for the elimination of the fully active, chemically unmodified immune-modulatory agent upon amide bond hydrolysis.

One self-immolative spacer can be a bifunctional para-aminobenzyl alcohol group, which can link to the peptide through the amino group, forming an amide bond, while amine containing immune-modulatory agents can be attached through carbamate functionalities to the benzylic hydroxyl group of the linker (to give a p-amidobenzylcarbamate, PABC). The resulting pro-immune-modulatory agent can be activated upon protease-mediated cleavage, leading to a 1,6-elimination reaction releasing the unmodified immune-modulatory agent, carbon dioxide, and remnants of the linker. The following scheme depicts the fragmentation of p-amidobenzyl carbamate and release of the immune-modulatory agent:

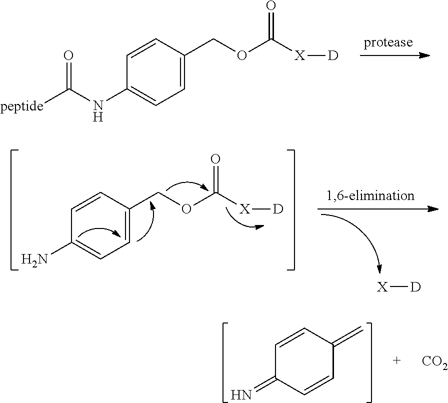

wherein X-D represents the unmodified immune-modulatory agent.

Additionally, immune-modulatory agents containing a phenol group can be covalently bonded to a linker through the phenolic oxygen. One such linker relies on a methodology in which a diamino-ethane “Space Link” is used in conjunction with traditional “PABO”-based self-immolative groups to deliver phenol-containing compounds.

Immune-modulatory agents containing an aromatic or aliphatic hydroxyl group can be covalently bonded to a linker through the hydroxyl group using a methodology that relies on a methylene carbamate linkage, as described in WO 2015/095755.

The cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more protease cleavable groups.

A linker can comprise an protease cleavable peptide moiety, for example, a linker comprising structural formula (IVa), (IVb), (IVc), or (IVd):

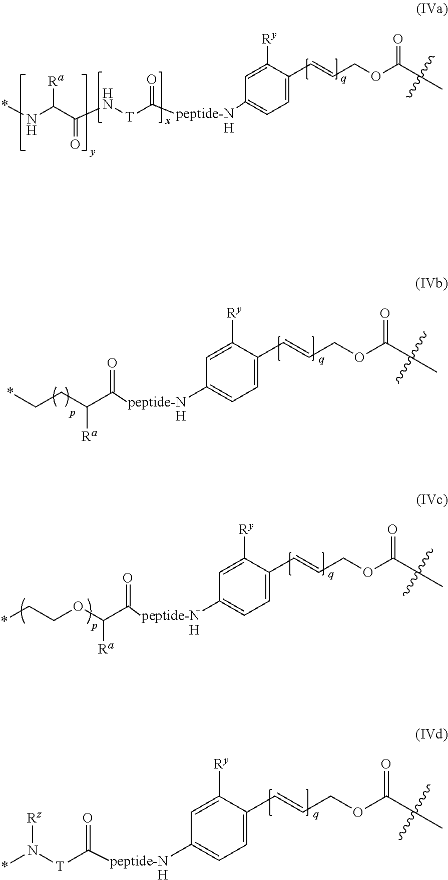

or a salt thereof, wherein: peptide represents a peptide (illustrated N→C, wherein peptide includes the amino and carboxy “termini”) a cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—C₁₋₄ alkylene)ₛ-G¹ or C₁₋₄ alkyl-(N)—[(C₁₋₄ alkylene)-G¹]₂; R_{z} is C₁₋₄ alkyl-(O)ᵣ—(C₁₋₄ alkylene)ₛ-G²; G¹ is SO₃H, CO₂H, PEG 4-32, or sugar moiety; G² is SO₃H, CO₂H, or PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to the immune-modulatory agent; and represents the point of attachment to the remainder of the linker.

Attachment groups that are used to attach the linkers in a conjugate can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

One example of a “self-stabilizing” maleimide group that hydrolyzes spontaneously under conjugation conditions to give a conjugate species with improved stability is depicted in the schematic below. Thus, the maleimide attachment group is reacted with a sulfhydryl of an antibody construct to give an intermediate succinimide ring. The hydrolyzed (open ring) form of the attachment group is resistant to deconjugation in the presence of plasma proteins.

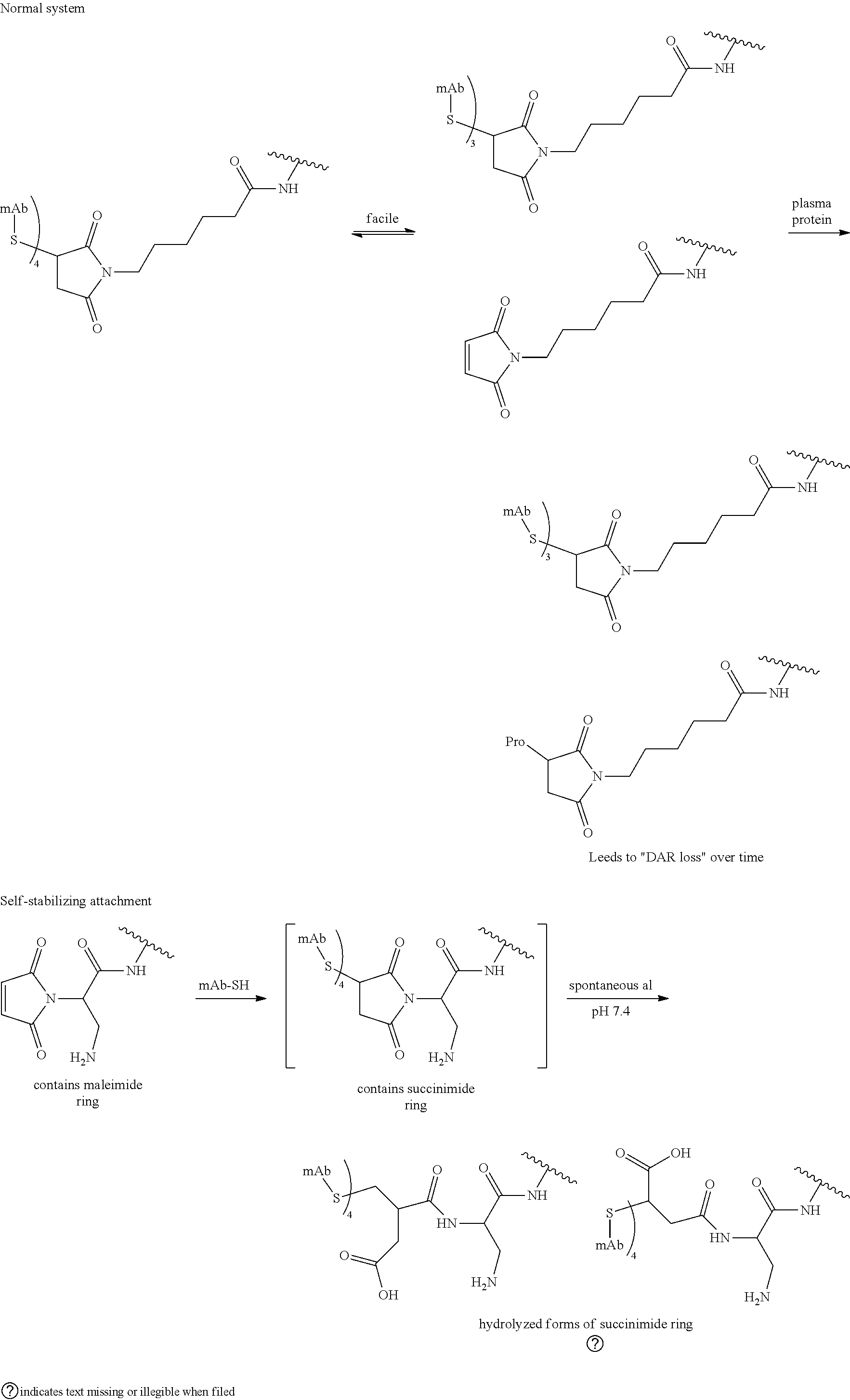

A method for bridging a pair of sulfhydryl groups derived from reduction of a native hinge disulfide bond has been disclosed and is depicted in the schematic below. An advantage of this methodology can be the ability to synthesize homogenous DAR4 conjugates by full reduction of IgGs (to give 4 pairs of sulfhydryls) followed by reaction with 4 equivalents of the alkylating agent. Conjugates containing “bridged disulfides” can also have increased stability.

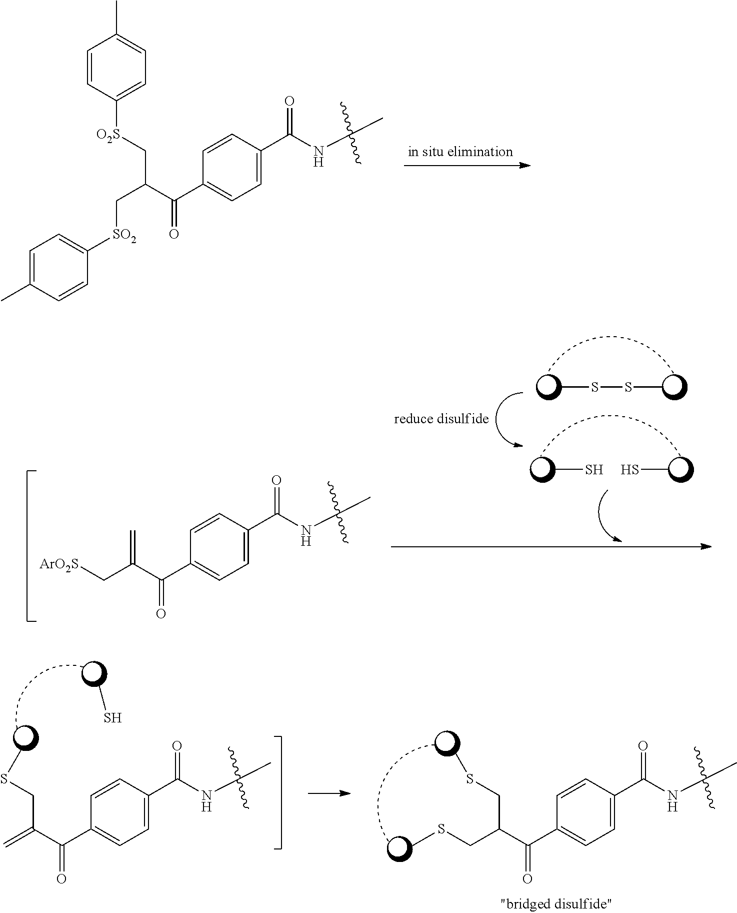

Similarly, as depicted below, a maleimide derivative that can bridge a pair of sulfhydryl groups has been developed.

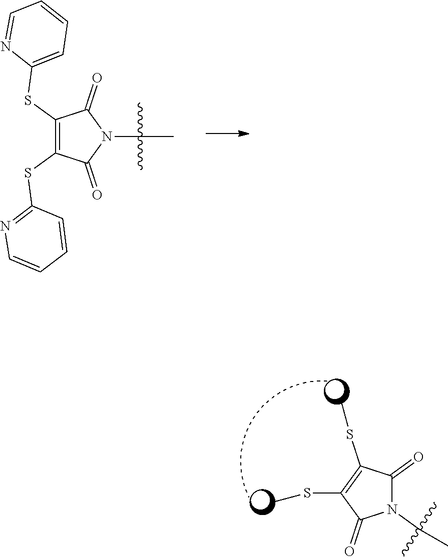 [SMILES: *N1C(=O)C(Sc2ccccn2)=C(Sc2ccccn2)C1=O ; *N1C(=O)C(SC)=C(SC)C1=O]

The attachment moiety can contain the following structural formulas (VIIa), (VIIb), or (VIIc):

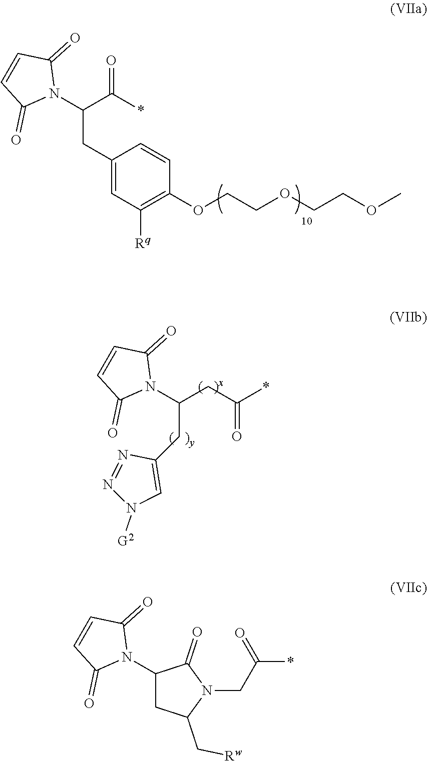

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

### Linkers of Conjugates Having Engineered Fc Domains for Selective Delivery of Immuno-Modulatory Agents

In some embodiments, a conjugate has an engineered Fc domain for selective delivery of an immune-modulatory agent(s). An immune-modulatory agent can be attached to the antibody construct by way of cleavable or non-cleavable linkers. Such a linker attaching an immune-modulatory agent to the antibody construct can be short, long, hydrophobic, hydrophilic, flexible or rigid, or may be composed of segments that each independently have one or more of the above-mentioned properties such that the linker may include segments having different properties. The linkers can be polyvalent such that they covalently attach more than one immune-modulatory agent to a single site on the antibody construct, or monovalent such that covalently they attach a single immune-modulatory agent to a single site on the antibody construct.

In some embodiments, the linker is specifically cleaved by a protease that is present in the extracellular portion of the disease microenvironment, as described herein. For example, in some embodiments, a linker contains a protease cleavage site for a protease selected from the group consisting of TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA. In some embodiments, a linker contains a protease cleavage site for a matrix metalloprotease selected from the group consisting of MMP1, MMP2, MMP-3, MMP-9, MMP-8, MMP-14, and MT1-MMP. In some embodiments, a linker contains a protease cleavage site for a caspace protease selected from the group consisting of Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and Caspase-14. In some embodiments, a linker contains a protease cleavage site for a protease selected from the group consisting of TMPRSS3, TMPRSS4, and TMPRSS6. In some embodiments, the linker contains a protease cleavage site for a protease selected from the group consisting of TACE, human neutrophil elastase, beta-secretase, uPA, fibroblast associated protein, matriptase, PSMA and PSA.

A cleavable linker can also include a valine-citrulline peptide, a valine-alanine peptide, a phenylalanine-lysine or other peptide, such as a peptide that forms a protease recognition and cleavage site. Such a peptide-containing linker can contain a pentafluorophenyl group. A peptide-containing linker can include a succimide or a maleimide group. A peptide-containing linker can include a para aminobenzoic acid (PABA) group. A peptide-containing linker can include an aminobenzyloxycarbonyl (PABC) group. A peptide-containing linker can include a PABA or PABC group and a maleimide group. A peptide-containing linker can include a PABA or PABC group and a pentafluorophenyl group. A peptide-containing linker can include a PABA or PABC group and a succinimide group. A linker can be a (maleimidocaproyl)-(valine-alanine)-(para-aminobenzyloxycarbonyl) linker. A linker can be a (maleimidocaproyl)-(valine-citrulline)-(para-aminobenzyloxycarbonyl) linker. A linker can be a (maleimidocaproyl)-(phenylalanine-lysine)-(para-aminobenzyloxycarbonyl) linker.

A non-cleavable linker is generally protease-insensitive and insensitive to intracellular processes. A non-cleavable linker can include a maleimide group. A non-cleavable linker can include a succinimide group. A non-cleavable linker can be maleimido-alkyl-C(O)— linker. A non-cleavable linker can be maleimidocaproyl linker. A maleimidocaproyl linker can be N-maleimidomethylcyclohexane-1-carboxylate. A maleimidocaproyl linker can include a succinimide group. A maleimidocaproyl linker can include pentafluorophenyl group.

A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain a maleimide(s) linked to polyethylene glycol molecules in which the polyethylene glycol can allow for more linker flexibility or can be used lengthen the linker.

A linker can also contain segments of alkylene, alkenylene, alkynylene, polyether, polyester, polyamide, polyamino acids, peptides, polypeptides, cleavable peptides, and/or aminobenzyl-carbamates. A linker can contain a maleimide at one end and an N-hydroxysuccinimidyl ester at the other end. A linker can contain a lysine with an N-terminal amine acetylated, and a valine-citrulline, valine-alanine or phenylalanine-lysine cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 811) to an N-terminal GGG motif to regenerate a native amide bond. The linker created can therefore link to a moiety attached to the LXPTG recognition motif (SEQ ID NO: 811) with a moiety attached to the N-terminal GGG motif. A linker can be a link created between an unnatural amino acid on one moiety reacting with oxime bond that was formed by modifying a ketone group with an alkoxyamine on another moiety. A moiety can be part of a conjugate. A moiety can be part of an antibody construct, such as an antibody. A moiety can be part of an immune-modulatory agent. A moiety can be part of a binding domain. A linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

A linker can be polyvalent such that it covalently links more than one Immune-modulatory agent to a single site on the antibody construct, or monovalent such that it covalently links a single Immune-modulatory agent to a single site on the antibody construct.

Exemplary polyvalent linkers that may be used to attach many Immune-modulatory agents to an antibody construct of the conjugate are described. For example, Fleximer® linker technology has the potential to enable high-DAR conjugate with good physicochemical properties. As shown below, the Fleximer® linker technology is based on incorporating molecules into a solubilizing poly-acetal backbone via a sequence of ester bonds. The methodology renders highly-loaded conjugates (DAR up to 20) whilst maintaining good physicochemical properties. This methodology can be utilized with an immune-modulatory agent as shown in the scheme below, where Drug’ refers to the immune-modulatory agent.

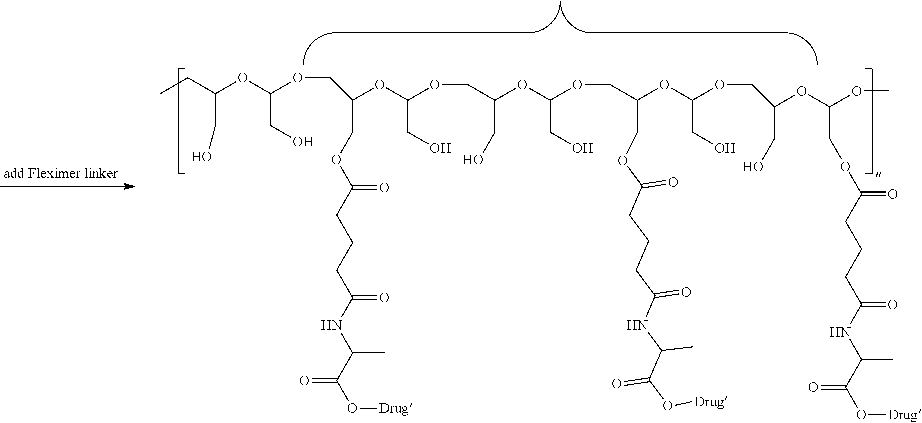

To utilize the Fleximer® linker technology depicted in the scheme above, an aliphatic alcohol can be present or introduced into the Immune-modulatory agent. The alcohol moiety is then attached to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates described herein are described below.

Cleavable linkers can be cleavable in vitro and in vivo. Cleavable linkers can include chemically or enzymatically unstable or degradable linkages. Cleavable linkers can rely on processes inside the cell to liberate an immune-modulatory agent, such as reduction in the cytoplasm, exposure to acidic conditions in the lysosome, or cleavage by specific proteases or other enzymes within the cell. Cleavable linkers can incorporate one or more chemical bonds that are chemically or enzymatically cleavable while the remainder of the linker can be non-cleavable.

A linker can contain a chemically labile group such as hydrazone and/or disulfide group. Linkers comprising chemically labile groups can exploit differential properties between the plasma and some cytoplasmic compartments. The intracellular conditions that can facilitate immune-modulatory agent release for hydrazine-containing linkers can be the acidic environment of endosomes and lysosomes, while disulfide-containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazones, can remain intact during systemic circulation in the blood's neutral pH environment (pH 7.3-7.5) and can undergo hydrolysis and can release an immune-modulatory agent once the conjugate is internalized into mildly acidic endosomal (pH 5.0-6.5) and lysosomal (pH 4.5-5.0) compartments of the cell. This pH dependent release mechanism can be associated with nonspecific release of the immune-modulatory agent. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

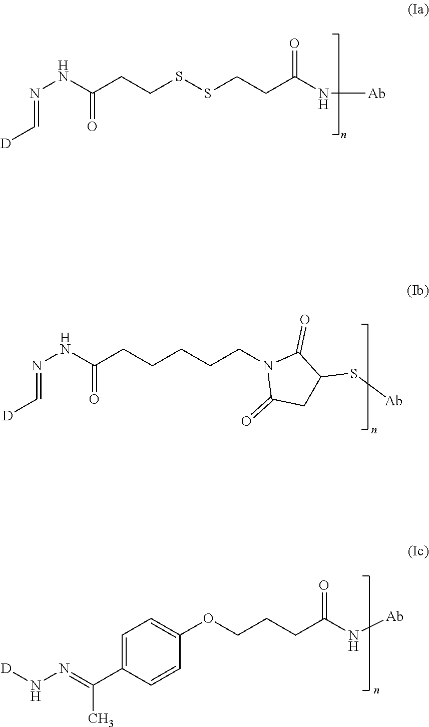

wherein D is an immune-modulatory agent and Ab is an antibody construct, respectively, and n represents the number of compound-bound linkers (LP) bound to the antibody construct. In certain linkers, such as linker (Ia), the linker can comprise two cleavable groups, a disulfide and a hydrazone moiety. For such linkers, effective release of the unmodified free immune-modulatory agent can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release an immune-modulatory agent upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the immune-modulatory agent in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Immune-modulatory conjugates including disulfide-containing linkers can include the following structures:

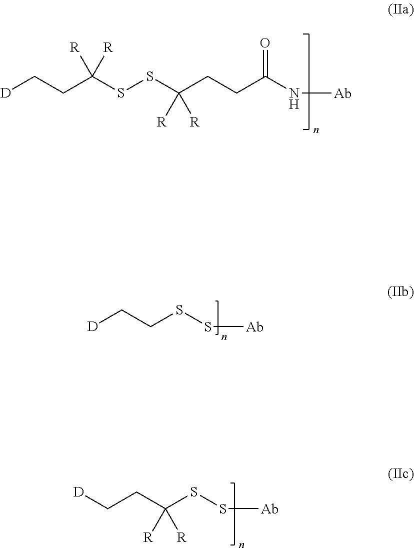

wherein D is an immune-modulatory agent and Ab is an antibody construct, respectively, n represents the number of compounds bound to linkers bound to the antibody construct and R is independently selected at each occurrence from hydrogen or alkyl, for example. Increasing steric hindrance adjacent to the disulfide bond can increase the stability of the linker. Structures such as (IIa) and (IIc) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorable pH value of blood compared to lysosomes. Release of an immune-modulatory agent from an antibody construct can occur due to the action of lysosomal proteases, e.g., cathepsin and plasmin. These proteases can be present at elevated levels in certain tumor tissues. A linker can be cleavable by a lysosomal enzyme. The lysosomal enzyme can be, for example, cathepsin B, cathepsin S, β-glucuronidase, or β-galactosidase.

The cleavable peptide can be selected from tetrapeptides such as Gly-Phe-Leu-Gly, Ala-Leu-Ala-Leu, dipeptides such as Val-Cit, Val-Ala, and Phe-Lys, or other peptides. Dipeptides can have lower hydrophobicity compared to longer peptides, depending on the composition of the peptide.

A variety of dipeptide-based cleavable linkers can be used in the immune-modulatory conjugates described herein.

Enzymatically cleavable linkers can include a self-immolative spacer to spatially separate the immune-modulatory agent from the site of enzymatic cleavage. The direct attachment of an immune-modulatory agent to a peptide linker can result in proteolytic release of the immune-modulatory agent or of an amino acid adduct of the immune-modulatory agent, thereby impairing its activity. The use of a self-immolative spacer can allow for the elimination of the fully active, chemically unmodified immune-modulatory agent upon amide bond hydrolysis.

One self-immolative spacer can be a bifunctional para-aminobenzyl alcohol group (PABA), which can link to the peptide through the amino group, forming an amide bond, while amine containing immune-modulatory agents can be attached through carbamate functionalities to the benzylic hydroxyl group of the linker (to give a p-amidobenzylcarbamate, PABC). The resulting pro-immune-modulatory agent can be activated upon protease-mediated cleavage, leading to a 1,6-elimination reaction releasing the unmodified immune-modulatory agent, carbon dioxide, and remnants of the linker. The following scheme depicts the fragmentation of p-amidobenzyl carbamate and release of the immune-modulatory agent:

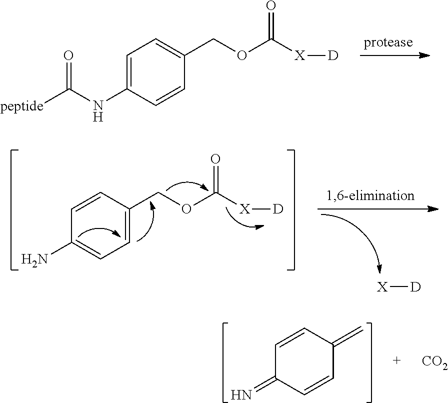

wherein X-D represents the unmodified immune-modulatory agent and the carbonyl group adjacent “peptide” is part of the peptide. Heterocyclic variants of this self-immolative group have also been described.

An enzymatically cleavable linker can be a ß-glucuronic acid-based linker. Facile release of an immune-modulatory agent can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of an immune-modulatory conjugate to undergo aggregation due to the hydrophilic nature of ß-glucuronides. In certain embodiments, ß-glucuronic acid-based linkers can link an antibody construct to a hydrophobic immune-modulatory agent. The following scheme depicts the release of an immune-modulatory agent (D) from an immune-modulatory conjugate containing a ß-glucuronic acid-based linker:

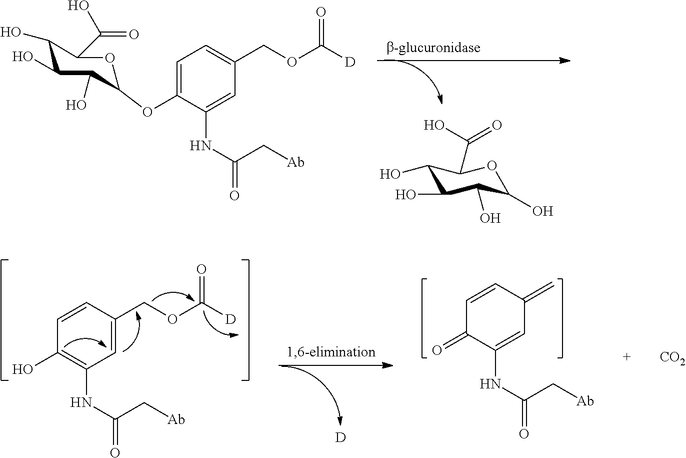

wherein Ab indicates the antibody construct.

A variety of cleavable ß-glucuronic acid-based linkers useful for linking drugs such as auristatins, camptothecin and doxorubicin analogues, CBI minor-groove binders, and psymberin to antibodies have been described. These ß-glucuronic acid-based linkers may be used in the conjugates described herein. In certain embodiments, the enzymatically cleavable linker is a β-galactoside-based linker. β-Galactoside is present abundantly within lysosomes, while the enzyme activity outside cells is low.

Additionally, immune-modulatory agents containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on an immune-modulatory agent, wherein such ester groups can hydrolyze under physiological conditions to release the immune-modulatory agent. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IIIa), (IIIb), (IIIc), or (IIId):

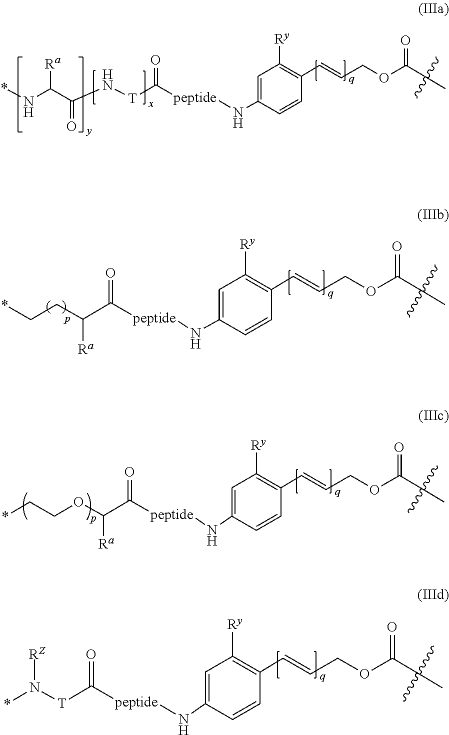

or a salt thereof, wherein: “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G₁]₂; R^{z} is C₁₋₄ alkyl-(O)ᵣ—(C₁₋₄ alkylene)ₛ-G²; G¹ is SO₃H, CO₂H, PEG 4-32, or a sugar moiety; G² is SO₃H, CO₂H, or a PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to an immune-modulatory agent; and * represents the point of attachment to the remainder of the linker.

In certain embodiments, the peptide can be selected from natural amino acids, unnatural amino acids or combinations thereof. In certain embodiments, the peptide can be selected from a tripeptide or a dipeptide. In particular embodiments, the dipeptide can comprise L-amino acids and be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IIIa) are illustrated below (as illustrated, the linkers include a reactive group suitable for covalently attaching a linker to an antibody construct):

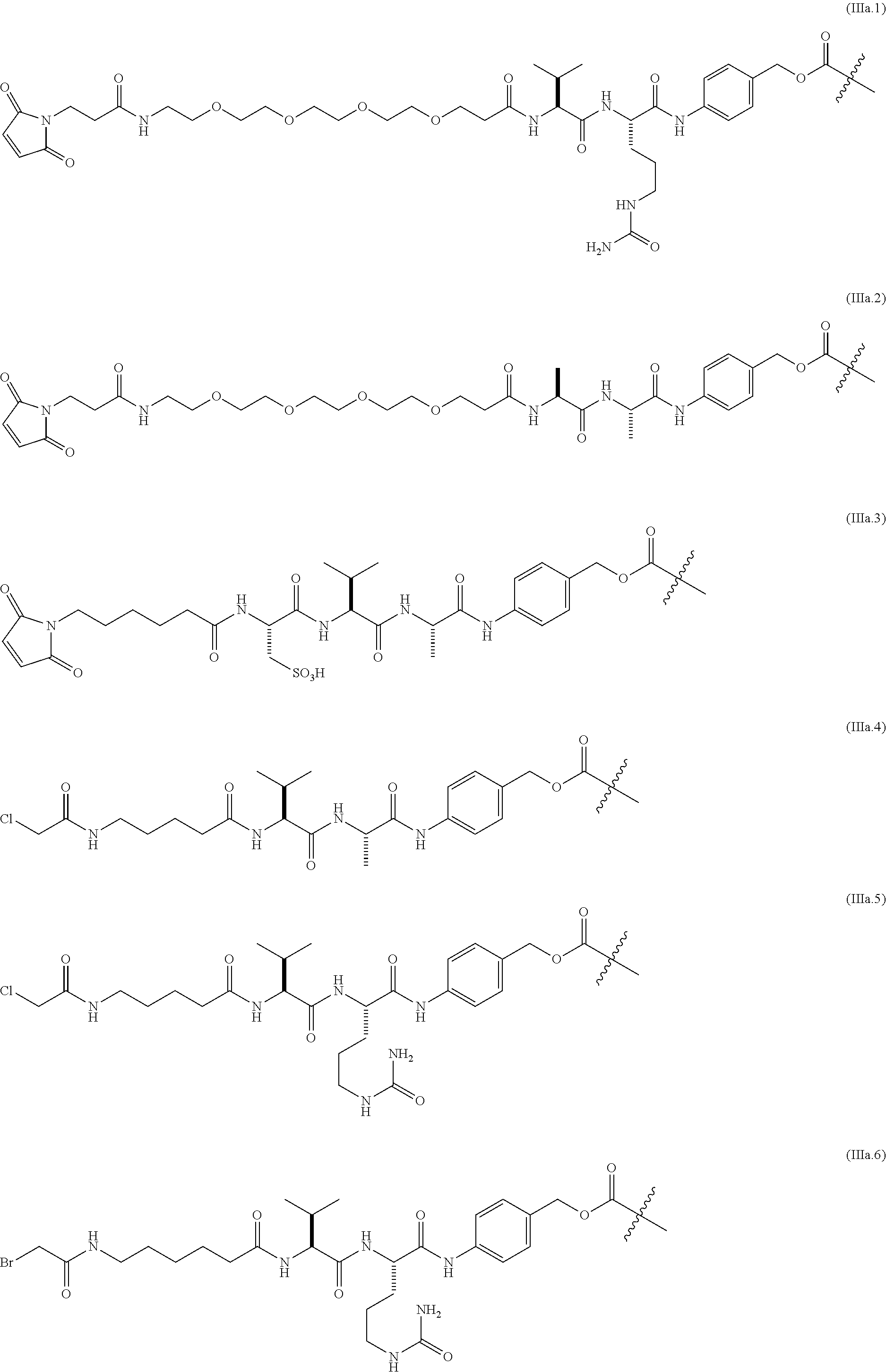

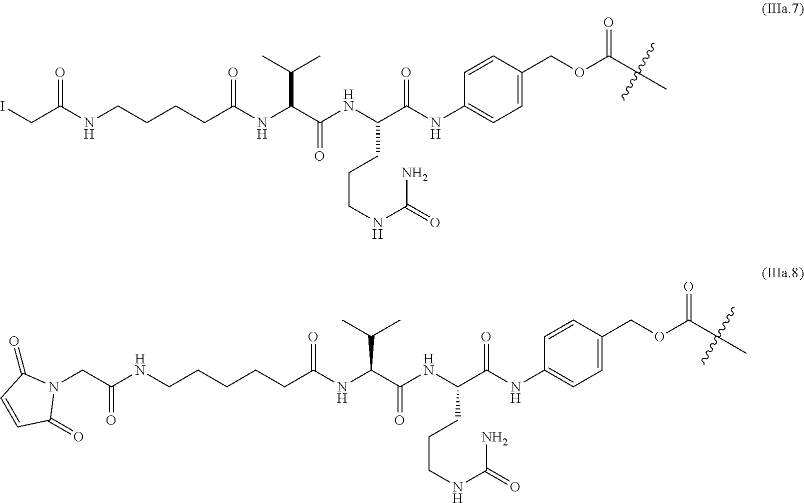 [SMILES: *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

wherein [symbol] indicates an attachment site of a linker to an immune-modulatory agent.

Exemplary embodiments of linkers according to structural formula (IIIb), (IIIc), or (IIId) that can be included in the conjugates described herein can include the linkers illustrated below (as illustrated, the linkers can include a reactive group suitable for covalently linking the linker to an antibody construct):

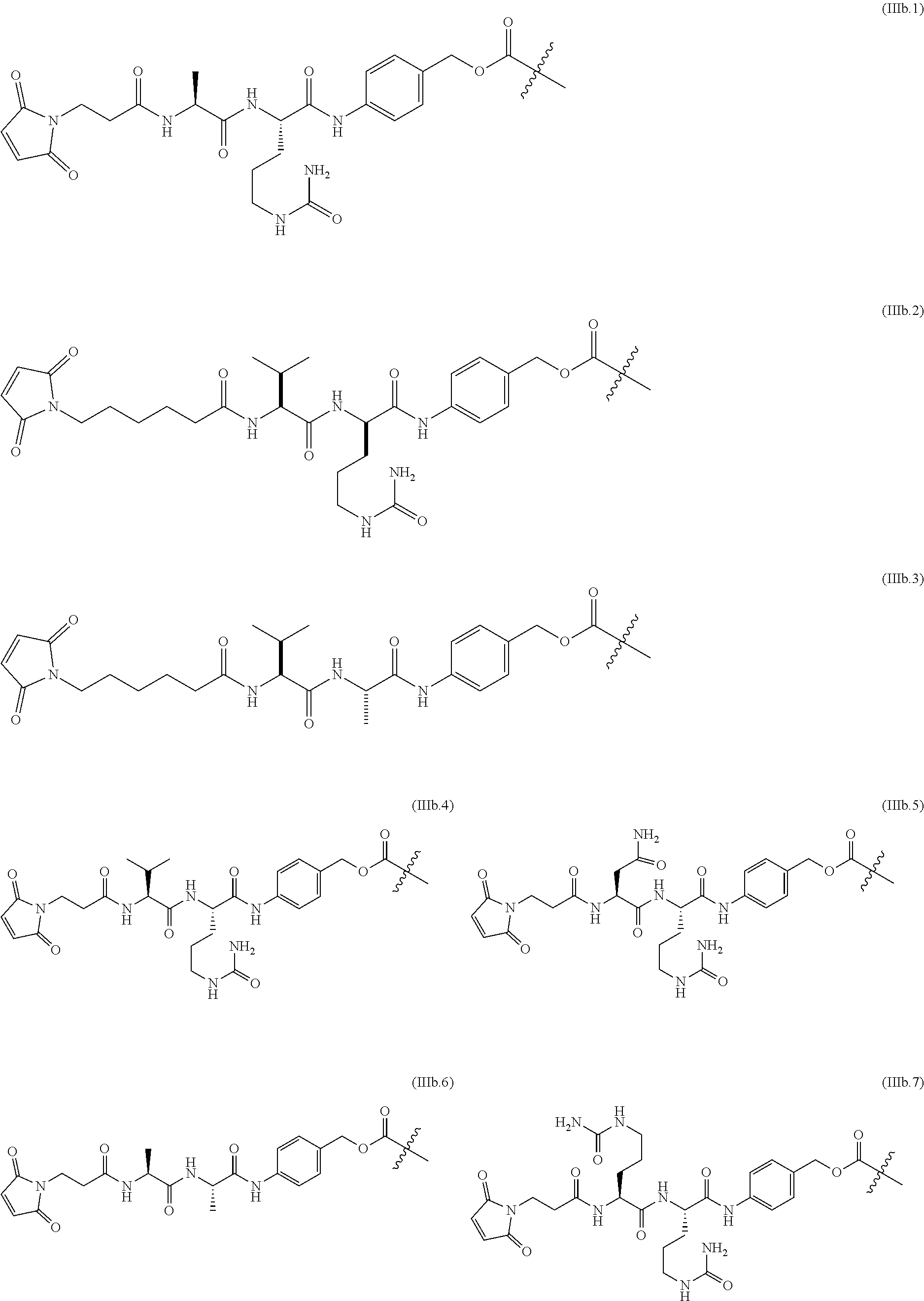

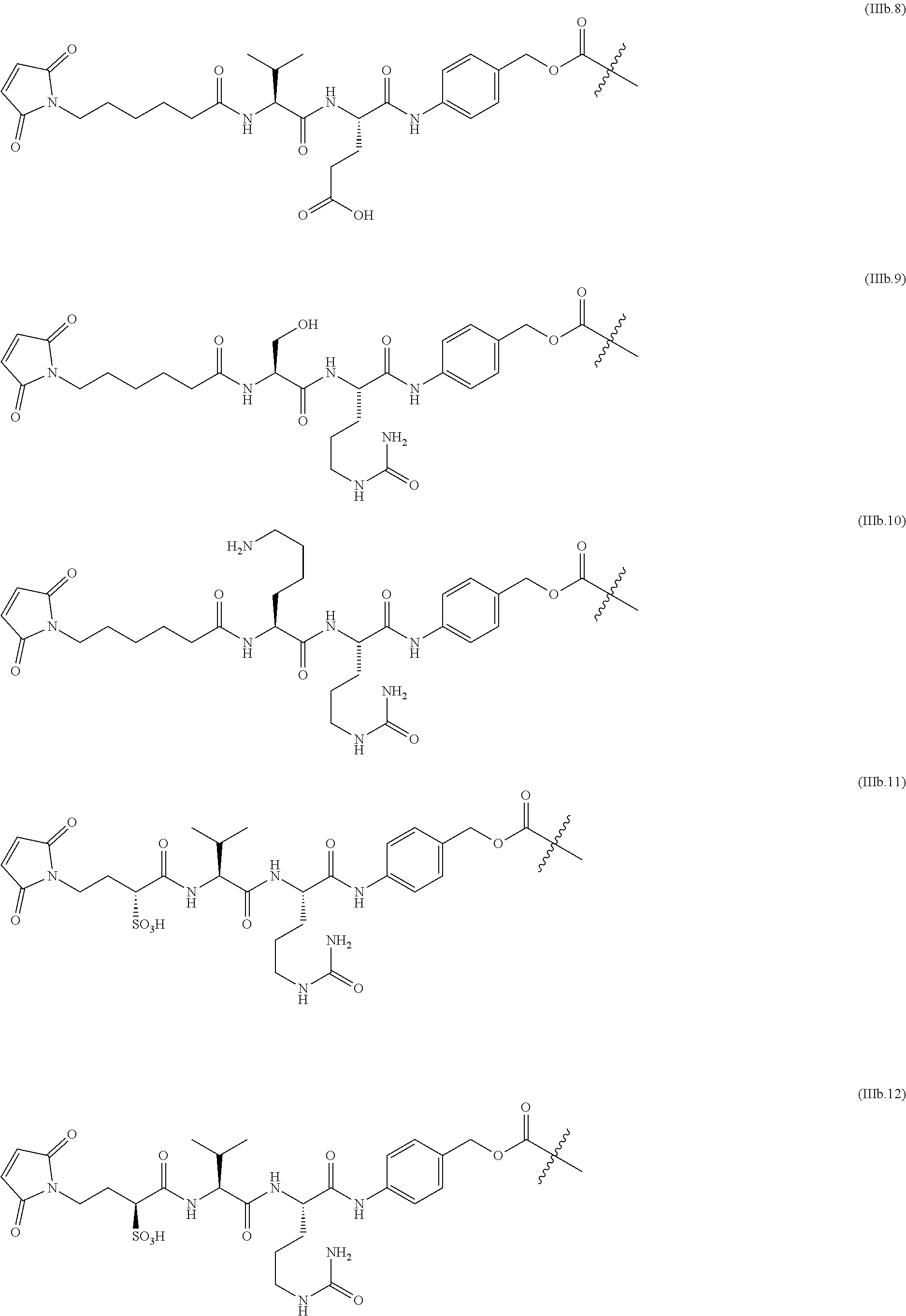 [SMILES: *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

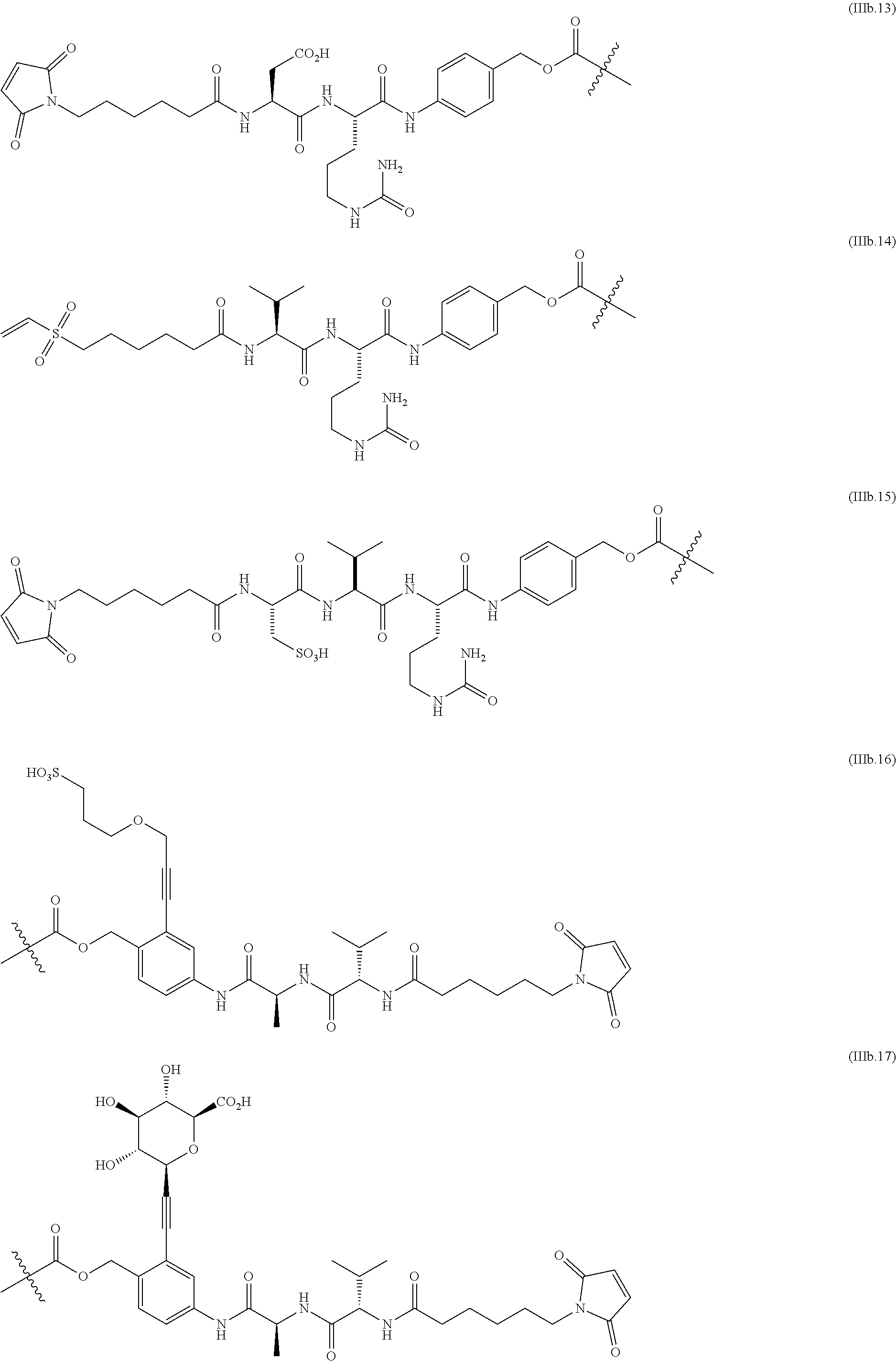

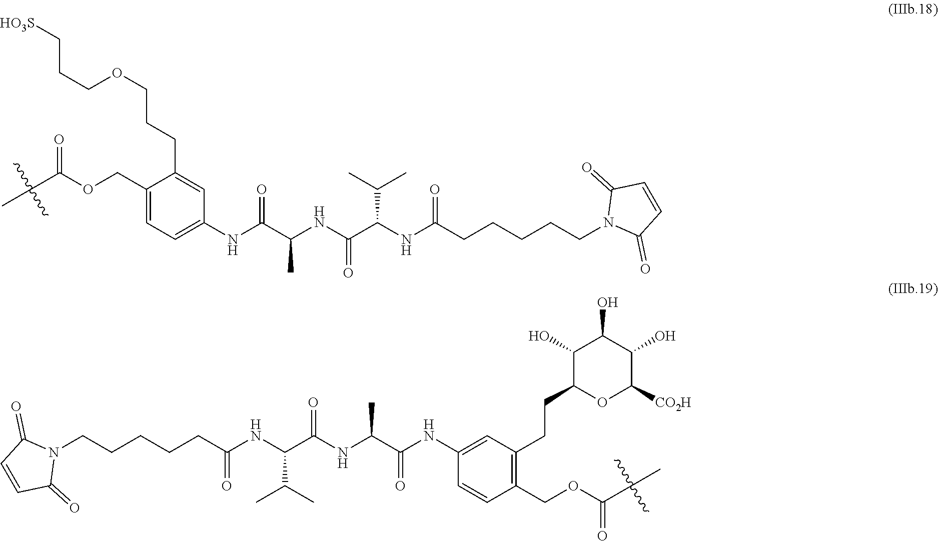 [SMILES: *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O]

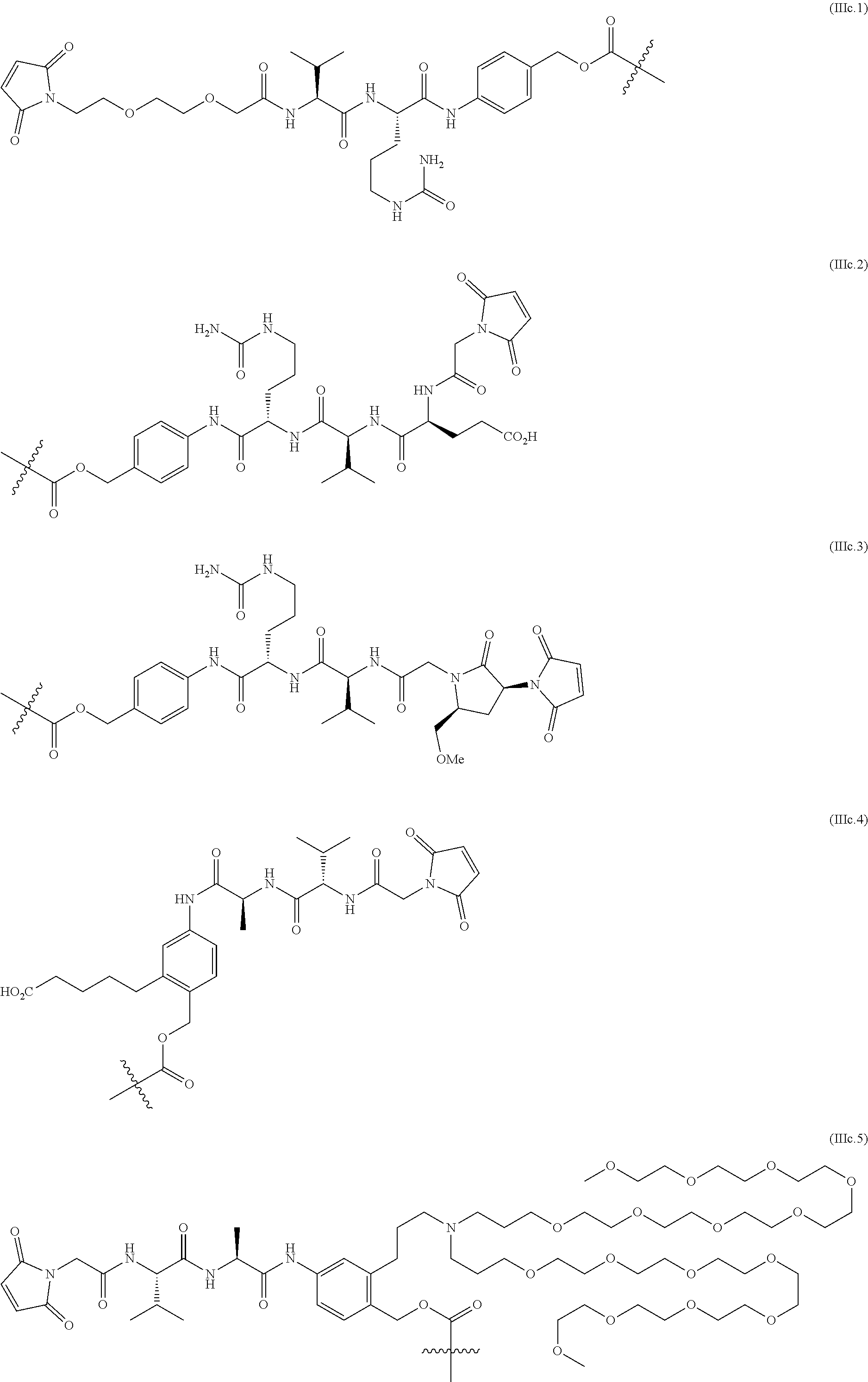

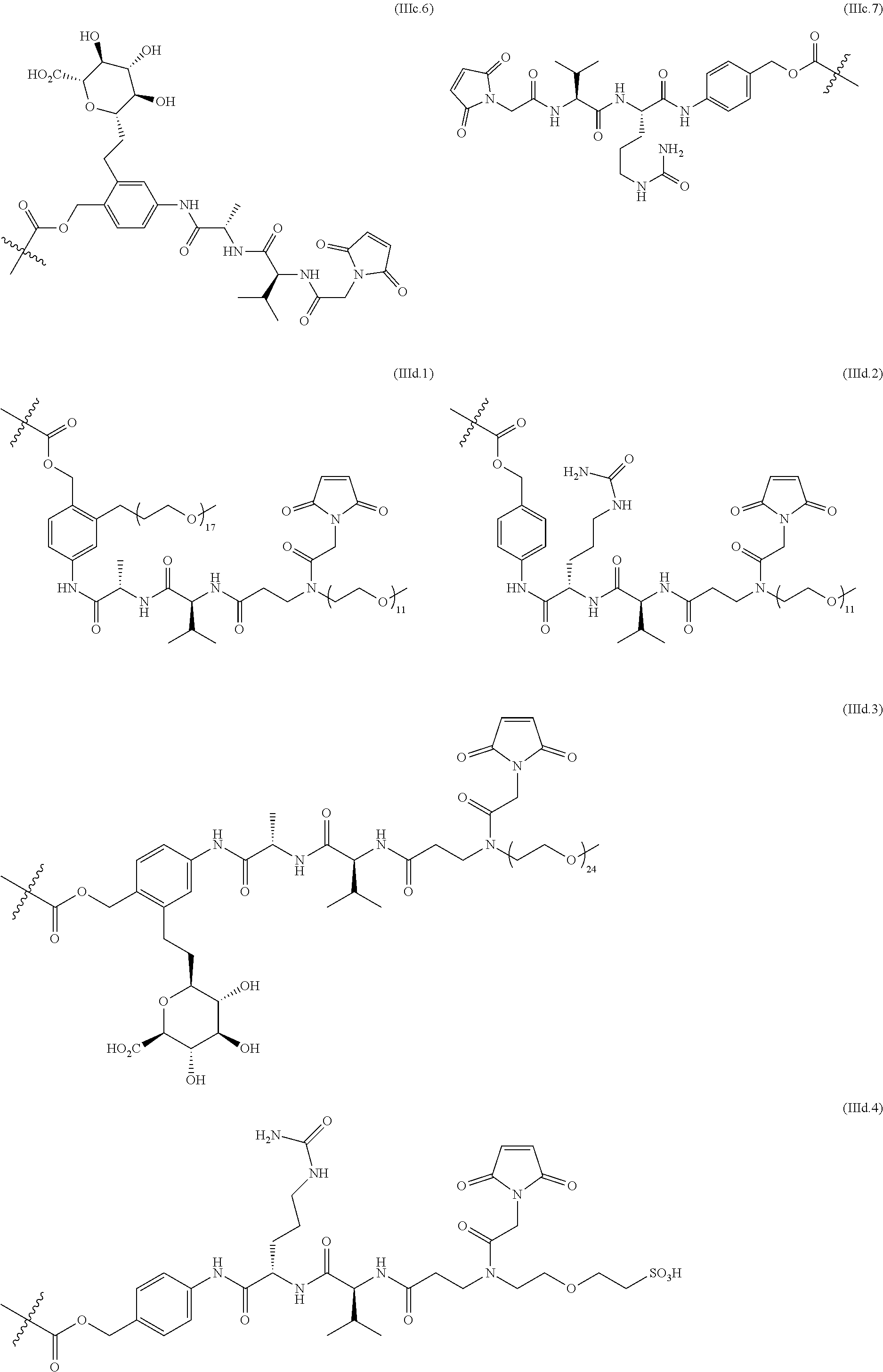

wherein [symbol] indicates an attachment site to an immune-modulatory agent.

The linker can contain an enzymatically cleavable sugar moiety, for example, a linker comprising structural formula (IVa), (IVb), (IVc), (IVd), or (IVe):

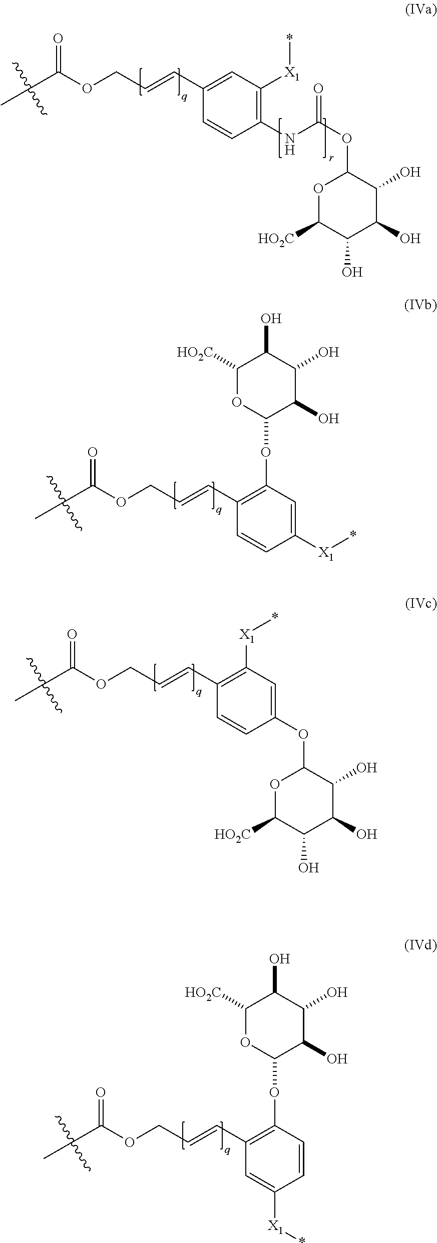

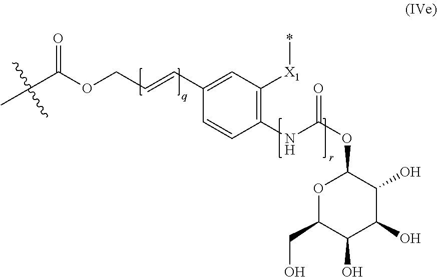

or a salt thereof, wherein: q is 0 or 1; r is 0 or 1; X¹ is CH₂, O or NH; [symbol] represents the point of attachment of the linker to an immune-modulatory agent; and * represents the point of attachment to the remainder of the linker.

Exemplary embodiments of linkers according to structural formula (IVa) that may be included in the immune-modulatory conjugates described herein can include the linkers illustrated below (as illustrated, the linkers include a group suitable for covalently linking the linker to an antibody construct):

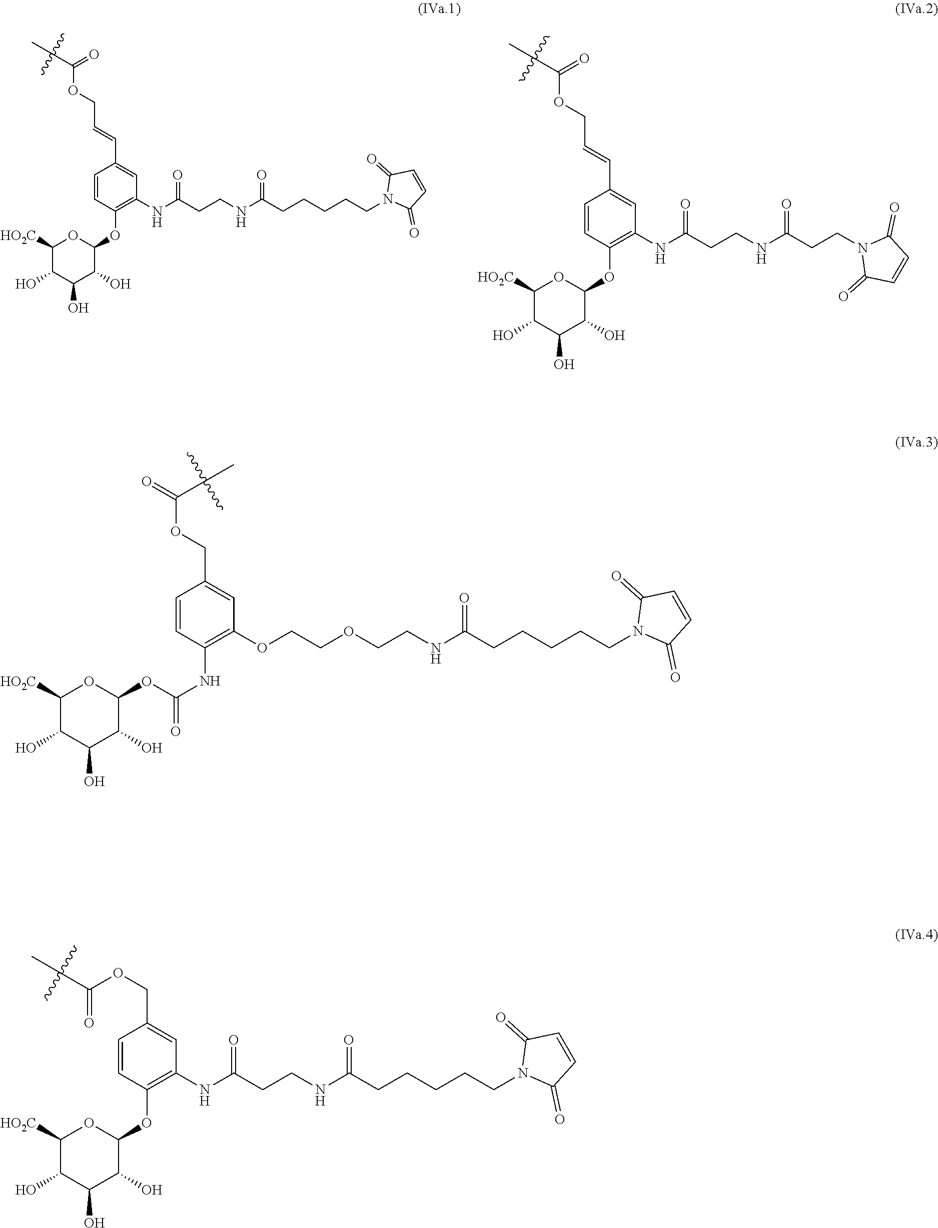

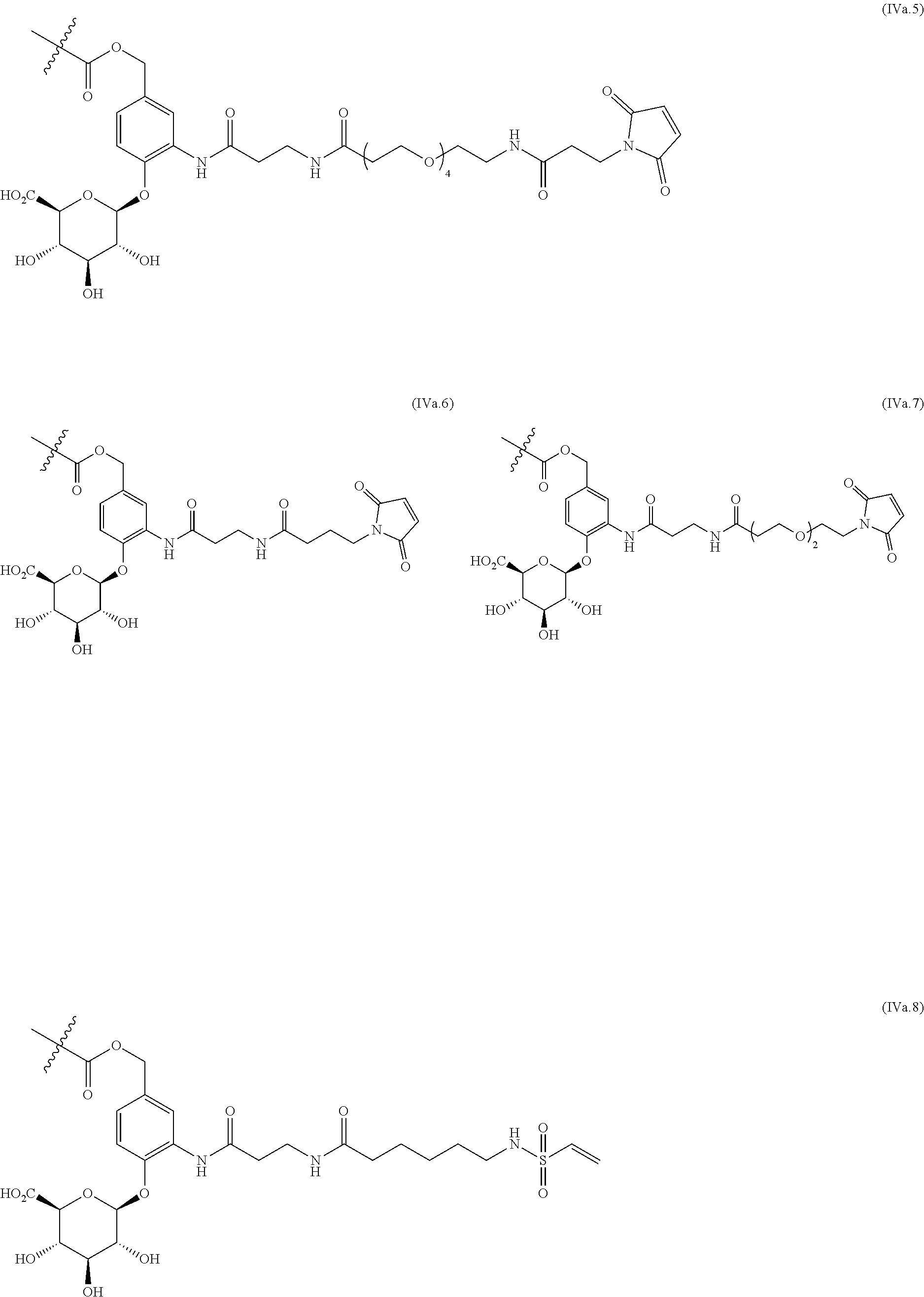

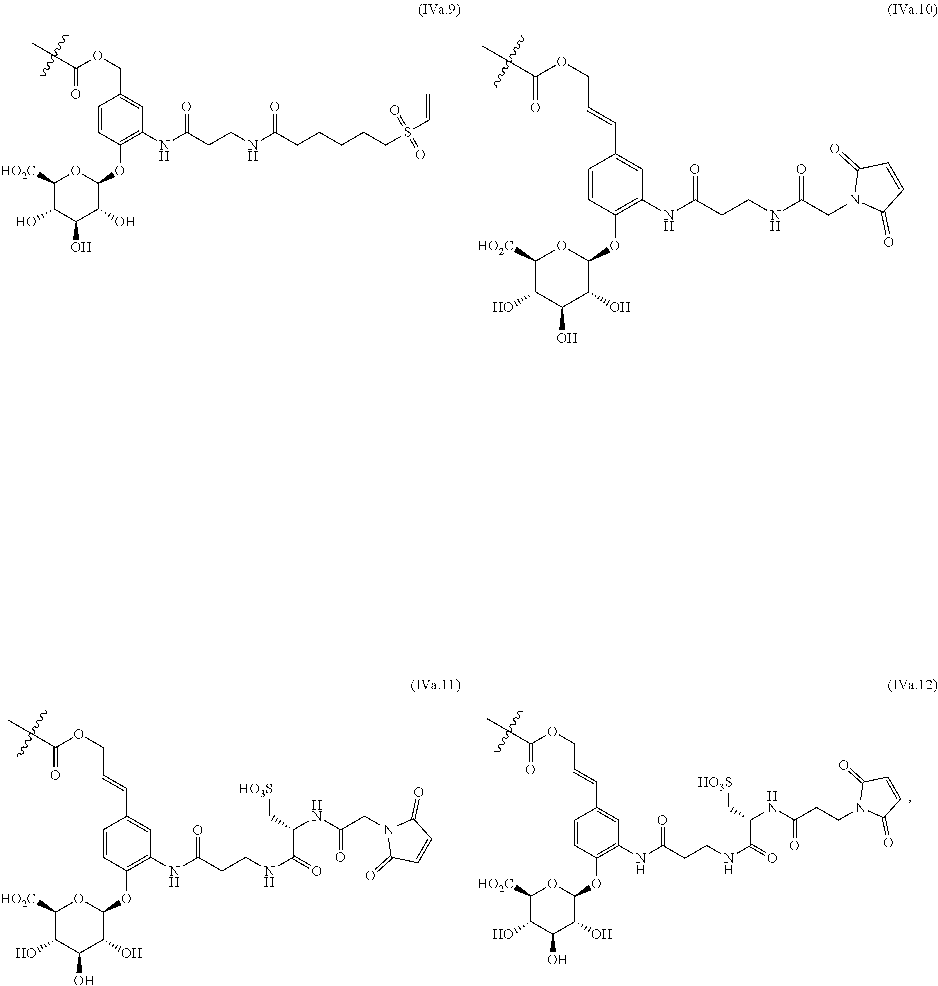

wherein [symbol] represents the point of attachment of a linker to an immune-modulatory agent.

Exemplary embodiments of linkers according to structural formula (IVb) that may be included in the conjugates described herein include the linkers illustrated below (as illustrated, the linkers include a group suitable for covalently linking the linker to an antibody construct):

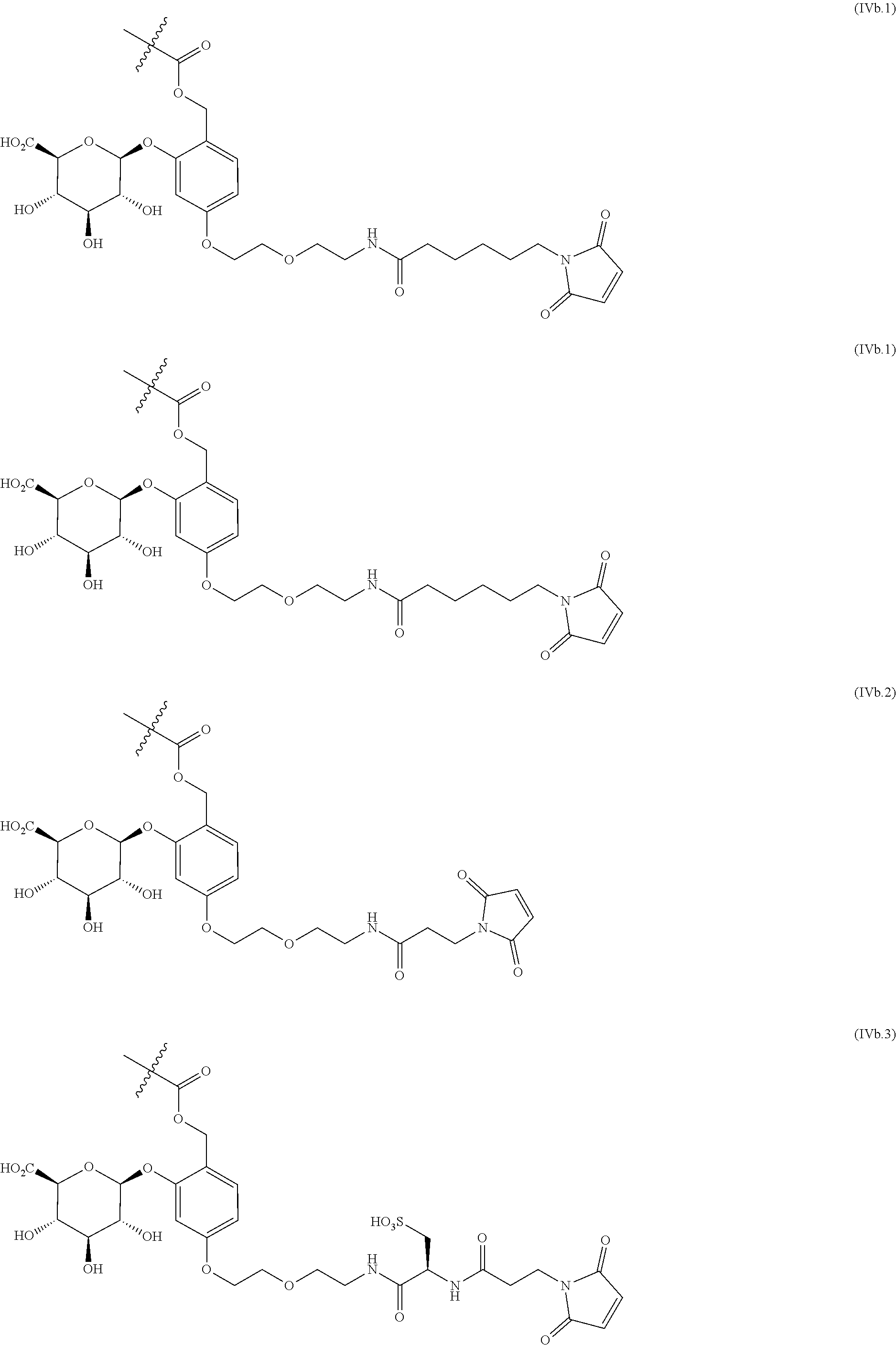

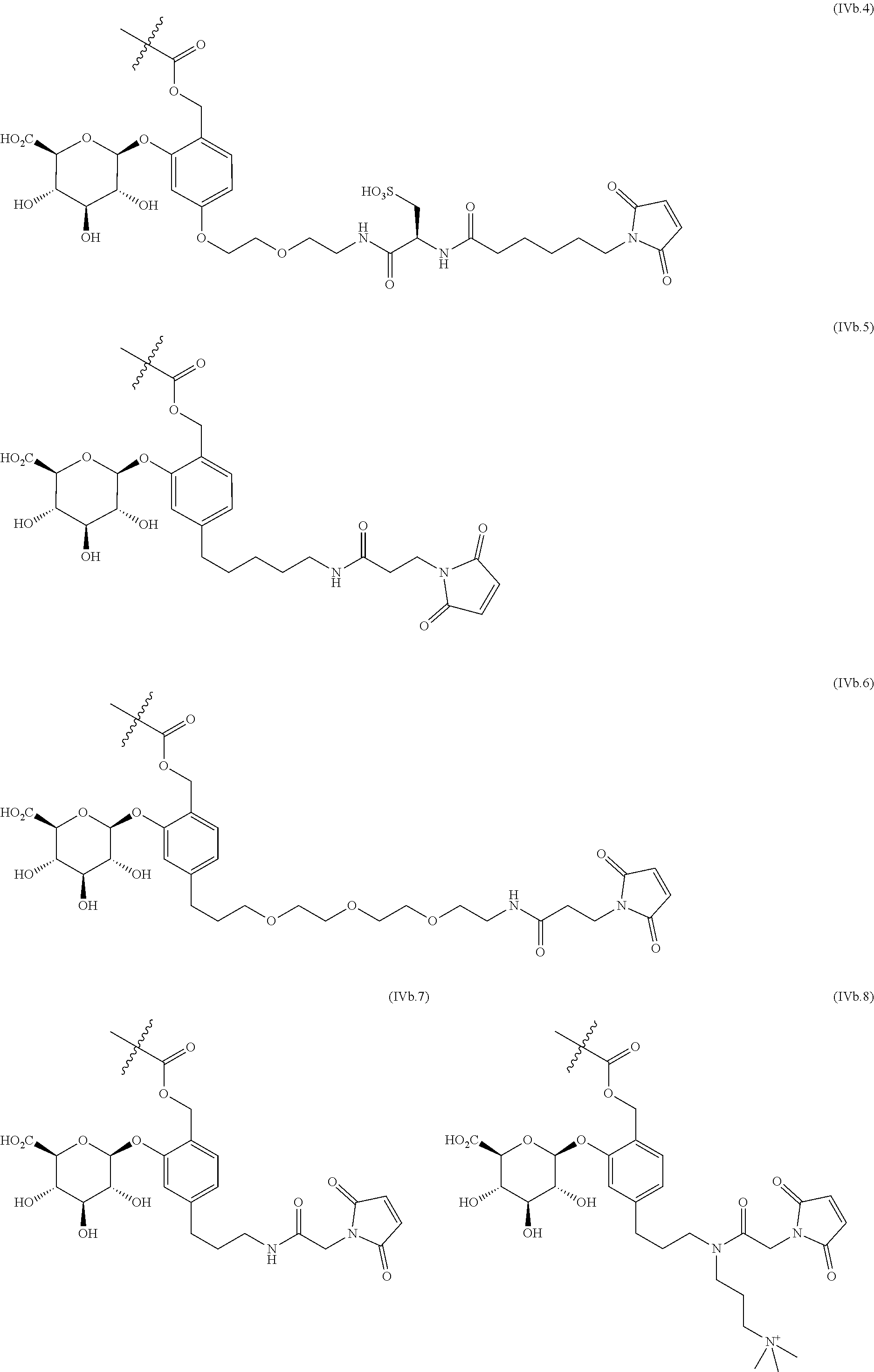

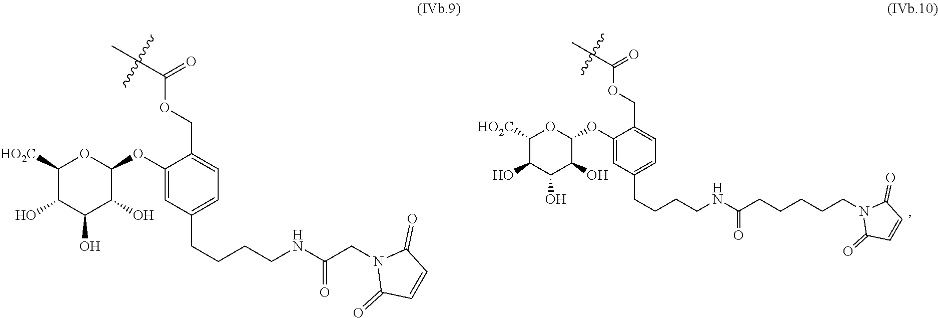

wherein [symbol] represents the point of attachment of a linker to an immune-modulatory agent.

Exemplary embodiments of linkers according to structural formula (IVc) that may be included in the conjugates described herein include the linkers illustrated below (as illustrated, the linkers include a group suitable for covalently linking the linker to an antibody construct):

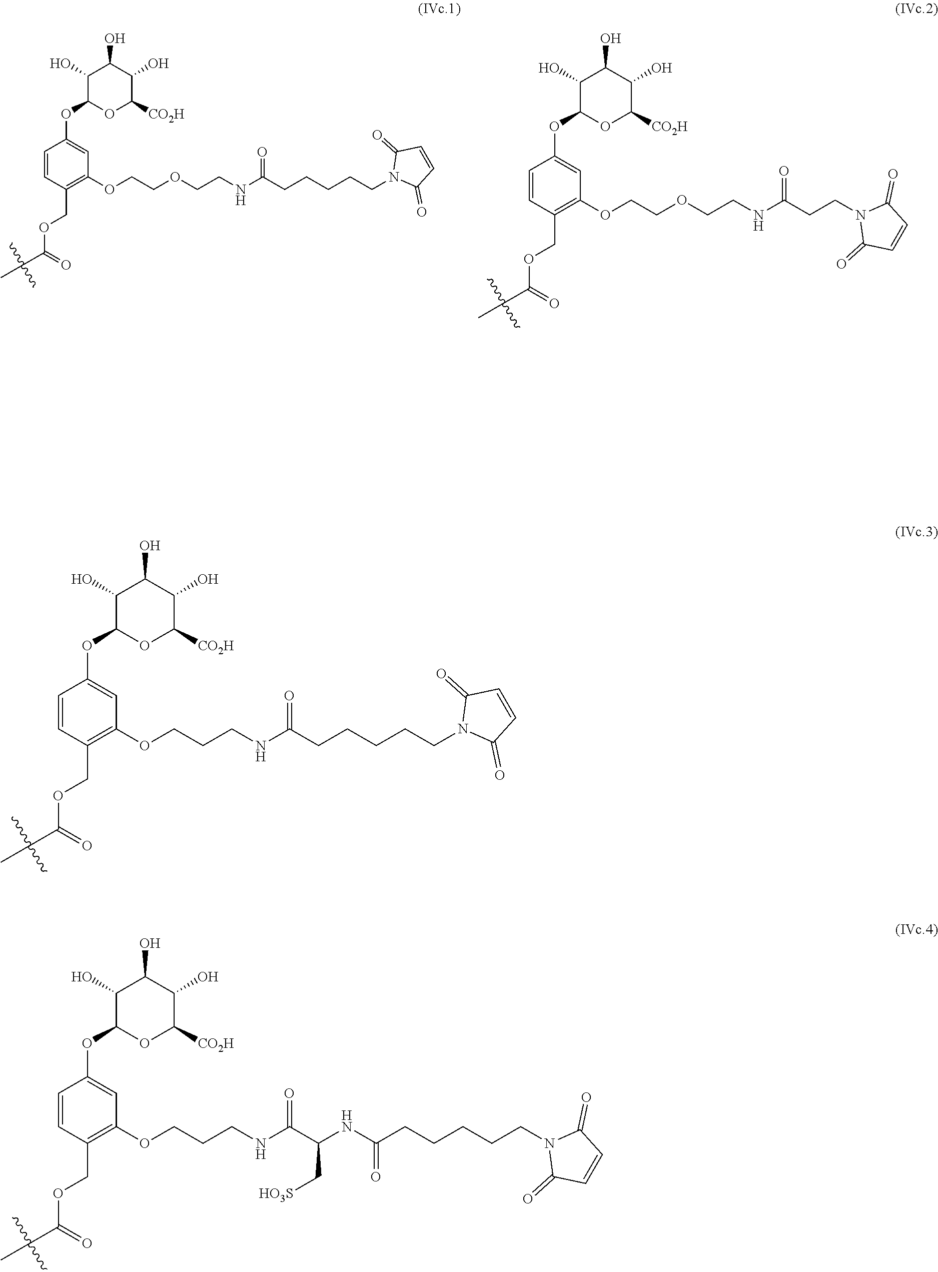

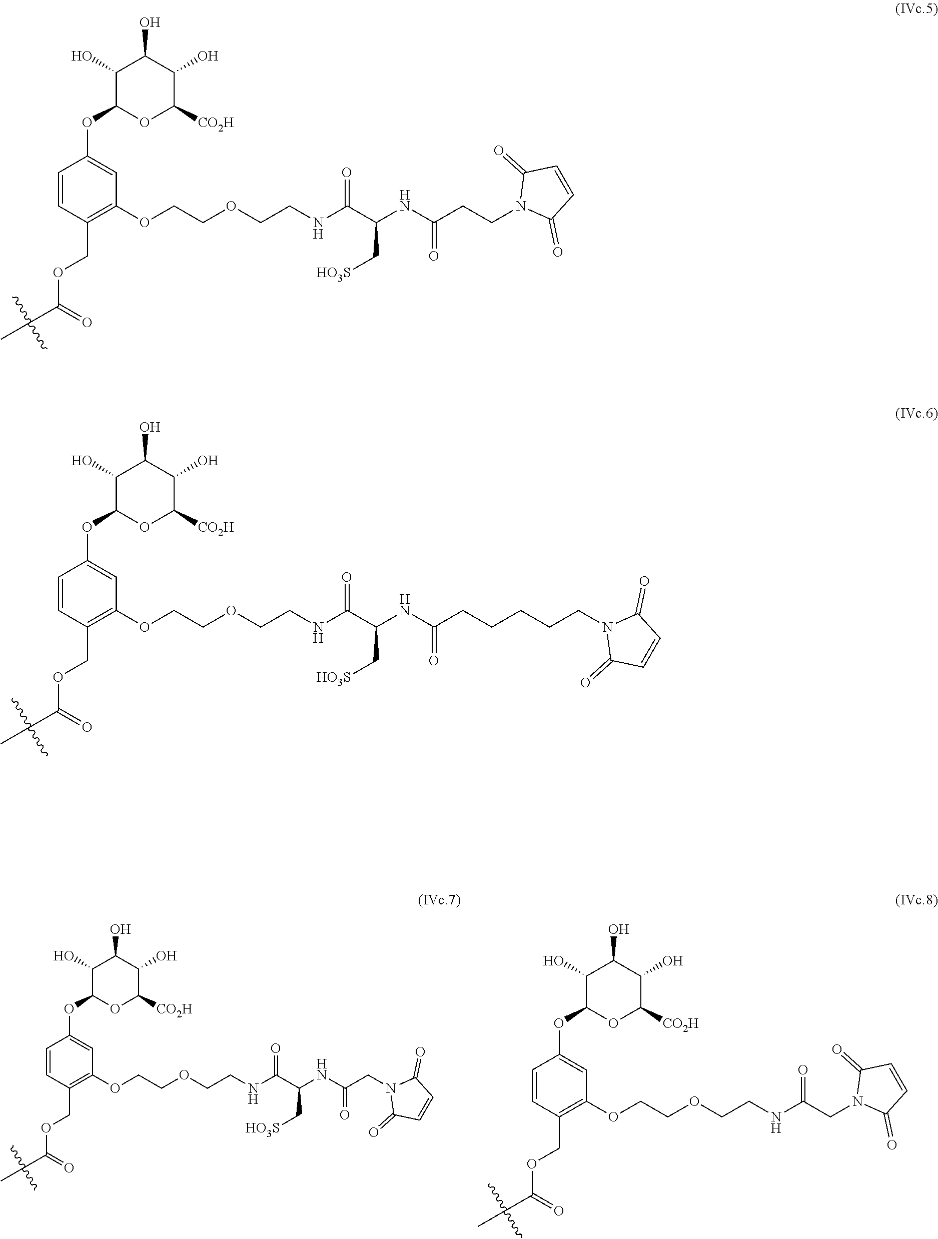

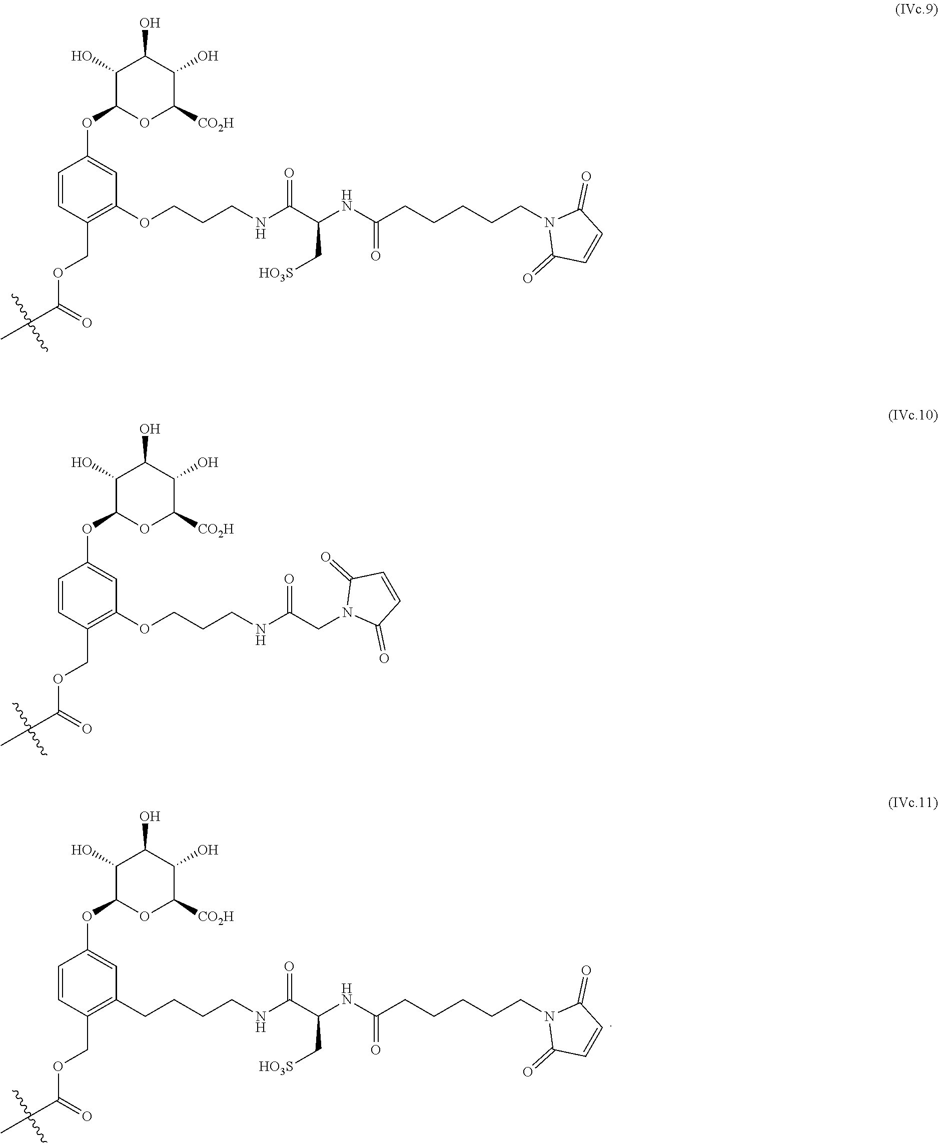

wherein [symbol] represents the point of attachment of a linker to an immune-modulatory agent.

Exemplary embodiments of linkers according to structural formula (IVd) that may be included in the conjugates described herein include the linkers illustrated below (as illustrated, the linkers include a group suitable for covalently attaching a linker to an antibody construct):

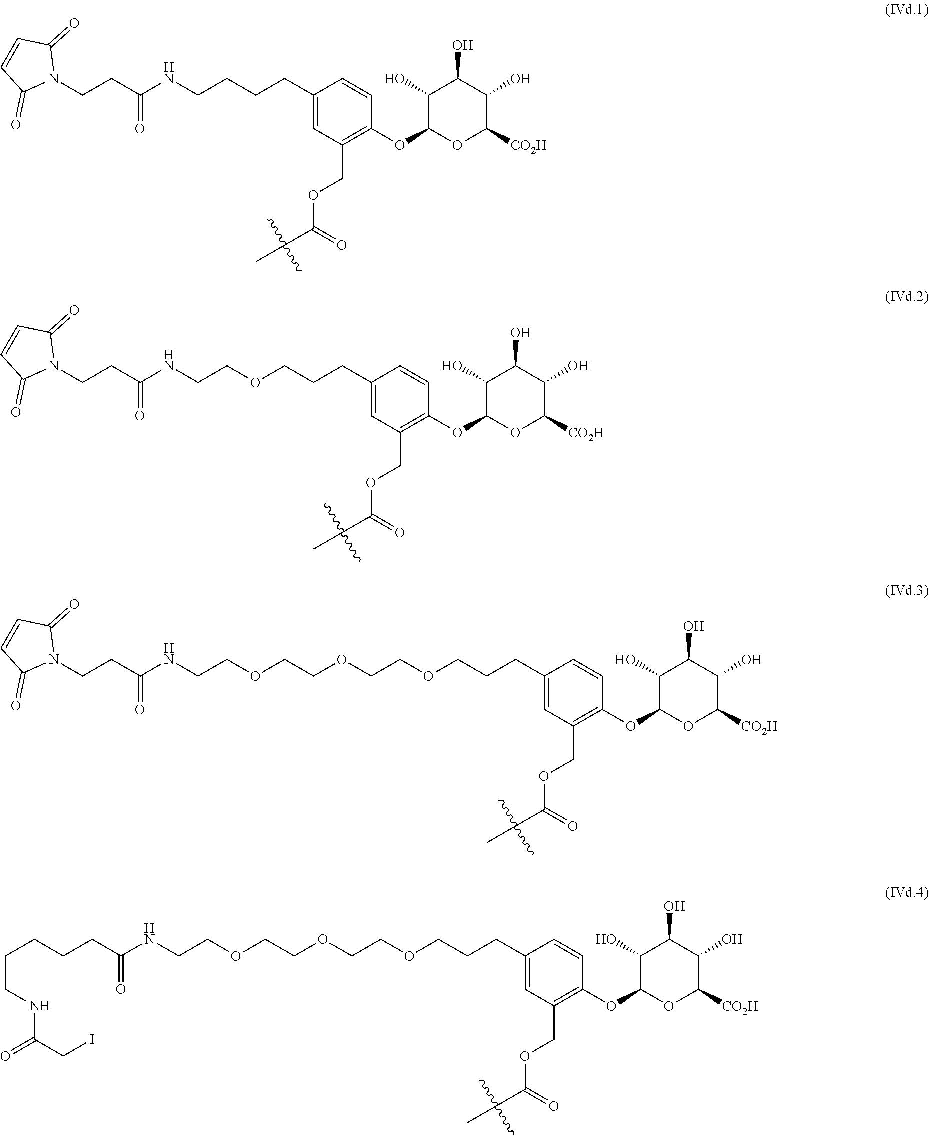

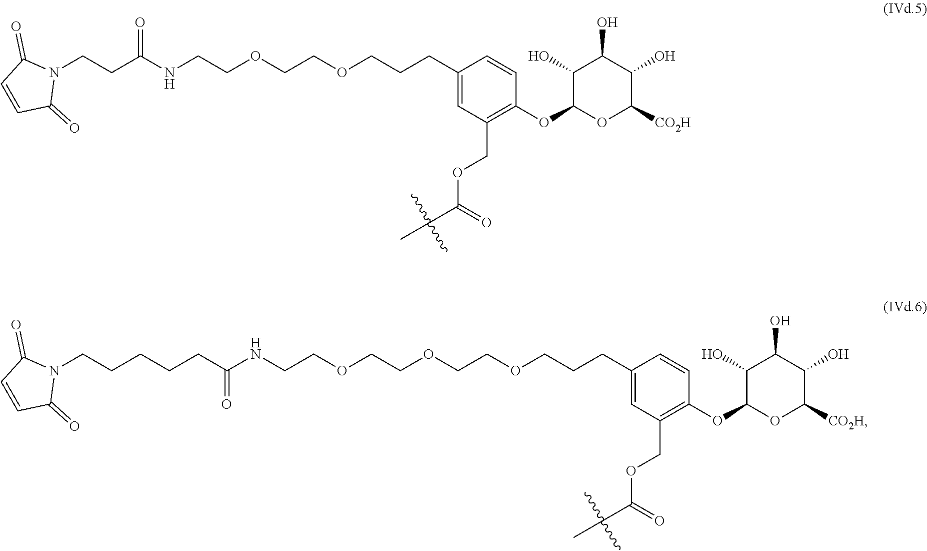

wherein [symbol] represents the point of attachment of a linker to an immune-modulatory agent.

Exemplary embodiments of linkers according to structural formula (IVe) that may be included in the conjugates described herein include the linkers illustrated below (as illustrated, the linkers include a group suitable for covalently attaching the linker to an antibody construct):

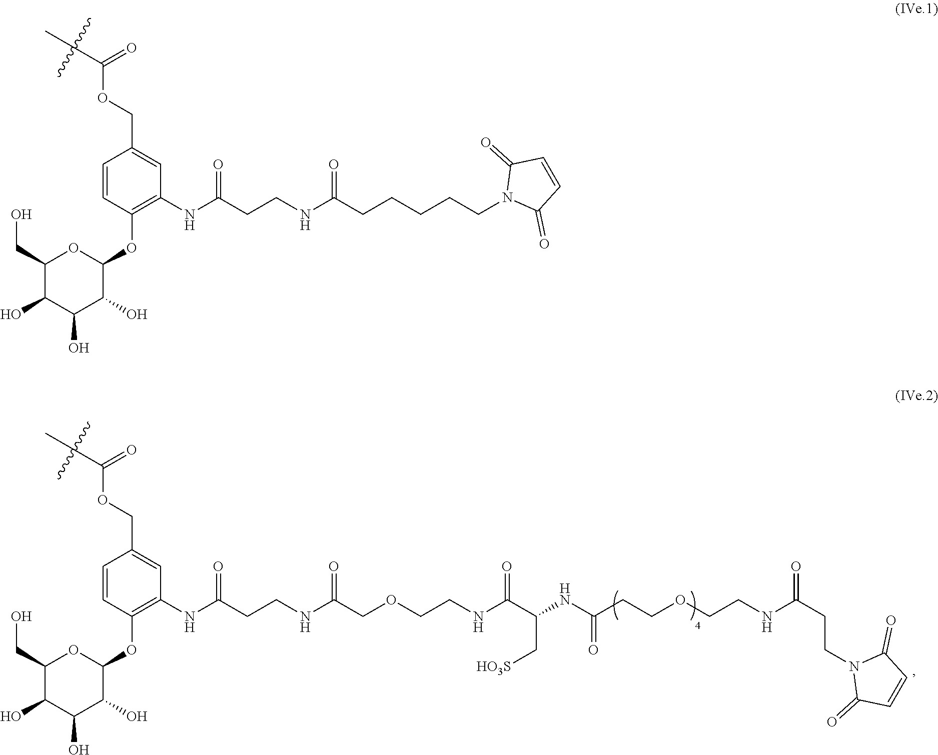

wherein [symbol] represents the point of attachment of a linker to an immune-modulatory agent.

Although cleavable linkers can provide certain advantages, the linkers comprising the conjugate described herein need not be cleavable. For non-cleavable linkers, the immune-modulatory agent release may not depend on the differential properties between the plasma and some cytoplasmic compartments. The release of the immune-modulatory agent can occur after internalization of the immune-modulatory conjugate via antigen-mediated endocytosis and delivery to lysosomal compartment, where the antibody construct can be degraded to the level of amino acids through intracellular proteolytic degradation. This process can release an immune-modulatory agent derivative, which is formed by the immune-modulatory agent, the linker, and the amino acid residue or residues to which the linker was covalently attached. The immune-modulatory agent derivative from immune-modulatory conjugates with non-cleavable linkers can be more hydrophilic and less membrane permeable, which can lead to less bystander effects and less nonspecific toxicities compared to immune-modulatory conjugates with a cleavable linker. Immune-modulatory conjugates with non-cleavable linkers can have greater stability in circulation than immune-modulatory conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

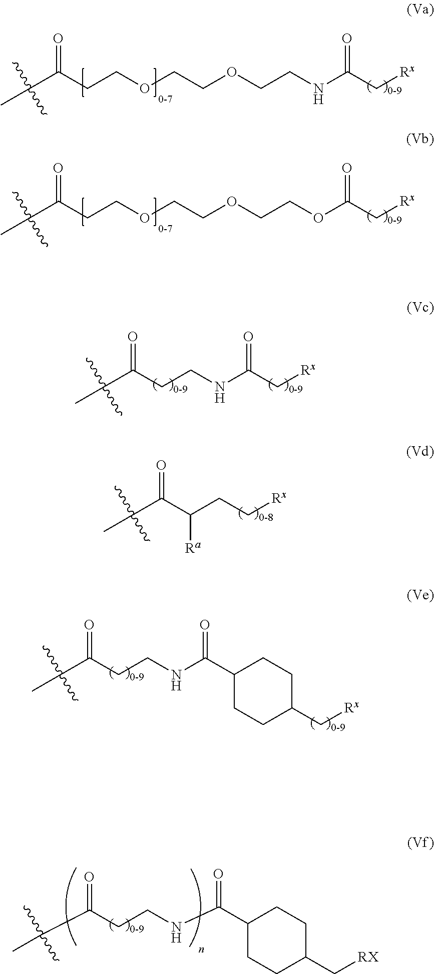

or salts thereof, wherein: R^{a} is selected from hydrogen, alkyl, sulfonate and methyl sulfonate; R^{x} is a reactive moiety including a functional group capable of covalently linking the linker to an antibody construct; and [symbol] represents the point of attachment of the linker to an immune-modulatory agent.

Exemplary embodiments of linkers according to structural formula (Va)-(Vf) that may be included in the conjugates described herein include the linkers illustrated below (as illustrated, the linkers include a group suitable for covalently linking the linker to an antibody construct, and [symbol] represents the point of attachment of the linker to an immune-modulatory agent:

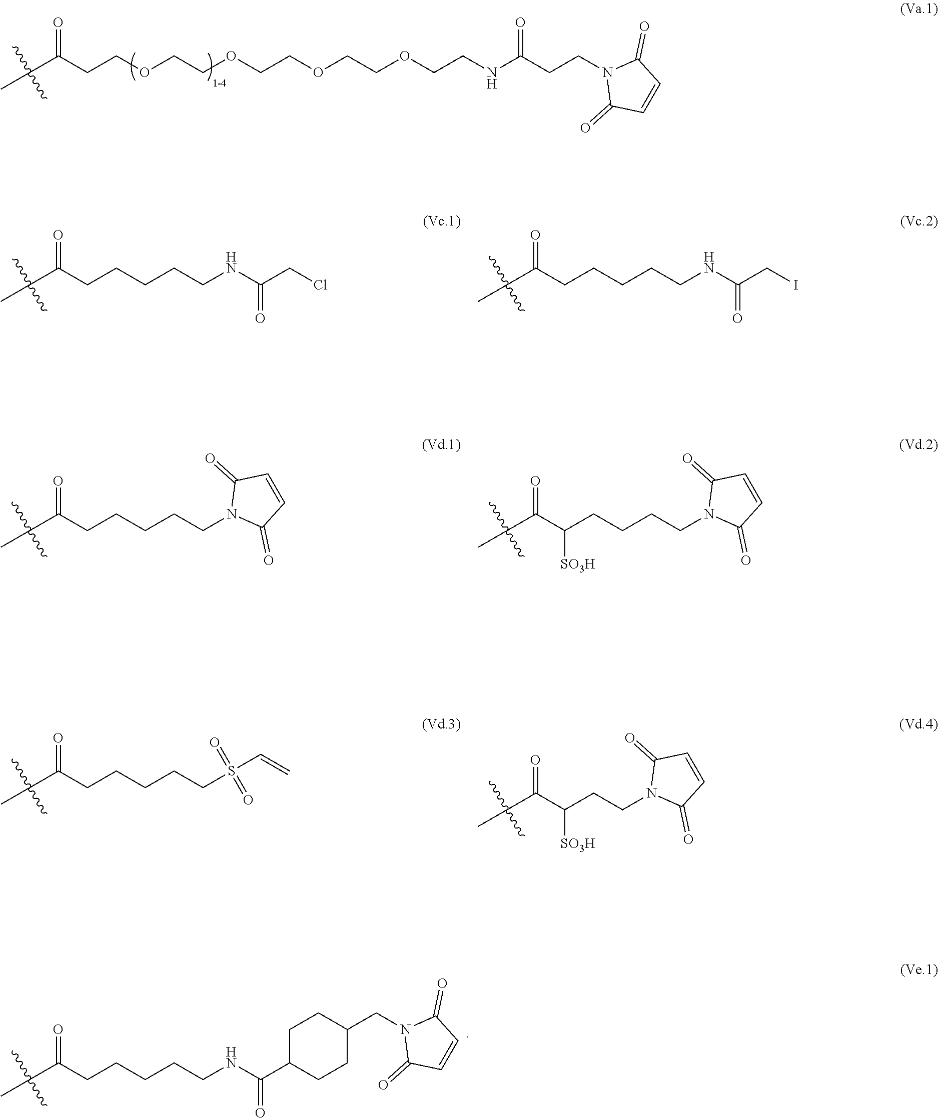

Attachment groups that are used to attach the linkers to an antibody construct can be electrophilic in nature and include, for example, maleimide groups, alkynes, alkynoates, allenes and allenoates, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody of a conjugate. The reaction between a thiol group of an antibody and a drug with a linker including a maleimide group proceeds according to the following scheme:

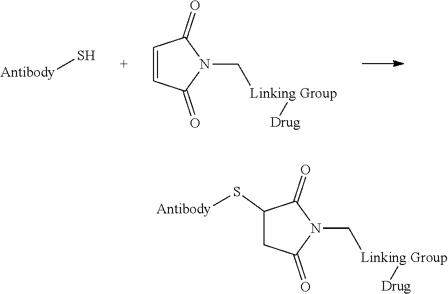

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic is shown below:

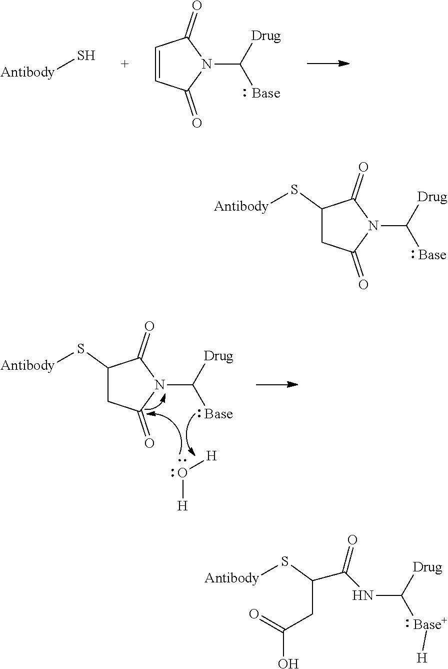

The hydrolysis reaction schematically represented above may occur at either carbonyl group of the succinimide group. Accordingly, two possible isomers may result, as shown below:

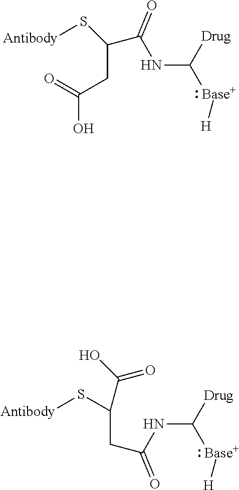

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker described herein, e.g., any linker described herein with a maleimide group prior to conjugating to an antibody construct, may facilitate hydrolysis of a nearby succinimide group formed after conjugation of the antibody construct to the linker. Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH₂)ₘ—, where m is an integer from 0 to 10. An alkylene chain may be optionally substituted with other functional groups as described herein.

A linker described herein with a maleimide group may include an electron withdrawing group such as, but not limited to, —C(O)R, ═O, —CN, —NO₂, —CX₃, —X, —COOR, —CONR₂, —COR, —COX, —SO₂R, —SO₂OR, —SO₂NHR, —SO₂NR₂, PO₃R₂, —P(O)(CH₃)NHR, —NO, —NR₃⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups such as those described herein.

Examples of self-stabilizing linkers are provided in, e.g., U.S. Patent Publication Number 2013/0309256, the linkers of which are incorporated by reference herein. It will be understood that a self-stabilizing linker useful in conjunction with immune-modulatory agents may be equivalently described as unsubstituted maleimide-including linkers, thio-substituted succinimide-including linkers, or hydrolyzed, ring-opened thio-substituted succinimide-including linkers.

In certain embodiments, a linker comprises a stabilizing linker moiety selected from:

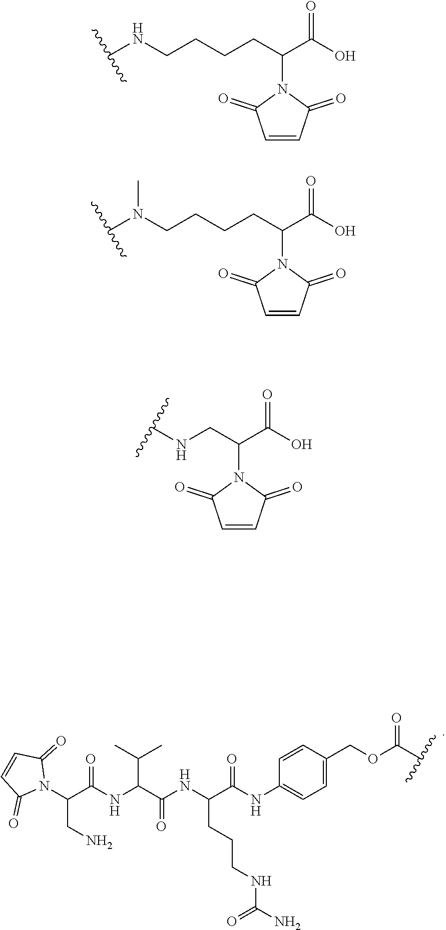

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represents the point of attachment to an immune-modulatory agent.

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls from interchain disulfides) followed by reaction with 4 equivalents of the alkylating agent. Conjugates containing “bridged disulfides” are also claimed to have increased stability.

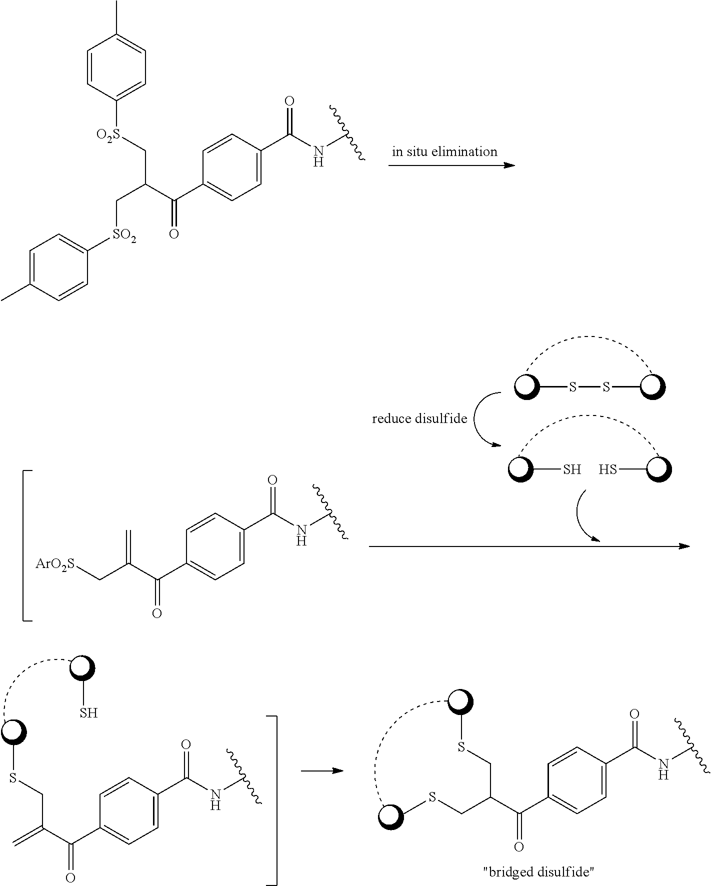

Similarly, as depicted below, a maleimide derivative that is capable of bridging a pair of sulfhydryl groups has been developed.

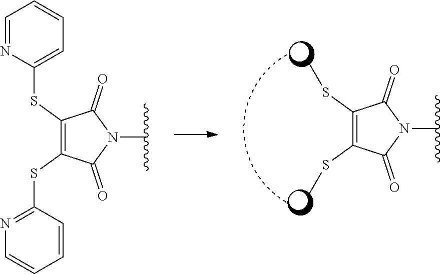 [SMILES: *N1C(=O)C(Sc2ccccn2)=C(Sc2ccccn2)C1=O ; *N1C(=O)C(SC)=C(SC)C1=O]

A linker can contain the following structural formulas (VIa), (VIb), or (VIc):

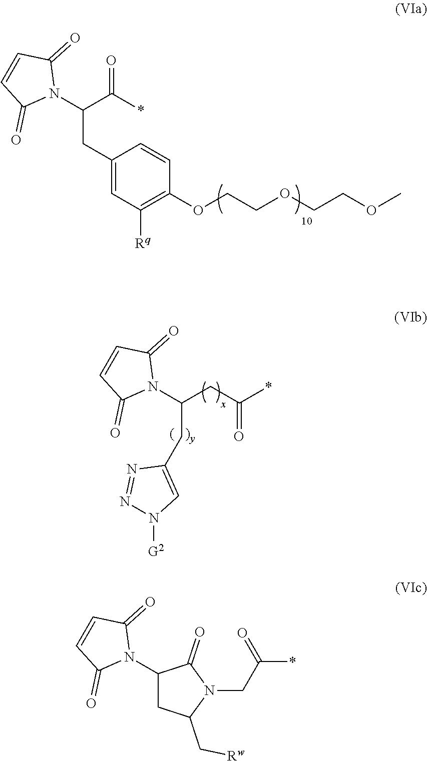

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIa) and (VIb) that can be included in the conjugates described herein can include the linkers illustrated below (as illustrated, the linkers can include a group suitable for covalently linking the linker to an antibody construct):

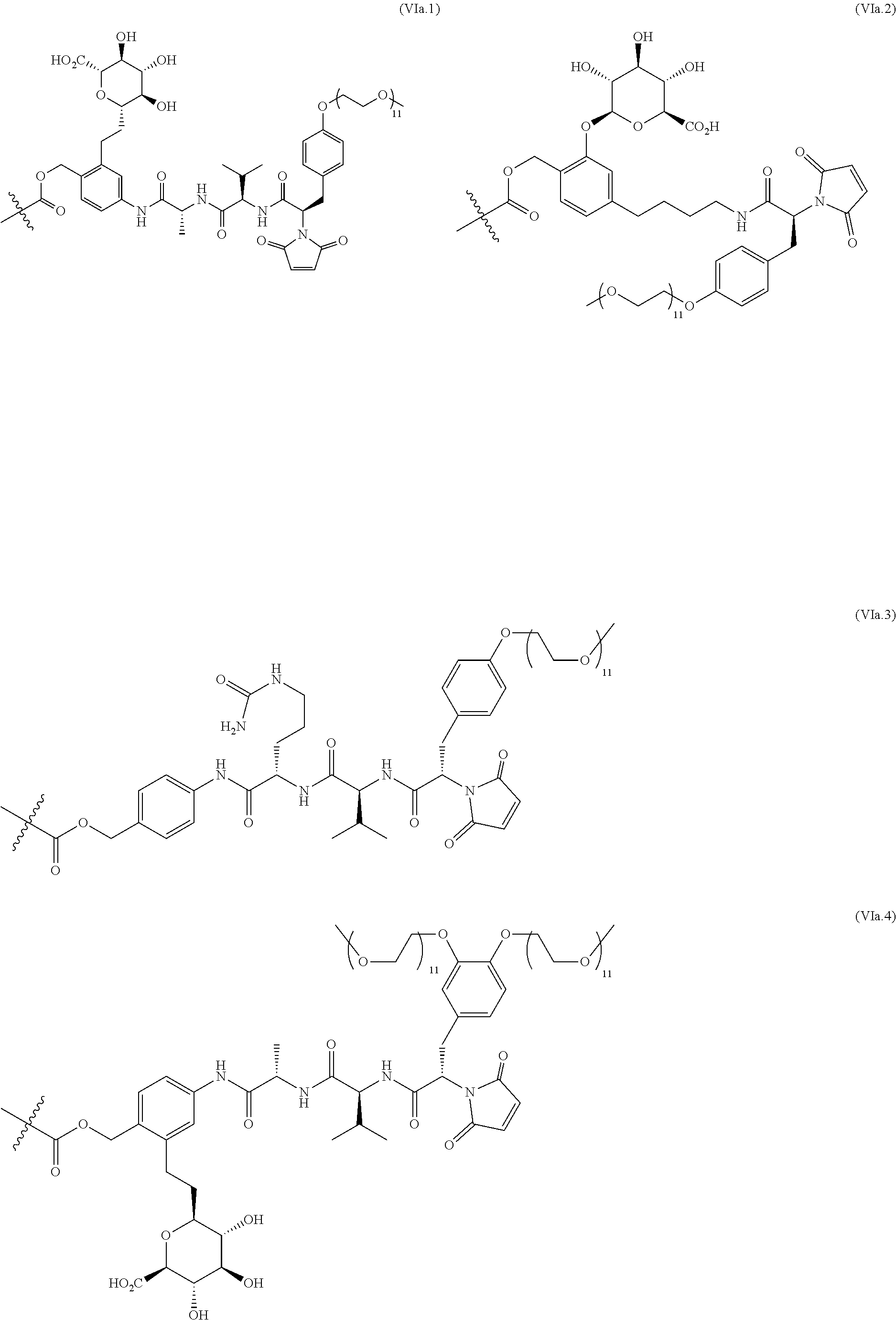

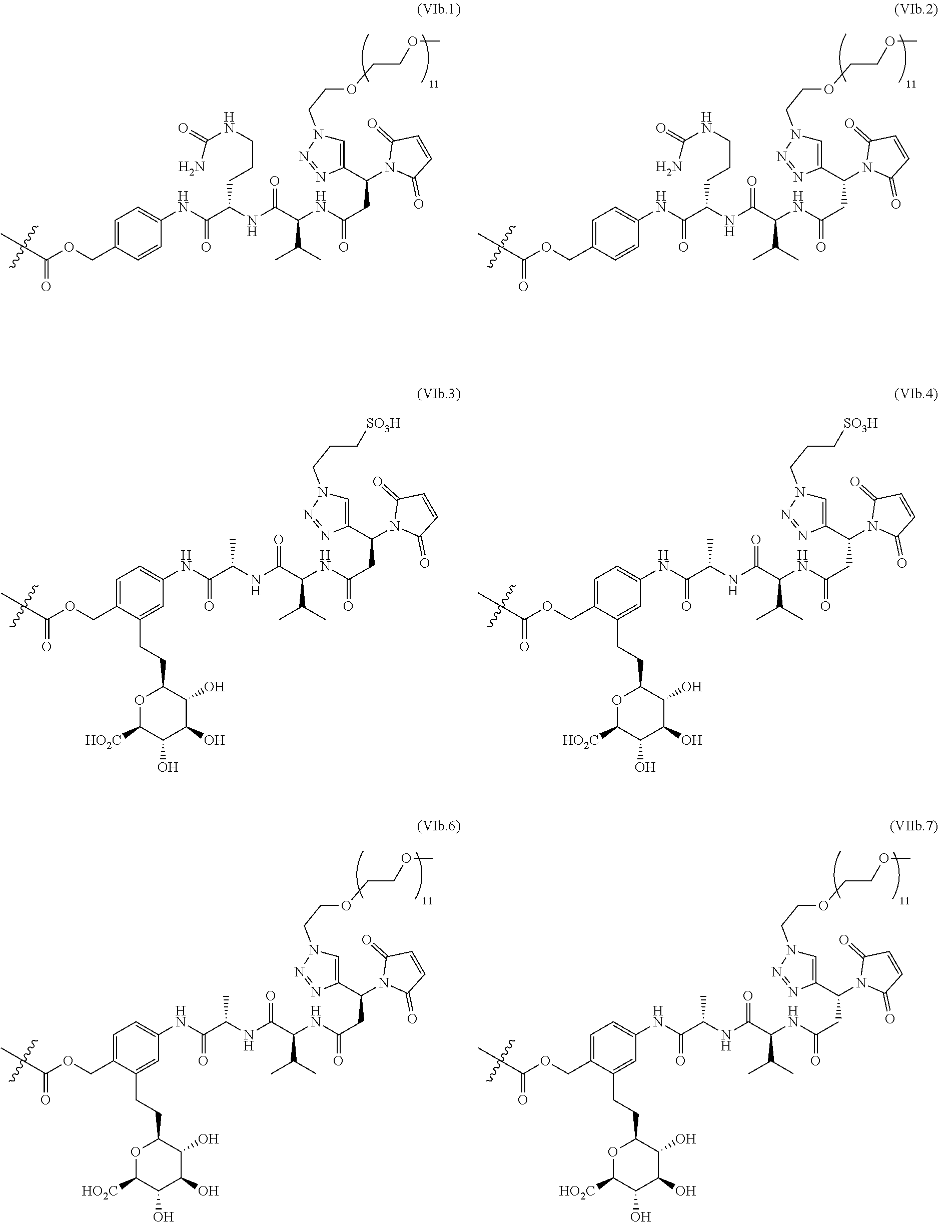

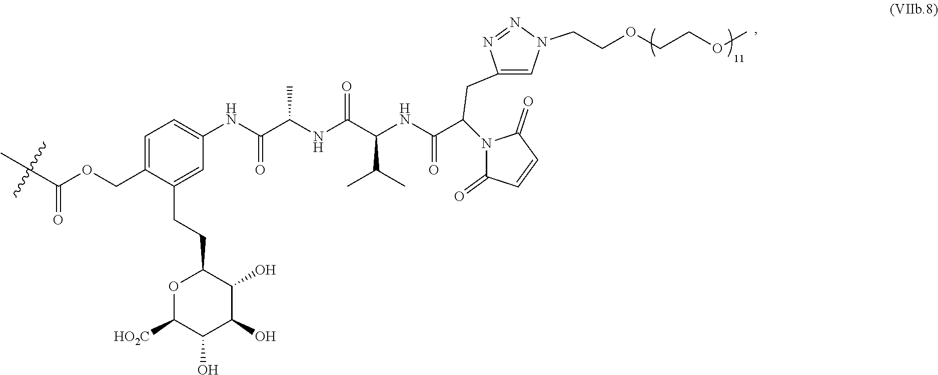

wherein [symbol] represents the point of attachment of the linker to an immune-modulatory agent.

Exemplary embodiments of linkers according to structural formula (VIc) that can be included in the immune-modulatory conjugates described herein can include the linkers illustrated below (as illustrated, the linkers can include a group suitable for covalently attaching the linker to an antibody construct):

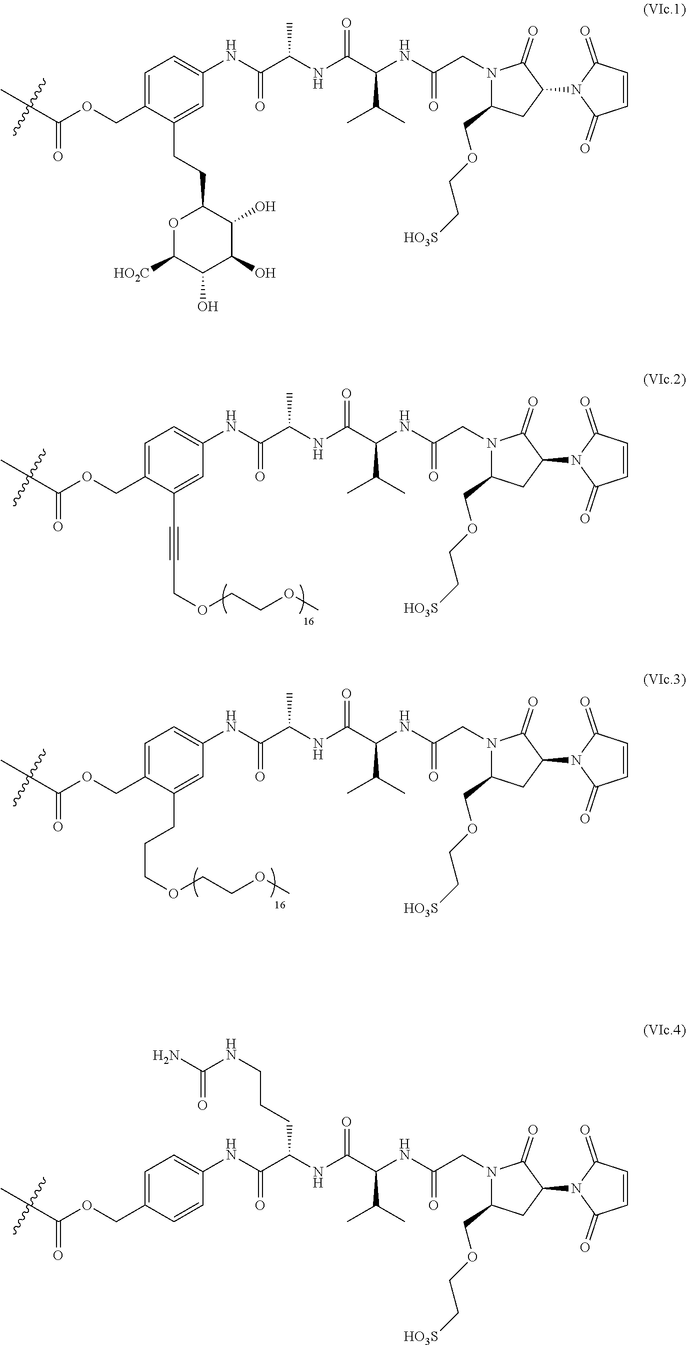

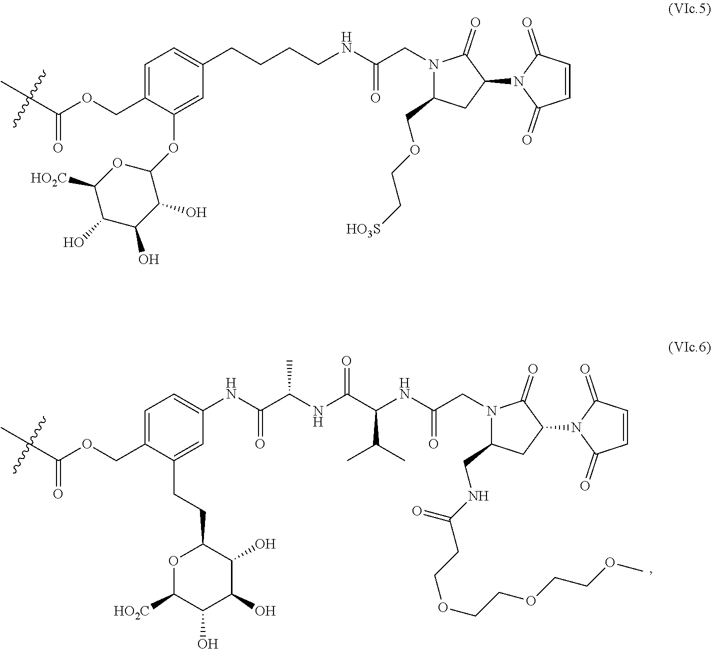

wherein [symbol] represents the point of attachment of the linker to an immune-modulatory agent.

### Conjugates

A conjugate as described herein includes an antibody construct and at least one linker attached to at least one immune-modulatory agent. In some aspects, a conjugate comprises an an antibody construct, at least one immune-modulatory agent, and at least one linker attaching the immune-modulatory agent to the antibody construct. In some embodiments, the linker contains a protease cleavage site specifically cleaved by a protease that is present in the extracellular portion of the disease microenvironment. A cleavable linker can contain protease cleavage site for a protease that is over-expressed in the disease microenvironment. Cleavage of the protease cleavage site allows selective release of the immune-modulatory agent in the disease. In some embodiments, a conjugate includes an engineered Fc domain for selective delivery of an immune-modulatory agent(s), based on the binding of the Fc domain to Fc receptors on the target cells, such as immune cells.

In some embodiments, an immune-modulatory conjugate comprises: (a) an antibody construct comprising an antigen binding domain and an Fc domain, the antigen binding domain specifically binding to an antigen expressed on a plurality of target cells; (b) at least one immune-modulatory agent; and (c) at least one linker, each linker being covalently attached to the antibody construct and to at least one immune-modulatory agent. The conjugate further comprises one or all of the following: (i) the antigen binding domain specifically binds to an antigen expressed in the extracellular microenvironment of a target disease, the linker is a cleavable linker having a protease cleavage site cleavable by a protease that is preferentially present in the extracellular microenvironment, and cleavage of the protease cleavage site releases an active form of the immune-modulatory agent; or (ii) the Fc domain comprises an amino acid sequence having a Kd for a first Fc receptor that is at least 10 fold higher than a Kd of the amino acid sequence for a wild-type IgG1 Fc domain for the first Fc receptor and having a Kd for a second F receptor that is the same or lower than a Kd of the amino acid sequence for a wild-type IgG1 Fc domain for the second Fc receptor, thereby allowing the Fc domain to preferentially bind to cells expressing the second Fc receptor; or (iii) both (i) and (ii).

In some embodiments, the immune-modulatory conjugate is represented by the following formula:

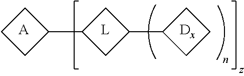

wherein:

A is the antibody construct having the antigen binding domain and the Fc domain;

L is the linker;

Dx is the immune-modulatory agent;

n is selected from 1 to 20; and

z is selected from 1 to 20.

In some embodiments, n is from 1 to 5 and z is from 1 to 8, n is 1 and z is from 1 to 8, or n is 1 and z is from 1 to 5.

In some embodiments, the linker has the following formula:

Rx-An-protease cleavage site-Y_{y}—,

wherein Rx is a reactive moiety attached to the antibody construct,

A is a stretcher unit and n is 0 or 1, and

Y is a self immolative unit and y is 0, 1, or 2.

Rx can be, for example, a succinimide group or a hydrolyzed succinmide group. In some embodiments, Y is a self-immolative group. A self-immolative group can be, for example, a PABA or PABC group. The linker can be, for example, maleimidocaproyl-protease cleave site-PABC-.

In some embodiments, the linker is a cleavable linker having a protease cleavage site cleavable by a protease that is preferentially present in the extracellular microenvironment of the target cells, whereby cleavage of the protease cleavage site releases an active form of the immune-modulatory agent in the extracellular microenvironment.

In some embodiments, the linker contains a protease-cleavage site for a protease selected from the group consisting of TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA. In some embodiments, the protease cleavage site is selected from a site cleavable by a protease selected from the group consisting of legumain, plasmin, TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, uPA, fibroblast associated protein, matriptase, PSMA and PSA. In some embodiments, the protease is selected from one of the groups consisting of: Legumain and plasmin; TMPRSS3, TMPRSS4, and TMPRSS6; MMP1, MMP2, MMP-3, MMP-9, MMP-8, MMP-14, and MT1-MMP; CATHEPSIN D, CATHEPSIN K, and CATHEPSIN S; ADAM10, ADAM12 and ADAMTS; Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and Caspase-14; and TACE, human neutrophil elastase, beta-secretase, uPA, fibroblast associated protein, matriptase, PSMA and PSA.

In some embodiments, the protease is preferentially localized in the extracellular microenvironment of the target cells. As used herein, preferential localization of a protease in an extracellular microenvironment refers to the increased presence of an active or activatable form of the protease in the vicinity of the target cells associated with a disease to be treated, such that the protease can cleave the protease cleavage site and release an active form of the immune-modulatory agent upon antigen binding by the conjugate, as compared to the amount of protease in an extracellular environment of normal cells (i.e., cells not associated with the disease. For example, tumor cells may express certain proteases on the cell surface or release them into the extracellular microenvironment, thereby selectively increasing the local amount of the protease, as compared to the extracellular microenvironment of normal (non-cancerous cells). Cells associated with other disease states involving significant remodeling may similarly have a localized increased amount of a protease in the extracellular microenvironment, as compared to the extracellular microenvironment of normal cells. Preferential localization of a protease in an extracellular environment can be determined, for example, as shown in Example 2.

In some embodiments, the protease is preferentially localized in the extracellular microenvironment of the target cells as compared to the extracellular microenvironment of normal cells by a factor of at least 5:1, 10:1, 25:1, 50:1 or 100:1. In some embodiments, the active form of the immune-modulatory agent is preferentially released in the extracellular microenvironment as compared to the extracellular microenvironment of normal cells by a factor of at least 5:1, 10:1, 25:1, 50:1 or 100:1.

In some embodiments, the Fc domain comprises an amino acid sequence having a Kd for a first Fc receptor that is at least 10 fold higher than a Kd of the amino acid sequence for a wild-type IgG1 Fc domain for the first Fc receptor and having a Kd for a second F receptor that is the same or lower than a Kd of the amino acid sequence for a wild-type IgG1 Fc domain for the second Fc receptor, thereby allowing the Fc domain to preferentially bind to cells expressing the second Fc receptor.

For example, the first Fc receptor can be an Fcγ receptor, such as an FcγRI, FcγRIIA, FcγRIIB, FcγRIIIA, or FcγRIIIB receptor and the second Fc receptor can an FcRn receptor. The Fc domain has an amino acid sequence that binds to the Fcγ receptor with a lower binding affinity (higher Kd) than the binding affinity (Kd) of a wildtype Fc domain that binds to the Fcγ receptor. The Fc domain has a binding affinity for an FcRn receptor that is the same or higher (lower Kd) as compared to the binding affinity of a wildtype Fc domain for the FcRn receptor. The wildtype Fc domain is typically of the same isotype as the Fc domain of the antibody construct for purposes of comparison. For example, if the Fc domain of the antibody construct is from an IgG1 or derived from an IgG1, the comparator Fc domain is a corresponding IgG1 Fc domain.

In some embodiments, the first Fc receptor is an FcγRI receptor and the second receptor is an FcRn receptor. In some embodiments, the first Fc receptor is an FcγRIIA receptor and the second receptor is an FcRn receptor. In some embodiments, the first Fc receptor is an FcγRIIB receptor and the second receptor is an FcRn receptor. In some embodiments, the first Fc receptor is an FcγRIIIA receptor and the second receptor is an FcRn receptor. In some embodiments, the first Fc receptor is an FcγRIIIB receptor and the second receptor is an FcRn receptor.

In some embodiments, the first Fc receptor is an FcRn receptor and the second receptor is an FcγRI receptor. In some embodiments, the first Fc receptor is an FcRn receptor and the second receptor is an FcγRIIA receptor. In some embodiments, the first Fc receptor is an FcRn receptor and the second receptor is an FcγRIIB receptor. In some embodiments, the first Fc receptor is an FcRn receptor and the second receptor is an FcγRIIIA receptor. In some embodiments, the first Fc receptor is an FcRn receptor and the second receptor is an FcγRIIIB receptor.

In some embodiments, the Kd of the Fc domain for the FcRn receptor is at least 5 fold lower than the Kd of a wild-type IgG1 Fc domain for the FcRn receptor. In some embodiments, an immune-modulatory conjugate can selectively deliver at least five times, at least ten times or at least 20 times as much immune-modulatory agent to cells expressing the second receptor, as compared to a conjugate having a wildtype Fc domain (e.g., a wildtype IgG1 Fc domain). For example, in some embodiments, the immune-modulatory conjugate having decreased binding affinity (increased kd) for one or more Fcγ receptors can selectively deliver at least five times, at least ten times or at least 20 times as much immune-modulatory agent to dendritic cells, as compared to a conjugate having a wildtype Fc domain (e.g., a wildtype IgG1 Fc domain).

In some embodiments, such an immune modulatory conjugate comprises a non-cleavable linker(s) attaching the immune-modulatory agent(s) to the antibody construct. For example, non-cleavable linkers having formulae Va-Vf, as described herein can be used. In some embodments, a non-cleavable linker is a substituted maleimidyl-alkylene group.

In other embodiments, such an immune modulatory conjugate comprises a cleavable linker(s) attaching the immune-modulatory agent(s) to the antibody construct, such as the linkers having formula I-IV and VI, as described herein. For example, a cleavable linker can further include one or more of the following groups: a succimide group, a hydrolyzed succimide group, a PABA group, a PABC group, and/or one or more polyethylene glycol molecules. A linker can be a (maleimidocaproyl)-(protease cleavage site)-(para-aminobenzyloxycarbonyl) linker prior to attachment to the antibody construct. A linker can contain a maleimide at one end, a protease cleavage site and an N-hydroxysuccinimidyl ester at the other end, prior to attachment to the antibody construct. A linker can contain a lysine with an N-terminal amine acetylated, and a protease cleavage site. A linker can be a link created by a microbial transglutaminase, wherein the link is created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 811) to an N-terminal GGG motif to regenerate a native amide bond. The linker created can therefore link a moiety attached to the LXPTG recognition motif (SEQ ID NO: 811) with a moiety attached to the N-terminal GGG motif. A linker can be a link created between an unnatural amino acid on one moiety reacting with oxime bond that was formed by modifying a ketone group with an alkoxyamine on another moiety. A moiety can be a portion of an antibody construct. A moiety can be a binding domain. A moiety can be a portion of an antibody. A moiety can be an immune-modulatory agent.

In some embodiments, an immune-modulatory conjugate comprises an antibody construct, at least one pattern recognition receptor (PRR) agonist, and at least one linker. In some embodiments, an immune-modulatory conjugate comprises an antibody construct, at least one pattern-associated molecular pattern (PAMP) molecule, and at least one linker. In some embodiments, an immune-modulatory conjugate comprises an antibody construct, at least one damage-associated molecular pattern (DAMP) molecule, and at least one linker.

In some embodiments, an immune-modulatory conjugate comprises an antibody construct, at least one TLR2 agonist, and at least one linker. In some embodiments, an immune-modulatory conjugate comprises an antibody construct, at least one TLR3 agonist, and at least one linker. In some embodiments, an immune-modulatory conjugate comprises an antibody construct, at least one TLR4 agonist, and at least one linker. In some embodiments, an immune-modulatory conjugate comprises an antibody construct, at least one TLR5 agonist, and at least one linker. In some embodiments, an immune-modulatory conjugate comprises an antibody construct, at least one TLR6 agonist, and at least one linker. In some embodiments, an immune-modulatory conjugate comprises an antibody construct, at least one TLR7 agonist, and at least one linker. In some embodiments, an immune-modulatory conjugate comprises an antibody construct, at least one TLR8 agonist, and at least one linker. In some embodiments, an immune-modulatory conjugate comprises an antibody construct, at least one TLR9 agonist, and at least one linker. In some embodiments, an immune-modulatory conjugate comprises an antibody construct, at least one TLR10 agonist, and at least one linker. In some embodiments, an immune-modulatory conjugate comprises an antibody construct, at least one STING agonist, and at least one linker. In some embodiments, an immune-modulatory conjugate comprises an antibody construct, at least one RIG-I ligand, and at least one linker. In some embodiments, an immune-modulatory conjugate comprises an antibody construct, at least one least NOD-like receptor ligand, and at least one linker.

An antigen binding domain(s) of a conjugate can be specifically bind to a tumor antigen, or an antigen on cells associated with a fibrotic or inflammatory disease.

In some embodiments, an antigen binding domain(s) specifically binds to a tumor antigen. The tumor antigen can be selected from, for example, GD2, GD3, GM2, Le^{y}, sLe, polysialic acid, fucosyl GM1, Tn, STn, BM3, or GloboH, or from CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, STN1, TNC, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), VISTA, or a fragment thereof.

In certain embodiments, an antigen binding domain specifically binds to a tumor antigen, such as those selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In certain embodiments, an antigen binding domain specifically binds to an antigen associated with fibrotic or inflammatory disease, such as those selected from Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, MMP14, GPX8, and F2RL2.

An immune-modulatory conjugate can recognize a single antigen. In other embodiments, a conjugate can recognize two or more antigens. In some embodiments, a conjugate can recognize three or more antigens. In some embodiments, the binding affinity (Kd) for binding of a first antigen binding domain of a conjugate to an antigen in the presence of an immune-modulatory agent (i.e, when the immune-modulatory agent(s) are attached to the antibody construct via a linker(s)) can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the first binding domain to the antigen in the absence of the immune-modulatory agent (i.e., the antibody construct alone). In some embodiments, the binding affinity (Kd) for binding of a first antigen binding domain of a conjugate to an antigen in the presence of the immune-modulatory agent (i.e, when the immune-modulatory agent(s) are attached to the antibody construct via a linker(s)) can be less than 10 nM. In some embodiments, the binding affinity (Kd) for binding of a first antigen binding domain of a conjugate to an antigen in the presence of the immune-modulatory agent can be less than 100 nM, less than 50 nM, less than 20 nM, less than 5 nM, less than 1 nM, or less than 0.1 nM.

In some embodiments, the binding affinity (Kd) for binding of a second antigen binding domain of a conjugate to an antigen in the presence of an immune-modulatory agent can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of the second antigen binding domain to the antigen in the absence of the immune-modulatory agent (i.e., the antibody construct alone). In some embodiments, the binding affinity (Kd) for binding of a second antigen binding domain of a conjugate to an antigen in the presence of the immune-modulatory agent can be less than 10 nM. In some embodiments, the binding affinity (Kd) for binding of a second antigen binding domain of a conjugate to an antigen in the presence of the immune-modulatory agent can be less than 100 nM, less than 50 nM, less than 20 nM, less than 5 nM, less than 1 nM, or less than 0.1 nM.

In some embodiments, an immune-modulatory conjugate comprises one or more antigen binding domains, typically two antigen binding domains, that specifically bind to an antigen, such as a tumor antigen or an antigen on cells associated with a fibrotic or an inflammatory disease. In some embodiments, an immune-modulatory conjugate comprises one or more antigen binding domains, typically two antigen binding domains, that specifically bind to an antigen, such as a tumor antigen or an antigen on cells associated with a fibrotic or an inflammatory disease, and further comprises an antigen binding domain(s) that specifically binds to an antigen on an immune cells. In some embodiments, an immune-modulatory conjugate comprises one or more antigen binding domains, typically two antigen binding domains, that specifically bind to an antigen, such as an antigen expressed on an immune cell. An immune cell can be an antigen-presenting cell, such as a dendritic cell, a macrophage or a monocyte. An immune cell can be an antigen-presenting cell, such as a dendritic cell, a macrophage, a monocyte, or a B cell. An immune cell can be a T cell, a B cell, an NKT cell, or an NK cell.

In some embodiments, the Fc domain of the antibody construct of the conjugate can be a modified Fc domain that has a modified binding affinity for an Fc receptor, as described herein. The modified Fc domain can comprise a substitution at more than one amino acid residue, as described herein. The Kd for binding of an Fc domain to an Fc receptor can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the Kd for binding of a wildtype Fc domain to the Fc receptor. The Kd for binding of an Fc domain to an Fc receptor can be less than 10 nM. The Kd for binding of an Fc domain to an Fc receptor can be less than 100 nM, less than 50 nM, less than 20 nM, less than 5 nM, less than 1 nM, or less than 0.1 nM.

A linker can be attached to an antibody construct of a conjugate by a direct linkage between the antibody construct and the linker. A direct linkage is a covalent bond. A linker can be attached to an antibody construct at any suitable site, such as for example at a terminus of an amino acid sequence or at a side chain of a cysteine residue, an engineered cysteine residue, a lysine residue, a serine residue, a threonine residue, a tyrosine residue, an aspartic acid residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment site can also be at a residue containing an oxime bond that was formed by modifying a ketone group with an alkoxyamine on another moiety, and a reactive primary amine, such as a reactive primary amine at a C-terminal end of a protein or peptide, such as by using Sortase A linker, which can be created by a Sortase A enzyme fusing an LXPTG recognition motif (SEQ ID NO: 811) to an N-terminal GGG motif to regenerate a native amide bond. The linker created can therefore link a moiety attached to the LXPTG recognition motif (SEQ ID NO: 811) with a moiety attached to the N-terminal GGG motif.

An attachment can be via any of a number of types of bonds, for example but not limited to, an amide bond, an ester bond, an ether bond, a carbon-nitrogen bond, a carbon-carbon single, double or triple bond, a disulfide bond, or a thioether bond. A linker can have at least one functional group, which can be linked to the antibody construct or the antibody. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

A linker can be connected to an antibody construct at a hinge cysteine. A linker can be connected to an antibody construct at a light chain constant domain lysine. A linker can be connected to an antibody construct at an engineered cysteine in the light chain. A linker can be connected to an antibody construct at an engineered light chain glutamine. A linker can be connected to an antibody construct at an unnatural amino acid engineered into the light chain. A linker can be connected to an antibody construct at a heavy chain constant domain lysine. A linker can be connected to an antibody construct at an engineered cysteine in the heavy chain. A linker can be connected to an antibody construct at an engineered heavy chain glutamine. A linker can be connected to an antibody construct an unnatural amino acid engineered into the heavy chain. Amino acids can be engineered into an amino acid sequence of an antibody construct as described herein, for example, and can be connected to a linker of a conjugate. Engineered amino acids can be added to a sequence of existing amino acids. Engineered amino acids can be substituted for one or more existing amino acids of a sequence of amino acids.

A linker can be conjugated to an antibody construct via a sulfhydryl group. A linker can be conjugated to an antibody construct via a primary amine. A linker can be a link created between an unnatural amino acid on an antibody construct reacting with an oxime bond that was formed by modifying a ketone group with an alkoxyamine on an immune-modulatory agent. In some embodiments, when a linker is connected to an antibody construct at the sites described herein, an Fc domain of the conjugate can bind to Fc receptors. In some embodiments, when a linker is connected to an antibody construct at the sites described herein, the antigen binding domain of the conjugate can bind its antigen. When a linker is connected to an antibody construct at the sites described herein, a binding domain of the conjugate can bind its antigen.

In a conjugate, the drug loading is represented by z, the number of immune-modulatory agent-linker molecules per antibody construct, or the number of immune-modulatory agents per antibody construct, depending on the particular conjugate. Depending on the context, z can represent the average number of immune-modulatory agent (-linker) molecules per antibody construct, also referred to the average drug loading. Z can range from 1 to 20, from 1 to 50 or from 1 to 100. In some conjugates, z is preferably from 1 to 8. In some preferred embodiments, when z represents the average drug loading, z ranges from about 2 to about 5. In some embodiments, z is about 2, about 3, about 4, or about 5. The average number of immune-modulatory agents per antibody construct in a preparation may be characterized by conventional means such as mass spectroscopy, HIC, ELISA assay, and HPLC.

### Pharmaceutical Formulations

The conjugates and methods described herein can be considered useful as pharmaceutical compositions for administration to a subject in need thereof. Pharmaceutical compositions can comprise at least the conjugates described herein and one or more pharmaceutically acceptable carriers, diluents, excipients, stabilizers, dispersing agents, suspending agents, and/or thickening agents. A pharmaceutical composition can comprise a conjugate comprising an antibody construct and an immune-modulatory agent, such as an agonist. A pharmaceutical composition can comprise a conjugate comprising an antibody construct having at least one antigen binding domain, an Fc domain, at least one immune-modulatory agent, such as an agonist and at least one linker. The pharmaceutical composition can comprise any conjugate described herein.

A pharmaceutical composition can further comprise buffers, antibiotics, steroids, carbohydrates, drugs (e.g., chemotherapy drugs), radiation, polypeptides, chelators, adjuvants and/or preservatives.

In a pharmaceutical composition, the conjugates can have an average drug loading. The drug loading, z, is the average number of immune-modulatory agent-linker molecules per antibody construct, or the number of immune-modulatory agents per antibody construct. Z can range from an average of 1 to 20, or 1-100. In some compositions, z is preferably from 1 to 8, from 2 to 5, from 3-5, about 2 or about 4. The average number of immune-modulatory agents per antibody construct in a preparation may be characterized by conventional means such as mass spectroscopy, HIC, ELISA assay, and HPLC.

Pharmaceutical compositions can be formulated using one or more physiologically-acceptable carriers comprising excipients and auxiliaries. Formulation can be modified depending upon the route of administration chosen. Pharmaceutical compositions comprising a conjugate as described herein can be manufactured, for example, by lyophilizing the conjugate, mixing, dissolving, emulsifying, encapsulating or entrapping the conjugate. Pharmaceutical compositions comprising a conjugate as described herein can be manufactured, for example, by lyophilizing the conjugate, mixing, dissolving, emulsifying, encapsulating or entrapping the conjugate. The pharmaceutical compositions can also include the conjugates described herein in a free-base form or pharmaceutically-acceptable salt form.

Methods for formulation of the pharmaceutical compositions can include formulating any of the conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for reconstitution with a suitable vehicle, e.g., sterile pyrogen-free water, before use

Pharmaceutical compositions of the conjugates described herein can comprise at least a conjugate as an active ingredient, respectively. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise more than one active compound as necessary for the particular indication being treated. The active compounds can have complementary activities that do not adversely affect each other. For example, the pharmaceutical composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the pharmaceutical compositions described herein can be lyophilized or in powder form for reconstitution with a suitable vehicle, e.g., sterile pyrogen-free water, before use.

For parenteral administration, the conjugates can be formulated in a unit dosage injectable form (e.g., a solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the conjugate, and these matrices can be in the form of shaped articles (e.g., films or microcapsules). Examples of sustained-release matrices can include polyesters, hydrogels (e.g., poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

### Therapeutic Applications

The conjugates, pharmaceutical compositions, and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The conjugates, pharmaceutical compositions, and methods described herein can be useful as a therapeutic, for example a treatment that can be administered to a subject in need thereof. A therapeutic effect can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the conjugates or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically-effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like can refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

Prevent, preventing and the like also can refer to the prevention of relapse of the disease or condition, e.g., tumor formation, in the patient. For example, an individual at risk of relapse of a tumor or other form of cancer after treatment and obtaining a state of remission can be treated with the methods of the present disclosure to prevent relapse.

A therapeutically effective amount can be the amount of a conjugate or pharmaceutical compositions or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates or pharmaceutical compositions described herein that can be used in therapy can be formulated and dosages established in a fashion consistent with good medical practice taking into account the disorder to be treated, the condition of the individual patient, the site of delivery of the conjugate or pharmaceutical composition, the method of administration and other factors known to practitioners. The conjugates or pharmaceutical compositions can be prepared according to the description of preparation described herein.

One of ordinary skill in the art would understand that the amount, duration and frequency of administration of a pharmaceutical composition or conjugate described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, conjugates and pharmaceutical compositions described herein can be for administration to a subject in need thereof. Often, administration of the conjugates or pharmaceutical compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition or conjugate can be administered to a subject by additional routes of administration, for example, by inhalation, oral, dermal, intranasal, or intrathecal administration.

Pharmaceutical compositions and conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The conjugates or pharmaceutical compositions can be administered to the subject in need thereof in cycles of 21 days, 14 days, 10 days, 7 days, 4 days or daily over a period of one to seven days.

### Diseases, Conditions and the Like

The conjugates and pharmaceutical compositions, and methods provided herein can be useful for the treatment of a plurality of diseases, conditions, preventing a disease or a condition in a subject or other therapeutic applications for subjects in need thereof. Often the conjugates, pharmaceutical compositions, and methods provided herein can be useful for treatment of hyperplastic conditions, including but not limited to, neoplasms, cancers, tumors and the like. A condition, such as a cancer, can be associated with expression of a molecule on the cancer cells. Often, the molecule expressed by the cancer cells can comprise an extracellular portion capable of recognition by the antibody portion of the antibody construct. A molecule expressed by the cancer cells can be a tumor antigen. An antibody construct of a conjugate or pharmaceutical composition thereof can recognize a tumor antigen. A tumor antigen can be, for example, GD2, GD3, GM2, Le^{y}, sLe, polysialic acid, fucosyl GM1, Tn, STn, BM3, or GloboH, or CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, STN1, TNC, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), VISTA, or a fragment thereof.

In certain embodiments, a tumor antigen can be selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

Additionally, such tumor antigens can be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sézary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenström macroglobulinemia; Wilms tumor and any combination thereof.

An antibody construct of the conjugate or composition may recognize a fibrotic associated antigen, autoimmune associated antigen, or autoinflammatory associated antigen. For example, an antigen may be Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. An antigen may be Cadherin 11, LRRC15, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. An antigen may be Cadherin 11, LRRC15, or FAP. An antigen may be Cadherin 11, TNFR2, or FAP. An antigen may be TNFR2.

As described herein, an antigen binding domain portion of the conjugate may be configured to recognize an antigen expressed by a disease cell, such as for example, a disease antigen. Often such antigens are known to those of ordinary skill in the art, or newly found to be associated with such a condition, to be commonly associated with, and/or specific to, such conditions. For example, a disease antigen is, but is not limited to, Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. A disease antigen may also be Cadherin 11, LRRC15, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. A disease antigen also may be FAP, LRRC15, or Cadherin 11. A disease antigen may be Cadherin 11, TNFR2, or FAP. A disease antigen may be TNFR2.

Non-limiting examples of fibrosis or fibrotic diseases include adhesive capsulitis, arterial stiffness, arthrofibrosis, atrial fibrosis, cirrhosis, Crohn's disease, collagenous fibroma, cystic fibrosis, Desmoid-type fibromatosis, Dupuytren's contracture, elastofibroma, endomyocardial fibrosis, fibroma of tendon sheath, glial scar, idiopathic pulmonary fibrosis, keloid, mediastinal fibrosis, myelofibrosis, nuchal fibroma, nephrogenic systemic fibrosis, old myocardial infarction, Peyronie's disease, pulmonary fibrosis, progressive massive fibrosis, radiation-induced lung injury, retroperitoneal fibrosis, scar, and scleroderma/systemic sclerosis.

Non-limiting examples of diseases that can be treated using a method according to the disclosure include acute disseminated encephalomyelitis (ADEM), acute necrotizing hemorrhagic leukoencephalitis, Addison's disease, agammaglobulinemia, alopecia, amyloidosis, ankylosing spondylitis (AS), Anti-GBM/Anti-TBM nephritis, antiphospholipid syndrome (APS), arthritis, autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, avascular Necrosis (Osteonecrosis)\Back Pain, axonal and neuronal neuropathy (AMAN), Balo disease, Behçet's Disease, bursitis and other soft tissue diseases, Bullous pemphigoid, cardiomyopathy, carpal tunnel syndrome, Castleman disease (CD), celiac disease, Chagas disease, chronic fatigue syndrome, chronic inflammatory demyelinating polyneuropathy (CIDP), chronic recurrent multifocal osteomyelitis (CRMO), Churg-Strauss, Cicatricial pemphigoid/benign mucosal pemphigoid, Cogan's syndrome, cold agglutinin disease, congenital heart block, Coxsackie myocarditis, CREST syndrome, collagen vascular disease, CPDD (Calcium Pyrophosphate Dihydrate Crystal Deposition Disease), Crohn's Disease, demyelinating neuropathies, degenerative joint disease, dermatitis herpetiformis, dermatomyositis, Devic's disease (neuromyelitis optica), diabetes (Type I), discoid lupus, DISH (Diffuse Idiopathic Skeletal Hypertosis), Dressler's syndrome, endometriosis, eosinophilic esophagitis (EoE), eosinophilic fasciitis, erythema nodosum, essential mixed cryoglobulinemia, dupuytren, EDS (Ehlers-Danlos Syndrome), EMS (Eosinophilia-Myalgia Syndrome), Evans syndrome, experimental allergic encephalomyelitis, Felty's Syndrome, fibromyalgia, fibromyositis, fibrosing alveolitis, giant cell arteritis (temporal arteritis), giant cell myocarditis, glomerulonephritis, Goodpasture's syndrome, gout, granulomatosis with Polyangiitis, Graves' Disease, Guillain-Barr-syndrome, Hashimoto's thyroiditis, hemolytic anemia, Henoch-Schonlein purpura (HSP), herpes gestationis or pemphigoid gestationis (PG), hypogammalglobulinemia, idiopathic pulmonary fibrosis, idiopathic thrombocytopenic purpura, IgA Nephropathy, IgG4-related sclerosing disease, immunoregulatory lipoproteins, inclusion body myositis (IBM), infectious arthritis, inflammatory bowel disease, interstitial cystitis (IC), JH (Joint Hypermobility), joint inflammation, juvenile rheumatoid arthritis, juvenile arthritis—other types and related conditions, juvenile dermatomyositis, juvenile diabetes (Type 1 diabetes), juvenile idiopathic arthritis (JIA), juvenile myositis (JM), juvenile non-inflammatory disorders, juvenile psoriatic arthritis, juvenile scleroderma, juvenile spondyloarthropathy syndromes, juvenile systemic lupus erythematosis (SLE), juvenile vasculitis, Kawasaki disease, Ledderhose Disease (Dupuytren of the feet), Lambert-Eaton syndrome, leukocytoclastic vasculitis, lichen planus, lichen sclerosus, ligneous conjunctivitis, linear IgA disease (LAD), lupus, Discoid, lupus erythematosis, lyme Disease, Marfan Syndrome, MCTD (Mixed Connective Tissue Disease), Meniere's disease, microscopic polyangiitis (MPA), mixed connective tissue disease (MCTD), Mooren's ulcer, Mucha-Habermann disease, multiple sclerosis, myasthenia gravis, myocarditis, myofascial pain, narcolepsy, neuromyelitis optica, neutropenia, ocular cicatricial pemphigoid, optic neuritis, osteoarthritis, osteogenesis imperfecta, osteonecrosis (Avascular Necrosis), osteoporosis, Paget's Disease, palindromic rheumatism (PR), PANDAS (Pediatric Autoimmune Neuropsychiatric Disorders Associated with Streptococcus), paraneoplastic cerebellar degeneration (PCD), paroxysmal nocturnal hemoglobinuria (PNH), Parry Romberg syndrome, Parsonnage-Turner syndrome, pars planitis (peripheral uveitis), pemphigus, pemphigus/pemphigoid, peripheral neuropathy, perivenous encephalomyelitis, pernicious anemia, Peyronie's Disease, POEMS syndrome (polyneuropathy, organomegaly, endocrinopathy, monoclonal gammopathy, skin changes), PMR (polymyalgia rheumatica), polyarteritis nodossa, polyarthritis, polymyalgia rheumatic, polymyositis, postmyocardial infarction syndrome, postpericardiotomy syndrome, primary biliary cirrhosis, primary sclerosing cholangitis, progesterone dermatitis, pseudogout, Pseudoxanthoma Elasticum (PXE), psoriatic arthritis, psoriasis, pure red cell aplasia (PRCA), pyoderma gangrenosum, Raynaud's, reactive arthritis, Reiter's (Reactive Arthritis), relapsing polychondritis, retroperitoneal fibrosis, rheumatic fever, rheumatoid Arthritis, RLD (Restless Leg Syndrome), RSD (Reflex Sympathetic Dystrophy), Sarcoidosis, Schmidt syndrome, scleritis, Sjögren's Syndrome, soft tissue disease, sperm and testicular autoimmunity, spinal stenosis, stiff person syndrome (SPS), Still's Disease, subacute bacterial endocarditis (SBE), Susac's syndrome, sympathetic ophthalmia (SO), Takayasu's arteritis, temporal arteritis/Giant cell arteritis, thrombocytopenic purpura (TTP), Tolosa-Hunt syndrome (THS), transverse myelitis, temporal arteritis, TMJ (Tempero-Mandibular Joint) problems, thyroiditis, Type I, II, & III autoimmune polyglandular syndromes, ulcerative colitis, undifferentiated connective tissue disease (UCTD), undifferentiated spondylarthropathy, uveitis, Wegener's Granulomatosis, vasculitis, vesiculobullous dermatosis, and vitiligo.

### EXAMPLES

The following examples are included to further describe some embodiments of the present disclosure, and should not be used to limit the scope of the disclosure.

### Example 1

### Synthesis of Linkers with Immune-Modulatory Agents

A protease cleavable linker is attached with an immune-modulatory agent. A linker attached to an immune-modulatory agent is formed to make a linker-immune modulatory compound construct. Subsequently, the construct is conjugated to an antibody.

A linker is linked with an antibody or antibody construct, in which the linker includes a protease cleavage site. The linker can be a PEGylated linker or a non-PEGylated linker. Subsequently, an immune-modulatory agent is conjugated to the linker linked with the antibody or antibody construct.

### Example 2

### General Process for Identifying Tumor Antigen Antigens (TAAs) and Proteases for Selective Local Delivery

A biological state is identified which characterizes targetable disease populations. A gene signature or signatures is next identified which can be used to segregate the targetable population from irrelevant populations based on the above biological state. This can be accomplished via either:
a. Identification and utilization of existing signatures from the literature, or
b. Derivation of signatures de novo via differential transcriptomic expression analysis by comparing gene expression levels in disease tissue samples which exhibit the desired phenotype against those which do not.
   Genes which are statistically significantly differentially expressed, as determined for example via a Kruskal-Wallis test between the two sample sets, are then grouped into sets of signatures based on further analysis of the annotated pathways in which those genes are expressed.

Once a gene signature has been identified, a tumor-associated antigen (TAA) is identified by first performing hierarchical clustering on the expression of the genes in the signature to partition disease samples into those which have high expression of the gene signature and those which have reduced expression of the signature. This identifies both potential disease indications as well as target populations within diseases suitable for intervention. To identify specific TAA candidates, the expression levels of surface-localized proteins within the disease-signature-high samples are then evaluated against normal tissues using again, for example, a Kruskal-Wallis test to generate a list of statistically significant surface-localized proteins preferentially expressed on disease samples. This list then serves as potential tumor-associated antigen targets.

To identify proteases for linker cleavage, a similar process to that outlined above is followed. In this case proteases are identified that are expressed in general in the disease tissues of interest, or (a more restricted set) those that are significantly upregulated over normal in disease tissues of interest.

Once the candidate sets of tumor antigen antigens and proteases have been identified, expression data sets are then queried again, either using bulk tissue expression data (e.g., TCGA) or single-cell expression data to identify those pairs of TAA and protease genes which show either correlated expression overall, or at least high co-expression in the disease tissues of interest.

To identify cell lines which can be used as reagents for validation of the identified TAA and protease, available public cancer cell line transcriptomic data bases are queried to sort and stack rank those cell lines which show the highest co-expression of the TAA gene and the protease. If no cell lines are found co-expressing both genes to a satisfactory level, the option to engineer one starting from a cell line which either expresses high levels of the TAA or high levels of the protease is a possibility, in this case introducing the gene for the missing component.

### Example 3

### Immune-Modulatory Conjugates

LRRC15 was identified through bioinformatics experiments as a desirable targeting antigen for combination with a conjugate for inhibiting TGFβR2 signaling for immuno-oncology and fibrosis. LRRC15 was identified by the following properties: a) high expression in cancer associated fibroblasts (CAF), b) association w/a TGFβ activated pathway signature for metastatic cancers, c) association w/FAP and ADAM12 expression in these cancers, and d) elevated expression in cancers compared to most normal tissues including tissues such as heart/heart valve, bone and skin known to be associated with toxicities upon TGFβ pathway inhibition.correlated expression overall, or at least high co-expression in the disease tissues of interest.

LRRC15-TGFβR2 inhibitor conjugates are suitable for treatment of subsets of metastatic pancreatic adenocarcinoma, metastatic colorectal adenocarcinoma, breast invasive carcinoma, squamous cell lung cancer, metastatic head & neck squamous cell carcinoma. Such conjugates are also suitable for treatment of fibrotic diseases of the liver, kidney, lung, and systemic scleroderma. LRRC15, FAP and ADAM12 are also elevated on activated myofibroblasts that promote fibrotic disease through TGFβ signaling.

The protease cleavable linkers of the conjugates include protease cleavage sites recognized by FAP or ADAM12. The FAP protease cleavage site can be optimized for cleavage by FAP endopeptidase activity starting w/SPRY2 derived “consensus” hexapeptide of SSGPVA. The ADAM12 protease cleavage site can be optimized for cleavage by ADAM12 endopeptidase starting with the decapeptide substrate FFLAQA(homoF)RSK. The linker is conjugated to interchain disulfide residues or to lysine residues. The TGFβR2 inhibitors can include degradative inhibitors, such as PROTACs. The antibody construct has an Fcnun domain.

MUC16 was identified through bioinformatics experiments as a desirable targeting antigen for combination with a conjugate for inhibiting the beta catenin pathway by inhibiting TNKS1/2 for immuno-oncology. The antibody construct can be an anti-MUC16 antibody. The Fc domain is an FcγR null domain. The protease cleavable linker includes a TMPRSS4 endopeptidase cleavage site. The cleavage site can be optimized for cleavage by TMPRSS4 endopeptidase activity starting w/ “consensus” octapeptide of IQSRGLFG. The linker-TNKS1/2 inhibitor construct is conjugated to the antibody construct at lysine residues.

CEACAM5 was identified through bioinformatics experiments as a desirable targeting antigen for combination with a conjugate for inhibiting the beta catenin pathway by inhibiting TNIK for immuno-oncology. The conjugate can be used in treating metastatic colorectal adenocarcinoma. The antibody construct is an anti-CEACAM5 antibody, and the Fc domain is an FcγR null. The immune-modulatory agent is a TNIK inhibitor.

### Example 4

### Determination of K_{d} Values

K_{d} is measured by a radiolabeled antigen binding assay (RIA) performed with an antibody construct of conjugate comprising the antigen binding domain of interest, and its antigen as described by the following assay.

Solution binding affinity of Fabs, antibody constructs, or conjugates comprising the antigen binding domain for the antigen of interest is measured by equilibrating the Fab, antibody construct, or conjugate with a minimal concentration of (¹²⁵I)-labeled antigen in the presence of a titration series of unlabeled antigen, then capturing bound antigen with an anti-Fab, anti-antibody construct, or anti-conjugate antibody-coated plate (see, e.g., Chen et al., J. Mol. Biol. 293:865-881 (1999)). To establish conditions for the assay, multi-well plates are coated overnight with 5 μg/mL of a capturing anti-Fab, anti-antibody construct, or anti-conjugate antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab, antibody construct, or conjugate of interest (e.g., consistent with assessment of the anti-VEGF antibody, Fab-12, in Presta et al., Cancer Res. 57:4593-4599 (1997)). The Fab, antibody construct, or conjugate of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant is added, and the plates are counted on a TOPCOUNT™ gamma counter (Packard) for ten minutes. Concentrations of each Fab, antibody construct, or conjugate that give less than or equal to 20% of maximal binding are chosen for use in competitive binding assays.

### Example 5

### Determination of K_{d} Values

K_{d} is measured using surface plasmon resonance assays using a BIACORE®-2000 or a BIACORE®-3000 (BIAcore, Inc., Piscataway, N.J.) at 25° C. with immobilized antigen CMS chips at ^{˜}10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NETS) according to the supplier's instructions. Antigen is diluted with 10 mM sodium acetate, pH 4.8, to 5 μg/mL C0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab, antibody construct, or conjugate (0.78 nM to 500 nM) are injected in PBS with 0.05% polysorbate 20 (TWEEN-20™) surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 106 M−1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form, antibody construct form, or conjugate form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Example 6

### Lysine-based Bioconjugation

The antibody is exchanged into an appropriate buffer, for example, phosphate, borate, PBS, histine, or Tris-Acetate at a concentration of about 2 mg/mL to about 10 mg/mL. An appropriate number of equivalents of the immune-modulatory agent-linker construct are added as a solution with stirring. Dependent on the physical properties of the immune-modulatory agent-linker construct, a co-solvent is introduced prior to the addition of the immune-modulatory agent-linker construct to facilitate solubility. The reaction is stirred at room temperature for 2 hours to about 12 hours depending on the observed reactivity. The progression of the reaction is monitored by LC-MS. Once the reaction is deemed complete, the remaining immune-modulatory agent-linker constructs are removed by applicable methods and the lysine-linked immune-modulatory conjugate is exchanged into the desired formulation buffer.

Lysine-linked immune-modulatory conjugates are synthesized starting with 10 mg of antibody (mAb) and 10 equivalents of linker-immune-modulatory agent (ATAC) using the conditions described in Scheme below (ADC=antibody immune-modulatory agent conjugate).

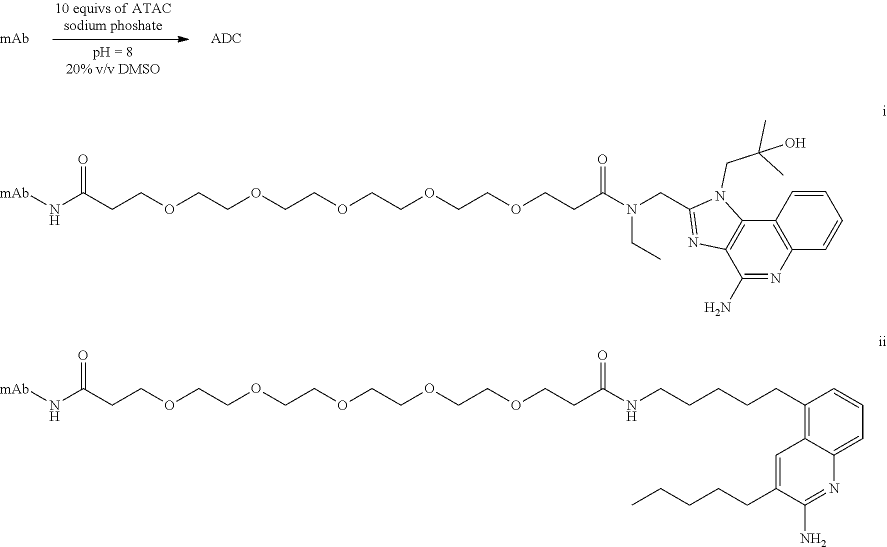

### Example 7

### Cysteine-Based Bioconjugation

The antibody is exchanged into an appropriate buffer, for example, phosphate, borate, PBS, histidine or Tris-Acetate at a concentration of about 2 mg/mL to about 10 mg/mL with an appropriate number of equivalents of a reducing agent, for example, dithiothreitol or tris(2-carboxyethyl)phosphine. The resultant solution is stirred for an appropriate amount of time and temperature to effect the desired reduction. The immune-modulatory agent-linker construct is added as a solution with stirring. Dependent on the physical properties of the immune-modulatory agent-linker construct, a co-solvent is introduced prior to the addition of the immune-modulatory agent-linker construct to facilitate solubility. The reaction is stirred at room temperature for about 1 hour to about 12 hours depending on the observed reactivity. The progression of the reaction is monitored by liquid chromatography-mass spectrometry (LC-MS). Once the reaction is deemed complete, the remaining free immune-modulatory agent-linker construct is removed by applicable methods and the conjugate is exchanged into the desired formulation buffer. Such cysteine-based conjugates are synthesized starting with 10 mg of antibody (mAb) and 7 equivalents of linker-immune-modulatory agent using the conditions described in Scheme below (ADC=antibody immune-modulatory agent conjugates). Monomer content and drug-antibody ratios can be determined by methods described in the EXAMPLES.

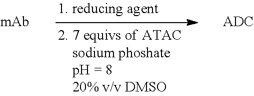

### Example 8

### Determination of Molar Ratio

This example illustrates one method by which the molar ratio is determined. One microgram of conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact conjugates is calculated by the software, which can use top peak height at 25% for the calculation. This data is then imported into another program to calculate the molar ratio of the immune-modulatory agent:conjugate, such as Agilent molar ratio calculator.

### Example 9

### Additional Method for Determination of Molar Ratio

Another method for determination of molar ratio is as follows. First, 10 μL of a 5 mg/mL solution of a conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 10

### Generation of a Bispecific Antibody Construct

A single chain Fv (scFv) against a target antigen in the VH-VL orientation is linked to the C-terminus of an antibody heavy chain (VH-CH1-CH2-CH3) against a second antigen to generate the anti-antigen 2 VH-CH1-CH2-CH3-anti-antigen 1 scFv heavy chain. This heavy chain is co-expressed with the light chain of anti-antigen 2 (VL-Ck) in the transient CHO system in 180 mL of CHO media. The supernatant is harvested 7 days after the transfection and the bispecific antibody construct is purified over HiScreen Mab Select SuRe Protein A column on GE AKTA Pure machine. The bispecific antibody construct is recovered.

### Example 11

### General Assay for Detection of Protease Cleavage Activity of Immune-Modulatory Conjugates

This example describes a cell-based assay for testing the activity of immune-modulatory conjugates, and in particular proteolytic release of the immune-modulatory agent in the presence of cells expressing a target antigen and associated protease.

A target antigen and associated protease are identified as described in Example 3. A conjugate is prepared that includes an antibody construct or an antibody that specifically binds to the target antigen (TA). The antibody has an Fcγ null Fc region. A linker is prepared that contain a protease cleavage site for the identified protease. A cell line expressing the target antigen and the protease (TA+ Endopeptidase+) on the cell surface, or in the culture medium in the case of the protease, is co-cultured with a bystander cell line that does not express the target antigen or the protease. The bystander cell line can be, for example, a payload target pathway reporter cell line, such as a HEK293 SMAD Luciferase reporter cell line. The immune-modulatory conjugate is added to the cultured cells. Release of the immune-modulatory agent from the conjugate is detected as a signal from the reporter cell line. Unconjugated antibody to the target antigen, a conjugate that does not bind to the target antigen, and unconjugated immune-modulatory agent are used as controls.

### Example 12

### The Anti-Tumor Response is Enhanced by Bispecific Tumor Targeting Conjugates

This example shows the anti-tumor immune response is enhanced by tumor targeting conjugates.

A human immune cell mediated anti-tumor response in a tumor xenograft model using immunocompromised mice is enhanced by bispecific tumor targeting conjugates with a TGFbeta inhibitor or a TNIK inhibitor. In immunocompromised mice, 1×10⁷ cells/animal human tumor cells from lines expressing LRRC15, FAP or MUC16 are co-injected subcutaneously into the flanks of mice from strain NSG (NOD.Cg-Prkd^{scid}IL-2^{tm1Wj1}/SzJ) along with donor matched human T cells (1×10⁶/animal) and mDCs (5×10⁵/animal). The incorporation of human mDCs in this model allows for the assessment of in vivo immune-mediated activity of the conjugates with the TGFbeta inhibitor or the TNIK inhibitor. Tumor targeting conjugates with the TGFbeta inhibitor or the TNIK inhibitor bearing one of the following antigen specificities, LRRC15, FAP or MUC16, are injected intraperitoneally after tumor injection and formation. The conjugate cleavable linker with a protease-cleavage site, wherein the protease is over-expressed and found in the microenvironment of the tumors as they form.

As a control, conjugates with a TGFbeta inhibitor or a TNIK inhibitor in which the antigen binding domain is not directed to antigen on the injected tumor is injected intraperitoneally after tumor injection and formation. Tumor growth is measured using calipers twice per week, beginning seven days post tumor cell transfer and ending at study termination, approximately 3 weeks after tumor cell inoculation. As is shown by the results, tumor growth is reduced only when the injected tumor cells express the antigen specificity of the tumor targeting conjugates with a TGFbeta inhibitor or a TNIK inhibitor.

### Example 13

### Treatment of LRRC15 Expressing Cancer by Administering a Bispecific Tumor Targeting Conjugate

This example describes treatment of a LRRC15 expressing cancer with a tumor targeting conjugate. A human patient is diagnosed with a cancer that expresses the tumor-associated antigen LRRC15, such as metastatic pancreatic adenocarcinoma, metastatic colorectal adenocarcinoma, breast invasive carcinoma, squamous cell lung cancer, or metastatic head and neck squamous cell carcinoma. A LRRC15 tumor targeting conjugate a TGFbeta inhibitor, which specifically binds the tumor-associated antigen LRRC15, is administered to the patient. The conjugate comprises a protease cleavable linker having a protease cleavage site cleavable by ADAM12 or FAP. An anti-tumor response is induced against tumor cells expressing LRRC15, such as a T cell-mediated immune response and an innate immune response, leading to control and eradication of tumor cells.

### Example 14

### Stability of Bispecific Tumor Targeting Conjugate Containing an Immune-Modulatory Agent in IgG Depleted Human Serum

This example shows the stability of an immune-modulatory conjugate in IgG depleted human serum. Stability of the conjugate in human serum (IgG depleted) is measured over 96 hours at 37° C. using either a direct HIC-UV analysis approach (Method A) or an affinity capture approach (Method B). Conjugates are spiked in IgG-depleted human serum (BBI solutions #SF142-2) in sterile tubes (75% final serum concentration) and samples are split into 4 aliquots of equal size then transferred to a 37° C. incubator. One of the aliquots of each sample is taken from the incubator at each time-point (T=0h, 24h, 48h, 96h) and the stability of the conjugate, or the average immune-modulatory agent:antibody construct ratios (DAR), is recorded.

### Method A: Direct HIC-UV Analysis

At 0, 24, 48, and 96 hours after the beginning of incubation, immune-modulatory conjugate spiked in IgG depleted human serum is analyzed by analytical hydrophobic interaction chromatography (HIC) using a TOSOH TSKgel Butyl-NPR 4.6 mm×35 mm HIC column (TOSOH Bioscience, #14947) connected to a Dionex Ultimate 300016 HPLC system (ThermoFisher Scientific, Hemel Hemstead, UK). The stability of the conjugate is assessed and is found to remain stable at each time point. The average DAR is found to remain stable at each timepoint.

### Method B: Affinity Capture, De-Glycosylation and RP-ESI-MS Analysis

An immune-modulatory conjugate is immunocaptured from the IgG depleted human serum using an anti-Human IgG (Fc specific) biotin antibody immobilized on streptavidin beads at 0, 24, 48 and 96 hours after the beginning of incubation. After elution from the beads, the samples are de-glycosylated using agarose-immobilized EndoS (Genovis Inc, USA). The de-glycosylated immune-modulatory conjugate is analyzed by reverse phase chromatography hyphenated to electrospray ionization mass spectrometry (RP-ESI-MS) using an Acquity nano UPLC in line with a Xevo G2S Q-TOF (Waters, Elstree, UK). The separation is performed using an Acquity UPLC online coupled to an ESI-MS mass spectrometer. Mass spectrometric analysis is performed in positive ion mode, scanning from 1000 to 4000 m/z in high mass operating mode. The ion envelope produced by each sample is deconvoluted using the MaxEntl algorithm provided within the MassLynx software (Waters, Elstree, UK). The stability of the conjugate is assessed and is found to remain stable at each time point. The average DAR is found to remain stable at each timepoint.

### Example 15

### Activity of a TLR8 Agonist Conjugate on Monocytes and Macrophages is Mediated by FcγR Interaction

This example illustrates that the activity of a TLR8 benzazepine agonist on monocytes and macrophages is mediated by Fey receptor interactions.

Monocyte-Tumor Cell Co-Culture Assay. Human monocytes were isolated from fresh PBMCs by magnetic bead enrichment and plated in 96-well flat bottom microtiter plates (40,000/well). HER2-expressing tumor cells were then added (40,000/well) along with titrating concentrations of antibody conjugates with differing abilities to engage Fc receptors. After overnight culture, supernatants were harvested, and TNFα levels were determined by AlphaLISA.

Macrophage-Tumor Cell Co-Culture Assay. Human monocytes were isolated from fresh PBMCs by magnetic bead enrichment and differentiated into macrophages by culturing in the presence of M-CSF for 7 days. After differentiation, the macrophages were harvested and plated in 96-well flat bottom microtiter plates (40,000/well). HER2-expressing tumor cells were then added (40,000/well) along with titrating concentrations of antibody-drug conjugates with differing abilities to engage Fc receptors. After overnight culture, supernatants were harvested, and TNFα levels were determined by AlphaLISA.

Her2 FcγR null mutant antibody was prepared by making the following amino acid substitutions in a human IgG1 Fc domain: L234A, L235A, G237A, and K322A. Her2 FcRn null mutant antibody was prepared by making a H435A substitution in an IgG1 Fc domain. The double Her2 FcγR FcRn null mutant was prepared by making both set of amino acid substitutions in an IgG1 Fc domain of pertuzumab. Conjugates were prepared by conjugation of the monoclonal antibodies to a TLR8 benzazepine agonist (2-amino-N⁸-(5-((2-amino-3-phenylpropanamido)methyl)pyridin-3-yl)-N⁴,N⁴-dipropyl-3H-benzo[b]azepine-4, 8-dicarboxamide) and an mc-val-cit-PABC linker, as generally described in Example 7. The average drug loading was about 4.

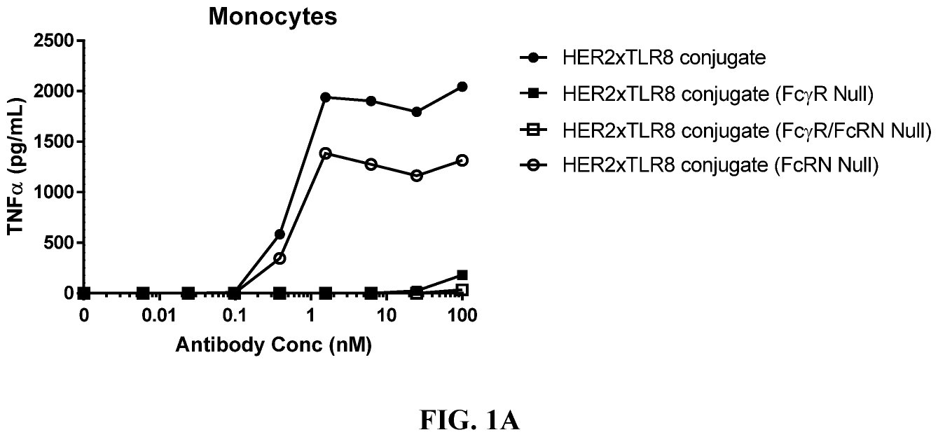

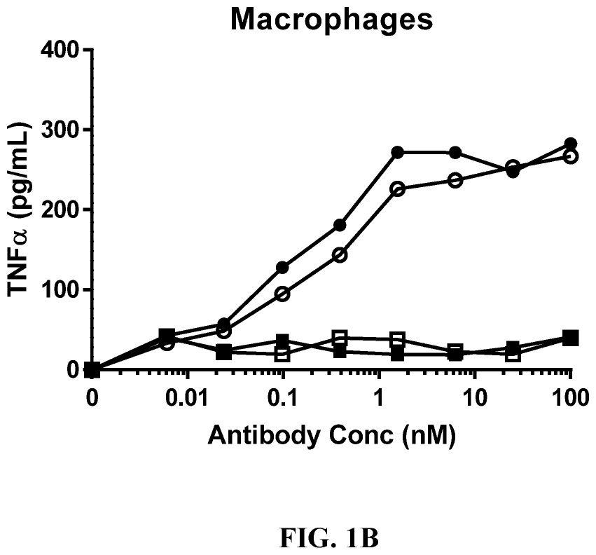

Referring to FIG. 1A, stimulation of TNFα production by monocytes in response to a TLR8 benzazepine agonist was dependent on FcγR interaction with a functional antibody Fc domain. Her2 conjugate with a wildtype IgG1 Fc region stimulated TNFα production by monocytes. Mutant Her2 conjugate having an FcRn null mutation was also able to stimulate TNFα production, but to a lesser extent. In contrast, Her2 antibody conjugate having an FcγR null mutation stimulated minimal TNFα production.

Referring to FIG. 1B, similar results were obtained with macrophages. A functional FcγR-Fc binding domain interaction was needed to stimulate TNFα production by macrophages.

### Example 16

### Activity of a TLR8 Benzazepine Agonist Conjugate on Macrophaes can be Modulated by Modifying the Interaction of an Antibody Fc Fomain with Various Fcγ Receptors on Macrophages

This example illustrates that the activity of a TLR8 benzazepine agonist conjugate on macrophages is modulated by interaction with certain Fcγ receptors.

Macrophage-Tumor Cell Co-Culture Assay: Human monocytes were isolated from fresh PBMCs by magnetic bead enrichment and differentiated into macrophages by culturing in the presence of M-CSF for 7 days. After differentiation, macrophages were harvested and plated in 96-well flat bottom microtiter plates (40,000/well). HER2-expressing tumor cells were added (40,000/well) along with FcγR blocking Abs and incubated for 1 hr prior to adding titrating concentrations of Her2 antibody conjugate or an unconjugated control antibody. After overnight culture, supernatants were harvested, and TNFα levels were determined by AlphaLISA.

Her2x TLR8 conjugate was prepared by conjugation of a TLR8 benzazepine agonist to Her2 monoclonal antibody having a wildtype IgG1 Fc region, as described in Example 15.

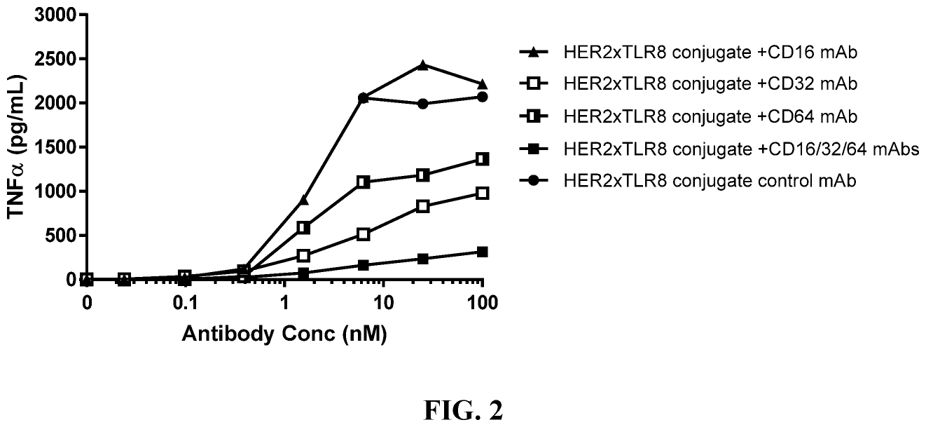

Referring to FIG. 2, Her2 antibody conjugate was able to stimulate TNFα production in the presence of control antibody and in the presence of anti-CD16 antibody (FcγRIII). Blocking the interaction of the antibody Fc region minimally affected TNFα production. In contrast, addition of anti-CD64 (FcγR1) antibody (FcγR1) or anti-CD32 (FcγRII) antibody decreased TNFα production, with anti-CD32 antibody having the greater inhibition. Addition of all three anti-FcγR antibodies further diminished TNFα production.

These results indicate that the activity of a TLR8 benzazepine agonist conjugate on macrophages can be modulated by modifying the interaction of an antibody Fc domain with various Fcγ receptors on macrophages.

### Example 17

### Activation of Dendritic Cells by TLR8 Agonist Antibody Conjugate can be Modulated by Modifying the Interaction of an Antibody Fc Fomain with Various Fcγ Receptors

This example illustrates that the activity of a TLR8 benzazepine agonist on dendritic cells can be modulated by Fcγ receptor interaction.

mDC Fc Blocking Stimulation Method: PBMC were isolated from normal human leukapheresis product using Ficoll purification, and monocytes were enriched from PBMC using Stem Cell Technologies Human Monocyte without CD16 Depletion Negative Selection Kits according to the manufacturer's instructions. Monocytes were frozen and stored in liquid nitrogen. Seven days prior to assay, monocytes were thawed and differentiated into monocyte derived dendritic cells (mDC). For differentiation, monocytes were plated at a concentration of 1×10⁶ monocytes/mL in X-Vivo15 media supplemented with 10% fetal bovine serum (FBS), 100 ng/mL recombinant human GM-CSF and 10 ng/mL recombinant human IL-4 and incubated at 37° C. in 5% CO₂. On day 4 of differentiation, half of the media was removed and replaced with fresh X-Vivo15 media supplemented with 10% fetal bovine serum (FBS), 100 ng/mL recombinant human GM-CSF and 10 ng/mL recombinant human IL-4 and returned to 37° C. in 5% CO₂.

mDC were lifted and re-plated on day 7 of differentiation at 4×10⁴ cell/well with 4×10⁴ cell/well BT474 Her2 positive tumor cells in 96-well flat bottom plates in assay media (RPMI 1640 base media supplemented with 10% FBS, 50 μg/mL Penicillin, 50 U/mL Streptomycin, 1 mM HEPES, 1× non-essential amino acids, 0.1 mM sodium pyruvate). Mouse monoclonal blocking antibodies to human CD16, CD32, CD64 or mouse isotype control were added to co-cultures at 10 μg/mL and pre-incubated at 37° C. in 5% CO₂for 30 minutes. Antibody-drug conjugate was diluted in assay media and then added to co-cultures at final concentrations starting at 100 nM and diluted 4-fold to 0.024 nM in 100 μL/well total volume. Co-cultures were incubated for 24 hours at 37° C. in 5% CO₂ before cell culture supernatants were harvested. IL-12p40 production was assessed in culture supernatants using the Perkin Elmer Human IL-12/IL-23 p40 AlphaLISA Kit according to the manufacturer's instructions. Plates were read using a Perkin Elmer Envision plate reader.

Her2×TLR8 conjugate was prepared by conjugation of TLR8 benzazepine agonist and a linker to Her2 monoclonal antibody having a wildtype IgG1 Fc region, as generally described in Example 15.

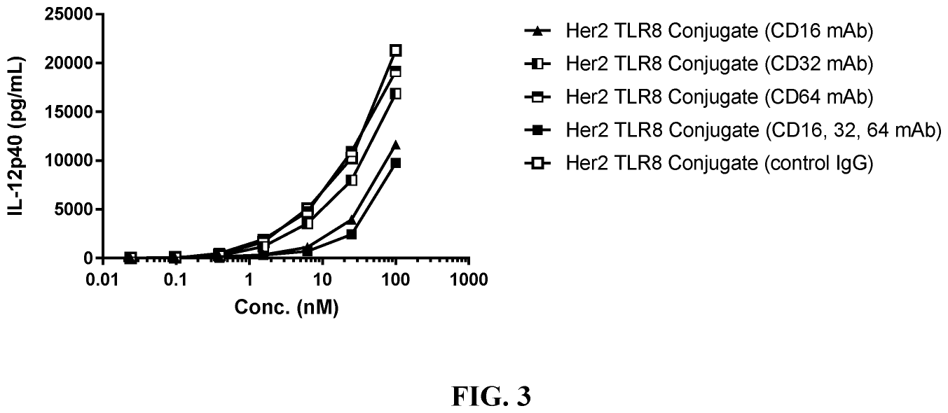

Referring to FIG. 3, Her2 antibody conjugate was able to stimulate IL-12 production in the presence of control antibody and in the presence of anti-CD32 antibody (FcγRII) and anti-CD64 (FcγRI) antibody. Addition of anti-CD16 (FcγRIII) antibody or all three anti-FcγR antibodies reduced IL-12 production, but did not eliminate IL-12 production by DCs.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. An immune-modulatory conjugate, comprising:
(a) an antibody construct comprising an antigen binding domain and an Fc domain, wherein the antigen binding domain specifically binds to a first antigen expressed on target cells associated with a disease;
(b) at least one immune-modulatory agent, wherein the immune-modulatory agent is:
(1) a TLR8 agonist compound represented by the structure of Formula (IIIA): 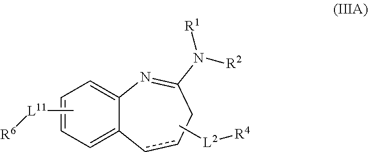
or a pharmaceutically acceptable salt thereof, wherein: [symbol]
represents an optional double bond;
L¹¹ is —C(O)N(R¹⁰)—*, wherein * represents where L¹¹ is bound to R⁶;
L² is —C(O)—;
R¹ and R² are each hydrogen;
R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰;
R⁶ is selected from phenyl and 5- or 6-membered heteroaryl, any one of which is substituted with one or more substituents selected from R⁷;
R⁷ is selected from —C(O)NHNH₂, —C(O)NH—C₁₋₃alkylene-NH(R¹⁰), —C(O)CH₃, —C₁₋₃alkylene-NHC(O)OR¹⁰, —C₁₋₃alkylene-NHC(O)R¹⁰, —C₁₋₃alkylene-NHC(O)NHR¹⁰, and —C₁₋₃alkylene-NHC(O)—C₁₋₃alkylene-(R¹⁰)₂;
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is independently optionally substituted at each occurrence with one or more substituents selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is selected from C₃₋₁₂ carbocycle and 3- to 12-membered heterocycle, each of which is independently optionally substituted with one or more substituents selected from R¹²; and
wherein any substitutable carbon on the benzazepine core is optionally substituted by a substituent independently selected from R¹², wherein R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R, —NO₂, ═O, ═S, ═N(R¹⁰), and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —NO₂, ═O, ═S, ═N(R¹⁰), —CN, C₃₋₁₀ carbocycle and 3- to 10-membered heterocycle; or
(2) a TLR7 agonist selected from the group consisting of an imidazoquinoline, an imidazoquinoline amine, a thiazoquinoline, an aminoquinoline, an aminoquinazoline, a pyrido[3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, and a benzonaphthyridine; or
(3) an inhibitor of a GCPR, an ion channel, a membrane transporter, a phosphatase, an ER protein, TGFβ, a TGFβ signaling pathway, or β-Catenin pathway; and
(c) at least one linker, wherein each linker is covalently attached to the antibody construct and to at least one immune-modulatory agent,
wherein in the conjugate:
(i) the linker is a cleavable linker having a protease cleavage site cleavable by a protease that is preferentially localized in the extracellular microenvironment of the target cells, whereby cleavage of the protease cleavage site releases an active form of the immune-modulatory agent in the extracellular microenvironment; or
(ii) the Fc domain comprises an amino acid sequence having a Kd for a first Fc receptor that is at least 10 fold higher than a Kd of the amino acid sequence for a wild-type IgG1 Fc domain for the first Fc receptor and having a Kd for a second Fc receptor that is the same or lower than a Kd of the amino acid sequence for a wild-type IgG1 Fc domain for the second Fc receptor, thereby allowing the Fc domain to preferentially bind to cells expressing the second Fc receptor; or
(iii) the linker is as set forth in (i) and the Fc domain is as set forth in (ii).

2. The immune-modulatory conjugate of claim 1, wherein the conjugate is represented by the following formula: 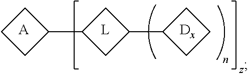
wherein:
A is the antibody construct having the antigen binding domain and the Fc domain;
L is the linker;
Dₓ is the immune-modulatory agent;
n is selected from 1 to 20; and
z is selected from 1 to 20.

3. The immune-modulatory conjugate of claim 2, wherein n is from 1 to 5 and z is from 1 to 8, n is 1 and z is from 1 to 8, or n is 1 and z is from 1 to 5.

4. The immune-modulatory conjugate of claim 1, wherein the conjugate is as set forth in (iii).

5. The immune-modulatory conjugate of claim 2, wherein the linker, L, has the following formula:
Rx-Aₙ-protease cleavage site-Y_{y}—,
wherein Rx is a reactive moiety attached to the antibody construct;
A is a stretcher group and n is 0 or 1; and
Y is a self immolative group and y is 0, 1, or 2.

6. The immune-modulatory conjugate of claim 5, wherein Rx is a succinimide group or a hydrolyzed succinmide group.

7. (canceled)

8. The immune-modulatory conjugate of claim 5, wherein the self-immolative group is a PABA or PABC group.

9. The immune-modulatory conjugate of claim 5, wherein the linker is -maleimidocaproyl-protease cleave site-PABC-.

10.-13. (canceled)

14. The immune-modulatory conjugate of claim 1, wherein the protease is preferentially localized in the extracellular microenvironment of the target cells associated with a disease as compared to the extracellular microenvironment of normal cells by a factor of at least 5:1, 10:1, 25:1, 50:1 or 100:1.

15. (canceled)

16. The immune-modulatory conjugate of claim 1, wherein the linker is not a cleavable linker as set forth in (i) of claim 1.

17.-19. (canceled)

20. The immune-modulatory conjugate of claim 1, wherein the first Fc receptor is an FcγRI, FcγRIIA and FcγRIIIA and the second Fc receptor is an FcRn receptor.

21. The immune-modulatory conjugate of claim 20, wherein the Kd of the Fc domain for the FcRn is at least 5-fold lower than the binding of a wild-type IgG1 to FcRn.

22.-25. (canceled)

26. The immune-modulatory conjugate of claim 1, wherein the first antigen is selected from the group consisting of GD2, GD3, GM2, Le^{y}, sLe, polysialic acid, fucosyl GM1, Tn, STn, BM3, GloboH, CD5, CD19, CD20, CD25, CD37, CD30, CD33, CD45, CAMPATH-1, BCMA, CS-1, PD-L1, B7-H3, B7-DC (PD-L2), HLA-DR, carcinoembryonic antigen (CEA), TAG-72, MUC1, MUC15, MUC16, folate-binding protein, A33, G250, prostate-specific membrane antigen (PSMA), STN1, TNC, CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WT1, LMP2, HPV E6, HPV E7, p53 nonmutant, NY-ESO-1, GLP-3, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, MYCN, RhoC, TRP-2, mesothelin (MSLN), PSCA, MAGE A1, MAGE-A3, CYP1B1, PLAV1, BORIS, Tn, ETV6-AML, NY-BR-1, RGS5, SART3, Carbonic anhydrase IX, PAX5, OY-TES1, Sperm protein 17, LCK, MAGE C2, MAGE A4, GAGE, TRAIL1, HMWMAA, AKAP-4, SSX2, XAGE 1, B7H3, Legumain, Tie 3, PAGE4, VEGFR2, MAD-CT-1, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-1B (UPK1B), LIV1, ROR1, STRA6, TMPRSS3, TMPRSS4, TMEM238, Clorf186, Fos-related antigen 1, VEGFR1, endoglin, VTCN1 (B7-H4), and VISTA.

27.-28. (canceled)

29. The immune-modulatory conjugate of claim 1, wherein the first antigen is selected from the group consisting of Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, MMP14, GPX8, and F2RL2.

30.-58. (canceled)

59. The immune-modulatory conjugate of claim 1, wherein the antigen binding domain comprises a variable region comprising V_{H} and V_{L} sequences at least 90% sequence identity to a pair of V_{H} and V_{L} sequences set forth in TABLE 2 or TABLE 6.

60. (canceled)

61. A pharmaceutical composition comprising the immune-modulatory conjugate of claim 1 and a pharmaceutically acceptable carrier.

62. A method of treating a subject having cancer or a fibrotic or inflammatory disease of the liver, kidney or lung, or systemic scleroderma, or idiopathic pulmonary fibrosis (IPF), or NASH, or cardiomyopathy, comprising administering a therapeutic dose of the pharmaceutical composition of claim 61.

63. (canceled)

64. The method of claim 62, wherein the conjugate is administered intravenously or subcutaneously.

65.-68. (canceled)
